SEQUENCE LISTING (55 sequences)

SEQ ID NO: 1 - DNA, 825 bp, synthetic construct
  misc_feature 1..825: note = Description of Artificial Sequence: Synthetic polynucleotide
  source 1..825: mol_type = other DNA
atgaaaaagatttggctggcgctggctggtttagttttagcgtttagcgcatcggcggcgcagtatgaagatcaccatcaccaccaccaccatcaccactctggctcgagcctggtgccgcgcggcagccatatgggtccgcagggtgttgttggtgcagatggtaaagacggtaccccgggtgaaaaaggagaacagggacgtacaggtgcagcaggtaaacagggcagcccgggtgccgatggtgcccgtggcccgctgggtagcattggtcagcagggtgcaagaggcgaaccgggcgatccgggtagtccgggcctgcgtggtgatacgggtctggccggtgttaaaggcgttgcaggtccttcaggtcgtccaggtcaaccgggtgcaaatggtctgccgggtgttaatggtcgtggcggtctggaacgtggtctggcaggaccgccgggtcctgatggtcgccgcggtgaaacgggttcaccgggtattgccggtgccctgggtaaaccaggtctggaaggtccgaaaggttatcctggtctgcgcggtcgtgatggtaccaatggcaaacgtggcgaacagggcgaaaccggtccagatggtgttcgtggtattccgggtaacgatggtcagagcggtaaaccgggcattgatggtattgatggcaccaatggtcagcctggcgaagcaggttatcagggtggtcgcggtacccgtggtcagctgggtgaaacaggtgatgttggtcagaatggtgatcgcggcgcaccgggtccggatggtagcaaaggtagcgccggtcgtccgggtttacgttaa

SEQ ID NO: 2 - protein, 274 aa, synthetic construct
  REGION 1..274: note = Description of Artificial Sequence: Synthetic polypeptide
MKKIWLALAGLVLAFSASAAQYEDHHHHHHHHHSGSSLVPRGSHMGPQGVVGADGKDGTPGEKGEQGRTGAAGKQGSPGADGARGPLGSIGQQGARGEPGDPGSPGLRGDTGLAGVKGVAGPSGRPGQPGANGLPGVNGRGGLERGLAGPPGPDGRRGETGSPGIAGALGKPGLEGPKGYPGLRGRDGTNGKRGEQGETGPDGVRGIPGNDGQSGKPGIDGIDGTNGQPGEAGYQGGRGTRGQLGETGDVGQNGDRGAPGPDGSKGSAGRPGLR

SEQ ID NO: 3 - DNA, 689 bp, synthetic construct
  misc_feature 1..689: note = Description of Artificial Sequence: Synthetic polynucleotide
  source 1..689: mol_type = other DNA
gtccgcagggtgttgttggtgcagatggtaaagacggtaccccgggtgaaaaaggagaacagggacgtacaggtgcagcaggtaaacagggcagcccgggtgccgatggtgcccgtggcccgctgggtagcattggtcagcagggtgcaagaggcgaaccgggcgatccgggtagtccgggcctgcgtggtgatacgggtctggccggtgttaaaggcgttgcaggtccttcaggtcgtccaggtcaaccgggtgcaaatggtctgccgggtgttaatggtcgtggcggtctggaacgtggtctggcaggaccgccgggtcctgatggtcgccgcggtgaaacgggttcaccgggtattgccggtgccctgggtaaaccaggtctggaaggtccgaaaggttatcctggtctgcgcggtcgtgatggtaccaatggcaaacgtggcgaacagggcgaaaccggtccagatggtgttcgtggtattccgggtaacgatggtcagagcggtaaaccgggcattgatggtattgatggcaccaatggtcagcctggcgaagcaggttatcagggtggtcgcggtacccgtggtcagctgggtgaaacaggtgatgttggtcagaatggtgatcgcggcgcaccgggtccggatggtagcaaaggtagcgccggtcgtccgggtttacgttaa

SEQ ID NO: 4 - protein, 229 aa, synthetic construct
  REGION 1..229: note = Description of Artificial Sequence: Synthetic polypeptide
GPQGVVGADGKDGTPGEKGEQGRTGAAGKQGSPGADGARGPLGSIGQQGARGEPGDPGSPGLRGDTGLAGVKGVAGPSGRPGQPGANGLPGVNGRGGLERGLAGPPGPDGRRGETGSPGIAGALGKPGLEGPKGYPGLRGRDGTNGKRGEQGETGPDGVRGIPGNDGQSGKPGIDGIDGTNGQPGEAGYQGGRGTRGQLGETGDVGQNGDRGAPGPDGSKGSAGRPGLR

SEQ ID NO: 5 - protein, 189 aa, synthetic construct
  REGION 1..189: note = Description of Artificial Sequence: Synthetic polypeptide
GPQGVVGADGKDGTPGNAGQKGPSGEPGSPGKAGSAGEQGPPGKDGSNGEPGSPGKEGERGLAGPPGPDGRRGETGSPGIAGALGKPGLEGPKGYPGLRGRDGTNGKRGEQGETGPDGVRGIPGNDGQSGKPGIDGIDGTNGQPGEAGYQGGRGTRGQLGETGDVGQNGDRGAPGPDGSKGSAGRPGLR

SEQ ID NO: 6 - DNA, 642 bp, synthetic construct
  misc_feature 1..642: note = Description of Artificial Sequence: Synthetic polynucleotide
  source 1..642: mol_type = other DNA
atgaaaaagatttggctggcgctggctggtttagttttagcgtttagcgcatcggcggcgcagtatgaagatggtccgcagggtgttgttggtgcagatggtaaagacggtaccccgggtaatgcaggtcagaaaggtccgtcaggtgaacctggcagccctggtaaagcaggtagtgccggtgagcagggtccgccgggcaaagatggtagtaatggtgagccgggtagccctggcaaagaaggtgaacgtggtctggcaggaccgccgggtcctgatggtcgccgcggtgaaacgggttcaccgggtattgccggtgccctgggtaaaccaggtctggaaggtccgaaaggttatcctggtctgcgcggtcgtgatggtaccaatggcaaacgtggcgaacagggcgaaaccggtccagatggtgttcgtggtattccgggtaacgatggtcagagcggtaaaccgggcattgatggtattgatggcaccaatggtcagcctggcgaagcaggttatcagggtggtcgcggtacccgtggtcagctgggtgaaacaggtgatgttggtcagaatggtgatcgcggcgcaccgggtccggatggtagcaaaggtagcgccggtcgtccgggtttacgttaa

SEQ ID NO: 7 - protein, 213 aa, synthetic construct
  REGION 1..213: note = Description of Artificial Sequence: Synthetic polypeptide
MKKIWLALAGLVLAFSASAAQYEDGPQGVVGADGKDGTPGNAGQKGPSGEPGSPGKAGSAGEQGPPGKDGSNGEPGSPGKEGERGLAGPPGPDGRRGETGSPGIAGALGKPGLEGPKGYPGLRGRDGTNGKRGEQGETGPDGVRGIPGNDGQSGKPGIDGIDGTNGQPGEAGYQGGRGTRGQLGETGDVGQNGDRGAPGPDGSKGSAGRPGLR

SEQ ID NO: 8 - DNA, 570 bp, synthetic construct
  misc_feature 1..570: note = Description of Artificial Sequence: Synthetic polynucleotide
  source 1..570: mol_type = other DNA
ggtccgcagggtgttgttggtgcagatggtaaagacggtaccccgggtaatgcaggtcagaaaggtccgtcaggtgaacctggcagccctggtaaagcaggtagtgccggtgagcagggtccgccgggcaaagatggtagtaatggtgagccgggtagccctggcaaagaaggtgaacgtggtctggcaggaccgccgggtcctgatggtcgccgcggtgaaacgggttcaccgggtattgccggtgccctgggtaaaccaggtctggaaggtccgaaaggttatcctggtctgcgcggtcgtgatggtaccaatggcaaacgtggcgaacagggcgaaaccggtccagatggtgttcgtggtattccgggtaacgatggtcagagcggtaaaccgggcattgatggtattgatggcaccaatggtcagcctggcgaagcaggttatcagggtggtcgcggtacccgtggtcagctgggtgaaacaggtgatgttggtcagaatggtgatcgcggcgcaccgggtccggatggtagcaaaggtagcgccggtcgtccgggtttacgttaa

SEQ ID NO: 9 - DNA, 2232 bp, synthetic construct
  misc_feature 1..2232: note = Description of Artificial Sequence: Synthetic polynucleotide
  source 1..2232: mol_type = other DNA
atgaaaaagatttggctggcgctggctggtttagttttagcgtttagcgcatcggcggcgcagtatgaagatcaccatcaccaccaccaccatcaccactctggctcgagcctggtgccgcgcggcagccatatgtctggctcgagcagtaaaggtgaagaactgttcaccggtgttgttccgatcctggttgaactggatggtgatgttaacggccacaaattctctgttcgtggtgaaggtgaaggtgatgcaaccaacggtaaactgaccctgaaattcatctgcactaccggtaaactgccggttccatggccgactctggtgactaccctgacctatggtgttcagtgtttttctcgttacccggatcacatgaagcagcatgatttcttcaaatctgcaatgccggaaggttatgtacaggagcgcaccatttctttcaaagacgatggcacctacaaaacccgtgcagaggttaaatttgaaggtgatactctggtgaaccgtattgaactgaaaggcattgatttcaaagaggacggcaacatcctgggccacaaactggaatataacttcaactcccataacgtttacatcaccgcagacaaacagaagaacggtatcaaagctaacttcaaaattcgccataacgttgaagacggtagcgtacagctggcggaccactaccagcagaacactccgatcggtgatggtccggttctgctgccggataaccactacctgtccacccagtctaaactgtccaaagacccgaacgaaaagcgcgaccacatggtgctgctggagttcgttactgcagcaggtatcacgcacggcatggatgaactctacaaatctggcgcgccgggcggtccgcagggtgttgttggtgcagatggtaaagacggtaccccgggtaatgcaggtcagaaaggtccgtcaggtgaacctggcagccctggtaaagcaggtagtgccggtgagcagggtccgccgggcaaagatggtagtaatggtgagccgggtagccctggcaaagaaggtgaacgtggtctggcaggaccgccgggtcctgatggtcgccgcggtgaaacgggttcaccgggtattgccggtgccctgggtaaaccaggtctggaaggtccgaaaggttatcctggtctgcgcggtcgtgatggtaccaatggcaaacgtggcgaacagggcgaaaccggtccagatggtgttcgtggtattccgggtaacgatggtcagagcggtaaaccgggcattgatggtattgatggcaccaatggtcagcctggcgaagcaggttatcagggtggtcgcggtacccgtggtcagctgggtgaaacaggtgatgttggtcagaatggtgatcgcggcgcaccgggtccggatggtagcaaaggtagcgccggtcgtccgggtttacgtcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaa

SEQ ID NO: 10 - protein, 743 aa, synthetic construct
  REGION 1..743: note = Description of Artificial Sequence: Synthetic polypeptide
MKKIWLALAGLVLAFSASAAQYEDHHHHHHHHHSGSSLVPRGSHMSGSSSKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSKLSKDPNEKRDHMVLLEFVTAAGITHGMDELYKSGAPGGPQGVVGADGKDGTPGNAGQKGPSGEPGSPGKAGSAGEQGPPGKDGSNGEPGSPGKEGERGLAGPPGPDGRRGETGSPGIAGALGKPGLEGPKGYPGLRGRDGTNGKRGEQGETGPDGVRGIPGNDGQSGKPGIDGIDGTNGQPGEAGYQGGRGTRGQLGETGDVGQNGDRGAPGPDGSKGSAGRPGLRHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHW

SEQ ID NO: 11 - DNA, 2232 bp, synthetic construct; identical to SEQ ID NO: 9
  misc_feature 1..2232: note = Description of Artificial Sequence: Synthetic polynucleotide
  source 1..2232: mol_type = other DNA

SEQ ID NO: 12 - protein, 743 aa, synthetic construct; identical to SEQ ID NO: 10
  REGION 1..743: note = Description of Artificial Sequence: Synthetic polypeptide

SEQ ID NO: 13 - DNA, 636 bp, synthetic construct
  misc_feature 1..636: note = Description of Artificial Sequence: Synthetic polynucleotide
  source 1..636: mol_type = other DNA
atgaaaaagatttggctggcgctggctggtttagttttagcgtttagcgcatcggcggcgcagtatgaagatgcaggttttccgggtctgcctggtccggcaggcgaaccgggtcgtcatggtaaagatggtctgatgggtagtccgggttttaaaggtgaagcaggttcaccgggtgcacctggtcaggatggcacccgtggtgaaccgggtattccgggatttccgggtaatcgtggcctgatgggtcagaaaggtgaaattggtccgcctggtcagcagggtaaaaaaggcgcaccgggtatgccaggactgatgggttcaaatggcagtccgggtcagccaggcacaccgggttcaaaaggtagcaaaggcgaacctggtattcagggtatgcctggtgcaagcggtctgaaaggcgagccaggtgccaccggttctccgggtgaaccaggttatatgggtctgccaggtatccaaggcaaaaaaggtgataaaggtaatcagggcgaaaaaggcattcagggccagaaaggcgaaaatggccgtcagggtattccaggccagcagggcatccagggtcatcatggtgcaaaaggtgaacgtggtgaaaagggcgaaccaggtgttcgttta

SEQ ID NO: 14 - protein, 211 aa, synthetic construct
  REGION 1..211: note = Description of Artificial Sequence: Synthetic polypeptide
MKKIWLALAGLVLAFSASAAQYEDAGFPGLPGPAGEPGRHGKDGLMGSPGFKGEAGSPGAPGQDGTRGEPGIPGFPGNRGLMGQKGEIGPPGQQGKKGAPGMPGLMGSNGSPGQPGTPGSKGSKGEPGIQGMPGASGLKGEPGATGSPGEPGYMGLPGIQGKKGDKGNQGEKGIQGQKGENGRQGIPGQQGIQGHHGAKGERGEKGEPGVR

SEQ ID NO: 15 - DNA, 565 bp, synthetic construct
  misc_feature 1..565: note = Description of Artificial Sequence: Synthetic polynucleotide
  source 1..565: mol_type = other DNA
tgcaggttttccgggtctgcctggtccggcaggcgaaccgggtcgtcatggtaaagatggtctgatgggtagtccgggttttaaaggtgaagcaggttcaccgggtgcacctggtcaggatggcacccgtggtgaaccgggtattccgggatttccgggtaatcgtggcctgatgggtcagaaaggtgaaattggtccgcctggtcagcagggtaaaaaaggcgcaccgggtatgccaggactgatgggttcaaatggcagtccgggtcagccaggcacaccgggttcaaaaggtagcaaaggcgaacctggtattcagggtatgcctggtgcaagcggtctgaaaggcgagccaggtgccaccggttctccgggtgaaccaggttatatgggtctgccaggtatccaaggcaaaaaaggtgataaaggtaatcagggcgaaaaaggcattcagggccagaaaggcgaaaatggccgtcagggtattccaggccagcagggcatccagggtcatcatggtgcaaaaggtgaacgtggtgaaaagggcgaaccaggtgttcgttaa

SEQ ID NO: 16 - protein, 187 aa, synthetic construct
  REGION 1..187: note = Description of Artificial Sequence: Synthetic polypeptide
AGFPGLPGPAGEPGRHGKDGLMGSPGFKGEAGSPGAPGQDGTRGEPGIPGFPGNRGLMGQKGEIGPPGQQGKKGAPGMPGLMGSNGSPGQPGTPGSKGSKGEPGIQGMPGASGLKGEPGATGSPGEPGYMGLPGIQGKKGDKGNQGEKGIQGQKGENGRQGIPGQQGIQGHHGAKGERGEKGEPGVR

SEQ ID NO: 17 - DNA, 639 bp, synthetic construct
  misc_feature 1..639: note = Description of Artificial Sequence: Synthetic polynucleotide
  source 1..639: mol_type = other DNA
atgaaaaagatttggctggcgctggctggtttagttttagcgtttagcgcatcggcggcgcagtatgaagatatgggtccgcctggtagccgtggtgcaagtggtccggcaggcgttcgtggtccgaatggtgatgcaggtcgtccgggtgaaccgggtctgatgggtcctcgtggtctgcctggttcaccgggtaatattggtcctgcaggtaaagaaggtccggttggtctgccaggtattgatggccgtccgggtccgattggtccagccggtgcacgtggtgaacctggcaatattggttttccgggtcctaaaggtccgaccggtgatccgggtaaaaatggtgataaaggtcatgcaggtctggcaggcgcacgcggtgcacctggtccggatggtaataatggtgcacagggtccaccgggtccgcagggtgttcaaggtggtaaaggcgaacagggtcctgccggtcctccgggttttcagggactgcctggtccgagcggtcctgcgggtgaagttggtaaacctggtgaacgcggtctgcatggtgaatttggcctgcctgggcctgcaggtccgcgtggcgaacgtggtccgccaggtgaaagcggtgcagcaggtccgacaggttaa

SEQ ID NO: 18 - protein, 212 aa, synthetic construct
  REGION 1..212: note = Description of Artificial Sequence: Synthetic polypeptide
MKKIWLALAGLVLAFSASAAQYEDMGPPGSRGASGPAGVRGPNGDAGRPGEPGLMGPRGLPGSPGNIGPAGKEGPVGLPGIDGRPGPIGPAGARGEPGNIGFPGPKGPTGDPGKNGDKGHAGLAGARGAPGPDGNNGAQGPPGPQGVQGGKGEQGPAGPPGFQGLPGPSGPAGEVGKPGERGLHGEFGLPGPAGPRGERGPPGESGAAGPTG

SEQ ID NO: 19 - DNA, 567 bp, synthetic construct
  misc_feature 1..567: note = Description of Artificial Sequence: Synthetic polynucleotide
  source 1..567: mol_type = other DNA
atgggtccgcctggtagccgtggtgcaagtggtccggcaggcgttcgtggtccgaatggtgatgcaggtcgtccgggtgaaccgggtctgatgggtcctcgtggtctgcctggttcaccgggtaatattggtcctgcaggtaaagaaggtccggttggtctgccaggtattgatggccgtccgggtccgattggtccagccggtgcacgtggtgaacctggcaatattggttttccgggtcctaaaggtccgaccggtgatccgggtaaaaatggtgataaaggtcatgcaggtctggcaggcgcacgcggtgcacctggtccggatggtaataatggtgcacagggtccaccgggtccgcagggtgttcaaggtggtaaaggcgaacagggtcctgccggtcctccgggttttcagggactgcctggtccgagcggtcctgcgggtgaagttggtaaacctggtgaacgcggtctgcatggtgaatttggcctgcctgggcctgcaggtccgcgtggcgaacgtggtccgccaggtgaaagcggtgcagcaggtccgacaggttaa

SEQ ID NO: 20 - protein, 188 aa, synthetic construct
  REGION 1..188: note = Description of Artificial Sequence: Synthetic polypeptide
MGPPGSRGASGPAGVRGPNGDAGRPGEPGLMGPRGLPGSPGNIGPAGKEGPVGLPGIDGRPGPIGPAGARGEPGNIGFPGPKGPTGDPGKNGDKGHAGLAGARGAPGPDGNNGAQGPPGPQGVQGGKGEQGPAGPPGFQGLPGPSGPAGEVGKPGERGLHGEFGLPGPAGPRGERGPPGESGAAGPTG

SEQ ID NO: 21 - DNA, 612 bp, synthetic construct
  misc_feature 1..612: note = Description of Artificial Sequence: Synthetic polynucleotide
  source 1..612: mol_type = other DNA
atgaaaaagatttggctggcgctggctggtttagttttagcgtttagcgcatcggcggcgcagtatgaagatggttttcagggtcctgccggtgaaccgggtgaacctggtcagacaggtccggcaggcgcacgtggtcctgcaggtcctcctggtaaagccggtgaagatggtcatccgggtaaaccgggtcgtcctggtgaacgtggtgttgttggtccgcagggtgcccgtggttttccgggtactccgggtctgccaggttttaaaggtattcgtggtcataatggtctggatggtctgaaaggtcagcctggtgcaccgggtgttaaaggtgaaccaggtgctccgggtgaaaatggcacaccgggtcagaccggtgcgcgtggtctgcctggcgaacgcggtcgtgttggtgcacctggtccagccggtgcacgcggtagtgatggtagcgttggtccggttggtccagcgggtccgattggtagcgcaggtccaccgggttttccaggcgcaccgggtccgaaaggtgaaattggtgcagttggtaatgcaggccctgccggtccagcaggaccgcgtggtgaagttggcctgcctggtctgtaa

SEQ ID NO: 22 - protein, 203 aa, synthetic construct
  REGION 1..203: note = Description of Artificial Sequence: Synthetic polypeptide
MKKIWLALAGLVLAFSASAAQYEDGFQGPAGEPGEPGQTGPAGARGPAGPPGKAGEDGHPGKPGRPGERGVVGPQGARGFPGTPGLPGFKGIRGHNGLDGLKGQPGAPGVKGEPGAPGENGTPGQTGARGLPGERGRVGAPGPAGARGSDGSVGPVGPAGPIGSAGPPGFPGAPGPKGEIGAVGNAGPAGPAGPRGEVGLPGL

SEQ ID NO: 23 - DNA, 540 bp, synthetic construct
  misc_feature 1..540: note = Description of Artificial Sequence: Synthetic polynucleotide
  source 1..540: mol_type = other DNA
ggttttcagggtcctgccggtgaaccgggtgaacctggtcagacaggtccggcaggcgcacgtggtcctgcaggtcctcctggtaaagccggtgaagatggtcatccgggtaaaccgggtcgtcctggtgaacgtggtgttgttggtccgcagggtgcccgtggttttccgggtactccgggtctgccaggttttaaaggtattcgtggtcataatggtctggatggtctgaaaggtcagcctggtgcaccgggtgttaaaggtgaaccaggtgctccgggtgaaaatggcacaccgggtcagaccggtgcgcgtggtctgcctggcgaacgcggtcgtgttggtgcacctggtccagccggtgcacgcggtagtgatggtagcgttggtccggttggtccagcgggtccgattggtagcgcaggtccaccgggttttccaggcgcaccgggtccgaaaggtgaaattggtgcagttggtaatgcaggccctgccggtccagcaggaccgcgtggtgaagttggcctgcctggtctgtaa

SEQ ID NO: 24 - protein, 179 aa, synthetic construct
  REGION 1..179: note = Description of Artificial Sequence: Synthetic polypeptide
GFQGPAGEPGEPGQTGPAGARGPAGPPGKAGEDGHPGKPGRPGERGVVGPQGARGFPGTPGLPGFKGIRGHNGLDGLKGQPGAPGVKGEPGAPGENGTPGQTGARGLPGERGRVGAPGPAGARGSDGSVGPVGPAGPIGSAGPPGFPGAPGPKGEIGAVGNAGPAGPAGPRGEVGLPGL

SEQ ID NO: 25 - protein, 228 aa, synthetic construct
  REGION 1..228: note = Description of Artificial Sequence: Synthetic polypeptide
MKKIWLALAGLVLAFSASAGDQGPVGRTGEVGAVGPPGFAGEKGPSGEAGTAGPPGTPGPQGLLGAPGILGLPGSRGERGLPGVAGAVGEPGPLGIAGPPGARGPPGAVGSPGVNGAPGEAGRDGNPGNDGPPGRDGQPGHKGERGYPGNIGPVGAAGAPGPHGPVGPAGKHGNRGETGPSGPVGPAGAVGPRGPSGPQGIRGDKGEPGEKGPRGLPGLGDYKDDDDK

SEQ ID NO: 26 - DNA, 687 bp, synthetic construct
  misc_feature 1..687: note = Description of Artificial Sequence: Synthetic polynucleotide
  source 1..687: mol_type = other DNA
atgaaaaagatttggctggcgctggctggtttagttttagcgtttagcgcatcggcgggtgatcagggtccggttggtcgtaccggtgaagttggtgcagtcgggccgccgggttttgcgggtgaaaaaggcccgtcaggtgaagcaggcaccgctggccctcctggcacgcctggcccacagggtttactgggcgcacctggaattctgggactgccgggcagccgtggagaacgcggtttaccaggtgttgccggtgccgttggtgaacctggtccactgggcattgcagggccgcctggcgcacggggaccgcctggtgctgttggtagtccgggtgtgaatggtgctccgggtgaagccggtcgtgacggtaatccgggaaatgacggcccgccaggccgcgatggtcagccgggtcataaaggtgagcgtggttacccaggtaatattggtccagtcggtgccgccggtgcgccgggtcctcatggccctgtcggtccagccggtaaacatggtaatcgcggtgagacaggtccgtcaggaccagtgggccctgctggcgcagtcggtccgcgcgggccgagtggccctcagggtattcgtggcgataaaggggaaccgggcgaaaaagggccgcggggtctgccaggcctgggtgactacaaagacgacgacgacaaataa

SEQ ID NO: 27 - protein, 228 aa, synthetic construct
  REGION 1..228: note = Description of Artificial Sequence: Synthetic polypeptide
MKKIWLALAGLVLAFSASAKGHNGLQGLPGIAGHHGDQGAPGSVGPAGPRGPAGPSGPAGKDGRTGHPGTVGPAGIRGPQGHQGPAGPPGPPGPPGPPGVSGGGYDFGYDGDFYRADQPRSAPSLRPKDYEVDATLKSLNNQIETLLTPEGSRKNPARTCRDLRLSHPEWSSGYYWIDPNQGCTMDAIKVYCDFSTGETCIRAQPENIPAKNWYRSSKDGDYKDDDDK

SEQ ID NO: 28 - DNA, 687 bp, synthetic construct
  misc_feature 1..687: note = Description of Artificial Sequence: Synthetic polynucleotide
  source 1..687: mol_type = other DNA
atgaaaaagatttggctggcgctggctggtttagttttagcgtttagcgcatcggcgaaaggtcacaatggactgcaaggcctgccaggtattgcaggtcatcatggtgatcaaggtgccccgggaagcgttggtccggcggggccgagaggccctgcgggaccttcaggtccggcaggcaaagatggtcggacaggccatccgggcaccgttggccctgcaggaattcgtggaccgcagggtcatcagggacctgctggtccgccaggtcccccgggccctccgggaccaccgggtgttagtggtggtggttatgattttggctatgatggtgatttttatcgtgcagatcagccgcgtagcgcaccgagcctgcgtcctaaagattatgaagttgatgcaaccctgaaaagcctgaataatcagattgaaacactgctgacaccggaaggtagccgtaaaaatccggcccgtacctgtcgtgatctgcgtctgagccacccggaatggagcagcggttattattggattgatccgaatcaaggttgtaccatggatgcaattaaagtttattgtgattttagcacaggtgaaacatgtatccgtgcacagccggaaaatattccggccaaaaattggtatcgtagtagcaaagatggtgactacaaagacgacgacgacaaataa

SEQ ID NO: 29 - protein, 262 aa, synthetic construct
  REGION 1..262: note = Description of Artificial Sequence: Synthetic polypeptide
MKKIWLALAGLVLAFSASAYEVDATLKSLNNQIETLLTPEGSRKNPARTCRDLRLSHPEWSSGYYWIDPNQGCTMDAIKVYCDFSTGETCIRAQPENIPAKNWYRSSKDKKHVWLGETINAGSQFEYNVEGVTSKEMATQLAFMRLLANYASQNITYHCKNSIAYMDEETGNLKKAVILQGSNDVELVAEGNSRFTYTVLVDGCSKKTNEWGKTIIEYKTNKPSRLPFLDIAPLDIGGADQEFFVDIGPVCFKGDYKDDDDK

SEQ ID NO: 30 - DNA, 788 bp, synthetic construct
  misc_feature 1..788: note = Description of Artificial Sequence: Synthetic polynucleotide
  source 1..788: mol_type = other DNA
tgaaaaagatttggctggcgctggctggtttagttttagcgtttagcgcatcggcgtatgaagttgatgcaaccctgaaaagcctgaataatcagattgaaacactgctgacaccggaaggtagccgtaaaaatccggcccgtacctgtcgtgatctgcgtctgagccacccggaatggagcagcggttattattggattgatccgaatcaaggttgtaccatggatgcaattaaagtttattgtgattttagcacaggtgaaacatgtatccgtgcacagccggaaaatattccggccaaaaattggtatcgtagtagcaaagataaaaaacatgtgtggctgggtgaaaccattaatgcaggtagccagtttgaatacaatgttgaaggtgttaccagcaaagaaatggcaacacagctggcatttatgcgtctgctggcaaattatgcaagccagaatattacatatcattgtaaaaatagcattgcatatatggatgaagaaaccggtaatctgaaaaaagcagttattctgcagggtagcaatgatgttgaactggttgccgaaggtaatagccgttttacatataccgttctggttgatggttgtagcaaaaaaaccaatgaatggggtaaaaccatcattgaatataaaaccaacaaaccgagccgtctgccgtttctggatatcgctccgctggatattggtggtgccgatcaggaattttttgtcgatatcggtcctgtgtgttttaaaggtgactacaaagacgacgacgacaaataa

SEQ ID NO: 31 - protein, 957 aa, Homo sapiens
MAHYITFLCMVLVLLLQNSVLAEDGEVRSSCRTAPTDLVFILDGSYSVGPENFEIVKKWLVNITKNFDIGPKFIQVGVVQYSDYPVLEIPLGSYDSGEHLTAAVESILYLGGNTKTGKAIQFALDYLFAKSSRFLTKIAVVLTDGKSQDDVKDAAQAARDSKITLFAIGVGSETEDAELRAIANKPSSTYVFYVEDYIAISKIREVMKQKLCEESVCPTRIPVAARDERGFDILLGLDVNKKVKKRIQLSPKKIKGYEVTSKVDLSELTSNVFPEGLPPSYVFVSTQRFKVKKIWDLWRILTIDGRPQIAVTLNGVDKILLFTTTSVINGSQVVTFANPQVKTLFDEGWHQIRLLVTEQDVTLYIDDQQIENKPLHPVLGILINGQTQIGKYSGKEETVQFDVQKLRIYCDPEQNNRETACEIPGFNGECLNGPSDVGSTPAPCICPPGKPGLQGPKGDPGLPGNPGYPGQPGQDGKPGYQGIAGTPGVPGSPGIQGARGLPGYKGEPGRDGDKGDRGLPGFPGLHGMPGSKGEMGAKGDKGSPGFYGKKGAKGEKGNAGFPGLPGPAGEPGRHGKDGLMGSPGFKGEAGSPGAPGQDGTRGEPGIPGFPGNRGLMGQKGEIGPPGQQGKKGAPGMPGLMGSNGSPGQPGTPGSKGSKGEPGIQGMPGASGLKGEPGATGSPGEPGYMGLPGIQGKKGDKGNQGEKGIQGQKGENGRQGIPGQQGIQGHHGAKGERGEKGEPGVRGAIGSKGESGVDGLMGPAGPKGQPGDPGPQGPPGLDGKPGREFSEQFIRQVCTDVIRAQLPVLLQSGRIRNCDHCLSQHGSPGIPGPPGPIGPEGPRGLPGLPGRDGVPGLVGVPGRPGVRGLKGLPGRNGEKGSQGFGYPGEQGPPGPPGPEGPPGISKEGPPGDPGLPGKDGDHGKPGIQGQPGPPGICDPSLCFSVIARRDPFRKGPNY

SEQ ID NO: 32 - protein, 1366 aa, Homo sapiens
MLSFVDTRTLLLLAVTLCLATCQSLQEETVRKGPAGDRGPRGERGPPGPPGRDGEDGPTGPPGPPGPPGPPGLGGNFAAQYDGKGVGLGPGPMGLMGPRGPPGAAGAPGPQGFQGPAGEPGEPGQTGPAGARGPAGPPGKAGEDGHPGKPGRPGERGVVGPQGARGFPGTPGLPGFKGIRGHNGLDGLKGQPGAPGVKGEPGAPGENGTPGQTGARGLPGERGRVGAPGPAGARGSDGSVGPVGPAGPIGSAGPPGFPGAPGPKGEIGAVGNAGPAGPAGPRGEVGLPGLSGPVGPPGNPGANGLTGAKGAAGLPGVAGAPGLPGPRGIPGPVGAAGATGARGLVGEPGPAGSKGESGNKGEPGSAGPQGPPGPSGEEGKRGPNGEAGSAGPPGPPGLRGSPGSRGLPGADGRAGVMGPPGSRGASGPAGVRGPNGDAGRPGEPGLMGPRGLPGSPGNIGPAGKEGPVGLPGIDGRPGPIGPAGARGEPGNIGFPGPKGPTGDPGKNGDKGHAGLAGARGAPGPDGNNGAQGPPGPQGVQGGKGEQGPPGPPGFQGLPGPSGPAGEVGKPGERGLHGEFGLPGPAGPRGERGPPGESGAAGPTGPIGSRGPSGPPGPDGNKGEPGVVGAVGTAGPSGPSGLPGERGAAGIPGGKGEKGEPGLRGEIGNPGRDGARGAPGAVGAPGPAGATGDRGEAGAAGPAGPAGPRGSPGERGEVGPAGPNGFAGPAGAAGQPGAKGERGAKGPKGENGVVGPTGPVGAAGPAGPNGPPGPAGSRGDGGPPGMTGFPGAAGRTGPPGPSGISGPPGPPGPAGKEGLRGPRGDQGPVGRTGEVGAVGPPGFAGEKGPSGEAGTAGPPGTPGPQGLLGAPGILGLPGSRGERGLPGVAGAVGEPGPLGIAGPPGARGPPGAVGSPGVNGAPGEAGRDGNPGNDGPPGRDGQPGHKGERGYPGNIGPVGAAGAPGPHGPVGPAGKHGNRGETGPSGPVGPAGAVGPRGPSGPQGIRGDKGEPGEKGPRGLPGLKGHNGLQGLPGIAGHHGDQGAPGSVGPAGPRGPAGPSGPAGKDGRTGHPGTVGPAGIRGPQGHQGPAGPPGPPGPPGPPGVSGGGYDFGYDGDFYRADQPRSAPSLRPKDYEVDATLKSLNNQIETLLTPEGSRKNPARTCRDLRLSHPEWSSGYYWIDPNQGCTMDAIKVYCDFSTGETCIRAQPENIPAKNWYRSSKDKKHVWLGETINAGSQFEYNVEGVTSKEMATQLAFMRLLANYASQNITYHCKNSIAYMDEETGNLKKAVILQGSNDVELVAEGNSRFTYTVLVDGCSKKTNEWGKTIIEYKTNKPSRLPFLDIAPLDIGGADQEFFVDIGPVCFK

SEQ ID NO: 33 - protein, 429 aa, Hydractinia carnea
GPQGVVGADGKDGTPGEKGEQGRTGAAGKQGSPGADGARGPLGSIGQQGARGEPGDPGSPGLRGDTGLAGVKGVAGPSGRPGQPGANGLPGVNGRGGLRGKPGAKGIAGSDGEAGESGAPGQSGPTGPRGQRGPSGEDGNPGLQGLPGSDGEPGEEGQPGRSGQPGQQGPRGSPGEVGPRGSKGPSGDRGDRGERGVPGQTGSAGNVGEDGEQGGKGVDGASGPSGALGARGPPGSRGDTGAVGPPGPTGRSGLPGNAGQKGPSGEPGSPGKAGSAGEQGPPGKDGSNGEPGSPGKEGERGLAGPPGPDGRRGETGSPGIAGALGKPGLEGPKGYPGLRGRDGTNGKRGEQGETGPDGVRGIPGNDGQSGKPGIDGIDGTNGQPGEAGYQGGRGTRGQLGETGDVGQNGDRGAPGPDGSKGSAGRPGLR

SEQ ID NO: 34 - protein, 6 aa, Homo sapiens
VGVAPG

SEQ ID NO: 35 - protein, 12 aa, Homo sapiens
VGVAPGVGVAPG

SEQ ID NO: 36 - protein, 18 aa, Homo sapiens
VGVAPGVGVAPGVGVAPG

SEQ ID NO: 37 - DNA, 930 bp, synthetic construct
  misc_feature 1..930: note = Description of Artificial Sequence: Synthetic polynucleotide
  source 1..930: mol_type = other DNA
atgaaaaagatttggctggcgctggctggtttagttttagcgtttagcgcatcggcgggtccgcaacctggggttccgttaggttatccgattaaagcaccgaaactgcccggcggttatggtctgccgtacacaaccggtaaactgccgtatggttatggcccgggtggagttgcgggtgcagcaggtaaagcgggttatcctaccggaaccggtgtaggtccgcaggccgctgctgccgccgccgcaaaagcagcggctaaatttggcgccggagcagcgggtgttctgcctggagttggtggtgcgggcgtgccaggggtacctggtgcaattccgggtattggtggtattgccggtgtcggcaccccggccgcggcagctgcggcagcggcggctgccaaagctgctaaatacggtgccgcggcgggtctggtgccaggaggtccgggttttggtccgggagtggttggcgtgcctggcgcaggcgttcctggtgtgggcgttccaggtgcagggattcctgttgtgcctggtgccggtattcccggcgcggccgttccgggggtggttagcccggaagccgcagcgaaggctgcggcaaaggcagcaaagtatggcgcacgcccaggagtcggcgtgggtggtatcccgacctatggggtgggcgcagggggttttcctggtttcggcgtaggtgtaggaggtataccgggcgtggccggtgtaccaggggttggtggcgtccctggtgttggcggtgtgccaggtgttggtatttcaccggaagcacaggcagcagccgcagctaaggcagcgaaatatggtgccgccggcgcaggagttttaggtgggctggttccgggcccgcaggcagctgtgccgggggttccaggcaccggtggtgtccctggagtcggtacgccgtaa

SEQ ID NO: 38 - protein, 309 aa, synthetic construct
  REGION 1..309: note = Description of Artificial Sequence: Synthetic polypeptide
MKKIWLALAGLVLAFSASAGPQPGVPLGYPIKAPKLPGGYGLPYTTGKLPYGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPEAAAKAAAKAAKYGARPGVGVGGIPTYGVGAGGFPGFGVGVGGIPGVAGVPGVGGVPGVGGVPGVGISPEAQAAAAAKAAKYGAAGAGVLGGLVPGPQAAVPGVPGTGGVPGVGTP

SEQ ID NO: 39 - DNA, 873 bp, synthetic construct
  misc_feature 1..873: note = Description of Artificial Sequence: Synthetic polynucleotide
  source 1..873: mol_type = other DNA
ggtccgcaacctggggttccgttaggttatccgattaaagcaccgaaactgcccggcggttatggtctgccgtacacaaccggtaaactgccgtatggttatggcccgggtggagttgcgggtgcagcaggtaaagcgggttatcctaccggaaccggtgtaggtccgcaggccgctgctgccgccgccgcaaaagcagcggctaaatttggcgccggagcagcgggtgttctgcctggagttggtggtgcgggcgtgccaggggtacctggtgcaattccgggtattggtggtattgccggtgtcggcaccccggccgcggcagctgcggcagcggcggctgccaaagctgctaaatacggtgccgcggcgggtctggtgccaggaggtccgggttttggtccgggagtggttggcgtgcctggcgcaggcgttcctggtgtgggcgttccaggtgcagggattcctgttgtgcctggtgccggtattcccggcgcggccgttccgggggtggttagcccggaagccgcagcgaaggctgcggcaaaggcagcaaagtatggcgcacgcccaggagtcggcgtgggtggtatcccgacctatggggtgggcgcagggggttttcctggtttcggcgtaggtgtaggaggtataccgggcgtggccggtgtaccaggggttggtggcgtccctggtgttggcggtgtgccaggtgttggtatttcaccggaagcacaggcagcagccgcagctaaggcagcgaaatatggtgccgccggcgcaggagttttaggtgggctggttccgggcccgcaggcagctgtgccgggggttccaggcaccggtggtgtccctggagtcggtacgccgtaa

SEQ ID NO: 40 - protein, 290 aa, synthetic construct
  REGION 1..290: note = Description of Artificial Sequence: Synthetic polypeptide
GPQPGVPLGYPIKAPKLPGGYGLPYTTGKLPYGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPEAAAKAAAKAAKYGARPGVGVGGIPTYGVGAGGFPGFGVGVGGIPGVAGVPGVGGVPGVGGVPGVGISPEAQAAAAAKAAKYGAAGAGVLGGLVPGPQAAVPGVPGTGGVPGVGTP

SEQ ID NO: 41 - protein, 238 aa, synthetic construct
  REGION 1..238: note = Description of Artificial Sequence: Synthetic polypeptide
GPGGVAAAAKSAAKVAAKAQLRAAAGLGAGIPGLGVGVGVPGLGVGAGVPGLGVGAGVPGFGAGADEGVRRSLSPELREGDPSSSQHLPSTPSSPRVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 42 - DNA, 717 bp, synthetic construct
  misc_feature 1..717: note = Description of Artificial Sequence: Synthetic polynucleotide
  source 1..717: mol_type = other DNA
ggtccgggcggtgtcgcagcagcagctaaaagcgcggcgaaagttgcggccaaagcccaactgcgcgccgccgcgggcctcggtgcaggtattccggggctgggtgtcggagttggagtcccgggtttgggcgtgggcgcgggagttccgggactgggagtgggtgccggagttcctggctttggtgcaggcgcagatgaaggtgttcgtcgtagcctgagtccggaactgcgtgaaggtgatccgagtagcagccagcatctgccgagcaccccgagcagcccgcgtgttccgggtgcattagctgcagcaaaagccgccaagtatggtgcagccgtgccgggcgtcttaggtggtctgggcgccctgggtggtgtaggcattccgggaggtgttgtgggtgcaggaccggccgccgcagctgcggccgccaaagcagctgcaaaagcggcccagtttggtttagtgggcgccgcaggtttaggcggtttaggtgtgggtggactgggtgtacctggcgtaggcggtctgggtggaattccgcccgcagcggccgcgaaagcggcaaaatatggcgcggcaggcctgggcggcgtgctgggtggggcaggtcagtttccgctgggcggggttgccgcacgtccgggatttggtctgagcccgattttccctggcggcgcatgtctgggtaaagcatgtggtcgtaaacgtaaataa

SEQ ID NO: 43 - protein, 693 aa, synthetic construct
  REGION 1..693: note = Description of Artificial Sequence: Synthetic polypeptide
GGVPGAIPGGVPGGVFYPGAGLGALGGGALGPGGKPLKPVPGGLAGAGLGAGLGAFPAVTFPGALVPGGVADAAAAYKAAKAGAGLGGVPGVGGLGVSAGAVVPQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPEAAAKAAAKAAKYGARPGVGVGGIPTYGVGAGGFPGFGVGVGGIPGVAGVPGVGGVPGVGGVPGVGISPEAQAAAAAKAAKYGAAGAGVLGGLVPGPQAAVPGVPGTGGVPGVGTPAAAAKSAAKVAAKAQLRAAAGLGAGIPGLGVGVGVPGLGVGAGVPGLGVGAGVPGFGAGADEGVRRSLSPELREGDPSSSQHLPSTPSSPRVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 44 - DNA, 2082 bp, synthetic construct
  misc_feature 1..2082: note = Description of Artificial Sequence: Synthetic polynucleotide
  source 1..2082: mol_type = other DNA
ggtggcgtaccaggcgcaattcctgggggtgtcccaggcggtgttttttatccgggcgccggtcttggcgcactgggtggcggtgcactgggcccgggcggcaaaccgctgaaaccggtaccaggtggtttagcaggcgccggcttaggcgcaggtctgggagcatttccggcagttacctttccaggggcactggttcctggaggtgtggccgatgcagccgcggcatataaagccgctaaagccggtgcgggtttaggaggcgtcccaggtgtcggtggcctgggtgttagcgccggtgcagttgttccgcagccgggagcaggggttaaacctggtaaagtgccgggagtaggtctgccaggcgtttatcctggtggtgttttgccgggtgcccgttttccgggcgttggtgttcttccaggcgtgccgaccggagccggtgttaaaccgaaagcccccggtgttggaggtgcatttgcaggcatcccgggagttggcccgtttggtggtccgcaacctggggttccgttaggttatccgattaaagcaccgaaactgcccggcggttatggtctgccgtacacaaccggtaaactgccgtatggttatggcccgggtggagttgcgggtgcagcaggtaaagcgggttatcctaccggaaccggtgtaggtccgcaggccgctgctgccgccgccgcaaaagcagcggctaaatttggcgccggagcagcgggtgttctgcctggagttggtggtgcgggcgtgccaggggtacctggtgcaattccgggtattggtggtattgccggtgtcggcaccccggccgcggcagctgcggcagcggcggctgccaaagctgctaaatacggtgccgcggcgggtctggtgccaggaggtccgggttttggtccgggagtggttggcgtgcctggcgcaggcgttcctggtgtgggcgttccaggtgcagggattcctgttgtgcctggtgccggtattcccggcgcggccgttccgggggtggttagcccggaagccgcagcgaaggctgcggcaaaggcagcaaagtatggcgcacgcccaggagtcggcgtgggtggtatcccgacctatggggtgggcgcagggggttttcctggtttcggcgtaggtgtaggaggtataccgggcgtggccggtgtaccaggggttggtggcgtccctggtgttggcggtgtgccaggtgttggtatttcaccggaagcacaggcagcagccgcagctaaggcagcgaaatatggtgccgccggcgcaggagttttaggtgggctggttccgggcccgcaggcagctgtgccgggggttccaggcaccggtggtgtccctggagtcggtacgccggcagcagcagctaaaagcgcggcgaaagttgcggccaaagcccaactgcgcgccgccgcgggcctcggtgcaggtattccggggctgggtgtcggagttggagtcccgggtttgggcgtgggcgcgggagttccgggactgggagtgggtgccggagttcctggctttggtgcaggcgcagatgaaggtgttcgtcgtagcctgagtccggaactgcgtgaaggtgatccgagtagcagccagcatctgccgagcaccccgagcagcccgcgtgttccgggtgcattagctgcagcaaaagccgccaagtatggtgcagccgtgccgggcgtcttaggtggtctgggcgccctgggtggtgtaggcattccgggaggtgttgtgggtgcaggaccggccgccgcagctgcggccgccaaagcagctgcaaaagcggcccagtttggtttagtgggcgccgcaggtttaggcggtttaggtgtgggtggactgggtgtacctggcgtaggcggtctgggtggaattccgcccgcagcggccgcgaaagcggcaaaatatggcgcggcaggcctgggcggcgtgctgggtggggcaggtcagtttccgctgggcggggttgccgcacgtccgggatttggtctgagcccgattttccctggcggcgcatgtctgggtaaagcatgtggtcgtaaacgtaaataa

SEQ ID NO: 45 - DNA, 846 bp, synthetic construct
  misc_feature 1..846: note = Description of Artificial Sequence: Synthetic polynucleotide
  source 1..846: mol_type = other DNA
atgaaaaagatttggctggcgctggctggtttagttttagcgtttagcgcatcggcggccggtctgggtggcgtgcctggcgttggtggcctgggcgttagcgccggtgcagttgttccgcagcctggtgcgggtgttaaaccgggtaaagttcctggtgttggtctgcctggtgtttatccaggcggtgttctgccaggtgcgcgttttcctggcgtgggtgttctgccgggtgttccgaccggtgcaggcgtgaaaccgaaagcaccaggtgttggtggtgcatttgcaggtattccaggtgtgggtccgtttggtggtccgcagccaggcgttccgctgggttatccgattaaagcaccgaaactgcctggcggttatggtctgccgtataccaccggtaaactgccgtatggttatggccctggcggtgttgccggtgcggcaggtaaagcaggctatccgaccggtaccggtgtaggtccgcaggcagcagccgcagcagcggcaaaagcagcagcgaaatttggtgcgggtgcagccggtgtgctgccaggcgtaggtggcgctggtgtacctggtgtccctggcgcaattccaggtattggcggtattgcaggcgttggtactccggcagctgcagccgctgccgcagccgcagctaaagcagccaaatatggtgcagcggcaggcctggttcctggcggtccaggttttggtccgggtgttgttggcgtcccaggtgccggtgtgcctggtgtgggtgttccaggtgcgggtattccggttgttcctggcgcaggcattccgggtgccgcagttccgggtgtagttagcccggaataa

SEQ ID NO: 46 - protein, 281 aa, synthetic construct
  REGION 1..281: note = Description of Artificial Sequence: Synthetic polypeptide
MKKIWLALAGLVLAFSASAAGLGGVPGVGGLGVSAGAVVPQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPE

SEQ ID NO: 47 - DNA, 789 bp, synthetic construct
  misc_feature 1..789: note = Description of Artificial Sequence: Synthetic polynucleotide
  source 1..789: mol_type = other DNA
gccggtctgggtggcgtgcctggcgttggtggcctgggcgttagcgccggtgcagttgttccgcagcctggtgcgggtgttaaaccgggtaaagttcctggtgttggtctgcctggtgtttatccaggcggtgttctgccaggtgcgcgttttcctggcgtgggtgttctgccgggtgttccgaccggtgcaggcgtgaaaccgaaagcaccaggtgttggtggtgcatttgcaggtattccaggtgtgggtccgtttggtggtccgcagccaggcgttccgctgggttatccgattaaagcaccgaaactgcctggcggttatggtctgccgtataccaccggtaaactgccgtatggttatggccctggcggtgttgccggtgcggcaggtaaagcaggctatccgaccggtaccggtgtaggtccgcaggcagcagccgcagcagcggcaaaagcagcagcgaaatttggtgcgggtgcagccggtgtgctgccaggcgtaggtggcgctggtgtacctggtgtccctggcgcaattccaggtattggcggtattgcaggcgttggtactccggcagctgcagccgctgccgcagccgcagctaaagcagccaaatatggtgcagcggcaggcctggttcctggcggtccaggttttggtccgggtgttgttggcgtcccaggtgccggtgtgcctggtgtgggtgttccaggtgcgggtattccggttgttcctggcgcaggcattccgggtgccgcagttccgggtgtagttagcccggaataa

SEQ ID NO: 48 - protein, 262 aa, synthetic construct
  REGION 1..262: note = Description of Artificial Sequence: Synthetic polypeptide
AGLGGVPGVGGLGVSAGAVVPQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPE

SEQ ID NO: 49 - DNA, 492 bp, synthetic construct
  misc_feature 1..492: note = Description of Artificial Sequence: Synthetic polynucleotide
  source 1..492: mol_type = other DNA
atgaaaaagatttggctggcgctggctggtttagttttagcgtttagcgcatcggcggccggtctgggtggcgtgcctggcgttggtggcctgggcgttagcgccggtgcagttgttccgcagcctggtgcgggtgttaaaccgggtaaagttcctggtgttggtctgcctggtgtttatccaggcggtgttctgccaggtgcgcgttttcctggcgtgggtgttctgccgggtgttccgaccggtgcaggcgtgaaaccgaaagcaccaggtgttggtggtgcatttgcaggtattccaggtgtgggtccgtttggtggtccgcagccaggcgttccgctgggttatccgattaaagcaccgaaactgcctggcggttatggtctgccgtataccaccggtaaactgccgtatggttatggccctggcggtgttgccggtgcggcaggtaaagcaggctatccgaccggtaccggtgtaggtccgcagtaa

SEQ ID NO: 50 - protein, 163 aa, synthetic construct
  REGION 1..163: note = Description of Artificial Sequence: Synthetic polypeptide
MKKIWLALAGLVLAFSASAAGLGGVPGVGGLGVSAGAVVPQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQ

SEQ ID NO: 51 - DNA, 435 bp, synthetic construct
  misc_feature 1..435: note = Description of Artificial Sequence: Synthetic polynucleotide
  source 1..435: mol_type = other DNA
gccggtctgggtggcgtgcctggcgttggtggcctgggcgttagcgccggtgcagttgttccgcagcctggtgcgggtgttaaaccgggtaaagttcctggtgttggtctgcctggtgtttatccaggcggtgttctgccaggtgcgcgttttcctggcgtgggtgttctgccgggtgttccgaccggtgcaggcgtgaaaccgaaagcaccaggtgttggtggtgcatttgcaggtattccaggtgtgggtccgtttggtggtccgcagccaggcgttccgctgggttatccgattaaagcaccgaaactgcctggcggttatggtctgccgtataccaccggtaaactgccgtatggttatggccctggcggtgttgccggtgcggcaggtaaagcaggctatccgaccggtaccggtgtaggtccgcagtaa

SEQ ID NO: 52 - protein, 144 aa, synthetic construct
  REGION 1..144: note = Description of Artificial Sequence: Synthetic polypeptide
AGLGGVPGVGGLGVSAGAVVPQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQ

SEQ ID NO: 53 - protein, 760 aa, Homo sapiens
GGVPGAIPGGVPGGVFYPGAGLGALGGGALGPGGKPLKPVPGGLAGAGLGAGLGAFPAVTFPGALVPGGVADAAAAYKAAKAGAGLGGVPGVGGLGVSAGAVVPQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPEAAAKAAAKAAKYGARPGVGVGGIPTYGVGAGGFPGFGVGVGGIPGVAGVPGVGGVPGVGGVPGVGISPEAQAAAAAKAAKYGAAGAGVLGGLVPGPQAAVPGVPGTGGVPGVGTPAAAAAKAAAKAAQFGLVPGVGVAPGVGVAPGVGVAPGVGLAPGVGVAPGVGVAPGVGVAPGIGPGGVAAAAKSAAKVAAKAQLRAAAGLGAGIPGLGVGVGVPGLGVGAGVPGLGVGAGVPGFGAGADEGVRRSLSPELREGDPSSSQHLPSTPSSPRVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 54 - protein, 30 aa, synthetic construct
  REGION 1..30: note = Description of Artificial Sequence: Synthetic His tag
  SITE 1..30: note = This sequence may encompass 2-30 residues
HHHHHHHHHHHHHHHHHHHHHHHHHHHHHH

SEQ ID NO: 55 - protein, 20 aa, synthetic construct
  REGION 1..20: note = Description of Artificial Sequence: Synthetic His tag
  SITE 1..20: note = This sequence may encompass 2-20 residues
  REGION 1..20: note = See specification as filed for detailed description of substitutions and preferred embodiments
HHHHHHHHHHHHHHHHHHHH

(19) United States
(12) Patent Application Publication
(10) Pub. No.: US 2023/0364000 A1
(43) Pub. Date: Nov. 16, 2023

(54) COMPOSITIONS COMPRISING COMBINATIONS OF COLLAGEN OR ELASTIN POLYPEPTIDES WITH ACTIVE INGREDIENTS AND METHODS OF USING SAME

(71) Applicant: Geltor, Inc., Emeryville, CA (US)

(72) Inventors: Julia CO, Oakland, CA (US); Dawn SIMPSON, Bell Canyon, CA (US); Nikolay OUZOUNOV, San Ramon, CA (US)

(21) Appl. No.: 18/191,556

(22) Filed: Mar. 28, 2023

Related U.S. Application Data
(63) Continuation of application No. PCT/US2021/052966, filed on Sep. 30, 2021.
(60) Provisional application No. 63/091,799, filed on Oct. 14, 2020.
(60) Provisional application No. 63/091,791, filed on Oct. 14, 2020.
(60) Provisional application No. 63/085,857, filed on Sep. 30, 2020.
(60) Provisional application No. 63/085,829, filed on Sep. 30, 2020.

(51) Int. Cl. A61K 8/65; A61Q 19/08; A61Q 19/00; A61K 8/67; A61K 8/73; A61K 8/368; A61K 8/38; A61K 8/64; A61Q 5/00; A61K 38/39; A61K 31/728; A61K 9/00

(52) U.S. Cl. CPC A61K 8/65; A61Q 19/08; A61Q 19/007; A61K 8/671; A61K 8/676; A61K 8/735; A61K 8/368; A61K 8/38; A61K 8/675; A61K 8/64; A61Q 5/00; A61K 38/39; A61K 31/728; A61K 9/0095; A61K 2800/91

(57) ABSTRACT

This disclosure provides compositions comprising a collagen polypeptide (e.g., a human type 21 collagen polypeptide or a truncate thereof) or an elastin polypeptide (e.g., a human elastin polypeptide or a truncate thereof), and one or more active ingredient. This disclosure also provides compositions comprising an elastin polypeptide (e.g., a human elastin polypeptide or a truncate thereof) in combination with a collagen polypeptide or truncate thereof. This disclosure further provides formulations comprising said composition, such as personal care products. This disclosure further provides methods of using the compositions and formulations provided herein (e.g., for application to the skin and/or hair a subject).

### CROSS-REFERENCE

This application is a continuation of International Application No. PCT/US2021/052966, filed Sep. 30, 2021, which claims the benefit of U.S. Provisional Application No. 63/085,829, filed Sep. 30, 2020, U.S. Provisional Application No. 63/085,857, filed Sep. 30, 2020, U.S. Provisional Application No. 63/091,791, filed Oct. 14, 2020, and U.S. Provisional Application No. 63/091,799, filed Oct. 14, 2020, each of which is incorporated herein by reference, for all purposes, in its entirety.

### SEQUENCE LISTING

The instant application contains a Sequence Listing which has been submitted electronically in XML format and is hereby incorporated by reference in its entirety. Said XML copy, created on Mar. 23, 2023, is named 57607_714_301_SL.xml and is 99,923 bytes in size.

### BACKGROUND

Collagens and similar proteins are the most abundant proteins in the biosphere. Collagens are structural proteins found in the skin, connective tissue, and bones of animals and other tissues. In humans, the amount of collagen present in the body is approximately one third of the total proteins and accounts for about three fourths of the dry weight of skin.

The structure of natural collagen can be a triple helix in which three polypeptide strands together form a helical coil. The individual polypeptide strands are composed of repeating triplet amino acid sequences designated as GLY-X-Y. X and Y can be any amino acid and the first amino acid is glycine. The amino acids proline and hydroxyproline are found in high concentrations in collagen. The most common triplet is glycine-proline-hydroxyproline (Gly-Pro-Hyp) accounting for approximately 10.5% of the triplets in collagen.

Gelatin is a product obtained by partial hydrolysis of certain (e.g., natural) collagen. Typically, gelatin is produced by acid hydrolysis, alkaline hydrolysis, and enzymatic hydrolysis or by exposing collagen to heat in an aqueous solution (e.g., boiling the bones and skins of animal, boiling fish scales, etc.).

Gelatin is used in many products including cosmetics, foods, pharmaceuticals, medical devices, photographic films, adhesives, binders, and many others. The physical and chemical properties of gelatin are tuned to the particular application. These physical/chemical properties include gel strength, melting point temperature, viscosity, color, turbidity, pH, isoelectric point, and others.

Elastin is an elastic protein that is crucial for the proper functioning of arteries, lung, tendons, ligament, skin, and other tissue. Elastin provides the tissues with the ability to stretch and return to their original shape. The protein tropoelastin is the building block of elastin. In contrast to other structural proteins, like collagen, that include a family of genes, there is one tropoelastin gene in humans. When the gene encoding tropoelastin is expressed, the single tropoelastin gene is spliced to produce different forms of the tropoelastin protein. Many tropoelastin molecules associate together to form elastin.

The structure of natural elastin is similar to collagen with an amino acid sequence rich in glycine and proline. The individual polypeptide strands are composed of repeating triplet amino acid sequences designated as GLY-X-Y interspersed with strings of alanines that provide flexibility. X and Y can be any amino acid and the first amino acid is glycine. The amino acids proline and hydroxyproline are found in high concentrations in elastin. Elastin is normally covalently crosslinked to other elastin peptides by the action of lysyl oxidase enzyme. Because of the extensive crosslinking of elastin peptides, it is difficult to extract elastins from animal tissues.

Certain domains of elastin may act as signaling domains for intracellular communication. In addition, certain elastin domains may function as binding domains for polypeptides and other biological molecules. Degradation products of elastin have been demonstrated to activate cellular processes such as phagocytosis. These cell interacting domains are interspaced with stretches of alanine that produce the flexible stretchy structure that is characteristic of elastin. The stretches of alanine are also rich in lysines that, in tissues, get converted to allysine by lysyl oxidase and crosslink together into a fishing net-like structure, making extraction of elastin from animal tissues very difficult which requires the breaking down of the elastin molecule for release. Due to this difficult extraction process, commercially available elastins are only 3-5 kDa in size.

In human natural elastin, there are two sections of the molecule, each of which contains three contiguous repeats of VGVAPG (SEQ ID NO: 34), namely, VGVAPGVGVAPGVGVAPG (SEQ ID NO: 36). The amino acid sequence VGVAPG (SEQ ID NO: 34) has been reported to promote extracellular matrix degradation.

Both collagen and elastin have typically been prepared from animal sources for manufacturing purposes. What is needed in the field is a source of both of these useful proteins that is not prepared from animal sources.

### SUMMARY

In certain embodiments herein are various polypeptides, compositions comprising such polypeptides, and methods of using such polypeptides and/or compositions thereof. In certain embodiments, such polypeptides comprise non-natural and/or recombinant polypeptides, such as comprising one or more amino acid sequence that is truncated relative to a natural (e.g., full-length) collagen or elastin.

In one aspect, a composition comprising a non-naturally occurring recombinant polypeptide such as a human collagen or a truncate thereof, a jellyfish collagen or a truncate thereof, or an elastin or truncate thereof, is provided, further including one or more active ingredient, where the polypeptide and the one or more active ingredient provide a benefit to the skin and/or hair. The one or more active ingredient can be, for example, a retinoid or vitamin A compound, a vitamin C compound, an alpha hydroxy acid, hyaluronic acid, coenzyme Q10, a tea extract, a grape seed extract, niacinamide, an antibacterial or acne preventative, or any combination of two or more of these. The benefit to the skin can be, for example, (i) increasing the firmness, elasticity, brightness, hydration, tactile texture, visual texture, collagen content, or elastin content of skin; (ii) decreasing skin damage, lines or wrinkles present on skin, or erythema or redness of skin; (iii) preventing or treating ultraviolet radiation damage to skin; (iv) promoting the repair of damaged skin; (v) protecting skin cells against the effects of exposure to urban dust; (vi) stimulating collagen production or elastin production in skin; or (vii) any combination of two or more of (i)-(vi). The non-naturally occurring recombinant polypeptide can be a truncated collagen polypeptide or a truncated elastin polypeptide, truncated at the C-terminal end, the N-terminal end, internally truncated, or truncated at both the C-terminal end and the N-terminal end, relative to the naturally occurring collagen or elastin. The non-naturally occurring recombinant polypeptide can be truncated at the C-terminal end by 50 to 800 amino acids, truncated at the N-terminal end by 50 to 800 amino acids, or both. The non-naturally occurring recombinant polypeptide can be internally truncated by 50 to 800 amino acids. The non-naturally occurring recombinant truncated collagen polypeptide can be a human collagen or a jellyfish collagen. The non-naturally occurring recombinant truncated collagen polypeptide can be 50 to 900 amino acids in length, 50 to 800 amino acids in length, 50 to 700 amino acids in length, 50 to 600 amino acids in length, 50 to 500 amino acids in length, 50 to 400 amino acids in length, 50 to 300 amino acids in length, 50 to 200 amino acids in length, or 50 to 100 amino acids in length. The non-naturally occurring recombinant truncated collagen polypeptide can be a polypeptide having a N-terminal truncation of 50 to 650 amino acids and a C-terminal truncation of 50 to 250 amino acids.

In an aspect, the non-naturally occurring recombinant truncated collagen polypeptide can be a human type 21 collagen. The non-naturally occurring recombinant truncated collagen polypeptide can have at least 80% sequence identity relative to SEQ ID NO: 31. The non-naturally occurring recombinant truncated collagen polypeptide can have at least 80% sequence identity to SEQ ID NO: 14 or SEQ ID NO: 16. The non-naturally occurring recombinant truncated collagen polypeptide can comprise SEQ ID NO: 14 or SEQ ID NO: 16. The non-naturally occurring recombinant truncated collagen polypeptide can be a human type 1 collagen.

The non-naturally occurring recombinant truncated collagen polypeptide can have at least 80% sequence identity relative to SEQ ID NO: 32. The non-naturally occurring recombinant truncated collagen polypeptide can have at least about 80% sequence identity to SEQ ID NO: 18, SEQ ID NO: 20, SEQ ID NO: 22, SEQ ID NO: 24, SEQ ID NO: 25, SEQ ID NO: 27, or SEQ ID NO: 29. The non-naturally occurring recombinant truncated collagen polypeptide can comprise an amino acid sequence of SEQ ID NO: 18, SEQ ID NO: 20, SEQ ID NO: 22, SEQ ID NO: 24, SEQ ID NO: 25, SEQ ID NO: 27, or SEQ ID NO: 29.

The non-naturally occurring recombinant truncated collagen polypeptide can be a jellyfish collagen. The non-naturally occurring recombinant truncated collagen polypeptide can be a polypeptide comprising an amino acid sequence having at least 80% sequence identity relative to SEQ ID NO: 33. The non-naturally occurring recombinant truncated collagen polypeptide can be an amino acid sequence having at least 80% sequence identity relative to SEQ ID NO: 2, SEQ ID NO: 4, SEQ ID NO: 5, SEQ ID NO: 7, SEQ ID NO: 10, or SEQ ID NO: 12. The non-naturally occurring recombinant truncated collagen polypeptide can comprise SEQ ID NO: 2, SEQ ID NO: 4, SEQ ID NO: 5, SEQ ID NO: 7, SEQ ID NO: 10, or SEQ ID NO: 12. The non-naturally occurring recombinant truncated collagen polypeptide can have at least 80% sequence identity to SEQ ID NO: 5.

The non-naturally occurring recombinant truncated elastin polypeptide can be a human elastin. The elastin can be 50 to 700 amino acids in length, 50 to 600 amino acids in length, 50 to 500 amino acids in length, 50 to 400 amino acids in length, 50 to 300 amino acids in length, 50 to 200 amino acids in length, or 50 to 100 amino acids in length. The elastin can have a truncation at the C-terminal end, a truncation at the N-terminal end, an internal truncation, or any combination thereof, relative to a full-length elastin polypeptide. The elastin can have an N-terminal truncation of between 50 and 200 amino acids and a C-terminal truncation of between 50 and 600 amino acids. The elastin can be a polypeptide having at least 80% sequence identity relative to SEQ ID NO: 53. The elastin can have at least 80% sequence identity to an amino acid sequence of SEQ ID NO: 38, SEQ ID NO: 40, SEQ ID NO: 41, SEQ ID NO: 43, SEQ ID NO: 46, SEQ ID NO: 48, SEQ ID NO: 50, or SEQ ID NO: 52. The elastin can have at least 80% sequence identity SEQ ID NO: 52. The elastin can have an amino acid sequence comprising SEQ ID NO: 38, SEQ ID NO: 40, SEQ ID NO: 41, SEQ ID NO: 43, SEQ ID NO: 46, SEQ ID NO: 48, SEQ ID NO: 50, or SEQ ID NO: 52. One or more additional ingredients or carriers, such as water, oil glycereth-8 esters, glycerin, coconut alkanes, hydroxyethyl acrylate/sodium acryloyldimethyl taurate copolymer, pentylene glycol, disodium EDTA, caprylyl glycol, chlorphenesin, phenoxyethanol, or vegetable oil, can be provided.

In an aspect, a formulation is provided. The formulation can be a personal care product formulated for application to the skin and/or hair of a subject, and can have one or more carriers. The carrier can be a topical carrier, such as a liposome, a biodegradable microcapsule, a lotion, a spray, an aerosol, a dusting powder, a biodegradable polymer, mineral oil, triglyceride oil, silicone oil, glycerin, glycerin monostearate, alcohols, emulsifying agents, liquid petroleum, white petrolatum, propylene glycol, polyoxyethylene, polyoxypropylene, wax, sorbitan monostearate, polysorbate, cetyl ester wax, cetearyl alcohol, 2-octyldodecanol, benzyl alcohol, cyclomethicone, cyclopentasiloxane, or water. The formulation can also contain a preservative. The non-naturally occurring recombinant polypeptide or a truncate thereof can be present in the topical formulation in an amount from about 0.001% (w/w) to about 30% (w/w). The non-naturally occurring recombinant polypeptide or a truncate thereof can be present in the topical formulation in an amount from about 0.001% (w/w) to about 10% (w/w). The hyaluronic acid, when present, can be in an amount from about 0.001% (w/w) to about 10% (w/w). The retinol, when present, can be in an amount from about 0.001% (w/w) to about 5% (w/w). The ascorbic acid, when present, can be in an amount from about 0.01% (w/w) to about 50% (w/w). The salicylic acid, when present, can be in an amount from about 0.1% (w/w) to about 50% (w/w). The benzoyl peroxide, when present, can be in an amount from about 0.1% (w/w) to about 20% (w/w). The niacinamide, when present, can be in an amount from about 0.1% (w/w) to about 50% (w/w). The alpha hydroxy acid, when present, can be in an amount from about 0.1% (w/w) to about 50% (w/w).

In an aspect, the formulation can be a personal care product. The personal care product can be, for example, a cosmetic. The personal care product can be a mask, a skin cleaner a cleansing cream, a cleansing lotion, a facial cleanser, a cleansing milk, a cleansing pad, a facial wash, a facial cream, a body cream, a facial moisturizer, a body moisturizer, a facial serum, a facial mask, a body mask, a facial toner, a facial mist, an eye cream, an eye treatment, an exfoliator formula, a lip balm, a lipstick, a hair shampoo, a hair conditioner, a body shampoo, a hair serum, a scalp serum, a hair mist, a hair spray, an eye shadow, a concealer, a mascara, or a color cosmetic. The personal care product can be configured to form a film.

In yet another aspect, a method of providing a benefit to the skin of a subject is provided, by administering one of the above—described personal care products to the skin of the subject. The benefit to the skin can be, for example: (i) increasing the firmness, elasticity, brightness, hydration, tactile texture, visual texture, collagen content, or elastin content of skin; (ii) decreasing skin damage, lines or wrinkles present on skin, or erythema or redness of skin; (iii) preventing or treating ultraviolet radiation damage to skin; (iv) promoting the repair of damaged skin; (v) protecting skin cells against the effects of exposure to urban dust; (vi) stimulating collagen production or elastin production in skin; or (vii) any combination of two or more of (i)-(vi).

In another aspect, a method of providing a benefit to the hair of a subject is provided, by administering one of the above personal care products to the hair of the subject. The benefit to the hair can be, for example: (i) improving the appearance and/or quality of hair; (ii) improving hair growth or thickness; (iii) reducing hair loss; (iv) improving hair tensile strength, hair moisture, hydrophobicity, static control, fiber alignment, shine, wet combability, dry combability, resistance to hair breakage; (v) reducing split ends, frizz; or (vi) any combination of two or more of (i)-(v).

In yet another aspect, a composition comprising a non-naturally occurring recombinant elastin polypeptide or truncate thereof, and a non-naturally occurring recombinant collagen polypeptide or a truncate thereof, is provided. The composition can also contain one or more active ingredients. The active ingredients can be, for example, a retinoid or vitamin A compound, a vitamin C compound, an alpha hydroxy acid, hyaluronic acid, coenzyme Q10, a tea extract, a grape seed extract, niacinamide, an antibacterial or acne preventative, or any combination of two or more thereof.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates an effect of an exemplary polypeptide provided herein (comprising a truncated human collagen amino acid sequence) on collagen type 1 protein secretion in fibroblasts.

FIG. 2A illustrates expression of collagen type 1 mRNA in fibroblasts treated with an exemplary polypeptide provided herein (comprising a truncated human collagen amino acid sequence).

FIG. 2B illustrates expression of elastin mRNA in fibroblasts treated with an exemplary polypeptide provided herein (comprising a truncated human collagen amino acid sequence).

FIG. 2C illustrates expression of fibronectin mRNA in fibroblasts treated with an exemplary polypeptide provided herein (comprising a truncated human collagen amino acid sequence).

FIG. 3 illustrates expression of IL-la in human primary keratinocytes treated with an exemplary polypeptide provided herein (comprising a truncated human collagen amino acid sequence).

FIG. 4 illustrates antioxidant capacity of an exemplary polypeptide provided herein (comprising a truncated human collagen amino acid sequence).

FIG. 5 illustrates viability of UVB-irradiated keratinocytes treated with an exemplary polypeptide provided herein (comprising a truncated human collagen amino acid sequence).

FIG. 6 illustrates skin elasticity after treatment with an exemplary polypeptide provided herein (comprising a truncated human collagen amino acid sequence).

FIG. 7 illustrates skin collagen content after treatment with an exemplary polypeptide provided herein (comprising a truncated human collagen amino acid sequence).

FIG. 8 illustrates quantification of skin redness after treatment with an exemplary polypeptide provided herein (comprising a truncated human collagen amino acid sequence).

FIG. 9 illustrates quantification of skin wrinkles after treatment with an exemplary polypeptide provided herein (comprising a truncated human collagen amino acid sequence).

FIG. 10 illustrates collagen type 1 protein secretion by a human skin tissue model treated with an exemplary polypeptide provided herein (comprising a truncated jellyfish collagen amino acid sequence).

FIG. 11 illustrates UVB-induced TT dimers in keratinocytes with treatment with an exemplary polypeptide provided herein (comprising a truncated jellyfish collagen amino acid sequence).

FIG. 12 illustrates keratinocyte viability after UVB irradiation with treatment with an exemplary polypeptide provided herein (comprising a truncated jellyfish collagen amino acid sequence).

FIG. 13 illustrates cell viability when treated with urban dust and an exemplary polypeptide provided herein (comprising a truncated jellyfish collagen amino acid sequence).

FIG. 14 illustrates relative expression of IL-la induced by UVB after treatment with an exemplary polypeptide provided herein (comprising a truncated jellyfish collagen amino acid sequence).

FIG. 15 illustrates antioxidant capacity of an exemplary polypeptide provided herein (comprising a truncated jellyfish collagen amino acid sequence).

FIG. 16 illustrates skin hydration after treatment with an exemplary polypeptide provided herein (comprising a truncated jellyfish collagen amino acid sequence).

FIG. 17 illustrates skin elasticity after treatment with an exemplary polypeptide provided herein (comprising a truncated jellyfish collagen amino acid sequence).

FIG. 18 illustrates expression of Type 1 Collagen (ng/mL) in human full-thickness skin equivalents (containing human keratinocytes and human fibroblasts) treated with an exemplary polypeptide provided herein (comprising a truncated human type 21 collagen amino acid sequence) in combination with retinol, ascorbic acid, or hyaluronic acid.

FIG. 19 illustrates expression of fibronectin (ng/mL) in human full-thickness skin equivalents (containing human fibroblasts and human keratinocytes) treated with an exemplary polypeptide provided herein (comprising a truncated human type 21 collagen amino acid sequence) in combination with retinol, ascorbic acid, or hyaluronic acid.

FIG. 20 illustrates expression of elastin (ng/mL) in human full-thickness skin equivalents (containing human fibroblasts and human keratinocytes) treated with an exemplary polypeptide provided herein (comprising a truncated human type 21 collagen amino acid sequence) in combination with retinol, ascorbic acid, or hyaluronic acid.

FIG. 21 illustrates expression of Type III Collagen (ng/mL) in human full-thickness skin equivalents (containing human keratinocytes and human fibroblasts) treated with an exemplary polypeptide provided herein (comprising a truncated human type 21 collagen amino acid sequence) in combination with retinol, ascorbic acid, or hyaluronic acid.

FIG. 22 shows a photograph of a protein gel. Lane 1 is the size marker. Lanes 2 and 3 show that the 23 kDa truncated human elastin of Example 21 runs on the gel at the expected molecular weight. Lane 4 is blank with no proteins loaded. Lanes 5 and 6 show that the 13 kDa truncated human elastin of Example 22 runs on the gel at the expected molecular weight.

FIG. 23 depicts an SDS-PAGE protein gel showing high purity and homogeneity of a recombinantly produced polypeptide (comprising a non-naturally occurring truncated elastin amino acid sequence) described herein.

FIG. 24 depicts secretion levels of pro-collagen type I C-peptide after treatment of fibroblasts with an exemplary polypeptide (comprising a non-naturally occurring truncated elastin amino acid sequence) provided herein.

FIG. 25 depicts the anti-oxidative capacity of an exemplary polypeptide (comprising a non-naturally occurring truncated elastin amino acid sequence) provided herein.

FIG. 26 depicts expression levels of mRNA of various anti-oxidant genes after treatment of keratinocytes with an exemplary polypeptide (comprising a non-naturally occurring truncated elastin amino acid sequence) provided herein.

FIG. 27 depicts expression levels of mRNA of various pro-apoptotic genes after treatment of keratinocytes with an exemplary polypeptide (comprising a non-naturally occurring truncated elastin amino acid sequence) provided herein.

FIG. 28 depicts increased elastin production after treatment of keratinocytes with an exemplary polypeptide (comprising a non-naturally occurring truncated elastin amino acid sequence) provided herein.

### DESCRIPTION

In the following description, certain specific details are set forth in order to provide a thorough understanding of various embodiments of the disclosure. However, one skilled in the art will understand that the disclosure may be practiced without these details.

As used herein the term “about” generally refers to ±10%.

As used herein, the singular form “a”, “an” and “the” include plural references unless the context clearly dictates otherwise. For example, the term “a compound” or “at least one compound” may include a plurality of compounds, including mixtures thereof.

Throughout this document, various embodiments of this disclosure may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicated number and a second indicated number and “ranging/ranges from” a first indicated number “to” a second indicated number are used herein interchangeably and are meant to include the first and second indicated numbers and all the fractional and integral numerals there between.

The term “collagen polypeptide” or “collagen-like polypeptide” as used herein refers, in some instances, to a (e.g., monomeric) polypeptide that can associate with one or more collagen or collagen-like polypeptides to form a quaternary structure. Non-limiting examples of a collagen include a human type 21 alpha 1 collagen (e.g., SEQ ID NO: 31), a human type 1, alpha 2 collagen (e.g., SEQ ID NO: 32), and a jellyfish (Hydrozoan) collagen (e.g., SEQ ID NO: 33). In some instances, a collagen can be treated with acid, base, or heat to prepare a gelatin. The quaternary structure of natural collagen is a triple helix, typically composed of three polypeptides, but it should be noted that a “collagen polypeptide”, “collagen-like polypeptide”, “truncated collagen” or a “truncated collagen” provided herein may or may not have such a quaternary structure, and does not necessarily have such a quaternary structure. In certain instances, of the three polypeptides that form natural collagen, two are usually identical and are designated as the alpha chain. The third polypeptide is designated as the beta chain. In certain instances, a typical natural collagen can be designated as AAB, wherein the collagen is composed of two alpha (“A”) strands and one beta (“B”) strand. In certain instances, polypeptides comprising “truncated collagens” provided herein may or may not have such structural elements. The term “collagen polypeptide” or “collagen-like polypeptide” may refer to the alpha chain polypeptide, the beta chain polypeptide, or both the alpha and beta chain polypeptides. The term “procollagen” as used herein generally refers to polypeptides produced by cells that can be processed to naturally occurring collagen.

The term “elastin polypeptide” or “elastin-like polypeptide” as used herein refers, in some instances, to a (e.g., monomeric) polypeptide that can associate with one or more elastin or elastin-like polypeptides and/or can bind to another polypeptide, nucleic acids, polysaccharides, lipids or other molecules. Generally, the terms “elastin polypeptide” or “elastin-like polypeptide” refer to a polypeptide that has one or more functions associated with a naturally occurring elastin. The non-naturally occurring polypeptides provided herein (e.g., a truncated elastin) may be termed “elastin polypeptide” or “elastin-like polypeptide” when they have one or more functions associated with a naturally occurring elastin. The term “tropoelastin” as used herein generally refers to polypeptides that can be further processed (e.g., by cleavage, splicing, etc.) to produce an elastin polypeptide. A non-limiting example of an elastin is a naturally occurring human elastin having an amino acid sequence according to SEQ ID NO: 53.

The term “expression vector” or “vector” as used herein generally refers to a nucleic acid assembly which is capable of directing the expression of the exogenous gene. The expression vector may include a promoter which is operably linked to the exogenous gene, restriction endonuclease sites, nucleic acids that encode one or more selection markers, and other nucleic acids useful in the practice of recombinant technologies.

The term “extracellular matrix” as used herein generally refers to a network of extracellular macromolecules such as elastin, collagen, enzymes, and glycoproteins that provide the scaffolding for cells in multicellular organisms. The extracellular matrix may provide structural components that mediate cell adhesion, cell to cell communication, and other functions.

The term “fibroblast” as used herein generally refers to a cell that synthesizes procollagen and other structural proteins. Fibroblasts are widely distributed in the body and can be found in skin, connective tissue, and other tissues.

The term “fluorescent protein” generally refers to a protein that may be used in genetic engineering technologies as a reporter of expression of an exogenous polynucleotide. The protein when exposed to ultraviolet or blue light fluoresces and emits a bright visible light. Proteins that emit green light include green fluorescent protein (GFP) and proteins that emit red light include red fluorescent protein (RFP).

The term “gelatin” as used herein generally refers to collagen that has been further processed by exposure to acid, base or heat. In some instances, gelatin solutions form reversible gels used in foods, cosmetics, pharmaceuticals, industrial products, medical products, laboratory culture growth media, and many other applications.

The term “gene” as used herein generally refers to a polynucleotide that encodes a specific protein, and which may refer to the coding region alone or may include regulatory sequences preceding (5′ non-coding sequences) and following (3′ non-coding sequences) the coding sequence.

The term “histidine tag” generally refers to a 2-30 contiguous series of histidine residues on a recombinant polypeptide (SEQ ID NO: 54).

The term “host cell” generally refers to a cell that is engineered to express an introduced exogenous polynucleotide.

The term “keratinocyte” generally refers to a cell that produces keratins, tropoelastin, and other cellular components found in the epidermal layer of the skin.

The term “lactamase” as used herein generally refers to enzymes that hydrolyze antibiotics that contain a lactam (cyclic amide) moiety. “Beta-lactamase” or “β-lactamase” are enzymes that hydrolyze antibiotics that contain a β-lactam moiety.

The term “non-naturally occurring” as used herein generally refers to a gene, a polypeptide, or a protein, for example, a collagen or elastin polypeptide, that is not normally found in nature. The non-naturally occurring collagen or elastin polypeptide may be recombinantly produced (e.g., by expression by a recombinant host cell). The non-naturally occurring collagen or elastin polypeptide may be a recombinant polypeptide (e.g., produced by a recombinant host cell). The non-naturally occurring collagen or elastin polypeptide may be truncated. Other non-naturally occurring collagen or elastin polypeptides include chimeric collagen or chimeric elastin.

A chimeric collagen may be a polypeptide wherein one portion of a collagen polypeptide is contiguous with a second portion of a collagen polypeptide. In another embodiment, the non-naturally occurring collagen comprises a fusion polypeptide that includes additional amino acids such as a secretion tag, histidine tag, green fluorescent protein, and/or protease cleavage site, GEK repeats, GDK repeats, and/or beta-lactamase.

Similarly, a chimeric elastin may be a polypeptide wherein one portion of an elastin polypeptide is contiguous with a second portion of an elastin polypeptide. For example, a molecule comprising a portion of a human elastin contiguous with a portion of another human polypeptide may be a chimeric elastin. In another embodiment, the non-naturally occurring elastin comprises a fusion polypeptide that includes additional amino acids such as a secretion tag, histidine tag, green fluorescent protein, and/or protease cleavage site.

In some aspects, a collagen or elastin polypeptide provided herein has a non-naturally occurring amount of hydroxyprolines. In some cases, a collagen or elastin polypeptide provided herein lacks hydroxyprolines. In some cases, a collagen or elastin polypeptide provided herein comprises fewer hydroxyprolines than a naturally-occurring collagen or elastin. Hydroxyprolines include, without limitation, 3-hydroxyproline, 4-hydroxyproline, and 5-hydroxyproline. In some cases, less than about 50% (e.g., less than about 45%, less than about 40%, less than about 35%, less than about 30%, less than about 25%, less than about 20%, less than about 15%, less than about 10%, or less) of the prolines present in the amino acid sequence of collagen or elastin polypeptide provided herein are hydroxyprolines. In some cases, a recombinant collagen or elastin polypeptide as provided herein is recombinantly expressed in a host cell (e.g., bacterial cell) that lacks an enzyme that hydroxylates one or more amino acids (e.g., proline) of the collagen or elastin polypeptide. In some cases, a recombinant collagen or elastin polypeptide as provided herein is recombinantly expressed in a host cell (e.g., bacterial cell) that lacks prolyl 4-hydroxylase and/or prolyl 3-hydroxylase.

In general, disclosure of a collagen or elastin or a truncated collagen or elastin provided herein, such as having a specific amino acid sequence, includes polypeptides having or comprising that precise amino acid sequence and homologs thereof. In some instances, homologs of an amino acid sequence provided herein may have a longer or shorter sequence and may have a substitution of one or more amino acid residue of such amino acid sequence. Such homologs have a specific sequence identity to the recited sequence, such as in an amount provided herein. Sequence identity, such as for the purpose of assessing percent identity, may be measured by any suitable alignment algorithm, including but not limited to the Needleman-Wunsch algorithm (see e.g. the EMBOSS Needle aligner available at www.ebi.ac.uk/Tools/psa/emboss_needle/nucleotide.html, optionally with default settings), the BLAST algorithm (see e.g. the BLAST alignment tool available at blast.ncbi.nlm.nih.gov/Blast.cgi, optionally with default settings), or the Smith-Waterman algorithm (see e.g. the EMBOSS Water aligner available at www.ebi.ac.uk/Tools/psa/emboss water/nucleotide.html, optionally with default settings). Optimal alignment may be assessed using any suitable parameters of a chosen algorithm, including default parameters. In some cases, a non-naturally occurring collagen or elastin may have at least 60%, 65%, 70%, 75%, 80%, 85%, 90%, 95%, 97%, 98%, 99%, or 100% sequence identity to a sequence disclosed herein.

The term “protease cleavage site” generally refers to an amino acid sequence that is cleaved by a specific protease.

The term “secretion tag” or “signal peptide” generally refers to an amino acid sequence that recruits the host cell's cellular machinery to transport an expressed protein to a particular location or cellular organelle of the host cell.

The term “truncated collagen” generally refers to a monomeric polypeptide that is smaller than a full-length collagen wherein one or more portions of the full-length collagen is not present. Collagen polypeptides are truncated at the C-terminal end, the N-terminal end, truncated by removal of internal portion(s) of the full-length collagen polypeptide (e.g., an internal truncation), or truncated at both the C-terminal end and the N-terminal end. In a non-limiting embodiment, a truncated human collagen may comprise an amino acid sequence according to SEQ ID NO: 16, or a homolog thereof. In another non-limiting example, a truncated jellyfish collagen may comprise an amino acid sequence according to SEQ ID NO: 5, or a homolog thereof. Generally, a truncated collagen provided herein may have a function and/or provide a benefit (e.g., as provided herein) similar or substantially similar to that of a natural or a full-length collagen. In some cases, a truncated collagen provided herein may have improved or increased function and/or benefit (e.g., as provided herein) as compared to a natural or a full-length collagen.

Similarly, the term “truncated elastin” generally refers to a monomeric polypeptide that is smaller than a full-length elastin wherein one or more portions of the full-length elastin is not present. Elastin polypeptides may be truncated at the C-terminal end relative to a full-length elastin, the N-terminal end relative to a full-length elastin, truncated by removal of internal portion(s) of the full-length elastin polypeptide (e.g., an internal truncation), or truncated at both the C-terminal end and the N-terminal end relative to a full-length elastin. In a non-limiting embodiment, a truncated human elastin may comprise an amino acid sequence according to SEQ ID NO: 53, or a homolog thereof. Generally, a truncated elastin provided herein may have a function and/or provide a benefit (e.g., as provided herein) similar or substantially similar to that of a natural or a full-length elastin. In some cases, a truncated elastin provided herein may have improved or increased function and/or benefit (e.g., as provided herein) as compared to a natural or a full-length elastin.

When used in reference to an amino acid position, a “truncation” is inclusive of said amino acid position. For example, an N-terminal truncation at amino acid position 100 of a full-length protein means a truncation of 100 amino acids from the N-terminus of the full-length protein (i.e., the truncated protein is missing amino acid positions 1 through 100 of the full-length protein). Similarly, a C-terminal truncation at amino acid position 901 of a full-length protein (assuming a 1000 amino acid full-length protein) means a truncation of 100 amino acids from the C-terminus (i.e., the truncated protein is missing amino acid positions 901 through 1000 of the full-length protein). Similarly, an internal truncation at amino acid positions 101 and 200 means an internal truncation of 100 amino acids of the full-length protein (i.e., the truncated protein is missing amino acid positions 101 to 200 of the full-length protein).

In some embodiments, the cell culture may comprise one or more of: ammonium chloride, ammonium sulfate, calcium chloride, amino acids, iron(II) sulfate, magnesium sulfate, peptone, potassium phosphate, sodium chloride, sodium phosphate, and yeast extract.

The host bacterial cell may be cultured continuously or discontinuously; in a batch process, a fed-batch process or a repeated fed-batch process.

In general, the signal sequence may be a component of the expression vector, or it may be a part of the exogenous gene that is inserted into the vector. The signal sequence selected may be one that is recognized and processed (e.g., cleaved by a signal peptidase) by the host cell. For bacterial host cells that do not recognize and process the native signal sequence of the exogenous gene, the signal sequence may be substituted by any commonly known bacterial signal sequence. In some embodiments, recombinantly produced polypeptides can be targeted to the periplasmic space using the DsbA signal sequence. Dinh and Bernhardt, J Bacteriol, September 2011, 4984-4987.

In one aspect, a non-naturally occurring collagen that is produced by a host cell is provided. The non-naturally occurring collagen can be a jellyfish collagen or human collagen. The non-naturally occurring collagen may be a truncated collagen. The truncation may be an internal truncation (e.g., a truncation of an internal portion), a truncation at the N-terminal portion of the collagen, a truncation at the C-terminal portion of the collagen, or a truncation at both the C-terminal end and the N-terminal end. The collagen may be truncated by a truncation of from 50 amino acids to 1000 amino acids, from amino acids to 950 amino acids, from 50 amino acids to 900 amino acids, from 50 amino acids to 850 amino acids, from 50 amino acids to 800 amino acids, from 50 amino acids to 850 amino acids, from 50 amino acids to 800 amino acids, from 50 amino acids to 750 amino acids, from 50 amino acids to 700 amino acids, from 50 amino acids to 650 amino acids, from 50 amino acids to 600 amino acids, from 50 amino acids to 650 amino acids, from 50 amino acids to 500 amino acids, from 50 amino acids to 450 amino acids, from 50 amino acids to 400 amino acids, from 50 amino acids to 350 amino acids, from 50 amino acids to 300 amino acids, from 50 amino acids to 250 amino acids, from 50 amino acids to 200 amino acids, from 50 amino acids to 150 amino acids, or from 50 amino acids to 100 amino acids. In another embodiment, the collagen may be truncated by about 50, about 60, about 70, about 80, about 90, about 100, about 110, about 120, about 130, about 140, about 150, about 160, about 170, about 180, about 190, about 200, about 210, about 220, about 230, about 240, about 250, about 260, about 270, about 280, about 290, about 300, about 310, about 320, about 330, about 340, about 350, about 360, about 370, about 380, about 390, about 400, about 410, about 420, about 430, about 440, about 450, about 460, about 470, about 480, about 490, about 500, about 510, about 520, about 530, about 540, about 550, about 560, about 570, about 580, about 590, about 600, about 650, about 700, about 750, about 800, about 850, about 900, about 950, or about 1000 amino acids. The non-naturally occurring collagen may be encoded by a portion of a polynucleotide sequence or the entire polynucleotide sequence disclosed herein.

A truncated collagen disclosed herein may comprise a truncation relative to a full-length collagen. In some embodiments, a truncated collagen disclosed herein may comprise a truncation relative to a full-length human type 21 alpha 1 collagen. In some embodiments, a truncated collagen disclosed herein may comprise a truncation relative to a full-length human type 1 alpha 2 collagen. In some embodiments, a truncated collagen disclosed herein comprise a truncation relative to a full-length jellyfish (Hydrozoan) collagen. Non-limiting examples of full-length collagens are provided in Table 1 below.

TABLE 1
Full-length collagen amino acid sequences

| Collagen | Amino Acid Sequence |
|---|---|
| Human | MAHYITFLCMVLVLLLQNSVLAEDGEVRSSCRTAPTDLVFILDGSYSVGPENFEIVKKWL |
| type 21 | VNITKNFDIGPKFIQVGVVQYSDYPVLEIPLGSYDSGEHLTAAVESILYLGGNTKTGKAI |
| alpha 1 | QFALDYLFAKSSRFLTKIAVVLTDGKSQDDVKDAAQAARDSKITLFAIGVGSETEDAELR |
| collagen | AIANKPSSTYVFYVEDYIAISKIREVMKOKLCEESVCPTRIPVAARDERGFDILLGLDVN |
| | KKVKKRIQLSPKKIKGYEVTSKVDLSELTSNVFPEGLPPSYVFVSTQRFKVKKIWDLWRI |
| | LTIDGRPQIAVTLNGVDKILLFTTTSVINGSQVVTFANPQVKTLFDEGWHQIRLLVTEQD |
| | VTLYIDDQQIENKPLHPVLGILINGQTQIGKYSGKEETVQFDVQKLRIYCDPEQNNRETA |
| | CEIPGFNGECLNGPSDVGSTPAPCICPPGKPGLQGPKGDPGLPGNPGYPGQPGQDGKPGY |
| | QGIAGTPGVPGSPGIQGARGLPGYKGEPGRDGDKGDRGLPGFPGLHGMPGSKGEMGAKGD |
| | KGSPGFYGKKGAKGEKGNAGFPGLPGPAGEPGRHGKDGLMGSPGFKGEAGSPGAPGQDGT |
| | RGEPGIPGFPGNRGLMGQKGEIGPPGQQGKKGAPGMPGLMGSNGSPGQPGTPGSKGSKGE |
| | PGIQGMPGASGLKGEPGATGSPGEPGYMGLPGIQGKKGDKGNQGEKGIQGQKGENGRQGI |
| | PGQQGIQGHHGAKGERGEKGEPGVRGAIGSKGESGVDGLMGPAGPKGQPGDPGPQGPPGL |
| | DGKPGREFSEQFIRQVCTDVIRAQLPVLLQSGRIRNCDHCLSQHGSPGIPGPPGPIGPEG |
| | PRGLPGLPGRDGVPGLVGVPGRPGVRGLKGLPGRNGEKGSQGFGYPGEQGPPGPPGPEGP |
| | PGISKEGPPGDPGLPGKDGDHGKPGIQGQPGPPGICDPSLCFSVIARRDPFRKGPNY |
| | (SEQ ID NO: 31) |
| Human | MLSFVDTRTLLLLAVTLCLATCQSLQEETVRKGPAGDRGPRGERGPPGPPGRDGEDGPTG |
| type 1 | PPGPPGPPGPPGLGGNFAAQYDGKGVGLGPGPMGLMGPRGPPGAAGAPGPQGFQGPAGEP |
| alpha 2 | GEPGQTGPAGARGPAGPPGKAGEDGHPGKPGRPGERGVVGPQGARGFPGTPGLPGFKGIR |
| collagen | GHNGLDGLKGQPGAPGVKGEPGAPGENGTPGQTGARGLPGERGRVGAPGPAGARGSDGSV |
| | GPVGPAGPIGSAGPPGFPGAPGPKGEIGAVGNAGPAGPAGPRGEVGLPGLSGPVGPPGNP |
| | GANGLTGAKGAAGLPGVAGAPGLPGPRGIPGPVGAAGATGARGLVGEPGPAGSKGESGNK |
| | GEPGSAGPQGPPGPSGEEGKRGPNGEAGSAGPPGPPGLRGSPGSRGLPGADGRAGVMGPP |
| | GSRGASGPAGVRGPNGDAGRPGEPGLMGPRGLPGSPGNIGPAGKEGPVGLPGIDGRPGPI |
| | GPAGARGEPGNIGFPGPKGPTGDPGKNGDKGHAGLAGARGAPGPDGNNGAQGPPGPQGVQ |
| | GGKGEQGPPGPPGFQGLPGPSGPAGEVGKPGERGLHGEFGLPGPAGPRGERGPPGESGAA |
| | GPTGPIGSRGPSGPPGPDGNKGEPGVVGAVGTAGPSGPSGLPGERGAAGIPGGKGEKGEP |
| | GLRGEIGNPGRDGARGAPGAVGAPGPAGATGDRGEAGAAGPAGPAGPRGSPGERGEVGPA |
| | GPNGFAGPAGAAGQPGAKGERGAKGPKGENGVVGPTGPVGAAGPAGPNGPPGPAGSRGDG |
| | GPPGMTGFPGAAGRTGPPGPSGISGPPGPPGPAGKEGLRGPRGDQGPVGRTGEVGAVGPP |
| | GFAGEKGPSGEAGTAGPPGTPGPQGLLGAPGILGLPGSRGERGLPGVAGAVGEPGPLGIA |
| | GPPGARGPPGAVGSPGVNGAPGEAGRDGNPGNDGPPGRDGQPGHKGERGYPGNIGPVGAA |
| | GAPGPHGPVGPAGKHGNRGETGPSGPVGPAGAVGPRGPSGPQGIRGDKGEPGEKGPRGLP |
| | GLKGHNGLQGLPGIAGHHGDQGAPGSVGPAGPRGPAGPSGPAGKDGRTGHPGTVGPAGIR |
| | GPQGHQGPAGPPGPPGPPGPPGVSGGGYDFGYDGDFYRADQPRSAPSLRPKDYEVDATLK |
| | ETCIRAQPENIPAKNWYRSSKDKKHVWLGETINAGSQFEYNVEGVTSKEMATQLAFMRLL |
| | ANYASQNITYHCKNSIAYMDEETGNLKKAVILQGSNDVELVAEGNSRFTYTVLVDGCSKK |
| | TNEWGKTIIEYKTNKPSRLPFLDIAPLDIGGADQEFFVDIGPVCFK |
| | (SEQ ID NO: 32) |
| Jellyfish | GPQGVVGADGKDGTPGEKGEQGRTGAAGKQGSPGADGARGPLGSIGQQGARGEPGDPGSP |
| (Hydrozoan) | GLRGDTGLAGVKGVAGPSGRPGQPGANGLPGVNGRGGLRGKPGAKGIAGSDGEAGESGAP |
| collagen | GQSGPTGPRGQRGPSGEDGNPGLQGLPGSDGEPGEEGQPGRSGQPGQQGPRGSPGEVGPR |
| | GSKGPSGDRGDRGERGVPGQTGSAGNVGEDGEQGGKGVDGASGPSGALGARGPPGSRGDT |
| | GAVGPPGPTGRSGLPGNAGQKGPSGEPGSPGKAGSAGEQGPPGKDGSNGEPGSPGKEGER |
| | GLAGPPGPDGRRGETGSPGIAGALGKPGLEGPKGYPGLRGRDGTNGKRGEQGETGPDGVR |
| | GIPGNDGQSGKPGIDGIDGTNGQPGEAGYQGGRGTRGQLGETGDVGQNGDRGAPGPDGSK |
| | GSAGRPGLR (SEQ ID NO: 33) |

In some cases, a truncated collagen as described herein may comprise an N-terminal truncation at any amino acid position from amino acid positions 1 to 548; from amino acid positions 1 to 553; from amino acid positions 1 to 558; from amino acid positions 1 to 563; from amino acid positions 1 to 568; or from amino acid positions 1 to 573 of SEQ ID NO: 31. In some cases, a truncated collagen as described herein may comprise a C-terminal truncation at any amino acid position from amino acid positions 726 to 957; from amino acid positions 731 to 957; from amino acid positions 736 to 957; from amino acid positions 741 to 957; from amino acid positions 746 to 957; from amino acid positions 751 to 957; or from amino acid positions 756 to 957 of SEQ ID NO: 31. In some cases, a truncated collagen as described herein may comprise both an N-terminal truncation and a C-terminal truncation. For example, a truncated collagen as described herein may comprise an N-terminal truncation at any amino acid position from amino acid positions 1 to 548; from amino acid positions 1 to 553; from amino acid positions 1 to 558; from amino acid positions 1 to 563; from amino acid positions 1 to 568; or from amino acid positions 1 to 573 of SEQ ID NO: 31; and a C-terminal truncation at any amino acid position from amino acid positions 726 to 957; from amino acid positions 731 to 957; from amino acid positions 736 to 957; from amino acid positions 741 to 957; from amino acid positions 746 to 957; from amino acid positions 751 to 957; or from amino acid positions 756 to 957. In a specific embodiment, a truncated collagen disclosed herein may comprise an N-terminal truncation at amino acid position 558 of SEQ ID NO: 31; and a C-terminal truncation at amino acid position 746 of SEQ ID NO: 31.

In some cases, a truncated collagen as described herein may comprise an N-terminal truncation at any amino acid position from amino acid positions 1 to 401; from amino acid positions 1 to 406; from amino acid positions 1 to 411; from amino acid positions 1 to 416; from amino acid positions 1 to 421; from amino acid positions 1 to 426; or from amino acid positions 1 to 431 of SEQ ID NO: 32. In some cases, a truncated collagen as described herein may comprise a C-terminal truncation at any amino acid position from amino acid positions 585 to 1366; from amino acid positions 590 to 1366; from amino acid positions 595 to 1366; from amino acid positions 600 to 1366; from amino acid positions 605 to 1366; from amino acid positions 610 to 1366; from amino acid positions 615 to 1366; or from amino acid positions 620 to 1366 of SEQ ID NO: 32. In some cases, a truncated collagen as described herein may comprise both an N-terminal truncation and a C-terminal truncation. For example, a truncated collagen as described herein may comprise an N-terminal truncation at any amino acid position from amino acid positions 1 to 401; from amino acid positions 1 to 406; from amino acid positions 1 to 411; from amino acid positions 1 to 416; from amino acid positions 1 to 421; from amino acid positions 1 to 426; or from amino acid positions 1 to 431 of SEQ ID NO: 32; and a C-terminal truncation at any amino acid position from amino acid positions 585 to 1366; from amino acid positions 590 to 1366; from amino acid positions 595 to 1366; from amino acid positions 600 to 1366; from amino acid positions 605 to 1366; from amino acid positions 610 to 1366; from amino acid positions 615 to 1366; or from amino acid positions 620 to 1366 of SEQ ID NO: 32. In a specific embodiment, a truncated collagen as provided herein may comprise an N-terminal truncation at amino acid position 416 of SEQ ID NO: 32; and a C-terminal truncation at amino acid position 605 of SEQ ID NO: 32.

In some cases, a truncated collagen as described herein may comprise an N-terminal truncation at any amino acid position from amino acid positions 1 to 101; from amino acid positions 1 to 106; from amino acid positions 1 to 111; from amino acid positions 1 to 116; from amino acid positions 1 to 121; or from amino acid positions 1 to 126 of SEQ ID NO: 32. In some cases, a truncated collagen as described herein may comprise a C-terminal truncation at any amino acid position from amino acid positions 276 and 1366; from amino acid positions 281 to 1366; from amino acid positions 286 to 1366; from amino acid positions 291 to 1366; from amino acid positions 296 to 1366; from amino acid positions 301 to 1366; or from amino acid positions 306 to 1366 of SEQ ID NO: 32. In some cases, a truncated collagen as described herein may comprise both an N-terminal truncation and a C-terminal truncation. For example, a truncated collagen as described herein may comprise an N-terminal truncation at any amino acid position from amino acid positions 1 to 101; from amino acid positions 1 to 106; from amino acid positions 1 to 111; from amino acid positions 1 to 116; from amino acid positions 1 to 121; or from amino acid positions 1 to 126 of SEQ ID NO: 32; and a C-terminal truncation at any amino acid position from amino acid positions 276 to 1366; from amino acid positions 281 to 1366; from amino acid positions 286 to 1366; from amino acid positions 291 to 1366; from amino acid positions 296 to 1366; from amino acid positions 301 to 1366; or from amino acid positions 306 to 1366 of SEQ ID NO: 32. In a specific embodiment, a truncated collagen as provided herein may comprise an N-terminal truncation at amino acid position 111 of SEQ ID NO: 32; and a C-terminal truncation at amino acid position 291 of SEQ ID NO: 32.

In some cases, a truncated collagen as described herein may comprise an internal truncation at any amino acid position from amino acid positions 16 to 240; from amino acid positions 16 to 245; from amino acid positions 16 to 250; from amino acid positions 16 to 255; from amino acid positions 16 to 260; from amino acid positions 16 to 265; from amino acid positions 6 to 255; from amino acid positions 11 to 255; from amino acid positions 21 to 255; from amino acid positions 26 to 255; from amino acid positions 31 to 255; from amino acid positions 21 to 250; from amino acid positions 21 to 245; from amino acid positions 26 to 250; from amino acid positions 26 to 245; from amino acid positions 31 to 250; or from amino acid positions 31 to 245 of SEQ ID NO: 33. In a specific embodiment, a truncated collagen as described herein may comprise an internal truncation at amino acid positions 16 and 255 of SEQ ID NO: 33.

In some cases, a truncated collagen may comprise any amino acid sequence provided in Table 2 below. In some cases, a truncated collagen may consist of any amino acid sequence provided in Table 2 below. In some cases, a truncated collagen may consist essentially of any amino acid sequence provided in Table 2 below. In specific embodiments, the non-naturally occurring collagen is or comprises an amino acid sequence of any one of SEQ ID NO: 2, SEQ ID NO: 4, SEQ ID NO: 5, SEQ ID NO: 7, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 25, SEQ ID NO: 27, and SEQ ID NO: 29. In some embodiments, the truncated collagen comprises an amino acid sequence having at least 85%, at least 90%, at least 95%, or at least 98% sequence identity to any one of SEQ ID NO: 2, SEQ ID NO: 4, SEQ ID NO: 5, SEQ ID NO: 7, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 25, SEQ ID NO: 27, and SEQ ID NO: 29.

TABLE 2
Non-limiting examples of truncated collagens

| SEQ ID NO: | Amino acid sequence |
|---|---|
| SEQ ID NO: 2 | MKKIWLALAGLVLAFSASAAQYEDHHHHHHHHHSGSSLVPRGSHM |
| | GPQGVVGADGKDGTPGEKGEQGRTGAAGKQGSPGADGARGPLGSI |
| | GQQGARGEPGDPGSPGLRGDTGLAGVKGVAGPSGRPGQPGANGLP |
| | GVNGRGGLERGLAGPPGPDGRRGETGSPGIAGALGKPGLEGPKGY |
| | PGLRGRDGTNGKRGEQGETGPDGVRGIPGNDGQSGKPGIDGIDGT |
| | NGQPGEAGYQGGRGTRGQLGETGDVGQNGDRGAPGPDGSKGSAGR |
| | PGLR |
| SEQ ID NO: 4 | GPQGVVGADGKDGTPGEKGEQGRTGAAGKQGSPGADGARGPLGSI |
| | GQQGARGEPGDPGSPGLRGDTGLAGVKGVAGPSGRPGQPGANGLP |
| | GVNGRGGLERGLAGPPGPDGRRGETGSPGIAGALGKPGLEGPKGY |
| | PGLRGRDGTNGKRGEQGETGPDGVRGIPGNDGQSGKPGIDGIDGT |
| | NGQPGEAGYQGGRGTRGQLGETGDVGQNGDRGAPGPDGSKGSAGR |
| | PGLR |
| SEQ ID NO: 7 | MKKIWLALAGLVLAFSASAAQYEDGPQGVVGADGKDGTPGNAGQK |
| | GPSGEPGSPGKAGSAGEQGPPGKDGSNGEPGSPGKEGERGLAGPP |
| | GPDGRRGETGSPGIAGALGKPGLEGPKGYPGLRGRDGTNGKRGEQ |
| | GETGPDGVRGIPGNDGQSGKPGIDGIDGTNGQPGEAGYQGGRGTR |
| | GOLGETGDVGONGDRGAPGPDGSKGSAGRPGLR |
| SEQ ID NO: 5 | GPQGVVGADGKDGTPGNAGQKGPSGEPGSPGKAGSAGEQGPPGKD |
| | GSNGEPGSPGKEGERGLAGPPGPDGRRGETGSPGIAGALGKPGLE |
| | GPKGYPGLRGRDGTNGKRGEQGETGPDGVRGIPGNDGQSGKPGID |
| | GIDGTNGQPGEAGYQGGRGTRGQLGETGDVGQNGDRGAPGPDGSK |
| | GSAGRPGLR |
| SEQ ID NO: 10 | MKKIWLALAGLVLAFSASAAQYEDHHHHHHHHHSGSSLVPRGSHM |
| | SGSSSKGEELFTGVVPILVELDGDVNGHKFSVRGEGEGDATNGKL |
| | TLKFICTTGKLPVPWPTLVTTLTYGVQCFSRYPDHMKQHDFFKSA |
| | MPEGYVQERTISFKDDGTYKTRAEVKFEGDTLVNRIELKGIDFKE |
| | DGNILGHKLEYNFNSHNVYITADKQKNGIKANFKIRHNVEDGSVQ |
| | LADHYQQNTPIGDGPVLLPDNHYLSTQSKLSKDPNEKRDHMVLLE |
| | FVTAAGITHGMDELYKSGAPGGPQGVVGADGKDGTPGNAGQKGPS |
| | GEPGSPGKAGSAGEQGPPGKDGSNGEPGSPGKEGERGLAGPPGPD |
| | GRRGETGSPGIAGALGKPGLEGPKGYPGLRGRDGTNGKRGEQGET |
| | GPDGVRGIPGNDGQSGKPGIDGIDGTNGQPGEAGYQGGRGTRGQL |
| | GETGDVGONGDRGAPGPDGSKGSAGRPGLRHPETLVKVKDAEDQL |
| | GARVGYIELDLNSGKILESFRPEERFPMMSTFKVLLCGAVLSRID |
| | AGQEQLGRRIHYSQNDLVEYSPVTEKHLTDGMTVRELCSAAITMS |
| | DNTAANLLLTTIGGPKELTAFLHNMGDHVTRLDRWEPELNEAIPN |
| | DERDTTMPVAMATTLRKLLTGELLTLASRQQLIDWMEADKVAGPL |
| | LRSALPAGWFIADKSGAGERGSRGIIAALGPDGKPSRIVVIYTTG |
| | SQATMDERNRQIAEIGASLIKHW |
| SEQ ID NO: 12 | MKKIWLALAGLVLAFSASAAQYEDHHHHHHHHHSGSSLVPRGSHM |
| | SGSSSKGEELFTGVVPILVELDGDVNGHKFSVRGEGEGDATNGKL |
| | TLKFICTTGKLPVPWPTLVTTLTYGVQCFSRYPDHMKQHDFFKSA |
| | MPEGYVQERTISFKDDGTYKTRAEVKFEGDTLVNRIELKGIDFKE |
| | DGNILGHKLEYNFNSHNVYITADKQKNGIKANFKIRHNVEDGSVQ |
| | LADHYQQNTPIGDGPVLLPDNHYLSTQSKLSKDPNEKRDHMVLLE |
| | FVTAAGITHGMDELYKSGAPGGPQGVVGADGKDGTPGNAGQKGPS |
| | GEPGSPGKAGSAGEQGPPGKDGSNGEPGSPGKEGERGLAGPPGPD |
| | GRRGETGSPGIAGALGKPGLEGPKGYPGLRGRDGTNGKRGEQGET |
| | GPDGVRGIPGNDGQSGKPGIDGIDGTNGQPGEAGYQGGRGTRGQL |
| | GETGDVGONGDRGAPGPDGSKGSAGRPGLRHPETLVKVKDAEDQL |
| | GARVGYIELDLNSGKILESFRPEERFPMMSTFKVLLCGAVLSRID |
| | AGQEQLGRRIHYSQNDLVEYSPVTEKHLTDGMTVRELCSAAITMS |
| | DNTAANLLLTTIGGPKELTAFLHNMGDHVTRLDRWEPELNEAIPN |
| | DERDTTMPVAMATTLRKLLTGELLTLASRQQLIDWMEADKVAGPL |
| | LRSALPAGWFIADKSGAGERGSRGIIAALGPDGKPSRIVVIYTTG |
| | SQATMDERNRQIAEIGASLIKHW |
| SEQ ID NO: 14 | MKKIWLALAGLVLAFSASAAQYEDAGFPGLPGPAGEPGRHGKDGL |
| | MGSPGFKGEAGSPGAPGQDGTRGEPGIPGFPGNRGLMGQKGEIGP |
| | PGQQGKKGAPGMPGLMGSNGSPGQPGTPGSKGSKGEPGIQGMPGA |
| | SGLKGEPGATGSPGEPGYMGLPGIQGKKGDKGNQGEKGIQGQKGE |
| | NGRQGIPGQQGIQGHHGAKGERGEKGEPGVR |
| SEQ ID NO: 16 | AGFPGLPGPAGEPGRHGKDGLMGSPGFKGEAGSPGAPGQDGTRGE |
| | PGIPGFPGNRGLMGQKGEIGPPGQQGKKGAPGMPGLMGSNGSPGQ |
| | PGTPGSKGSKGEPGIQGMPGASGLKGEPGATGSPGEPGYMGLPGI |
| | QGKKGDKGNQGEKGIQGQKGENGRQGIPGQQGIQGHHGAKGERGE |
| | KGEPGVR |
| SEQ ID NO: 18 | MKKIWLALAGLVLAFSASAAQYEDMGPPGSRGASGPAGVRGPNGD |
| | AGRPGEPGLMGPRGLPGSPGNIGPAGKEGPVGLPGIDGRPGPIGP |
| | AGARGEPGNIGFPGPKGPTGDPGKNGDKGHAGLAGARGAPGPDGN |
| | NGAQGPPGPQGVQGGKGEQGPAGPPGFQGLPGPSGPAGEVGKPGE |
| | RGLHGEFGLPGPAGPRGERGPPGESGAAGPTG |
| SEQ ID NO: 20 | MGPPGSRGASGPAGVRGPNGDAGRPGEPGLMGPRGLPGSPGNIGP |
| | AGKEGPVGLPGIDGRPGPIGPAGARGEPGNIGFPGPKGPTGDPGK |
| | NGDKGHAGLAGARGAPGPDGNNGAQGPPGPQGVQGGKGEQGPAGP |
| | PGFQGLPGPSGPAGEVGKPGERGLHGEFGLPGPAGPRGERGPPGE |
| | SGAAGPTG |
| SEQ ID NO: 22 | MKKIWLALAGLVLAFSASAAQYEDGFQGPAGEPGEPGQTGPAGAR |
| | GPAGPPGKAGEDGHPGKPGRPGERGVVGPQGARGFPGTPGLPGFK |
| | GIRGHNGLDGLKGQPGAPGVKGEPGAPGENGTPGQTGARGLPGER |
| | GRVGAPGPAGARGSDGSVGPVGPAGPIGSAGPPGFPGAPGPKGEI |
| | GAVGNAGPAGPAGPRGEVGLPGL |
| SEQ ID NO: 24 | GFQGPAGEPGEPGQTGPAGARGPAGPPGKAGEDGHPGKPGRPGER |
| | GVVGPQGARGFPGTPGLPGFKGIRGHNGLDGLKGQPGAPGVKGEP |
| | GAPGENGTPGQTGARGLPGERGRVGAPGPAGARGSDGSVGPVGPA |
| | GPIGSAGPPGFPGAPGPKGEIGAVGNAGPAGPAGPRGEVGLPGL |
| SEQ ID NO: 25 | MKKIWLALAGLVLAFSASAGDOGPVGRTGEVGAVGPPGFAGEKGP |
| | SGEAGTAGPPGTPGPQGLLGAPGILGLPGSRGERGLPGVAGAVGE |
| | PGPLGIAGPPGARGPPGAVGSPGVNGAPGEAGRDGNPGNDGPPGR |
| | DGQPGHKGERGYPGNIGPVGAAGAPGPHGPVGPAGKHGNRGETGP |
| | SGPVGPAGAVGPRGPSGPQGIRGDKGEPGEKGPRGLPGLGDYKDD |
| | DDK |
| SEQ ID NO: 27 | MKKIWLALAGLVLAFSASAKGHNGLQGLPGIAGHHGDQGAPGSVG |
| | PAGPRGPAGPSGPAGKDGRTGHPGTVGPAGIRGPQGHQGPAGPPG |
| | PPGPPGPPGVSGGGYDFGYDGDFYRADQPRSAPSLRPKDYEVDAT |
| | LKSLNNQIETLLTPEGSRKNPARTCRDLRLSHPEWSSGYYWIDPN |
| | QGCTMDAIKVYCDFSTGETCIRAQPENIPAKNWYRSSKDGDYKDD |
| | DDK |
| SEQ ID NO: 29 | MKKIWLALAGLVLAFSASAYEVDATLKSLNNQIETLLTPEGSRKN |
| | PARTCRDLRLSHPEWSSGYYWIDPNQGCTMDAIKVYCDFSTGETC |
| | IRAQPENIPAKNWYRSSKDKKHVWLGETINAGSQFEYNVEGVTSK |
| | EMATQLAFMRLLANYASQNITYHCKNSIAYMDEETGNLKKAVILQ |
| | GSNDVELVAEGNSRFTYTVLVDGCSKKTNEWGKTIIEYKTNKPSR |
| | LPFLDIAPLDIGGADQEFFVDIGPVCFKGDYKDDDDK |

In some cases, a truncated collagen may be from 100 to 300 amino acids, from 150 to 250 amino acids, from 160 to 250 amino acids, from 160 to 220 amino acids, from 170 to 200 amino acids, from 180 to 190 amino acids, or from 185 to 190 amino acids in length.

In one aspect, a non-naturally occurring elastin is provided. In some cases, the non-naturally occurring elastin is a recombinant polypeptide that is produced by a host cell (e.g., a recombinant cell). In some cases, the non-naturally occurring elastin may be a human elastin. In some cases, the non-naturally occurring elastin may be a truncated elastin. The truncated elastin may be truncated relative to a full-length elastin (e.g., having an amino acid sequence according to SEQ ID NO: 53). The truncation may be an internal truncation relative to a full-length elastin, a truncation at the N-terminal portion relative to a full-length elastin, a truncation at the C-terminal portion relative to a full-length elastin, or a truncation at both the C-terminal end and the N-terminal end relative to a full-length elastin.

The truncated elastin may have a truncation of from 10 to 700 amino acids in length, from 10 to 600 amino acids in length, from 10 to 500 amino acids in length, from 10 to 400 amino acids in length, from 10 to 300 amino acids in length, from 10 to 200 amino acids in length, from 10 to 100 amino acids in length, from 10 to 50 amino acids in length, from 50 to 800 amino acids in length, from 50 to 700 amino acids in length, from 50 to 600 amino acids in length, from 50 to 500 amino acids in length, from 50 to 400 amino acids in length, from 50 to 300 amino acids in length, from 50 to 200 amino acids in length, or from 50 to 100 amino acids in length, relative to a full-length elastin. In another embodiment, a truncated elastin may be truncated by about 50, about 60, about 70, about 80, about 90, about 100, about 110, about 120, about 130, about 140, about 150, about 160, about 170, about 180, about 190, about 200, about 210, about 220, about 230, about 240, about 250, about 260, about 270, about 280, about 290, about 300, about 310, about 320, about 330, about 340, about 350, about 360, about 370, about 380, about 390, about 400, about 410, about 420, about 430, about 440, about 450, about 460, about 470, about 480, about 490, about 500, about 510, about 520, about 530, about 540, about 550, about 560, about 570, about 580, about 590, about 600, about 650, about 700, or about 750 amino acids relative to a full-length elastin. The truncated elastin may be encoded by a portion of a polynucleotide sequence or the entire polynucleotide sequence disclosed herein.

In some embodiments, a truncated elastin (e.g., amino acid sequence thereof) (e.g., of a polypeptide provided herein) may be truncated at the C-terminal end (relative to a full-length elastin) by any suitable number of amino acid residues, such as up to about 10, about 10 to about 800, about 10 to about 700, about 10 to about 500, about 10 to about 400, about 10 to about 300, about 10 to about 200, about 10 to about 100, about 50 to about 800, about 50 to about 700, about 50 to about 600, about 50 to about 500, about 50 to about 400, about 50 to about 300, about 50 to about 200, about 50 to about 100, or the like. In some cases, a truncated elastin may be truncated at the C-terminal end (relative to a full-length elastin) by about 10, about 20, about 30, about 40, about 50, about 60, about 70, about 80, about 90, about 100, about 110, about 120, about 130, about 140, about 150, about 160, about 170, about 180, about 190, about 200, about 210, about 220, about 230, about 240, about 250, about 260, about 270, about 280, about 290, about 300, about 310, about 320, about 330, about 340, about 350, about 360, about 370, about 380, about 390, about 400, about 410, about 420, about 430, about 440, about 450, about 460, about 470, about 480, about 490, about 500, about 510, about 520, about 530, about 540, about 550, about 560, about 570, about 580, about 590, about 600, about 610, about 620, about 630, about 640, about 650, about 660, about 670, about 680, about 690, about 700, about 710, about 720, about 730, about 740, about 750, about 760, about 770, about 780, about 790, about 800 or more amino acids.

In some embodiments, a truncated elastin (e.g., amino acid sequence thereof) (e.g., of a polypeptide provided herein) may be truncated at the N-terminal end (relative to a full-length elastin) by any suitable number of amino acid residues, such as up to 10, 10 to 800, 10 to 700, 10 to 500, 10 to 400, 10 to 300, 10 to 200, 10 to 100, 50 to 800, 50 to 700, 50 to 600, 50 to 500, 50 to 400, 50 to 300, 50 to 200, 50 to 100, or the like. In some cases, a truncated elastin may be truncated at the N-terminal end (relative to a full-length elastin) by about 10, about 20, about 30, about 40, about 50, about 60, about 70, about 80, about 90, about 100, about 110, about 120, about 130, about 140, about 150, about 160, about 170, about 180, about 190, about 200, about 210, about 220, about 230, about 240, about 250, about 260, about 270, about 280, about 290, about 300, about 310, about 320, about 330, about 340, about 350, about 360, about 370, about 380, about 390, about 400, about 410, about 420, about 430, about 440, about 450, about 460, about 470, about 480, about 490, about 500, about 510, about 520, about 530, about 540, about 550, about 560, about 570, about 580, about 590, about 600, about 610, about 620, about 630, about 640, about 650, about 660, about 670, about 680, about 690, about 700, about 710, about 720, about 730, about 740, about 750, about 760, about 770, about 780, about 790, about 800 or more amino acids.

In some embodiments, a truncated elastin (e.g., amino acid sequence thereof) (e.g., of a polypeptide provided herein) may be truncated at both the N-terminal end and the C-terminal end relative to a full-length elastin. In some instances, a truncated elastin may be truncated at the N-terminal end (relative to a full-length elastin) by any suitable number of amino acid residues, such as up to 10, 10 to 800, 10 to 700, 10 to 500, 10 to 400, 10 to 300, 10 to 200, 10 to 100, 50 to 800, 50 to 700, 50 to 600, 50 to 500, 50 to 400, 50 to 300, 50 to 200, 50 to 100, or the like; and may be truncated at the C-terminal end (relative to a full-length elastin) by any suitable number of amino acid residues, such as up to 10, 10 to 800, 10 to 700, 10 to 500, 10 to 400, 10 to 300, 10 to 200, 10 to 100, 50 to 800, 50 to 700, 50 to 600, 50 to 500, 50 to 400, 50 to 300, 50 to 200, 50 to 100, or the like. In some cases, a truncated elastin may be truncated at the N-terminal end (relative to a full-length elastin) by about 10, about 20, about 30, about 40, about 50, about 60, about 70, about 80, about 90, about 100, about 110, about 120, about 130, about 140, about 150, about 160, about 170, about 180, about 190, about 200, about 210, about 220, about 230, about 240, about 250, about 260, about 270, about 280, about 290, about 300, about 310, about 320, about 330, about 340, about 350, about 360, about 370, about 380, about 390, about 400, about 410, about 420, about 430, about 440, about 450, about 460, about 470, about 480, about 490, about 500, about 510, about 520, about 530, about 540, about 550, about 560, about 570, about 580, about 590, about 600, about 610, about 620, about 630, about 640, about 650, about 660, about 670, about 680, about 690, about 700, about 710, about 720, about 730, about 740, about 750, about 760, about 770, about 780, about 790, about 800 or more amino acids; and may be truncated at the C-terminal end (relative to a full-length elastin) by about 10, about 20, about 30, about 40, about 50, about 60, about 70, about 80, about 90, about 100, about 110, about 120, about 130, about 140, about 150, about 160, about 170, about 180, about 190, about 200, about 210, about 220, about 230, about 240, about 250, about 260, about 270, about 280, about 290, about 300, about 310, about 320, about 330, about 340, about 350, about 360, about 370, about 380, about 390, about 400, about 410, about 420, about 430, about 440, about 450, about 460, about 470, about 480, about 490, about 500, about 510, about 520, about 530, about 540, about 550, about 560, about 570, about 580, about 590, about 600, about 610, about 620, about 630, about 640, about 650, about 660, about 670, about 680, about 690, about 700, about 710, about 720, about 730, about 740, about 750, about 760, about 770, about 780, about 790, about 800 or more amino acids.

In some embodiments, a truncated elastin (e.g., amino acid sequence thereof) (e.g., of a polypeptide provided herein) may be internally truncated (relative to a full-length elastin) by any suitable number of amino acid residues, such as up to 10, 10 to 800, 10 to 700, 10 to 500, 10 to 400, 10 to 300, 10 to 200, 10 to 100, 50 to 800, 50 to 700, 50 to 600, 50 to 500, 50 to 400, 50 to 300, 50 to 200, 50 to 100, or the like. In some cases, a truncated elastin may be internally truncated (relative to a full-length elastin) by about 10, about 20, about 30, about 40, about 50, about 60, about 70, about 80, about 90, about 100, about 110, about 120, about 130, about 140, about 150, about 160, about 170, about 180, about 190, about 200, about 210, about 220, about 230, about 240, about 250, about 260, about 270, about 280, about 290, about 300, about 310, about 320, about 330, about 340, about 350, about 360, about 370, about 380, about 390, about 400, about 410, about 420, about 430, about 440, about 450, about 460, about 470, about 480, about 490, about 500, about 510, about 520, about 530, about 540, about 550, about 560, about 570, about 580, about 590, about 600, about 610, about 620, about 630, about 640, about 650, about 660, about 670, about 680, about 690, about 700, about 710, about 720, about 730, about 740, about 750, about 760, about 770, about 780, about 790, about 800 or more amino acids.

A truncated elastin disclosed herein may comprise a truncation relative to a full-length elastin. In some embodiments, a truncated elastin disclosed herein may comprise a truncation relative to a full-length human elastin. In some cases, a full-length human elastin has an amino acid sequence according to SEQ ID NO: 53 provided in Table 3 below.

TABLE 3
Full-length elastin amino acid sequences

| Elastin | Amino Acid Sequence |
|---|---|
| Human | GGVPGAIPGGVPGGVFYPGAGLGALGGGALGPGGKPL |
| elastin | KPVPGGLAGAGLGAGLGAFPAVTFPGALVPGGVADAA |
| (without | AAYKAAKAGAGLGGVPGVGGLGVSAGAVVPQPGAGVK |
| the | PGKVPGVGLPGVYPGGVLPGARFPGVGVLPGVPTGAG |
| native | VKPKAPGVGGAFAGIPGVGPFGGPQPGVPLGYPIKAP |
| secretion | KLPGGYGLPYTTGKLPYGYGPGGVAGAAGKAGYPTGT |
| tag) | GVGPQAAAAAAAKAAAKFGAGAAGVLPGVGGAGVPGV |
| | PGAIPGIGGIAGVGTPAAAAAAAAAAKAAKYGAAAGL |
| | VPGGPGFGPGVVGVPGAGVPGVGVPGAGIPVVPGAGI |
| | PGAAVPGVVSPEAAAKAAAKAAKYGARPGVGVGGIPT |
| | YGVGAGGFPGFGVGVGGIPGVAGVPGVGGVPGVGGVP |
| | GVGISPEAQAAAAAKAAKYGAAGAGVLGGLVPGPQAA |
| | VPGVPGTGGVPGVGTPAAAAAKAAAKAAQFGLVPGVG |
| | VAPGVGVAPGVGVAPGVGLAPGVGVAPGVGVAPGVGV |
| | APGIGPGGVAAAAKSAAKVAAKAQLRAAAGLGAGIPG |
| | LGVGVGVPGLGVGAGVPGLGVGAGVPGFGAGADEGVR |
| | RSLSPELREGDPSSSQHLPSTPSSPRVPGALAAAKAA |
| | KYGAAVPGVLGGLGALGGVGIPGGVVGAGPAAAAAAA |
| | KAAAKAAQFGLVGAAGLGGLGVGGLGVPGVGGLGGIP |
| | PAAAAKAAKYGAAGLGGVLGGAGQFPLGGVAARPGFG |
| | LSPIFPGGACLGKACGRKRK |
| | (SEQ ID NO: 53) |

In some cases, a truncated elastin as described herein may comprise an N-terminal truncation at any amino acid position from amino acid positions 1 to 68; from amino acid positions 1 to 73; from amino acid positions 1 to 78; from amino acid positions 1 to 83; from amino acid positions 1 to 88; from amino acid positions 1 to 93; or from amino acid positions 1 to 98 of SEQ ID NO: 53. In some cases, a truncated elastin as described herein may comprise a C-terminal truncation at any amino acid position from amino acid positions 213 to 760; from amino acid positions 218 to 760; from amino acid positions 223 to 760; from amino acid positions 228 to 760; from amino acid positions 233 to 760; from amino acid positions 238 to 760; or from 243 to 760 of SEQ ID NO: 53. In some cases, a truncated elastin as described herein may comprise both an N-terminal truncation and a C-terminal truncation. For example, a truncated elastin as described herein may comprise an N-terminal truncation at any amino acid position from amino acid positions 1 to 68; from amino acid positions 1 to 73; from amino acid positions 1 to 78; from amino acid positions 1 to 83; from amino acid positions 1 to 88; from amino acid positions 1 to 93; or from amino acid positions 1 to 98 of SEQ ID NO: 53; and a C-terminal truncation at any amino acid position from amino acid positions 213 to 760; from amino acid positions 218 to 760; from amino acid positions 223 to 760; from amino acid positions 228 to 760; from amino acid positions 233 to 760; from amino acid positions 238 to 760; or from 243 to 760 of SEQ ID NO: 53. In a specific embodiment, a truncated elastin disclosed herein may comprise an N-terminal truncation at amino acid position 83 of SEQ ID NO: 53; and a C-terminal truncation at amino acid position 228 of SEQ ID NO: 53.

In some cases, a truncated elastin as described herein may comprise an N-terminal truncation at any amino acid position from amino acid positions 1 to 68; from amino acid positions 1 to 73; from amino acid positions 1 to 78; from amino acid positions 1 to 83; from amino acid positions 1 to 88; from amino acid positions 1 to 93; or from amino acid positions 1 to 98 of SEQ ID NO: 53. In some cases, a truncated elastin as described herein may comprise a C-terminal truncation at any amino acid position from amino acid positions 331 to 760; from amino acid positions 336 to 760; from amino acid positions 341 to 760; from amino acid positions 346 to 760; from amino acid positions 351 to 760; from amino acid positions 356 to 760; or from amino acid positions 361 to 760 of SEQ ID NO: 53. In some cases, a truncated elastin as described herein may comprise both an N-terminal truncation and a C-terminal truncation. For example, a truncated elastin as described herein may comprise an N-terminal truncation at any amino acid position from amino acid positions 1 to 68; from amino acid positions 1 to 73; from amino acid positions 1 to 78; from amino acid positions 1 to 83; from amino acid positions 1 to 88; from amino acid positions 1 to 93; or from amino acid positions 1 to 98 of SEQ ID NO: 53; and a C-terminal truncation at any amino acid position from amino acid positions 331 to 760; from amino acid positions 336 to 760; from amino acid positions 341 to 760; from amino acid positions 346 to 760; from amino acid positions 351 to 760; from amino acid positions 356 to 760; or from amino acid positions 361 to 760 of SEQ ID NO: 53. In a specific embodiment, a truncated elastin disclosed herein may comprise an N-terminal truncation at amino acid position 83 of SEQ ID NO: 53; and a C-terminal truncation at amino acid position 346 of SEQ ID NO: 53.

In some cases, a truncated elastin may comprise any amino acid sequence provided in Table 4 below. In some cases, a truncated elastin may consist of any amino acid sequence provided in Table 4 below. In some cases, a truncated elastin may consist essentially of any amino acid sequence provided in Table 4 below. In specific embodiments, the non-naturally occurring elastin is or comprises an amino acid sequence of any one of SEQ ID NO: 38, SEQ ID NO: 40, SEQ ID NO: 41, SEQ ID NO: 43, SEQ ID NO: 46, SEQ ID NO: 48, SEQ ID NO: 50, or SEQ ID NO: 52. In some embodiments, the truncated elastin comprises an amino acid sequence having at least 80%, at least 85%, at least 90%, at least 95%, or at least 98% sequence identity to any one of SEQ ID NO: 38, SEQ ID NO: 40, SEQ ID NO: 41, SEQ ID NO: 43, SEQ ID NO: 46, SEQ ID NO: 48, SEQ ID NO: 50, or SEQ ID NO: 52.

In some embodiments, the truncated elastin may be a truncate (e.g., a polypeptide having at least 50, at least 60, at least 70, at least 80, at least 90, at least 100, at least 110, at least 120, at least 130, at least 140, or at least 150 amino acids) of any one of SEQ ID NO: 38, SEQ ID NO: 40, SEQ ID NO: 41, SEQ ID NO: 43, SEQ ID NO: 46, SEQ ID NO: 48, SEQ ID NO: 50, or SEQ ID NO: 52; or may be a truncate (e.g., a polypeptide having at least 50, at least 60, at least 70, at least 80, at least 90, at least 100, at least 110, at least 120, at least 130, at least 140, or at least 150 amino acids) of an amino acid sequence having at least 80%, at least 85%, at least 90%, at least 95%, or at least 98% sequence identity to any one of SEQ ID NO: 38, SEQ ID NO: 40, SEQ ID NO: 41, SEQ ID NO: 43, SEQ ID NO: 46, SEQ ID NO: 48, SEQ ID NO: 50, or SEQ ID NO: 52. For example, the truncated elastin may have an N-terminal truncation, a C-terminal truncation, and/or an internal truncation relative to any one of SEQ ID NO: 38, SEQ ID NO: 40, SEQ ID NO: 41, SEQ ID NO: 43, SEQ ID NO: 46, SEQ ID NO: 48, SEQ ID NO: 50, or SEQ ID NO: 52; or an N-terminal truncation, a C-terminal truncation, and/or an internal truncation relative to an amino acid sequence having at least 80%, at least 85%, at least 90%, at least 95%, or at least 98% sequence identity to any one of SEQ ID NO: 38, SEQ ID NO: 40, SEQ ID NO: 41, SEQ ID NO: 43, SEQ ID NO: 46, SEQ ID NO: 48, SEQ ID NO: 50, or SEQ ID NO: 52.

TABLE 4
Non-limiting examples of truncated elastins

| SEQ | |
|---|---|
| ID | |
| NO: | Amino acid sequence |
| SEQ | MKKIWLALAGLVLAFSASAGPQPGVPLGYPIKAPKLPGGYGLPY |
| ID | TTGKLPYGYGPGGVAGAAGKAGYPTGTGVGPQAAAAAAAKAAAK |
| NO: | FGAGAAGVLPGVGGAGVPGVPGAIPGIGGIAGVGTPAAAAAAAA |
| 38 | AAKAAKYGAAAGLVPGGPGFGPGVVGVPGAGVPGVGVPGAGIPV |
| | VPGAGIPGAAVPGVVSPEAAAKAAAKAAKYGARPGVGVGGIPTY |
| | GVGAGGFPGFGVGVGGIPGVAGVPGVGGVPGVGGVPGVGISPEA |
| | QAAAAAKAAKYGAAGAGVLGGLVPGPQAAVPGVPGTGGVPGVGT |
| | P |
| SEQ | GPQPGVPLGYPIKAPKLPGGYGLPYTTGKLPYGYGPGGVAGAAG |
| ID | KAGYPTGTGVGPQAAAAAAAKAAAKFGAGAAGVLPGVGGAGVPG |
| NO: | VPGAIPGIGGIAGVGTPAAAAAAAAAAKAAKYGAAAGLVPGGPG |
| 40 | FGPGVVGVPGAGVPGVGVPGAGIPVVPGAGIPGAAVPGVVSPEA |
| | AAKAAAKAAKYGARPGVGVGGIPTYGVGAGGFPGFGVGVGGIPG |
| | VAGVPGVGGVPGVGGVPGVGISPEAQAAAAAKAAKYGAAGAGVL |
| | GGLVPGPQAAVPGVPGTGGVPGVGTP |
| SEQ | GPGGVAAAAKSAAKVAAKAQLRAAAGLGAGIPGLGVGVGVPGLG |
| ID | VGAGVPGLGVGAGVPGFGAGADEGVRRSLSPELREGDPSSSQHL |
| NO: | PSTPSSPRVPGALAAAKAAKYGAAVPGVLGGLGALGGVGIPGGV |
| 41 | VGAGPAAAAAAAKAAAKAAQFGLVGAAGLGGLGVGGLGVPGVGG |
| | LGGIPPAAAAKAAKYGAAGLGGVLGGAGQFPLGGVAARPGFGLS |
| | PIFPGGACLGKACGRKRK |
| SEQ | GGVPGAIPGGVPGGVFYPGAGLGALGGGALGPGGKPLKPVPGGL |
| ID | AGAGLGAGLGAFPAVTFPGALVPGGVADAAAAYKAAKAGAGLGG |
| NO: | VPGVGGLGVSAGAVVPQPGAGVKPGKVPGVGLPGVYPGGVLPGA |
| 43 | RFPGVGVLPGVPTGAGVKPKAPGVGGAFAGIPGVGPFGGPQPGV |
| | PLGYPIKAPKLPGGYGLPYTTGKLPYGYGPGGVAGAAGKAGYPT |
| | GTGVGPQAAAAAAAKAAAKFGAGAAGVLPGVGGAGVPGVPGAIP |
| | GIGGIAGVGTPAAAAAAAAAAKAAKYGAAAGLVPGGPGFGPGVV |
| | GVPGAGVPGVGVPGAGIPVVPGAGIPGAAVPGVVSPEAAAKAAA |
| | KAAKYGARPGVGVGGIPTYGVGAGGFPGFGVGVGGIPGVAGVPG |
| | VGGVPGVGGVPGVGISPEAQAAAAAKAAKYGAAGAGVLGGLVPG |
| | PQAAVPGVPGTGGVPGVGTPAAAAKSAAKVAAKAQLRAAAGLGA |
| | GIPGLGVGVGVPGLGVGAGVPGLGVGAGVPGFGAGADEGVRRSL |
| | SPELREGDPSSSQHLPSTPSSPRVPGALAAAKAAKYGAAVPGVL |
| | GGLGALGGVGIPGGVVGAGPAAAAAAAKAAAKAAQFGLVGAAGL |
| | GGLGVGGLGVPGVGGLGGIPPAAAAKAAKYGAAGLGGVLGGAGQ |
| | FPLGGVAARPGFGLSPIFPGGACLGKACGRKRK |
| SEQ | MKKIWLALAGLVLAFSASAAGLGGVPGVGGLGVSAGAVVPQPGA |
| ID | GVKPGKVPGVGLPGVYPGGVLPGARFPGVGVLPGVPTGAGVKPK |
| NO: | APGVGGAFAGIPGVGPFGGPQPGVPLGYPIKAPKLPGGYGLPYT |
| 46 | TGKLPYGYGPGGVAGAAGKAGYPTGTGVGPQAAAAAAAKAAAKF |
| | GAGAAGVLPGVGGAGVPGVPGAIPGIGGIAGVGTPAAAAAAAAA |
| | AKAAKYGAAAGLVPGGPGFGPGVVGVPGAGVPGVGVPGAGIPVV |
| | PGAGIPGAAVPGVVSPE |
| SEQ | AGLGGVPGVGGLGVSAGAVVPQPGAGVKPGKVPGVGLPGVYPGG |
| ID | VLPGARFPGVGVLPGVPTGAGVKPKAPGVGGAFAGIPGVGPFGG |
| NO: | PQPGVPLGYPIKAPKLPGGYGLPYTTGKLPYGYGPGGVAGAAGK |
| 48 | AGYPTGTGVGPQAAAAAAAKAAAKFGAGAAGVLPGVGGAGVPGV |
| | PGAIPGIGGIAGVGTPAAAAAAAAAAKAAKYGAAAGLVPGGPGF |
| | GPGVVGVPGAGVPGVGVPGAGIPVVPGAGIPGAAVPGVVSPE |
| SEQ | MKKIWLALAGLVLAFSASAAGLGGVPGVGGLGVSAGAVVPQPGA |
| ID | GVKPGKVPGVGLPGVYPGGVLPGARFPGVGVLPGVPTGAGVKPK |
| NO: | APGVGGAFAGIPGVGPFGGPQPGVPLGYPIKAPKLPGGYGLPYT |
| 50 | TGKLPYGYGPGGVAGAAGKAGYPTGTGVGPQ |
| SEQ | AGLGGVPGVGGLGVSAGAVVPQPGAGVKPGKVPGVGLPGVYPGG |
| ID | VLPGARFPGVGVLPGVPTGAGVKPKAPGVGGAFAGIPGVGPFGG |
| NO: | PQPGVPLGYPIKAPKLPGGYGLPYTTGKLPYGYGPGGVAGAAGK |
| 52 | AGYPTGTGVGPQ |

In various aspects, the truncated human elastin does not comprise two or more contiguous amino acid sequences of VGVAPG (SEQ ID NO: 34). In some cases, the truncated human elastin does not comprise VGVAPGVGVAPG (SEQ ID NO: 35). In some cases, the truncated human elastin does not comprise VGVAPGVGVAPGVGVAPG (SEQ ID NO: 36). In some cases, the non-naturally occurring truncated human elastin reduces extracellular matrix degradation or does not cause extracellular matrix degradation.

In some cases, a truncated elastin may be from 100 to 150 amino acids, from 100 to 200 amino acids, from 100 to 300 amino acids, from 140 to 250 amino acids, from 140 to 200 amino acids, from 150 to 250 amino acids, from 160 to 250 amino acids, from 160 to 220 amino acids, from 170 to 200 amino acids, from 180 to 190 amino acids, or from 185 to 190 amino acids in length.

In some aspects, the truncated human elastin may have a molecular weight of at least about 5, 10, 15, 20, 25, 30, 35, 40, 45, 50 or more than 50 kDa. In some embodiments, the molecular weight is less than about 10, 15, 20, 25, 30, 35, 40, 45, or 50 kDa. In some embodiments, the molecular weight is from 1 kDa to 60 kDa, from 5 kDa to 55 kDa, from 5 kDa to 50 kDa, from 5 kDa to 45 kDa, from 5 kDa to 40 kDa, from 5 kDa to 35 kDa, from 5 kDa to 30 kDa, from 5 kDa to 25 kDa, from 5 kDa to 20 kDa, from 5 kDa to 15 kDa, from 5 kDa to 10 kDa, from 10 kDa to 40 kDa, from 10 kDa to 35 kDa, from 10 kDa to 30 kDa, from 10 kDa to 25 kDa, or from 10 kDa to 20 kDa.

The non-naturally occurring collagen or elastin may, in some embodiments, further comprise amino acid sequences including a secretion tag. The secretion tag may direct the collagen to the periplasmic space of the host cell. In particular embodiments, the signal peptide is derived from DsbA, PelB, OmpA, TolB, MalE, lpp, TorA, Hy1A, DegP, or a hybrid secretion tag that comprises a portion of one secretion tag fused to a portion of a second secretion tag. In one aspect the secretion tag may be attached to the non-naturally occurring collagen or elastin. In another aspect the secretion tag may be cleaved from the non-naturally occurring collagen or elastin.

In some embodiments, the non-naturally occurring collagen or elastin comprises a histidine (or polyhistidine) tag. In specific embodiments, the histidine tag or polyhistidine tag is or comprises a sequence of 2 to 20 histidine residues (SEQ ID NO: 55) that are attached to the collagen or elastin. In various embodiments, the histidine tag comprises 2 to 20 histidine residues, 5 to 15 histidine residues, 5 to 18 histidine residues, 5 to 16 histidine residues, 5 to 15 histidine residues, 5 to 14 histidine residues, 5 to 13 histidine residues, 5 to 12 histidine residues, 5 to 11 histidine residues, 5 to 10 histidine residues, 6 to 12 histidine residues, 6 to 11 histidine residues, or 7 to 10 histidine residues. The histidine tags may be useful in purification of proteins by chromatographic methods utilizing nickel based chromatographic media.

In some embodiments, the non-naturally occurring collagen or elastin further comprises a fluorescent protein. Exemplary fluorescent proteins include green fluorescent protein (GFP) or red fluorescent protein (RFP). Fluorescent proteins are well known in the art. In one embodiment, a non-naturally occurring collagen or elastin comprises a GFP and/or RFP. In one embodiment, a superfolder GFP is fused to a non-naturally occurring collagen. The superfolder GFP may be a GFP that folds properly even when fused to a poorly folded polypeptide. In one aspect, a histidine tag may be attached to the non-naturally occurring collagen. In another aspect, a histidine tag may be cleaved from the non-naturally occurring collagen or elastin.

In some embodiments, the non-naturally occurring collagen or elastin further comprises a protease cleavage site. The protease cleavage site may be useful to cleave the recombinantly produced collagen to remove one or more portions of the polypeptide. The portions of the polypeptide that may be removed include the secretion tag, the histidine tag, the fluorescent protein tag, and/or the Beta-lactamase. The proteases may comprise endoproteases, exoproteases, serine proteases, cysteine proteases, threonine proteases, aspartic proteases, glutamic proteases, and metalloproteases. Exemplary protease cleavage sites include amino acids that are cleaved by Thrombin, TEV protease, Factor Xa, Enteropeptidase, and Rhinovirus 3C Protease. In one aspect, the cleavage tag is attached to the non-naturally occurring collagen or elastin. In another aspect, the cleavage tag is removed by an appropriate protease from the non-naturally occurring collagen or elastin.

In some embodiments, the non-naturally occurring collagen or elastin further comprises an enzyme that is a Beta-lactamase. The beta-lactamase may be useful as a selection marker. In one aspect, the beta-lactamase is attached to the non-naturally occurring collagen or elastin. In another aspect, the beta-lactamase is cleaved from the non-naturally occurring collagen or elastin.

Provided in certain embodiments herein are (e.g., topical) compositions or formulations comprising one or more polypeptide provided herein. In some embodiments, the composition provides any suitable amount of polypeptide provided herein, such as in any suitable amount (e.g., an amount suitable to provide a benefit when given or administered to an individual or cell). In some specific embodiments, the composition comprises an amount suitable to provide a beneficial effect to the skin of an individual when (e.g., topically) administered to the skin of the individual. In specific embodiments, the composition comprises from about 0.001% w/w and about 30% w/w of a polypeptide (or non-naturally occurring collagen) such as provided herein. In more specific embodiments, the composition comprises between about 0.001% w/w and about 20% w/w of a polypeptide (or non-naturally occurring collagen) such as provided herein, between about 0.001% w/w and about 10% w/w of a polypeptide (or non-naturally occurring collagen) such as provided herein, between about 0.001% w/w and about 5% w/w of a polypeptide (or non-naturally occurring collagen) such as provided herein, between about 0.001% w/w and about 2% w/w of a polypeptide such as provided herein, between about 0.001% w/w and about 1% w/w of a polypeptide such as provided herein, between about 0.001% w/w and about 0.5% w/w of a polypeptide such as provided herein, and between about 0.001% w/w and about 0.2% w/w of a polypeptide (or non-naturally occurring collagen) such as provided herein.

In one aspect, the compositions that comprise non-naturally occurring collagen or elastin may be personal care products (e.g., a cosmetic). In some embodiments, the compositions are formulated for topical administration. The compositions can contain other cosmetic ingredients suitable for human use. The personal care products may be useful for preventing or treating ultraviolet radiation damage to human skin or hair. The personal care products may be useful for increasing the firmness, elasticity, brightness, hydration, tactile texture or visual texture of skin and/or stimulate collagen production. The personal care products may be useful for reducing redness of the skin. The personal care products may be applied to skin or hair. The compositions include, for example, masks, skin cleaners such as soap, cleansing creams, cleansing lotions, facial cleansers, cleansing milks, cleansing pads, facial washes, facial and body creams and moisturizers, facial serums, facial and body masks, facial toners and mists, eye creams and eye treatments, exfoliator formulas, lip balms and lipsticks, hair shampoo, hair conditioner and body shampoos, hair and scalp serums, hair mists and sprays, eye shadow, concealer, mascara and other color cosmetics.

The compositions that comprise the non-naturally occurring collagen or elastin can further comprise at least one additional ingredient comprising a topical carrier or a preservative. The topical carrier may comprise a topical carrier selected from the group consisting of liposome, biodegradable microcapsule, lotion, spray, aerosol, dusting powder, biodegradable polymer, mineral oil, triglyceride oil, silicone oil, glycerin, glycerin monostearate, alcohols, emulsifying agents, liquid petroleum, white petrolatum, propylene glycol, polyoxyethylene, polyoxypropylene, wax, sorbitan monostearate, polysorbate, cetyl ester wax, cetearyl alcohol, 2-octyldodecanol, benzyl alcohol, cyclomethicone, cyclopentasiloxane and water. The preservative may comprise a preservative selected from the group consisting of tocopherol, diiodomethyl-p-tolylsulfone, 2-Bromo-2-nitropropane-1,3-diol, cis isomer 1-(3-chloroallyl)-3,5,7-triaza-1-azoniaadamantane chloride, glutaraldehyde, 4,4-dimethyl oxazolidine, 7-Ethylbicyclooxazolidine, phenoxyethanol, butylene glycol, 1,2 Hexanediol, methyl paraben, sorbic acid, Germaben® II, rosemary extract, and EDTA

In additional embodiments, the compositions that comprise non-naturally occurring collagen or elastin may be formulated as injectables, for example, as soft tissue fillers, for example, dermal and subdermal fillers, based on hyaluronic acids (HA) and pharmaceutically acceptable salts of HA. Such formulations may be injected under one or more skin layers.

Also provided in certain embodiments herein, are methods of decreasing skin damage, promoting the repair of damaged skin, protecting skin against UV damage, and/or protecting skin cells against the effects of exposure to urban dust. In another embodiment, methods of increasing the firmness, elasticity, brightness, hydration, tactile texture, or visual texture of skin and/or stimulating collagen production are provided. The methods may comprise a step of applying a composition comprising a non-naturally occurring collagen or elastin to the skin of a subject. Without being bound to a particular theory or mechanism, the collagen or elastin in the composition may decrease skin damage by protecting against UV damage. In some cases, the collagen or elastin in the composition may promote the repair of damaged skin by increasing the viability of cells. In some cases, the collagen or elastin in the composition may decrease skin damage and/or promote repair of cells by increasing procollagen synthesis when applied to skin, and/or promoting the viability of skin cells. In some cases, the collagen or elastin decreases the formation of thymine-thymine (TT) dimers.

The methods provided herein encompass the use of a composition for treatment indicated in the method, such as by the steps provided herein. In embodiments, the disclosure provides the use of a composition provided herein in a method for decreasing skin damage, promoting the repair of damaged skin, protecting skin against UV damage, and/or protecting skin cells against the effects of exposure to urban dust (e.g., such as by administering to the skin of a subject a composition provided herein). In embodiments, the disclosure provides the use of a composition provided herein in a method for increasing the firmness, elasticity, brightness, hydration, tactile texture, or visual texture of skin and/or stimulating collagen production.

In some embodiments, a truncated collagen or elastin as provided herein may stimulate fibroblast and/or keratinocyte production of collagen type I (see, e.g., Example 4 and Example 6). In some cases, the levels of pro-collagen type I C-peptide (a read-out for collagen production) may be measured. In some cases, an in vitro MatTek full thickness human skin tissue model may be used (see, e.g., Example 6) to assess pro-collagen type I C-peptide levels. In some cases, collagen type I levels may be measured or determined by an enzyme-linked immunosorbent assay (ELISA). In some cases, a truncated collagen or elastin as provided herein may stimulate production of collagen type I at a higher level than untreated cells, cells treated with retinol, and/or cells treated with Vitamin B3.

In some embodiments, a truncated collagen or elastin as provided herein may stimulate fibroblast overexpression of extracellular matrix genes (see, e.g., Example 4). In some cases, the levels of extracellular matrix genes may be measured by RNA sequencing. In some cases, a truncated collagen or elastin as provided herein may stimulate fibroblast overexpression of one or more of the collagen type I gene (COL1A), the elastin gene (ELN), and the fibronectin gene (FN1). In some cases, the levels of extracellular matrix genes produced by fibroblasts treated with a truncated collagen or elastin provided herein may be higher than untreated fibroblasts, or fibroblasts treated with retinol. In some cases, the levels of extracellular matrix genes produced by fibroblasts treated with a truncated collagen provided herein may be similar to, or higher than, fibroblasts treated with Vitamin C.

In some embodiments, a truncated collagen or elastin as provided herein may reduce inflammation of keratinocytes irradiated with UVB light (see, e.g., Example 4 and Example 6). In some cases, keratinocytes may be irradiated with UVB light, and then treated with a truncated collagen as provided herein. In some cases, inflammation may be measured by measuring the levels of IL-la produced by UVB-irradiated keratinocytes (e.g., by ELISA). In some cases, UVB-irradiated keratinocytes may produce lower levels of IL-la when treated with a truncated collagen provided herein than untreated keratinocytes.

In some embodiments, a truncated collagen or elastin as provided herein may increase viability of keratinocytes irradiated with UVB light (see, e.g., Example 4). In some cases, keratinocytes may be pre-treated (prior to UVB irradiation) and post-treated (after UVB irradiation) with a truncated collagen or elastin provided herein. In some cases, cell viability may be measured using an MTT metabolic colorimetric assay. In some cases, keratinocytes treated with a truncated collagen or elastin provided herein may exhibit greater cell viability after UVB irradiation than untreated keratinocytes.

In some embodiments, a truncated collagen or elastin as provided herein may reduce DNA damage in keratinocytes after exposure to UVB light (see, e.g., Example 6). In some cases, DNA damage may be assessed by measuring the levels of thymine dimers (TT-dimers). In a non-limiting example, the OxiSelect UV-induced DNA damage ELISA kit may be used to measure TT-dimer levels. In some cases, UVB-irradiated keratinocytes treated with a truncated collagen or elastin provided herein may show lower levels of TT-dimers than untreated keratinocytes.

In some embodiments, a truncated collagen or elastin as provided herein may have anti-oxidative capacity (see, e.g., Example 4 and Example 6). In some cases, an oxygen radical absorbance capacity (ORAC) assay may be used to measure oxidative capacity of the truncated collagen or elastin. In a non-limiting example, a truncated collagen or elastin in the form of a 0.1% solution may have anti-oxidative properties of at least 10 μM Trolox (Vitamin E) equivalents (TEs), at least 50 μM TEs, at least 100 μM TEs, at least 150 μM TEs, at least 160 TEs, at least 170 μM TEs, at least 180 μM TEs, at least 190 μM TEs, or at least 200 μM TEs.

In some embodiments, a truncated collagen or elastin as provided herein may increase cell viability of keratinocytes exposed to urban dust pollution as compared to untreated cells (see, e.g., Example 6). In some cases, cell viability may be measured by an MTT metabolic colorimetric assay.

In some embodiments, topical administration of a truncated collagen or elastin provided herein to the face of a subject may result in increased facial skin elasticity, as compared to baseline, at 1 week, 2 weeks, 4 weeks, 8 weeks, or longer, post-treatment (see, e.g., Example 5 and Example 7). In some cases, facial skin elasticity may be measured by a cutometer.

In some embodiments, topical administration of a truncated collagen or elastin provided herein to the face of a subject may result in an increase in facial skin collagen content, as compared to baseline, at 1 week, at 2 weeks, at 4 weeks, at 8 weeks, or longer, post-treatment (see, e.g., Example 5). In some cases, facial skin collagen content may be measured by a SIAscope.

In some embodiments, topical administration of a truncated collagen or elastin provided herein may result in a reduction in facial skin redness (erythema), as compared to baseline, at 1 week, at 2 weeks, at 4 weeks, at 8 weeks, or longer, post-treatment (see, e.g., Example 5). In some cases, facial skin redness (erythema) may be scored by a blinded clinical grader (e.g., using a 5-point ordinal scale as provided in Table 6).

In some embodiments, topical administration of a truncated collagen or elastin provided herein may result in a reduction in facial wrinkles, as compared to baseline, at 1 week, at 2 weeks, at 4 weeks, at 8 weeks, or longer, post-treatment (see, e.g., Example 5). In some cases, facial wrinkles may be scored by a blinded clinical grader.

In some embodiments, topical administration of a truncated collagen or elastin provided herein may result in increased facial skin moisture, as compared to baseline, at 1 week, at 2 weeks, at 4 weeks, at 8 weeks, or longer, post-treatment (see, e.g., Example 7). In some cases, topical administration of a truncated collagen or elastin provided herein may result in increased facial skin moisture as compared to topical administration of a marine collagen or elastin. In some cases, skin hydration may be measured by a corneometer.

One aspect of this disclosure provides polynucleotides that encode a non-naturally occurring collagen. The polynucleotides may encode collagen from jellyfish or human. The polynucleotides may encode for a collagen that is full length or truncated. In various embodiments, the polynucleotide may comprise a polynucleotide according to any one of SEQ ID NO: 1, SEQ ID NO: 3, SEQ ID NO: 6, SEQ ID NO: 8, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 26, SEQ ID NO: 28, or SEQ ID NO: 30, or a homolog thereof (e.g., having at least 85%, at least 90%, at least 95%, or at least 98% sequence identity thereto). In some cases, the polynucleotide may be codon optimized (e.g., for expression in a host cell).

In some embodiments, the non-naturally occurring elastin may be encoded by a polynucleotide (e.g., a non-naturally occurring elastin; e.g., for expression in a host cell). The polynucleotides may encode for a full-length elastin or a truncated elastin. In various embodiments, the polynucleotide may comprise a polynucleotide according to any one of SEQ ID NO: 37, SEQ ID NO: 39, SEQ ID NO: 42, SEQ ID NO: 44, SEQ ID NO: 45, SEQ ID NO: 47, SEQ ID NO: 49, SEQ ID NO: 51, or a homolog thereof (e.g., having at least 80%, at least 85%, at least 90%, at least 95%, or at least 98% sequence identity thereto). In some cases, the polynucleotide may be codon optimized (e.g., for expression in a host cell).

In another aspect the present disclosure provides polynucleotides that encode collagen or elastin fusion proteins. The fusion proteins may comprise a secretion tag, a histidine tag, a fluorescent protein tag, a protease cleavage site, a Beta-lactamase along and/or GEK amino acid trimer repeats and/or GDK amino acid trimer repeats together with collagen or elastin.

In an aspect, vectors comprising the collagen or elastin encoding polynucleotides may be used to transform host cells and express the polynucleotides. The polynucleotides may further comprise nucleic acids that encode enzymes that permit the host organism to grow in the presence of a selection agent. The selection agents may include certain sugars including galactose containing sugars or antibiotics including ampicillin, hygromycin, G418, and others. Enzymes that can be used to confer resistance to the selection agent include β-galactosidase or a β-lactamase.

In one aspect, host cells that express the polynucleotides of the invention are provided. Host cells can be any host cell including gram negative bacterial cells, gram positive bacterial cells, yeast cells, insect cells, mammalian cells, plant cells, or any other cells used to express exogenous polynucleotides. An exemplary gram-negative host cell is E. coli.

Any desirable or necessary supplements besides carbon, nitrogen, and inorganic phosphate sources may also be included at appropriate concentrations introduced alone or as a mixture with another supplement or medium such as a complex nitrogen source. In certain embodiments, the medium further comprises one or more ingredients selected from: ammonium chloride, ammonium sulfate, calcium chloride, casamino acids, iron(II) sulfate, magnesium sulfate, peptone, potassium phosphate, sodium chloride, sodium phosphate, and yeast extract.

Beta-lactamases are enzymes that confer resistance to lactam antibiotics in prokaryotic cells. Typically when Beta-lactamases are expressed in bacterial host cells, the expressed Beta-lactamase proteins also include targeting sequences (secretion tag) that direct the Beta-lactamase proteins to the periplasmic space. Beta-lactamases are not functional unless they are transported to the periplasmic space. Beta-lactamases targeted to the periplasmic space without the use of an independent secretion tag that targets the enzyme to the periplasmic space are provided. By creating a fusion protein in which a periplasmic secretion tag is added to the N-terminus of a protein such as GFP, collagen or elastin, or GFP/collagen (or elastin) chimeras, the functionality of the Beta-lactamase lacking a native secretion tag can be used to select for full translation and secretion of the N-terminal fusion proteins. Using this approach, a DsbA-GFP-Collagen-Beta-lactamase fusion may be used to select for truncation products in the target collagens or elastins that favor translation and secretion.

Another embodiment provides methods of producing a polypeptide, such as provided herein. In some embodiments, the method comprises the steps of inoculating a culture medium with a recombinant host cell comprising polynucleotides that encode the polypeptide, cultivating the host cell, and isolating the polypeptide from the host cell.

A process for fermentative preparation of a polypeptide (or protein) is provided. The process comprises the steps of:
(a) culturing a recombinant Gram-negative bacterial cell in a medium comprising a magnesium salt, wherein the concentration of magnesium ions in the medium is at least about 6 mM, and wherein the bacterial cell comprises an exogenous gene encoding the protein; and
(b) harvesting the protein from the medium.

The bacteria may be cultured in any suitable manner, such as continuously—as described, for example, in WO 05/021772—or discontinuously in a batch process (batch cultivation) or in a fed-batch or repeated fed-batch process for the purpose of producing the target protein. In some embodiments, protein production is conducted on a large-scale. Various large-scale fermentation procedures are available for production of recombinant proteins. Large-scale fermentations have at least 1,000 liters of capacity, preferably about 1,000 to 100,000 liters of capacity. In some instances, fermenters use agitator impellers to distribute oxygen and nutrients, especially glucose (the preferred carbon/energy source). Small-scale fermentation refers generally to fermentation in a fermenter that is no more than approximately 20 liters in volumetric capacity.

For accumulation of the target protein, the host cell may be cultured under conditions sufficient for accumulation of the target protein. Such conditions include, e.g., temperature, nutrient, and cell-density conditions that permit protein expression and accumulation by the cell. Moreover, such conditions may be those under which the cell can perform basic cellular functions of transcription, translation, and passage of proteins from one cellular compartment to another for the secreted proteins, as are known to those skilled in the art.

Any suitable bacterial cell is optionally utilized in a method provided herein. The bacterial cells may be cultured at any suitable temperature. In specific embodiments, the bacterial cells are E. coli cells. For E. coli growth, for example, the typical temperature ranges from about 20° C. to about 39° C. In one embodiment, the temperature is from about 20° C. to about 37° C. In another embodiment, the temperature is at about 30° C. In one embodiment, the host cells, in the non-switched state or switched state may be cultivated at one temperature and switched to a different temperature to induce protein production. The host cells may be cultivated first at one temperature to propagate the cells, then to induce protein production the cells may be cultivated at a lower temperature. The first temperature may be about 23°, 24°, 25°, 26°, 27°, 28°, 29°, 30°, 31°, 32°, 33°, 34°, 35°, 36°, or 37° C. The second temperature may be about 20°, 21°, 22°, 23°, 24°, 25°, 26°, 27°, 28°, 29°, 30°, 31°, 32°, 33°, 34°, 35° or 36° C. The cultivation at the second temperature may be conducted between 1 hour and 100 hours, between 5 hours and 90 hours, between 5 hours and 80 hours, between 5 hours and 80 hours, between 5 hours and 70 hours, between 10 hours and 70 hours, between 15 hours and 70 hours, between 15 hours and 65 hours, between 15 hours and 60 hours, between 20 hours and 60 hours, between 20 hours and 55 hours, between 20 hours and 50 hours, between 24 hours and 50 hours, between 24 hours and 48 hours, between 30 hours and 50 hours, between 30 hours and 45 hours, or between 30 hours and 40 hours.

The pH of the culture medium may be any pH from about 5-9, depending mainly on the host organism. For E. coli, the pH may be from about 6.0 to about 7.4, about 6.2 to about 7.2, about 6.2 to about 7.0, about 6.2 to about 6.8, about 6.2 to about 6.6, about 6.4 or about 6.5.

For induction of gene expression, typically the cells may be cultured until a certain optical density is achieved, e.g., an OD600 of about 1.1, at which point induction is initiated (e.g., by addition of an inducer, by depletion of a repressor, suppressor, or medium component, etc.) to induce expression of the exogenous gene encoding the target protein. In some embodiments, expression of the exogenous gene may be inducible by an inducer selected from, e.g., isopropyl-β-d-1-thiogalactopyranoside, lactose, arabinose, maltose, tetracycline, anhydrotetracycline, vavlycin, xylose, copper, zinc, and the like. The induction of gene expression can also be accomplished by decreasing the dissolved oxygen levels during fermentation. The dissolved oxygen levels of the fermentation during cell propagation may be between 10% and 30%. To induce gene expression the dissolved oxygen level may be reduced to below 10%, 9%, 8%, 7%, 6%, 5%, 4%, 3%, 2%, 1%, or 0%. In host cells, in either the physiological state or the switched state, protein production can be induced by lowering the temperature of the fermentation as disclosed herein.

### Combinations of Collagen or Elastin Polypeptides and Other Actives

Provided in certain aspects herein are compositions or formulations (e.g., formulated for application to the skin or hair of a subject) comprising a collagen or elastin polypeptide (e.g., as described herein) and one or more of hyaluronic acid, retinol, ascorbic acid, salicylic acid, benzoyl peroxide, niacinamide, or alpha hydroxy acids (e.g., glycolic acid, lactic acid). In some embodiments, the collagen or elastin polypeptide is any collagen or elastin polypeptide disclosed herein (e.g., full-length polypeptide, truncated polypeptide), such as any collagen or elastin polypeptide that has one or more of the effects described herein (e.g., a synergistic effect, e.g., in combination with one or more of hyaluronic acid, retinol, ascorbic acid, salicylic acid, benzoyl peroxide, niacinamide, or alpha hydroxy acids (e.g., glycolic acid, lactic acid)). In some embodiments, the collagen or elastin polypeptide is any collagen or elastin polypeptide (e.g., full-length or truncated) disclosed in U.S. Provisional Patent Application No. 62/965,700, the disclosure of which is herein incorporated by reference with regards to such collagen polypeptides.

In some cases, the collagen polypeptide is a (e.g., full-length) human type 21 collagen polypeptide or a truncate thereof (e.g., truncated relative to a full-length human type 21 collagen polypeptide). In some cases, the composition comprises any truncated human type 21 collagen polypeptide as described herein. In some cases, the composition comprises a human type 21 collagen polypeptide comprising an amino acid sequence having at least about 80% (e.g., at least about 85%, at least about 90%, at least about 95%, at least about 96%, at least about 97%, at least about 98%, at least about 99%) sequence identity to SEQ ID NO: 31. In some cases, the composition comprises a human type 21 collagen polypeptide comprising an amino acid sequence of SEQ ID NO: 31. In some cases, the composition comprises a human type 21 collagen polypeptide consisting of an amino acid sequence of SEQ ID NO: 31.

In some aspects, the composition comprises a polypeptide comprising an amino acid sequence truncated relative to a full-length human type 21 collagen polypeptide (e.g., SEQ ID NO: 31). In some cases, the polypeptide comprises an amino acid sequence having a truncation at the C-terminal end, a truncation at the N-terminal end, an internal truncation, or any combination thereof, relative to a full-length human type 21 collagen polypeptide (e.g., SEQ ID NO: 31). In some cases, the polypeptide comprises an amino acid sequence having a truncation at both the C-terminal end and the N-terminal end relative to a full-length human type 21 collagen polypeptide (e.g., SEQ ID NO: 31).

In some cases, the polypeptide comprises an amino acid sequence having a truncation of from 10 amino acids to 800 amino acids relative to a full-length human type 21 collagen (e.g., SEQ ID NO: 31). In some cases, the polypeptide comprises an amino acid sequence having a from 10 amino acids and 800 amino acids, from 10 amino acids to 750 amino acids, from 10 amino acids to 700 amino acids, from 10 amino acids to 650 amino acids, from 10 amino acids to 600 amino acids, from 10 amino acids to 550 amino acids, from 10 amino acids to 500 amino acids, from 10 amino acids to 450 amino acids, from 10 amino acids to 400 amino acids, from 10 amino acids to 350 amino acids, from 10 amino acids to 300 amino acids, from 10 amino acids to 250 amino acids, from 10 amino acids to 200 amino acids, from 10 amino acids to 150 amino acids, or from 10 amino acids to 100 amino acids. In another embodiment, the polypeptide may be truncated by about 10, about 20, about 30, about 40, about 50, about 60, about 70, about 80, about 90, about 100, about 110, about 120, about 130, about 140, about 150, about 160, about 170, about 180, about 190, about 200, about 210, about 220, about 230, about 240, about 250, about 260, about 270, about 280, about 290, about 300, about 310, about 320, about 330, about 340, about 350, about 360, about 370, about 380, about 390, about 400, about 410, about 420, about 430, about 440, about 450, about 460, about 470, about 480, about 490, about 500, about 510, about 520, about 530, about 540, about 550, about 560, about 570, about 580, about 590, about 600, about 610, about 620, about 630, about 640, about 650, about 660, about 670, about 680, about 690, about 700, about 710, about 720, about 730, about 740, about 750, about 760, about 770, about 780, about 790, or about 800 amino acids.

In some cases, the polypeptide comprises an amino acid sequence having at least about 80% (e.g., at least about 85%, at least about 90%, at least about 95%, at least about 96%, at least about 97%, at least about 98%, at least about 99%) sequence identity to an amino acid sequence of SEQ ID NO: 14 or SEQ ID NO: 16. In some cases, the polypeptide comprises an amino acid sequence of SEQ ID NO: 14 or SEQ ID NO: 16. In some cases, the polypeptide consists of an amino acid sequence of SEQ ID NO: 14 or SEQ ID NO: 16.

In some aspects, the composition comprises a polypeptide as described herein and hyaluronic acid. In some cases, the hyaluronic acid has a molecular weight from about 1 kDa to about 50 MDa. In some cases, the hyaluronic acid is a low molecular weight hyaluronic acid. In some cases, the hyaluronic acid has a molecular weight of less than about 50 kDa (e.g., less than about 40 kDa, less than about 30 kDa, less than about 20 kDa, less than about 10 kDa, less than about 5 kDa). In some cases, the hyaluronic acid has a molecular weight of greater than about 50 kDa. In some cases, the hyaluronic acid has a molecular weight from about 15 kDa to about 40 kDa. In some cases, the hyaluronic acid has a molecular weight from about 50 kDa to about 200 kDa. In some cases, the hyaluronic acid has a molecular weight from about 200 kDa to about 1 MDa. In some cases, the hyaluronic acid has a molecular weight from about 1 MDa to about 5 MDa.

In some aspects, the composition comprises a polypeptide as described herein and ascorbic acid. In some aspects, the composition comprises a polypeptide as described herein and retinol. In some aspects, the composition comprises a polypeptide as described and salicylic acid. In some aspects, the composition comprises a polypeptide as described herein and benzoyl peroxide. In some aspects, the composition comprises a polypeptide as described herein and niacinamide. In some aspects, the composition comprises a polypeptide as described herein and alpha hydroxy acids (e.g., glycolic acid, lactic acid).

In some embodiments, the composition provides any suitable amount of polypeptide and one or more of hyaluronic acid, retinol, ascorbic acid, salicylic acid, benzoyl peroxide, niacinamide, or alpha hydroxy acids (e.g., glycolic acid, lactic acid), such as in an amount suitable to provide a benefit when given or administered to an individual or cell. In some specific embodiments, the composition comprises an amount of polypeptide and one or more of hyaluronic acid, retinol, ascorbic acid, salicylic acid, benzoyl peroxide, niacinamide, or alpha hydroxy acids (e.g., glycolic acid, lactic acid), suitable to provide a beneficial effect to the skin or hair of an individual when administered to the skin or hair of the individual.

In specific aspects, the composition comprises from about 0.001% w/w to about 30% w/w of a polypeptide provided herein. In more specific aspects, the composition comprises from about 0.001% w/w to about 20% w/w of a polypeptide provided herein, from about 0.001% w/w to about 10% w/w of a polypeptide provided herein, from about 0.001% w/w to about 5% w/w of a polypeptide provided herein, from about 0.001% w/w to about 2% w/w of a polypeptide provided herein, from about 0.001% w/w to about 1% w/w of a polypeptide provided herein, from about 0.001% w/w to about 0.5% w/w of a polypeptide provided herein, or from about 0.001% w/w to about 0.2% w/w of a polypeptide provided herein. In some aspects, the composition comprises from about 0.01% w/w to about 1% w/w of a polypeptide provided herein.

In various aspects, the composition comprises from about 0.01% w/w to about 50% w/w of ascorbic acid. For example, the composition may comprise from about 0.01% w/w to about 25% w/w, from about 0.01% w/w to about 15% w/w, from about 0.01% w/w to about 10% w/w, from about 0.01% w/w to about 5% w/w, from about 0.1% w/w to about 25% w/w, from about 0.1% to about 10% w/w, from about 1% w/w to about 50% w/w, or from about 1% w/w to about 25% w/w of ascorbic acid. In some cases, the composition comprises from about 0.1% w/w to about 25% w/w of ascorbic acid.

In various aspects, the composition comprises from about 0.001% w/w to about 10% w/w of hyaluronic acid. For example, the composition may comprise from about 0.001% w/w to about 5% w/w, from about 0.001% w/w to about 1% w/w, from about 0.1% w/w to about 10% w/w, from about 0.1% w/w to about 5% w/w, from about 0.1% w/w to about 1%, from about 1% w/w to about 10% w/w, or from about 1% w/w to about 5% w/w of hyaluronic acid. In some cases, the composition comprises from about 0.01% w/w to about 5% w/w of hyaluronic acid.

In various aspects, the composition comprises from about 0.001% w/w to about 5% w/w of retinol. For example, the composition may comprise from about 0.001% w/w to about 2.5% w/w, from about 0.001% w/w to about 1% w/w, from about 0.01% w/w to about 5% w/w, from about 0.01% w/w to about 2.5% w/w, from about 0.01% w/w to about 1% w/w, from about 1% w/w to about 5% w/w, or from about 1% w/w to about 2.5% w/w of retinol. In some cases, the composition comprises from about 0.01% w/w to about 5% w/w.

In various aspects, the composition comprises from about 0.1% w/w to about 50% w/w of salicylic acid. For example, the composition may comprise from about 0.1% w/w to about 30% w/w, from about 0.1% w/w to about 15% w/w, from about 0.1% w/w to about 5% w/w, from about 1% w/w to about 30% w/w, from about 1% w/w to about 15% w/w, from about 1% w/w to about 5% w/w, from about 10% w/w to about 50% w/w, from about 10% w/w to about 30% w/w, or from about 10% w/w to about 20% w/w of salicylic acid. In some cases, the composition comprises from about 2% w/w to about 30% w/w of salicylic acid.

In various aspects, the composition comprises from about 0.1% w/w to about 20% w/w of benzoyl peroxide. For example, the composition may comprise from about 0.1% w/w to about 10% w/w, from about 0.1% w/w to about 5% w/w, from about 0.1% w/w to about 1% w/w, from about 1% w/w to about 15% w/w, from about 1% w/w to about 10% w/w, from about 1% w/w to about 5% w/w, from about 5% w/w to about 15% w/w, or from about 5% w/w to about 10% w/w of benzoyl peroxide.

In various aspects, the composition comprises from about 0.1% w/w to about 50% w/w of niacinamide. For example, the composition may comprise from about 0.1% w/w to about 25% w/w, from about 0.1% w/w to about 15% w/w, from about 0.1% w/w to about 5% w/w, from about 1% w/w to about 30% w/w, from about 1% w/w to about 20% w/w, from about 1% w/w to about 10% w/w, from about 5% w/w to about 40% w/w, from about 5% w/w to about 20% w/w, from about 5% w/w to about 15% w/w, from about 10% w/w to about 50% w/w, or from about 10% w/w to about 25% w/w of niacinamide.

In various aspects, the composition comprises from about 0.1% w/w to about 50% w/w of alpha hydroxy acids (e.g., glycolic acid, lactic acid). For example, the composition may comprise from about 0.1% w/w to about 25% w/w, from about 0.1% w/w to about 15% w/w, from about 0.1% w/w to about 5% w/w, from about 1% w/w to about 30% w/w, from about 1% w/w to about 20% w/w, from about 1% w/w to about 10% w/w, from about 5% w/w to about 40% w/w, from about 5% w/w to about 20% w/w, from about 5% w/w to about 15% w/w, from about 10% w/w to about 50% w/w, or from about 10% w/w to about 25% w/w of alpha hydroxy acids (e.g., glycolic acid, lactic acid).

Further provided herein are formulations comprising any composition described herein (e.g., a collagen polypeptide or elastin polypeptide and one or more of hyaluronic acid, ascorbic acid, retinol, salicylic acid, benzoyl peroxide, niacinamide, or alpha hydroxy acids (e.g., glycolic acid, lactic acid)). In some cases, the formulation is a personal care product. The personal care product may be formulated for application to the skin (e.g., topical application) and/or to the hair of an individual. In some cases, the personal care product comprises any composition described herein (e.g., a collagen polypeptide or elastin polypeptide and one or more of hyaluronic acid, ascorbic acid, retinol, salicylic acid, benzoyl peroxide, niacinamide, or alpha hydroxy acids (e.g., glycolic acid, lactic acid)), and one or more carriers or excipients. The personal care product may contain other ingredients suitable for human use. A personal care product provided herein may include, without limitation, masks, skin cleaners such as soap, cleansing creams, cleansing lotions, facial cleansers, cleansing milks, cleansing pads, facial washes, facial and body creams and moisturizers, facial serums, facial and body masks, facial toners and mists, eye creams and eye treatments, exfoliator formulas, lip balms and lipsticks, hair shampoo, hair conditioner and body shampoos, hair and scalp serums, hair mists and sprays, eye shadow, concealer, mascara and other color cosmetics. In some aspects, the personal care product is a cosmetic.

In various embodiments formulations and compositions described herein comprise the collagen and/or elastin polypeptides in combination with additional active ingredients providing a benefit to skin or hair. Such active ingredients may include retinoids or vitamin A compounds, such as retinol and/or retinoic acid, e.g., for the repair of ultraviolet radiation damaged skin and/or the reduction of fine lines and wrinkles; vitamin C (ascorbic acid) as an antioxidant, e.g., for the protection of skin from ultraviolet radiation and/or the reduction of fine lines and wrinkles; alpha hydroxy acids (AHAs) such as glycolic, citric and lactic acids as exfoliants, e.g., for the stimulation of skin renewal and absorption of formulations and compositions applied to the skin; hyaluronic acid, e.g., for the improvement of skin hydration; coenzyme Q10, e.g., for the reduction of fine lines and wrinkles and/or the protection of skin from ultraviolet radiation; tea extracts, such as green, black and oolong tea extracts having antioxidant and/or anti-inflammatory properties, e.g., for the improvement of skin texture and/or the reduction of fine lines and wrinkles; grape seed extracts having antioxidant and/or anti-inflammatory properties, e.g., for the promotion of collagen production; niacinamide having antioxidant properties, e.g., for the improvement of skin hydration, skin brightness, and skin elasticity; and antibacterial or acne preventative active ingredients, such as benzoyl peroxide, salicylic acid or sulfur-containing ingredients. In various aspects, the personal care product may further comprise one or more additional ingredients. The one or more additional ingredient may be selected from the group consisting of: water, oil glycereth-8 esters, glycerin, coconut alkanes, hydroxyethyl acrylate/sodium acryloyldimethyl taurate copolymer, pentylene glycol, disodium EDTA, caprylyl glycol, chlorphenesin, phenoxyethanol, and vegetable oil (e.g., olive oil).

In various aspects, the personal care product may further comprise one or more topical carriers (e.g., suitable for topical application). The one or more topical carriers may be selected from the group consisting of: a liposome, a biodegradable microcapsule, a lotion, a spray, an aerosol, a dusting powder, a biodegradable polymer, mineral oil, triglyceride oil, silicone oil, olive oil, vegetable oil, glycerin, glycerin monostearate, alcohols, emulsifying agents, liquid petroleum, white petrolatum, propylene glycol, polyoxyethylene, polyoxypropylene, wax, sorbitan monostearate, polysorbate, cetyl ester wax, cetearyl alcohol, 2-octyldodecanol, benzyl alcohol, cyclomethicone, cyclopentasiloxane, and water.

In various aspects, the personal care product may further comprise one or more preservatives. The one or more preservatives may be selected from the group consisting of: tocopherol, diiodomethyl-p-tolylsulfone, 2-bromo-2-nitropropane-1,3-diol, cis isomer 1-(3-chloroallyl)-3,5,7-triaza-1-azoniaadamantane chloride, glutaraldehyde, 4,4-dimethyl oxazolidine, 7-ethylbicyclooxazolidine, phenoxyethanol, butylene glycol, 1,2 Hexanediol, methyl paraben, sorbic acid, Germaben® II, rosemary extract, and ethylenediaminetetraacetic acid (EDTA).

In various aspects, the personal care product may comprise ingredients for hydrogel formation for the composition alone or when combined with crosslinking materials (e.g., enzymes, polysaccharides, or small molecules). In various aspects, the personal care product may have a film-forming capacity.

In various aspects, the compositions and/or formulations provided herein (e.g., comprising a collagen or elastin polypeptide and one or more of hyaluronic acid, ascorbic acid, retinol, salicylic acid, benzoyl peroxide, niacinamide, or alpha hydroxy acids (e.g., glycolic acid, lactic acid)) is capable of increasing the viability of skin cells, increasing the proliferation of skin cells, or both, when applied to human skin cell cultures, human skin equivalent cultures, or ex vivo human skin (e.g., as measured by MTT viability assay) (e.g., as compared to pre-treatment, as compared to untreated (or vehicle) control, as compared to treatment with human type 21 collagen polypeptide or a truncate thereof alone, or an elastin polypeptide or truncate thereof alone, as compared to treatment with ascorbic acid alone, as compared to treatment with hyaluronic acid alone, as compared to treatment with retinol alone, as compared to treatment with salicylic acid alone, as compared to treatment with benzoyl peroxide alone, as compared to treatment with niacinamide alone, as compared to treatment with alpha hydroxy acids (e.g., glycolic acid, lactic acid) alone). In some cases, the composition and/or formulations may be capable of producing any of the aforementioned effects synergistically or additively (e.g., as compared to treatment with human type 21 collagen polypeptide or a truncate thereof alone or an elastin polypeptide or truncate thereof alone, as compared to treatment with ascorbic acid alone, as compared to treatment with hyaluronic acid alone, as compared to treatment with retinol alone, as compared to treatment with salicylic acid alone, as compared to treatment with benzoyl peroxide alone, as compared to treatment with niacinamide alone, as compared to treatment with alpha hydroxy acids (e.g., glycolic acid, lactic acid) alone).

In various aspects, the compositions and/or formulations provided herein (e.g., comprising a collagen or elastin polypeptide and one or more of hyaluronic acid, ascorbic acid, retinol, salicylic acid, benzoyl peroxide, niacinamide, or alpha hydroxy acids (e.g., glycolic acid, lactic acid)) are capable of increasing the production of extracellular matrix (ECM) components (e.g., collagen, elastin, fibronectin, fibrillin, hyaluronic acid), decreasing the production of matrix-degrading proteins (e.g., matrix metalloproteases (MMPs), proteases), or both, when applied to human skin cell cultures, human skin equivalent cultures, or ex vivo human skin (e.g., as measured by transcriptomic analysis, as measured by enzyme-linked immunosorbent assay (ELISA)) (e.g., as compared to pre-treatment, as compared to untreated (or vehicle) control, as compared to treatment with human type 21 collagen polypeptide or a truncate thereof alone, as compared to elastin or an elastin truncate alone, as compared to treatment with ascorbic acid alone, as compared to treatment with hyaluronic acid alone, as compared to treatment with retinol alone, as compared to treatment with salicylic acid alone, as compared to treatment with benzoyl peroxide alone, as compared to treatment with niacinamide alone, as compared to treatment with alpha hydroxy acids (e.g., glycolic acid, lactic acid) alone). In some cases, the composition and/or formulations may be capable of producing any of the aforementioned effects synergistically or additively (e.g., as compared to treatment with human type 21 collagen polypeptide or a truncate thereof alone, elastin polypeptide or a truncate thereof alone, as compared to treatment with ascorbic acid alone, as compared to treatment with hyaluronic acid alone, as compared to treatment with retinol alone, as compared to treatment with salicylic acid alone, as compared to treatment with benzoyl peroxide alone, as compared to treatment with niacinamide alone, as compared to treatment with alpha hydroxy acids (e.g., glycolic acid, lactic acid) alone).

In various aspects, the compositions and/or formulations provided herein (e.g., comprising a collagen or elastin polypeptide and one or more of hyaluronic acid, ascorbic acid, retinol, salicylic acid, benzoyl peroxide, niacinamide, or alpha hydroxy acids (e.g., glycolic acid, lactic acid)) are capable of increasing wound healing capacity when applied to skin cells (e.g., keratinocytes, fibroblasts) or human skin equivalent cultures (e.g., as measured by the wound healing scratch assay) (e.g., as compared to pre-treatment, as compared to untreated (or vehicle) control, as compared to treatment with a collagen or elastin polypeptide or a truncate thereof alone, as compared to treatment with ascorbic acid alone, as compared to treatment with hyaluronic acid alone, as compared to treatment with retinol alone, as compared to treatment with salicylic acid alone, as compared to treatment with benzoyl peroxide alone, as compared to treatment with niacinamide alone, as compared to treatment with alpha hydroxy acids (e.g., glycolic acid, lactic acid) alone). In some cases, the composition and/or formulations may be capable of producing any of the aforementioned effects synergistically or additively (e.g., as compared to treatment with a collagen or elastin polypeptide or a truncate thereof alone, as compared to treatment with ascorbic acid alone, as compared to treatment with hyaluronic acid alone, as compared to treatment with retinol alone, as compared to treatment with salicylic acid alone, as compared to treatment with benzoyl peroxide alone, as compared to treatment with niacinamide alone, as compared to treatment with alpha hydroxy acids (e.g., glycolic acid, lactic acid) alone).

In various aspects, the compositions and/or formulations provided herein (e.g., comprising a collagen or elastin polypeptide and one or more of hyaluronic acid, ascorbic acid, retinol, salicylic acid, benzoyl peroxide, niacinamide, or alpha hydroxy acids (e.g., glycolic acid, lactic acid)) are capable of improving the viability of cells (e.g., as measured by MTT viability assay), improving the proliferation of cells (e.g., as measured by MTT viability assay), reducing the cytotoxicity of cells (e.g., as measured by fluorescence microscopy), reducing apoptosis of cells (e.g., as measured by fluorescence microscopy), improving DNA repair of cells (e.g., as measured by thymidine-dimer ELISA detection), reducing inflammation (e.g., as measured by ELISA analysis or Luminex detection of cytokines), reducing reactive oxidative stress (ROS) (e.g., as measured by CM-H2DCFDA detection), or any combination thereof, when applied to skin cells (e.g., keratinocytes, fibroblasts) or human skin equivalent cultures after exposure to photodamage (e.g., UV irradiation, blue light exposure) (e.g., as compared to pre-treatment, as compared to untreated (or vehicle) control, as compared to treatment with a collagen or elastin polypeptide or a truncate thereof alone, as compared to treatment with ascorbic acid alone, as compared to treatment with hyaluronic acid alone, as compared to treatment with retinol alone, as compared to treatment with salicylic acid alone, as compared to treatment with benzoyl peroxide alone, as compared to treatment with niacinamide alone, as compared to treatment with alpha hydroxy acids (e.g., glycolic acid, lactic acid) alone). In some cases, the composition and/or formulations may be capable of producing any of the aforementioned effects synergistically or additively (e.g., as compared to treatment with a collagen or elastin polypeptide or a truncate thereof alone, as compared to treatment with ascorbic acid alone, as compared to treatment with hyaluronic acid alone, as compared to treatment with retinol alone, as compared to salicylic acid alone, as compared to treatment with benzoyl peroxide alone, as compared to treatment with niacinamide alone, as compared to treatment with alpha hydroxy acids (e.g., glycolic acid, lactic acid) alone).

In various aspects, the compositions and/or formulations provided herein (e.g., comprising a collagen or elastin polypeptide and one or more of hyaluronic acid, ascorbic acid, retinol, salicylic acid, benzoyl peroxide, niacinamide, or alpha hydroxy acids (e.g., glycolic acid, lactic acid)) are capable of improving viability of cells (e.g., as measured by MTT viability assay), improving proliferation of cells (e.g., as measured by MTT viability assay), reducing cytotoxicity of cells (e.g., as measured by fluorescence microscopy), reducing apoptosis of cells (e.g., as measured by fluorescence microscopy), improving DNA repair of cells (e.g., as measured by thymidine-dimer ELISA detection), reducing inflammation (e.g., as measured by ELISA analysis or Luminex detection of cytokines), reducing reactive oxidative stress (ROS) (e.g., as measured by CM-H2DCFDA detection), or any combination thereof, when applied to skin cells (e.g., keratinocytes, fibroblasts) or human skin equivalent cultures after exposure to pollution (e.g., urban dust exposure) (e.g., as compared to pre-treatment, as compared to untreated (or vehicle) control, as compared to treatment with a collagen or elastin polypeptide or a truncate thereof alone, as compared to treatment with ascorbic acid alone, as compared to treatment with hyaluronic acid alone, as compared to treatment with retinol alone, as compared to treatment with salicylic acid alone, as compared to treatment with benzoyl peroxide alone, as compared to treatment with niacinamide alone, as compared to treatment with alpha hydroxy acids (e.g., glycolic acid, lactic acid) alone). In some cases, the composition and/or formulations may be capable of producing any of the aforementioned effects synergistically or additively (e.g., as compared to treatment with a collagen or elastin polypeptide or a truncate thereof alone, as compared to treatment with ascorbic acid alone, as compared to treatment with hyaluronic acid alone, as compared to treatment with retinol alone, as compared to treatment with salicylic acid alone, as compared to treatment with benzoyl peroxide alone, as compared to treatment with niacinamide alone, as compared to treatment with alpha hydroxy acids (e.g., glycolic acid, lactic acid) alone).

In various aspects, the compositions and/or formulations provided herein (e.g., comprising a collagen or elastin polypeptide and one or more of hyaluronic acid, ascorbic acid, retinol, salicylic acid, benzoyl peroxide, niacinamide, or alpha hydroxy acids (e.g., glycolic acid, lactic acid)) are capable of improving viability of cells (e.g., as measured by MTT viability assay), improving proliferation of cells (e.g., as measured by MTT viability assay), reducing cytotoxicity of cells (e.g., as measured by fluorescence microscopy), reducing apoptosis of cells (e.g., as measured by fluorescence microscopy), improving DNA repair of cells (e.g., as measured by thymidine-dimer ELISA detection), reducing inflammation (e.g., as measured by ELISA analysis or Luminex detection of cytokines), reducing reactive oxidative stress (ROS) (e.g., as measured by CM-H2DCFDA detection), or any combination thereof, when applied to skin cells (e.g., keratinocytes, fibroblasts) or human skin equivalent cultures after and/or during exposure to retinoic acid or retinol, benzoyl peroxide, or salicylic acid (e.g., as compared to pre-treatment, as compared to untreated (or vehicle) control, as compared to treatment with a collagen or elastin polypeptide or a truncate thereof alone, as compared to treatment with ascorbic acid alone, as compared to treatment with hyaluronic acid alone, as compared to treatment with retinol alone, as compared to treatment with salicylic acid alone, as compared to treatment with benzoyl peroxide alone, as compared to treatment with niacinamide alone, as compared to treatment with alpha hydroxy acids (e.g., glycolic acid, lactic acid) alone). In some cases, the composition and/or formulations may be capable of producing any of the aforementioned effects synergistically or additively (e.g., as compared to treatment with a collagen or elastin polypeptide or a truncate thereof alone, as compared to treatment with ascorbic acid alone, as compared to treatment with hyaluronic acid alone, as compared to treatment with retinol alone, as compared to treatment with salicylic acid alone, as compared to treatment with benzoyl peroxide alone, as compared to treatment with niacinamide alone, as compared to treatment with alpha hydroxy acids (e.g., glycolic acid, lactic acid) alone).

In various aspects, the compositions and/or formulations provided herein (e.g., comprising a collagen or elastin polypeptide and one or more of hyaluronic acid, ascorbic acid, retinol, salicylic acid, benzoyl peroxide, niacinamide, or alpha hydroxy acids (e.g., glycolic acid, lactic acid)) are capable of improving the appearance and/or quality of the skin of the subject (e.g. as compared to pre-treatment, as compared to untreated (or vehicle) control, as compared to treatment with a collagen or elastin polypeptide or a truncate thereof alone, as compared to treatment with ascorbic acid alone, as compared to treatment with hyaluronic acid alone, as compared to treatment with retinol alone, as compared to treatment with salicylic acid alone, as compared to treatment with benzoyl peroxide alone, as compared to treatment with niacinamide alone, as compared to treatment with alpha hydroxy acids (e.g., glycolic acid, lactic acid) alone), when applied to the skin of a subject. In some cases, the composition and/or formulations may be capable of producing any of the aforementioned effects synergistically or additively (e.g., as compared to treatment with a collagen or elastin polypeptide or a truncate thereof alone, as compared to treatment with ascorbic acid alone, as compared to treatment with hyaluronic acid alone, as compared to treatment with retinol alone, as compared to treatment with salicylic acid alone, as compared to treatment with benzoyl peroxide alone, as compared to treatment with niacinamide alone, as compared to treatment with alpha hydroxy acids (e.g., glycolic acid, lactic acid) alone).

In various aspects, the compositions and/or formulations provided herein (e.g., comprising a collagen polypeptide and one or more of hyaluronic acid, ascorbic acid, retinol, salicylic acid, benzoyl peroxide, niacinamide, or alpha hydroxy acids (e.g., glycolic acid, lactic acid)) are capable of reducing wrinkles, reducing fine lines, reducing skin redness, reducing skin pigmentation/hyperpigmentation, increasing skin brightness, decreasing pore size, decreasing skin roughness, reducing acne (e.g., as measured using CLARITY analysis), or any combination thereof (e.g. as compared to pre-treatment, as compared to untreated (or vehicle) control, as compared to treatment with a collagen or elastin polypeptide or a truncate thereof alone, as compared to treatment with ascorbic acid alone, as compared to treatment with hyaluronic acid alone, as compared to treatment with retinol alone, as compared to treatment with salicylic acid alone, as compared to treatment with benzoyl peroxide alone, as compared to treatment with niacinamide alone, as compared to treatment with alpha hydroxy acids (e.g., glycolic acid, lactic acid) alone), when applied to the skin of a subject. In some cases, the composition and/or formulations may be capable of producing any of the aforementioned effects synergistically or additively (e.g., as compared to treatment with a collagen or elastin polypeptide or a truncate thereof alone, as compared to treatment with ascorbic acid alone, as compared to treatment with hyaluronic acid alone, as compared to treatment with retinol alone, as compared to treatment with salicylic acid alone, as compared to treatment with benzoyl peroxide alone, as compared to treatment with niacinamide alone, as compared to treatment with alpha hydroxy acids (e.g., glycolic acid, lactic acid) alone).

In various aspects, the compositions and/or formulations provided herein (e.g., comprising a collagen or elastin polypeptide and one or more of hyaluronic acid, ascorbic acid, retinol, salicylic acid, benzoyl peroxide, niacinamide, or alpha hydroxy acids (e.g., glycolic acid, lactic acid)) are capable of increasing skin elasticity, increasing skin firmness, increasing skin hydration, increasing skin barrier function, increasing skin collagen content, increasing skin elastin content, increasing dermal density, or any combination thereof (e.g. as compared to pre-treatment, as compared to untreated (or vehicle) control, as compared to treatment with a collagen or elastin polypeptide or a truncate thereof alone, as compared to treatment with ascorbic acid alone, as compared to treatment with hyaluronic acid alone, as compared to treatment with retinol alone, as compared to treatment with salicylic acid alone, as compared to treatment with benzoyl peroxide alone, as compared to treatment with niacinamide alone, as compared to treatment with alpha hydroxy acids (e.g., glycolic acid, lactic acid) alone), when applied to the skin of a subject. In some cases, the composition and/or formulations may be capable of producing any of the aforementioned effects synergistically or additively (e.g., as compared to treatment with a collagen or elastin polypeptide or a truncate thereof alone, as compared to treatment with ascorbic acid alone, as compared to treatment with hyaluronic acid alone, as compared to treatment with retinol alone, as compared to treatment with salicylic acid alone, as compared to treatment with benzoyl peroxide alone, as compared to treatment with niacinamide alone, as compared to treatment with alpha hydroxy acids (e.g., glycolic acid, lactic acid) alone).

In various aspects, the compositions and/or formulations provided herein (e.g., comprising a collagen or elastin polypeptide and one or more of hyaluronic acid, ascorbic acid, retinol, salicylic acid, benzoyl peroxide, niacinamide, or alpha hydroxy acids (e.g., glycolic acid, lactic acid)) are capable of improving skin microflora (e.g. as compared to pre-treatment, as compared to untreated (or vehicle) control, as compared to treatment with human type 21 collagen polypeptide or a truncate thereof alone, as compared to treatment with ascorbic acid alone, as compared to treatment with hyaluronic acid alone, as compared to treatment with retinol alone, as compared to treatment with salicylic acid alone, as compared to treatment with benzoyl peroxide alone, as compared to treatment with niacinamide alone, as compared to treatment with alpha hydroxy acids (e.g., glycolic acid, lactic acid) alone), when applied to the skin of a subject. In various aspects, the compositions and/or formulations provided herein (e.g., comprising a collagen or elastin polypeptide and one or more of hyaluronic acid, ascorbic acid, retinol, salicylic acid, benzoyl peroxide, niacinamide, or alpha hydroxy acids (e.g., glycolic acid, lactic acid)) are capable of reducing the abundance of harmful microbes on the skin, promoting beneficial microbes on the skin, increasing microbial diversity on the skin, or any combination thereof (e.g. as compared to pre-treatment, as compared to untreated (or vehicle) control, as compared to treatment with a collagen or elastin polypeptide or a truncate thereof alone, as compared to treatment with ascorbic acid alone, as compared to treatment with hyaluronic acid alone, as compared to treatment with retinol alone, as compared to treatment with salicylic acid alone, as compared to treatment with benzoyl peroxide alone, as compared to treatment with niacinamide alone, as compared to treatment with alpha hydroxy acids (e.g., glycolic acid, lactic acid) alone), when applied to the skin of a subject. In some cases, the composition and/or formulations may be capable of producing any of the aforementioned effects synergistically or additively (e.g., as compared to treatment with a collagen or elastin polypeptide or a truncate thereof alone, as compared to treatment with ascorbic acid alone, as compared to treatment with hyaluronic acid alone, as compared to treatment with retinol alone, as compared to treatment with salicylic acid alone, as compared to treatment with benzoyl peroxide alone, as compared to treatment with niacinamide alone, as compared to treatment with alpha hydroxy acids (e.g., glycolic acid, lactic acid) alone).

In various aspects, the compositions and/or formulations provided herein (e.g., comprising a collagen or elastin polypeptide and one or more of hyaluronic acid, ascorbic acid, retinol, salicylic acid, benzoyl peroxide, niacinamide, or alpha hydroxy acids (e.g., glycolic acid, lactic acid)) are capable of improving the appearance and/or quality of the hair of a subject (e.g. as compared to pre-treatment, as compared to untreated (or vehicle) control, as compared to treatment with a collagen or elastin polypeptide or a truncate thereof alone, as compared to treatment with ascorbic acid alone, as compared to treatment with hyaluronic acid alone, as compared to treatment with retinol alone, as compared to treatment with salicylic acid alone, as compared to treatment with benzoyl peroxide alone, as compared to treatment with niacinamide alone, as compared to treatment with alpha hydroxy acids (e.g., glycolic acid, lactic acid) alone), when applied to the hair of the subject. In some cases, the composition and/or formulations may be capable of producing any of the aforementioned effects synergistically or additively (e.g., as compared to treatment with a collagen or elastin polypeptide or a truncate thereof alone, as compared to treatment with ascorbic acid alone, as compared to treatment with hyaluronic acid alone, as compared to treatment with retinol alone, as compared to treatment with salicylic acid alone, as compared to treatment with benzoyl peroxide alone, as compared to treatment with niacinamide alone, as compared to treatment with alpha hydroxy acids (e.g., glycolic acid, lactic acid) alone).

In various aspects, the compositions and/or formulations provided herein (e.g., comprising a collagen or elastin polypeptide and one or more of hyaluronic acid, ascorbic acid, retinol, salicylic acid, benzoyl peroxide, niacinamide, or alpha hydroxy acids (e.g., glycolic acid, lactic acid)) are capable of improving hair growth, increasing the thickness of hair fiber diameter, increasing combability, reducing hair loss, increasing the growth rate, increasing hair tensile strength, or any combination thereof (e.g. as compared to pre-treatment, as compared to untreated (or vehicle) control, as compared to treatment with a collagen or elastin polypeptide or a truncate thereof alone, as compared to treatment with ascorbic acid alone, as compared to treatment with hyaluronic acid alone, as compared to treatment with retinol alone, as compared to treatment with salicylic acid alone, as compared to treatment with benzoyl peroxide alone, as compared to treatment with niacinamide alone, as compared to treatment with alpha hydroxy acids (e.g., glycolic acid, lactic acid) alone), when applied to the hair of a subject. In some cases, the composition and/or formulations may be capable of producing any of the aforementioned effects synergistically or additively (e.g., as compared to treatment with a collagen or elastin polypeptide or a truncate thereof alone, as compared to treatment with ascorbic acid alone, as compared to treatment with hyaluronic acid alone, as compared to treatment with retinol alone, as compared to treatment with salicylic acid alone, as compared to treatment with benzoyl peroxide alone, as compared to treatment with niacinamide alone, as compared to treatment with alpha hydroxy acids (e.g., glycolic acid, lactic acid) alone).

In various aspects, the compositions and/or formulations provided herein (e.g., comprising a collagen or elastin polypeptide and one or more of hyaluronic acid, ascorbic acid, retinol, salicylic acid, benzoyl peroxide, niacinamide, or alpha hydroxy acids (e.g., glycolic acid, lactic acid)) are capable of improving hair fiber thickness, improving hair fiber density, increasing moisture, increasing hydrophobicity, reducing split ends, reducing frizz, increasing static control, improving fiber alignment, improving shine, increasing wet combability, increasing dry combability, increasing resistance to hair breakage, or any combination thereof (e.g. as compared to pre-treatment, as compared to untreated (or vehicle) control, as compared to treatment with a collagen or elastin polypeptide or a truncate thereof alone, as compared to treatment with ascorbic acid alone, as compared to treatment with hyaluronic acid alone, as compared to treatment with retinol alone, as compared to treatment with salicylic acid alone, as compared to treatment with benzoyl peroxide alone, as compared to treatment with niacinamide alone, as compared to treatment with alpha hydroxy acids (e.g., glycolic acid, lactic acid) alone), when applied to the hair of a subject. In some cases, the composition and/or formulations may be capable of producing any of the aforementioned effects synergistically or additively (e.g., as compared to treatment with a collagen or elastin polypeptide or a truncate thereof alone, as compared to treatment with ascorbic acid alone, as compared to treatment with hyaluronic acid alone, as compared to treatment with retinol alone, as compared to treatment with salicylic acid alone, as compared to treatment with benzoyl peroxide alone, as compared to treatment with niacinamide alone, as compared to treatment with alpha hydroxy acids (e.g., glycolic acid, lactic acid) alone).

Further provided herein are methods of using the compositions and/or formulations (e.g., a personal care product) provided herein (e.g., comprising a collagen or elastin polypeptide and one or more of hyaluronic acid, ascorbic acid, retinol, salicylic acid, benzoyl peroxide, niacinamide, or alpha hydroxy acids (e.g., glycolic acid, lactic acid)). In some cases, the methods involve applying a composition or formulation (e.g., a personal care product) provided herein (e.g., comprising a collagen or elastin polypeptide or a truncate thereof) and one or more of hyaluronic acid, ascorbic acid, retinol, salicylic acid, benzoyl peroxide, niacinamide, or alpha hydroxy acids (e.g., glycolic acid, lactic acid)) to the skin of a subject. In some cases, the methods involve applying a composition or formulation (e.g., a personal care product) provided herein (e.g., comprising a collagen or elastin polypeptide and one or more of hyaluronic acid, ascorbic acid, retinol, salicylic acid, benzoyl peroxide, niacinamide, or alpha hydroxy acids (e.g., glycolic acid, lactic acid)) to the hair of a subject.

In various aspects, a method is provided for treating the skin of a subject, the method comprising administering any composition and/or formulation (e.g., a personal care product) as described herein (e.g., comprising a collagen or elastin polypeptide or a truncate thereof) and one or more of hyaluronic acid, ascorbic acid, retinol, salicylic acid, benzoyl peroxide, niacinamide, or alpha hydroxy acids (e.g., glycolic acid, lactic acid)) to the skin of the subject (e.g., topical application), thereby treating the skin of the subject. In some cases, treating includes cosmetic treatment (e.g., to improve or restore an individual's appearance).

In various aspects, the method results in an improvement in the appearance and/or quality of the skin of the subject (e.g. as compared to pre-treatment, as compared to untreated (or vehicle) control, as compared to treatment with a collagen or elastin polypeptide or a truncate thereof alone, as compared to treatment with ascorbic acid alone, as compared to treatment with hyaluronic acid alone, as compared to treatment with retinol alone, as compared to treatment with salicylic acid alone, as compared to treatment with benzoyl peroxide alone, as compared to treatment with niacinamide alone, as compared to treatment with alpha hydroxy acids (e.g., glycolic acid, lactic acid) alone). In various aspects, the method results in a reduction in wrinkles, a reduction in fine lines, a reduction in skin redness, a reduction in skin pigmentation/hyperpigmentation, an increase in skin brightness, a decrease in pore size, a decrease in skin roughness, a reduction in acne (e.g., as measured using CLARITY analysis), or any combination thereof (e.g. as compared to pre-treatment, as compared to untreated (or vehicle) control, as compared to treatment with a collagen or elastin polypeptide or a truncate thereof alone, as compared to treatment with ascorbic acid alone, as compared to treatment with hyaluronic acid alone, as compared to treatment with retinol alone, as compared to treatment with salicylic acid alone, as compared to treatment with benzoyl peroxide alone, as compared to treatment with niacinamide alone, as compared to treatment with alpha hydroxy acids (e.g., glycolic acid, lactic acid) alone). In various aspects, the method results in an increase in skin elasticity, an increase in skin firmness, an increase in skin hydration, an increase in skin barrier function, an increase in skin collagen content, an increase in skin elastin content, an increase in dermal density, or any combination thereof (e.g. as compared to pre-treatment, as compared to untreated (or vehicle) control, as compared to treatment with a collagen or elastin polypeptide or a truncate thereof alone, as compared to treatment with ascorbic acid alone, as compared to treatment with hyaluronic acid alone, as compared to treatment with retinol alone, as compared to treatment with salicylic acid alone, as compared to treatment with benzoyl peroxide alone, as compared to treatment with niacinamide alone, as compared to treatment with alpha hydroxy acids (e.g., glycolic acid, lactic acid) alone). In various aspects, the method results in an improvement of skin microflora (e.g. as compared to pre-treatment, as compared to untreated (or vehicle) control, as compared to treatment with a collagen or elastin polypeptide or a truncate thereof alone, as compared to treatment with ascorbic acid alone, as compared to treatment with hyaluronic acid alone, as compared to treatment with retinol alone, as compared to treatment with salicylic acid alone, as compared to treatment with benzoyl peroxide alone, as compared to treatment with niacinamide alone, as compared to treatment with alpha hydroxy acids (e.g., glycolic acid, lactic acid) alone). In various aspects, the method results in a reduction in the abundance of harmful microbes on the skin, a promotion of beneficial microbes on the skin, an increase in microbial diversity on the skin, or any combination thereof (e.g. as compared to pre-treatment, as compared to untreated (or vehicle) control, as compared to treatment with a collagen or elastin polypeptide or a truncate thereof alone, as compared to treatment with ascorbic acid alone, as compared to treatment with hyaluronic acid alone, as compared to treatment with retinol alone, as compared to treatment with salicylic acid alone, as compared to treatment with benzoyl peroxide alone, as compared to treatment with niacinamide alone, as compared to treatment with alpha hydroxy acids (e.g., glycolic acid, lactic acid) alone). In some cases, the method may synergistically or additively produce any of the aforementioned outcomes (e.g., as compared to treatment with a collagen or elastin polypeptide or a truncate thereof alone, as compared to treatment with ascorbic acid alone, as compared to treatment with hyaluronic acid alone, as compared to treatment with retinol alone, as compared to treatment with salicylic acid alone, as compared to treatment with benzoyl peroxide alone, as compared to treatment with niacinamide alone, as compared to treatment with alpha hydroxy acids (e.g., glycolic acid, lactic acid) alone).

In various aspects, a method is provided for treating the hair of a subject, the method comprising administering any composition and/or formulation (e.g., a personal care product) as described herein (e.g., comprising a collagen polypeptide (e.g., a collagen or elastin polypeptide or a truncate thereof) and one or more of hyaluronic acid, ascorbic acid, retinol, salicylic acid, benzoyl peroxide, niacinamide, or alpha hydroxy acids (e.g., glycolic acid, lactic acid)) to the hair of the subject, thereby treating the hair of the subject. In some cases, treating includes cosmetic treatment (e.g., to improve or restore an individual's appearance).

In various aspects, the method results in an improvement in the appearance and/or quality of the hair of the subject (e.g. as compared to pre-treatment, as compared to untreated (or vehicle) control, as compared to treatment with a collagen or elastin polypeptide or a truncate thereof alone, as compared to treatment with ascorbic acid alone, as compared to treatment with hyaluronic acid alone, as compared to treatment with retinol alone, as compared to treatment with salicylic acid alone, as compared to treatment with benzoyl peroxide alone, as compared to treatment with niacinamide alone, as compared to treatment with alpha hydroxy acids (e.g., glycolic acid, lactic acid) alone). In various aspects, the method results in an improvement in hair growth, thicker hair fiber diameter, an increase in combability, a reduction of hair loss, faster growth rate, an increase in hair tensile strength, or any combination thereof (e.g. as compared to pre-treatment, as compared to untreated (or vehicle) control, as compared to treatment with a collagen or elastin polypeptide or a truncate thereof alone, as compared to treatment with ascorbic acid alone, as compared to treatment with hyaluronic acid alone, as compared to treatment with retinol alone, as compared to treatment with salicylic acid alone, as compared to treatment with benzoyl peroxide alone, as compared to treatment with niacinamide alone, as compared to treatment with alpha hydroxy acids (e.g., glycolic acid, lactic acid) alone). In various aspects, the method results in improved hair fiber thickness, improved hair fiber density, an increase in moisture, an increase in hydrophobicity, a reduction of split ends, a reduction of frizz, an increase in static control, an improvement in fiber alignment, an improvement in shine, an increase in wet combability, an increase in dry combability, stronger resistance to hair breakage, or any combination thereof (e.g. as compared to pre-treatment, as compared to untreated (or vehicle) control, as compared to treatment with a collagen or elastin polypeptide or a truncate thereof alone, as compared to treatment with ascorbic acid alone, as compared to treatment with hyaluronic acid alone, as compared to treatment with retinol alone, as compared to treatment with salicylic acid alone, as compared to treatment with benzoyl peroxide alone, as compared to treatment with niacinamide alone, as compared to treatment with alpha hydroxy acids (e.g., glycolic acid, lactic acid) alone). In some cases, the method may synergistically or additively produce any of the aforementioned outcomes (e.g., as compared to treatment with a collagen or elastin polypeptide or a truncate thereof alone, as compared to treatment with ascorbic acid alone, as compared to treatment with hyaluronic acid alone, as compared to treatment with retinol alone, as compared to treatment with salicylic acid alone, as compared to treatment with benzoyl peroxide alone, as compared to treatment with niacinamide alone, as compared to treatment with alpha hydroxy acids (e.g., glycolic acid, lactic acid) alone).

In another aspect, a method is provided for increasing type I collagen production in a skin cell (e.g., keratinocyte, fibroblast), the method comprising: contacting the skin cell with a composition and/or a formulation comprising a collagen or elastin polypeptide or a truncate thereof, and hyaluronic acid, thereby increasing type I collagen production in the skin cell. In some cases, the method involves contacting the skin (e.g., containing a skin cell (e.g., keratinocyte, fibroblast)) of a subject in need thereof, with a composition and/or a formulation comprising a collagen or elastin polypeptide or a truncate thereof, and hyaluronic acid, to increase type I collagen production in the skin cell. The subject in need thereof may be an individual in need of an increase in type I collagen production in the skin. The subject in need thereof may be an individual in need of improvement of one or more of skin aging, skin elasticity, skin firmness, or wrinkles and fine lines. In some cases, the increase in type I collagen production may be synergistic or additive (e.g., as compared to treatment with a collagen or elastin polypeptide or a truncate thereof alone, as compared to treatment with hyaluronic acid alone).

In another aspect, a method is provided for increasing fibronectin production in a skin cell (e.g., keratinocyte, fibroblast), the method comprising: contacting the skin cell with a composition and/or a formulation comprising a collagen or elastin polypeptide or a truncate thereof, and hyaluronic acid, thereby increasing fibronectin production in the skin cell. In some cases, the method involves contacting the skin (e.g., containing a skin cell (e.g., keratinocyte, fibroblast)) of a subject in need thereof, with a composition and/or a formulation comprising a collagen or elastin polypeptide or a truncate thereof, and hyaluronic acid, to increase fibronectin production in the skin cell. The subject in need thereof may be an individual in need of an increase in fibronectin production in the skin. The subject in need thereof may be an individual in need of improvement of one or more of skin aging, skin elasticity, skin firmness, or wrinkles and fine lines. In some cases, the increase in fibronectin production may be synergistic or additive (e.g., as compared to treatment with a collagen or elastin polypeptide or a truncate thereof alone, as compared to treatment with hyaluronic acid alone).

In another aspect, a method is provided for increasing elastin production in a skin cell (e.g., keratinocyte, fibroblast), the method comprising: contacting the skin cell with a composition and/or a formulation comprising a collagen or elastin polypeptide or a truncate thereof, and ascorbic acid and/or retinol, thereby increasing elastin production in the skin cell. In some cases, the method involves contacting the skin (e.g., containing a skin cell (e.g., keratinocyte, fibroblast)) of a subject in need thereof, with a composition and/or a formulation comprising a collagen or elastin polypeptide or a truncate thereof, and ascorbic acid and/or retinol, to increase elastin production in the skin cell. The subject in need thereof may be an individual in need of an increase in elastin production in the skin. The subject in need thereof may be an individual in need of improvement of one or more of skin aging, skin elasticity, skin firmness, or wrinkles and fine lines. In some cases, the increase in elastin production may be synergistic or additive (e.g., as compared to treatment with a collagen or elastin polypeptide or a truncate thereof alone, as compared to treatment with ascorbic acid alone, as compared to treatment with retinol alone).

In another aspect, a method is provided for increasing type III collagen production in a skin cell (e.g., keratinocyte, fibroblast), the method comprising: contacting the skin cell with a composition and/or a formulation comprising a collagen or elastin polypeptide or a truncate thereof, and ascorbic acid, thereby increasing type III collagen production in the skin cell. In some cases, the method involves contacting the skin (e.g., containing a skin cell (e.g., keratinocyte, fibroblast)) of a subject in need thereof, with a composition and/or a formulation comprising a collagen or elastin polypeptide or a truncate thereof, and ascorbic acid, to increase type III collagen production in the skin cell. The subject in need thereof may be an individual in need of an increase in type III collagen production in the skin. The subject in need thereof may be an individual in need of improvement of one or more of skin aging, skin elasticity, skin firmness, or wrinkles and fine lines. In some cases, the increase in type III collagen production may be synergistic or additive (e.g., as compared to treatment with a collagen or elastin polypeptide or a truncate thereof alone, as compared to treatment with ascorbic acid alone).

### EXAMPLES

### Example 1. Production of Truncated Collagen

A codon optimized DNA sequence, optimized for expression in E. coli, encoding a jellyfish collagen with a truncation of 240 internal amino acids (relative to full-length jellyfish collagen (SEQ ID NO: 33)) was synthesized and expressed. The DNA sequence is shown below in SEQ ID NO: 1. In SEQ ID NO: 1, the DsbA secretion tag is encoded by nucleotides 1-72 and encodes amino acids 1-24 of SEQ ID NO: 2. The histidine tag comprising 9 histidine residues is encoded by nucleotides 73-99 of SEQ ID NO: 1 and encodes amino acids 25-33 of SEQ ID NO: 2. The linker is encoded by nucleotides 100-111 of SEQ ID NO: 1 and encodes amino acids 34-37 of SEQ ID NO: 2. The thrombin cleavage site is encoded by nucleotides 112-135 of SEQ ID NO: 1 and encodes amino acids 38-45 of SEQ ID NO: 2. The truncated collagen is encoded by nucleotides 136-822 of SEQ ID NO: 1 and encodes amino acids 46-274 of SEQ ID NO: 2.

(SEQ ID NO: 1)
ATGAAAAAGATTTGGCTGGCGCTGGCTGGTTTAGTTTTAGCGTTTAGCGC
ATCGGCGGCGCAGTATGAAGATCACCATCACCACCACCACCATCACCACT
CTGGCTCGAGCCTGGTGCCGCGCGGCAGCCATATGGGTCCGCAGGGTGTT
GTTGGTGCAGATGGTAAAGACGGTACCCCGGGTGAAAAAGGAGAACAGGG
ACGTACAGGTGCAGCAGGTAAACAGGGCAGCCCGGGTGCCGATGGTGCCC
GTGGCCCGCTGGGTAGCATTGGTCAGCAGGGTGCAAGAGGCGAACCGGGC
GATCCGGGTAGTCCGGGCCTGCGTGGTGATACGGGTCTGGCCGGTGTTAA
AGGCGTTGCAGGTCCTTCAGGTCGTCCAGGTCAACCGGGTGCAAATGGTC
TGCCGGGTGTTAATGGTCGTGGCGGTCTGGAACGTGGTCTGGCAGGACCG
CCGGGTCCTGATGGTCGCCGCGGTGAAACGGGTTCACCGGGTATTGCCGG
TGCCCTGGGTAAACCAGGTCTGGAAGGTCCGAAAGGTTATCCTGGTCTGC
GCGGTCGTGATGGTACCAATGGCAAACGTGGCGAACAGGGCGAAACCGGT
CCAGATGGTGTTCGTGGTATTCCGGGTAACGATGGTCAGAGCGGTAAACC
GGGCATTGATGGTATTGATGGCACCAATGGTCAGCCTGGCGAAGCAGGTT
ATCAGGGTGGTCGCGGTACCCGTGGTCAGCTGGGTGAAACAGGTGATGTT
GGTCAGAATGGTGATCGCGGCGCACCGGGTCCGGATGGTAGCAAAGGTAG
CGCCGGTCGTCCGGGTTTACGTTAA

The truncated collagen is approximately 54% of the full length jellyfish collagen (SEQ ID NO: 33) and is disclosed below in SEQ ID NO: 2.

(SEQ ID NO: 2)
MKKIWLALAGLVLAFSASAAQYEDHHHHHHHHHSGSSLVPRGSHMGPQGV
VGADGKDGTPGEKGEQGRTGAAGKQGSPGADGARGPLGSIGQQGARGEPG
DPGSPGLRGDTGLAGVKGVAGPSGRPGQPGANGLPGVNGRGGLERGLAGP
PGPDGRRGETGSPGIAGALGKPGLEGPKGYPGLRGRDGTNGKRGEQGETG
PDGVRGIPGNDGQSGKPGIDGIDGTNGQPGEAGYQGGRGTRGQLGETGDV
GQNGDRGAPGPDGSKGSAGRPGLR

The polynucleotide encoding the truncated jellyfish collagen without the DsbA secretion tag, the histidine tag, linker and thrombin cleavage site is disclosed in SEQ ID NO: 3.

(SEQ ID NO: 3)
GTCCGCAGGGTGTTGTTGGTGCAGATGGTAAAGACGGTACCCCGGGTGAA
AAAGGAGAACAGGGACGTACAGGTGCAGCAGGTAAACAGGGCAGCCCGGG
TGCCGATGGTGCCCGTGGCCCGCTGGGTAGCATTGGTCAGCAGGGTGCAA
GAGGCGAACCGGGCGATCCGGGTAGTCCGGGCCTGCGTGGTGATACGGGT
CTGGCCGGTGTTAAAGGCGTTGCAGGTCCTTCAGGTCGTCCAGGTCAACC
GGGTGCAAATGGTCTGCCGGGTGTTAATGGTCGTGGCGGTCTGGAACGTG
GTCTGGCAGGACCGCCGGGTCCTGATGGTCGCCGCGGTGAAACGGGTTCA
CCGGGTATTGCCGGTGCCCTGGGTAAACCAGGTCTGGAAGGTCCGAAAGG
TTATCCTGGTCTGCGCGGTCGTGATGGTACCAATGGCAAACGTGGCGAAC
AGGGCGAAACCGGTCCAGATGGTGTTCGTGGTATTCCGGGTAACGATGGT
CAGAGCGGTAAACCGGGCATTGATGGTATTGATGGCACCAATGGTCAGCC
TGGCGAAGCAGGTTATCAGGGTGGTCGCGGTACCCGTGGTCAGCTGGGTG
AAACAGGTGATGTTGGTCAGAATGGTGATCGCGGCGCACCGGGTCCGGAT
GGTAGCAAAGGTAGCGCCGGTCGTCCGGGTTTACGTTAA

The truncated jellyfish collagen amino acid sequence without the DsbA secretion tag, the histidine tag, linker and thrombin cleavage site is disclosed in SEQ ID NO: 4.

(SEQ ID NO: 4)
GPQGVVGADGKDGTPGEKGEQGRTGAAGKQGSPGADGARGPLGSIGQQGA
RGEPGDPGSPGLRGDTGLAGVKGVAGPSGRPGQPGANGLPGVNGRGGLER
GLAGPPGPDGRRGETGSPGIAGALGKPGLEGPKGYPGLRGRDGTNGKRGE
QGETGPDGVRGIPGNDGQSGKPGIDGIDGTNGQPGEAGYQGGRGTRGQLG
ETGDVGQNGDRGAPGPDGSKGSAGRPGLR

The polynucleotides of SEQ ID NO: 1 were codon optimized and synthesized by Gen9 DNA (now Ginkgo Bioworks) internal synthesis. Overlaps between the pET28 vector and SEQ ID NO: 1 were designed to be between 30 and 40 bp long and were added using PCR with the enzyme PrimeSTAR® GXL polymerase (www.clontech.com/US/Products/PCR/GC_Rich/PrimeSTAR_GXL_DNA_Polymerase?sitex=10020:22372:US). The opened pET28a vector and insert DNA (SEQ ID NO: 1) was then assembled together into the final plasmid using SGI Gibson Assembly® (us.vwr.com/store/product/17613857/gibson-assembly-hifi-1-step-kit-synthetic-genomics-inc). The plasmid sequence was then verified through Sanger sequencing through Eurofins Genomics (www.eurofinsgenomics.com).

The transformed cells were cultivated in minimal media and frozen in 1.5 ml aliquots with glycerol at a ratio of 50:50 of cells to glycerol. One vial of this frozen culture was revived in 50 ml of minimal media overnight at 37° C., 200 rpm. Cells were transferred into 300 ml of minimal media and grown for 6-9 hours to reach an OD600 of 5-10.

A bioreactor was prepared with 2.7 L of minimal media+glucose and 300 ml of OD600 of 5-10 culture was added to bring the starting volume to 3 L. Cells were grown at 28° C., pH 7 with Dissolved Oxygen maintained at 20% saturation using a cascade containing agitation, air, and oxygen. pH was controlled using 28% w/w ammonium hydroxide solution. Fermentation was run in a fed-batch mode using a DO-stat based feeding algorithm once the initial bolus of 40 g/L was depleted around 13 hours. After 24-26 hours of initial growth, the OD600 reached above 100. At this point, 300 mL of 500 g/L sucrose was added and temperature was reduced to 25° C. High density culture was induced for protein production using 1 mM IPTG. Fermentation was continued for another 20-24 hours and cells were harvested using a bench top centrifuge at 9000 rcf, 15° C. for 60 minutes. The cell pellet recovered from centrifugation was resuspended in a buffer containing 0.5 M NaCl and 0.1 M KH2PO4 at pH 8 in a weight by weight ratio of 2× buffer to 1× cells.

The harvested cells were disrupted in a homogenizer at 14,000 psi pressure in 2 passes. The resulting slurry contained the collagen protein along with other proteins.

The fermentations were performed at various temperature ranging from 25° to 28° C. For some fermentations, the temperature of the fermentation was maintained at a constant temperature and immediately upon completion of fermentation (OD600 of 5-10) the collagen was purified. For other fermentations, the temperature of the fermentations was maintained for a desired period of time and when cell densities of OD600 of 5-10 were reached, the temperature was reduced to induce protein production. Typically, the temperature was reduced from 28° C. to 25° C. After the fermentation at 25° C. was continued for 40-60 hours, the collagen was isolated.

The collagen was purified by acid treatment of homogenized cell broth. Additionally, acid treatment was also performed on non-homogenized whole cells recovered from the bioreactor after centrifugation and resuspension in the buffer described above. The pH of either the homogenized slurry or the resuspended whole cells was decreased to pH 3 using 6 M hydrochloric acid. Acidified cell slurry was incubated overnight at 4° C. with mixing, followed by centrifugation. Supernatant of the acidified slurry was tested on a polyacrylamide gel and found to contain collagen in relatively high abundance compared to starting pellet. The collagen slurry thus obtained was high in salts. To obtain volume and salt reduction, concentration and diafiltration steps were performed using an EMD Millipore Tangential Flow Filtration system with ultrafiltration cassettes of 0.1 m2 each. Total area of filtration was 0.2 m2 using 2 cassettes in parallel. A volume reduction of 5× and a salt reduction of 19×was achieved in the TFF stage. Final collagen slurry was run on an SDS-PAGE gel to confirm presence of the collagen. This slurry was dried using a multi-tray lyophilizer over 3 days to obtain a white, fluffy collagen powder.

The purified truncated collagen obtained from homogenized cell broth or non-homogenized cells were analyzed on an SDS-PAGE gel and a thick and clear band was observed at the expected size of 27 kilodaltons. The purified collagen was also analyzed by mass spectrometry and it was confirmed that the 27 kilodalton protein was jellyfish collagen.

An alternative purification method of the full length and truncated collagens is provided below.

The fermentation broth was mixed with 0.3-0.5% w/v of Poly Ethyl Imine (PEI). After 15 minutes of incubation with PEI, the fermentation broth was centrifuged at 9000 rcf for 15 minutes to recover the supernatant, which contained the collagen protein. The pellet containing the cells was discarded and the PEI-treated collagen containing supernatant was mixed with Sodium Bentonite (0.2% final w/v) (Wyopure®, Wyoming Bentonite) and centrifuged. The bentonite containing pellet was discarded and the supernatant was recovered.

The Bentonite treated supernatant was concentrated between 3-6 fold on a tangential flow filtration system (TFF) (EMD Millipore) using a 5 kDa cassette. The collagen was retained with almost no losses in the permeate stream. To remove salts, the retentate from the concentration step was diafiltered using the same TFF set-up. Final conductivity of the protein solution was <10 milliSiemens. The typical conductivity was between 400 microsiemens and 1.5 millisiemens. Highly concentrated collagen solutions had higher conductivities approaching 4 milliSiemens. A skilled artisan will understand that conductivities higher than 10 milliSiemens may be observed depending on the concentration of the collagen. Next, the desalted and concentrated protein was subjected to treatment with activated carbon using the W-L 9000 10×40 granulated resin (Carbon Activated Corporation). 5% w/v of the carbon resin was mixed with collagen containing protein feed and mixed at 45-50° C. with mild agitation. The carbon-treated slurry was filtered using a Buchner funnel lined with an Ertel Filter Press Pad M-953 (Ertel Alsop) in presence or absence of a filtration aid such as Diatomaceous Earth (Sigma Aldrich). Post-filtration, the collagen solution was filtered through a 0.2 micron filter followed by one to several hours of treatment with Sodium Bentonite (0.2% w/v final) (Wyopure®, Wyoming Bentonite) and centrifuged at 9000 rcf, 15-30 minutes to obtain a highly pure, clear and particulate free collagen solution. When removal of endotoxin proteins was desired, the protein was passed through a chromatographic filter like Sartobind-Q (Sartorius-Stedim) to specifically remove endotoxin proteins.

The purified collagen was analyzed on an SDS-PAGE gel and a thick and clear band was observed at 30 kilodaltons. The upshift in size is due to the structure of the collagen molecule and the high glycine/proline amino acid content. The purified collagen was also analyzed by mass spectrometry and it was confirmed that the 30 kilodalton protein was the truncated collagen.

The truncated collagens were further analyzed by HPLC using an Agilent 1100 series HPLC. The column was the 50 mm Agilent PLRP-S reverse phase column with an inner diameter of 4.6 mm, μM particle size and 1000 Angstrom pore size.

The sample was prepared by diluting 1:1 in a 0.04% sodium azide solution in HPLC-grade water. After dilution, the resulting mixture was filtered through a 0.45 μm filter to remove any large particles that can clog the HPLC column. For analysis, the samples are diluted appropriately with a 20 mM ammonium acetate buffer in HPLC-grade water at a pH of about 4.5. After mixing the sample, it was transferred to a 300 μL microvial that was then placed in the autosampler. Using ChemStation, the software that operates the HPLC, the analysis parameters such as sample flowrate, column temperature, mobile phase flowrate, mobile phase composition, etc. can be altered. In one exemplary, but non-limiting analysis the parameters were: sample flow rate of 1 mL/min, column temperature of 80° C., column pressure of 60-70 bar, mobile phase composition of 97.9% water/1.9% acetonitrile with 0.2% trifluoroacetic acid; UV wavelength for analysis of 214.4 nm, injection volume of 10 and sample run time of 10 minutes.

Under these conditions, the truncated jellyfish collation of SEQ ID NO: 5 has an elution time of about 5.4 minutes. ChemStation quantifies the peak area of the elution peak and calculates the protein concentration using a calibration curve that directly relates peak area to protein concentration. The calibration curve is generated using a known collagen solution that is serially diluted to contain collagen concentration ranges of 0.06 mg/mL to 1.00 mg/mL.

Truncated Collagen without His Tag-Linker-Thrombin Cleavage Site

A truncated jellyfish collagen without a His tag, linker, and thrombin cleavage site is disclosed below. The codon-optimized nucleotide sequence encoding this collagen is provided in SEQ ID NO: 6. The amino acid sequence is disclosed in SEQ ID NO: 7. The DsbA secretion tag is encoded by nucleotides 1-72 of SEQ ID NO: 6 and encodes amino acids 1-24 of SEQ ID NO: 7. The truncated collagen sequence is encoded by nucleotides 73-639 of SEQ ID NO: 6 and encodes amino acids 25-213 of SEQ ID NO: 7.

(SEQ ID NO: 6)
ATGAAAAAGATTTGGCTGGCGCTGGCTGGTTTAGTTTTAGCGTTTAGCGC
ATCGGCGGCGCAGTATGAAGATGGTCCGCAGGGTGTTGTTGGTGCAGATG
GTAAAGACGGTACCCCGGGTAATGCAGGTCAGAAAGGTCCGTCAGGTGAA
CCTGGCAGCCCTGGTAAAGCAGGTAGTGCCGGTGAGCAGGGTCCGCCGGG
CAAAGATGGTAGTAATGGTGAGCCGGGTAGCCCTGGCAAAGAAGGTGAAC
GTGGTCTGGCAGGACCGCCGGGTCCTGATGGTCGCCGCGGTGAAACGGGT
TCACCGGGTATTGCCGGTGCCCTGGGTAAACCAGGTCTGGAAGGTCCGAA
AGGTTATCCTGGTCTGCGCGGTCGTGATGGTACCAATGGCAAACGTGGCG
AACAGGGCGAAACCGGTCCAGATGGTGTTCGTGGTATTCCGGGTAACGAT
GGTCAGAGCGGTAAACCGGGCATTGATGGTATTGATGGCACCAATGGTCA
GCCTGGCGAAGCAGGTTATCAGGGTGGTCGCGGTACCCGTGGTCAGCTGG
GTGAAACAGGTGATGTTGGTCAGAATGGTGATCGCGGCGCACCGGGTCCG
GATGGTAGCAAAGGTAGCGCCGGTCGTCCGGGTTTACGTTAA
(SEQ ID NO: 7)
MKKIWLALAGLVLAFSASAAQYEDGPQGVVGADGKDGTPGNAGQKGPSGE
PGSPGKAGSAGEQGPPGKDGSNGEPGSPGKEGERGLAGPPGPDGRRGETG
SPGIAGALGKPGLEGPKGYPGLRGRDGTNGKRGEQGETGPDGVRGIPGND
GQSGKPGIDGIDGTNGQPGEAGYQGGRGTRGQLGETGDVGQNGDRGAPGP
DGSKGSAGRPGLR

A polynucleotide encoding a truncated jellyfish collagen without a His tag, linker and thrombin cleavage site is disclosed in SEQ ID NO: 8.

(SEQ ID NO: 8)
GGTCCGCAGGGTGTTGTTGGTGCAGATGGTAAAGACGGTACCCCGGGTAA
TGCAGGTCAGAAAGGTCCGTCAGGTGAACCTGGCAGCCCTGGTAAAGCAG
GTAGTGCCGGTGAGCAGGGTCCGCCGGGCAAAGATGGTAGTAATGGTGAG
CCGGGTAGCCCTGGCAAAGAAGGTGAACGTGGTCTGGCAGGACCGCCGGG
TCCTGATGGTCGCCGCGGTGAAACGGGTTCACCGGGTATTGCCGGTGCCC
TGGGTAAACCAGGTCTGGAAGGTCCGAAAGGTTATCCTGGTCTGCGCGGT
CGTGATGGTACCAATGGCAAACGTGGCGAACAGGGCGAAACCGGTCCAGA
TGGTGTTCGTGGTATTCCGGGTAACGATGGTCAGAGCGGTAAACCGGGCA
TTGATGGTATTGATGGCACCAATGGTCAGCCTGGCGAAGCAGGTTATCAG
GGTGGTCGCGGTACCCGTGGTCAGCTGGGTGAAACAGGTGATGTTGGTCA
GAATGGTGATCGCGGCGCACCGGGTCCGGATGGTAGCAAAGGTAGCGCCG
GTCGTCCGGGTTTACGTTAA

A truncated jellyfish collagen without a His tag, linker, and thrombin cleavage site is disclosed in SEQ ID NO: 5.

(SEQ ID NO: 5)
GPQGVVGADGKDGTPGNAGQKGPSGEPGSPGKAGSAGEQGPPGKDGSNGE
PGSPGKEGERGLAGPPGPDGRRGETGSPGIAGALGKPGLEGPKGYPGLRG
RDGTNGKRGEQGETGPDGVRGIPGNDGQSGKPGIDGIDGTNGQPGEAGYQ
GGRGTRGQLGETGDVGQNGDRGAPGPDGSKGSAGRPGLR

Truncated Collagen with DsbA Secretion Tag-His Tag-Linker-Thrombin Cleavage Site and GFP Beta-Lactamase Fusion (Version 1):

A jellyfish collagen with DsbA secretion tag-His tag-Linker-Thrombin cleavage site and GFP Beta-lactamase fusion is disclosed below. The codon-optimized nucleotide sequence encoding this collagen is provided in SEQ ID NO: 9. The amino acid sequence is disclosed in SEQ ID NO: 10. The DsbA secretion tag is encoded by nucleotides 1-72 of SEQ ID NO: 9 and encodes amino acids 1-24 of SEQ ID NO: 10. The His tag is encoded by nucleotides 73-99 of SEQ ID NO: 9 and encodes a 9 histidine tag of amino acids 25-33 of SEQ ID NO: 10. The linker is encoded by nucleotides 100-111 of SEQ ID NO: 9 and encodes amino acids 34-37 of SEQ ID NO: 10. The thrombin cleavage side is encoded by nucleotides 112-135 of SEQ ID NO: 9 and encodes amino acids 38-45 of SEQ ID NO: 10. The green fluorescent protein (GFP) with linker is encoded by nucleotides 136-873 of SEQ ID NO: 9 and encodes amino acids 46-291 of SEQ ID NO: 10. The truncated collagen sequence is encoded by nucleotides 874-1440 of SEQ ID NO: 9 and encodes amino acids 292-480 of SEQ ID NO: 10. The Beta-lactamase with linker is encoded by nucleotides 1441-2232 of SEQ ID NO: 9 and encodes amino acids 481-744 of SEQ ID NO: 10. The Beta-lactamase was properly targeted to the periplasmic space even though the polypeptide did not have an independent secretion tag. The DsbA secretion tag directed the entire transcript (Truncated Collagen with DsbA secretion tag-His tag-Linker-Thrombin cleavage site and GFP Beta-lactamase fusion protein) to the periplasmic space and the Beta-lactamase functioned properly.

(SEQ ID NO: 9)
ATGAAAAAGATTTGGCTGGCGCTGGCTGGTTTAGTTTTAGCGTTTAGCGC
ATCGGCGGCGCAGTATGAAGATCACCATCACCACCACCACCATCACCACT
CTGGCTCGAGCCTGGTGCCGCGCGGCAGCCATATGTCTGGCTCGAGCAGT
AAAGGTGAAGAACTGTTCACCGGTGTTGTTCCGATCCTGGTTGAACTGGA
TGGTGATGTTAACGGCCACAAATTCTCTGTTCGTGGTGAAGGTGAAGGTG
ATGCAACCAACGGTAAACTGACCCTGAAATTCATCTGCACTACCGGTAAA
CTGCCGGTTCCATGGCCGACTCTGGTGACTACCCTGACCTATGGTGTTCA
GTGTTTTTCTCGTTACCCGGATCACATGAAGCAGCATGATTTCTTCAAAT
CTGCAATGCCGGAAGGTTATGTACAGGAGCGCACCATTTCTTTCAAAGAC
GATGGCACCTACAAAACCCGTGCAGAGGTTAAATTTGAAGGTGATACTCT
GGTGAACCGTATTGAACTGAAAGGCATTGATTTCAAAGAGGACGGCAACA
TCCTGGGCCACAAACTGGAATATAACTTCAACTCCCATAACGTTTACATC
ACCGCAGACAAACAGAAGAACGGTATCAAAGCTAACTTCAAAATTCGCCA
TAACGTTGAAGACGGTAGCGTACAGCTGGCGGACCACTACCAGCAGAACA
CTCCGATCGGTGATGGTCCGGTTCTGCTGCCGGATAACCACTACCTGTCC
ACCCAGTCTAAACTGTCCAAAGACCCGAACGAAAAGCGCGACCACATGGT
GCTGCTGGAGTTCGTTACTGCAGCAGGTATCACGCACGGCATGGATGAAC
TCTACAAATCTGGCGCGCCGGGCGGTCCGCAGGGTGTTGTTGGTGCAGAT
GGTAAAGACGGTACCCCGGGTAATGCAGGTCAGAAAGGTCCGTCAGGTGA
ACCTGGCAGCCCTGGTAAAGCAGGTAGTGCCGGTGAGCAGGGTCCGCCGG
GCAAAGATGGTAGTAATGGTGAGCCGGGTAGCCCTGGCAAAGAAGGTGAA
CGTGGTCTGGCAGGACCGCCGGGTCCTGATGGTCGCCGCGGTGAAACGGG
TTCACCGGGTATTGCCGGTGCCCTGGGTAAACCAGGTCTGGAAGGTCCGA
AAGGTTATCCTGGTCTGCGCGGTCGTGATGGTACCAATGGCAAACGTGGC
GAACAGGGCGAAACCGGTCCAGATGGTGTTCGTGGTATTCCGGGTAACGA
TGGTCAGAGCGGTAAACCGGGCATTGATGGTATTGATGGCACCAATGGTC
AGCCTGGCGAAGCAGGTTATCAGGGTGGTCGCGGTACCCGTGGTCAGCTG
GGTGAAACAGGTGATGTTGGTCAGAATGGTGATCGCGGCGCACCGGGTCC
GGATGGTAGCAAAGGTAGCGCCGGTCGTCCGGGTTTACGTCACCCAGAAA
CGCTGGTGAAAGTAAAAGATGCTGAAGATCAGTTGGGTGCACGAGTGGGT
TACATCGAACTGGATCTCAACAGCGGTAAGATCCTTGAGAGTTTTCGCCC
CGAAGAACGTTTTCCAATGATGAGCACTTTTAAAGTTCTGCTATGTGGCG
CGGTATTATCCCGTATTGACGCCGGGCAAGAGCAACTCGGTCGCCGCATA
CACTATTCTCAGAATGACTTGGTTGAGTACTCACCAGTCACAGAAAAGCA
TCTTACGGATGGCATGACAGTAAGAGAATTATGCAGTGCTGCCATAACCA
TGAGTGATAACACTGCGGCCAACTTACTTCTGACAACGATCGGAGGACCG
AAGGAGCTAACCGCTTTTTTGCACAACATGGGGGATCATGTAACTCGCCT
TGATCGTTGGGAACCGGAGCTGAATGAAGCCATACCAAACGACGAGCGTG
ACACCACGATGCCTGTAGCAATGGCAACAACGTTGCGCAAACTATTAACT
GGCGAACTACTTACTCTAGCTTCCCGGCAACAATTAATAGACTGGATGGA
GGCGGATAAAGTTGCAGGACCACTTCTGCGCTCGGCCCTTCCGGCTGGCT
GGTTTATTGCTGATAAATCTGGAGCCGGTGAGCGTGGGTCTCGCGGTATC
ATTGCAGCACTGGGGCCAGATGGTAAGCCCTCCCGTATCGTAGTTATCTA
CACGACGGGGAGTCAGGCAACTATGGATGAACGAAATAGACAGATCGCTG
AGATAGGTGCCTCACTGATTAAGCATTGGTAA
(SEQ ID NO: 10)
MKKIWLALAGLVLAFSASAAQYEDHHHHHHHHHSGSSLVPRGSHMSGSSS
KGEELFTGVVPILVELDGDVNGHKFSVRGEGEGDATNGKLTLKFICTTGK
LPVPWPTLVTTLTYGVQCFSRYPDHMKQHDFFKSAMPEGYVQERTISFKD
DGTYKTRAEVKFEGDTLVNRIELKGIDFKEDGNILGHKLEYNFNSHNVYI
TADKQKNGIKANFKIRHNVEDGSVQLADHYQQNTPIGDGPVLLPDNHYLS
TQSKLSKDPNEKRDHMVLLEFVTAAGITHGMDELYKSGAPGGPQGVVGAD
GKDGTPGNAGQKGPSGEPGSPGKAGSAGEQGPPGKDGSNGEPGSPGKEGE
RGLAGPPGPDGRRGETGSPGIAGALGKPGLEGPKGYPGLRGRDGTNGKRG
EQGETGPDGVRGIPGNDGQSGKPGIDGIDGTNGQPGEAGYQGGRGTRGQL
GETGDVGQNGDRGAPGPDGSKGSAGRPGLRHPETLVKVKDAEDQLGARVG
YIELDLNSGKILESFRPEERFPMMSTFKVLLCGAVLSRIDAGQEQLGRRI
HYSQNDLVEYSPVTEKHLTDGMTVRELCSAAITMSDNTAANLLLTTIGGP
KELTAFLHNMGDHVTRLDRWEPELNEAIPNDERDTTMPVAMATTLRKLLT
GELLTLASRQQLIDWMEADKVAGPLLRSALPAGWFIADKSGAGERGSRGI
IAALGPDGKPSRIVVIYTTGSQATMDERNRQIAEIGASLIKHW

The polynucleotide of SEQ ID NO: 9 was constructed by assembling several DNA fragments. The collagen containing sequence was codon optimized and synthesized by Gen9 DNA (now Ginkgo Bioworks) internal synthesis. The GFP was also synthesized by Gen9. The Beta-lactamase was cloned out of the plasmid pKD46 (cgsc2.biology.yale.edu/Strain.php?ID=68099) using PCR with the enzyme PrimeSTAR® GXL polymerase (www.clontech.com/US/Products/PCR/GC_Rich/PrimeSTAR_GXL_DNA_Polymerase?sitex=1 0020:22372:US). Overlaps between the pET28 vector, GFP, Collagen, and Beta-lactamase was designed to be between 30 and 40 bp long and added using PCR with the enzyme PrimeSTAR® GXL polymerase. The opened pET28a vector and inserts were then assembled together into the final plasmid using SGI Gibson Assembly® (us.vwr.com/store/product/17613857/gibson-assembly-hifi-1-step-kit-synthetic-genomics-inc). The plasmid sequence was then verified through Sanger sequencing through Eurofins Genomics (www.eurofinsgenomics.com).

The transformed cells were cultivated in minimal media and frozen in 1.5 ml aliquots with glycerol at a ratio of 50:50 of cells to glycerol. One vial of this frozen culture was revived in 50 ml of minimal media overnight at 37° C., 200 rpm. Cells were transferred into 300 ml of minimal media and grown for 6-9 hours to reach an OD600 of 5-10.

A bioreactor was prepared with 2.7 L of minimal media+glucose and 300 ml of OD600 of 5-10 culture was added to bring the starting volume to 3 L. Cells were grown at 28° C., pH 7 with Dissolved Oxygen maintained at 20% saturation using a cascade containing agitation, air, and oxygen. pH was controlled using 28% w/w ammonium hydroxide solution. Fermentation was run in a fed-batch mode using a DO-stat based feeding algorithm once the initial bolus of 40 g/L was depleted around 13 hours. After 24-26 hours of initial growth, the OD600 reached above 100. At this point, 300 mL of 500 g/L sucrose was added and temperature was reduced to 25° C. High density culture was induced for protein production using 1 mM IPTG. Fermentation was continued for another 20-24 hours and cells were harvested using a bench top centrifuge at 9000 rcf, 15° C. for 60 minutes. Cell pellet recovered from centrifugation was resuspended in a buffer containing 0.5 M NaCl and 0.1 M KH2PO4 at pH 8 in a weight by weight ratio of 2× buffer to 1× cells.

The harvested cells were disrupted in a homogenizer at 14,000 psi pressure in 2 passes. The resulting slurry contained the collagen protein along with other proteins.

The collagen was purified by acid treatment of non-homogenized whole cells recovered from the bioreactor after centrifugation and resuspension in the buffer described above. The pH of the resuspended suspension was decreased to 3 using 6 M Hydrochloric acid. Acidified cell slurry was incubated overnight at 4° C. with mixing, followed by centrifugation. The pH was then raised to 9 using 10 N NaOH and the supernatant of the slurry was tested on a polyacrylamide gel and found to contain collagen in relatively high abundance compared to starting pellet. The collagen slurry thus obtained was high in salts. To obtain volume and salt reduction, concentration and diafiltration steps were performed using an EMD Millipore Tangential Flow Filtration system with ultrafiltration cassettes of 0.1 m2 each. Total area of filtration was 0.2 m2 using 2 cassettes in parallel. A volume reduction of 5× and a salt reduction of 19× was achieved in the TFF stage. Final collagen slurry was run on an SDS-PAGE gel to confirm presence of the collagen. This slurry was dried using a multi-tray lyophilizer over 3 days to obtain a white, fluffy collagen powder.

The purified collagen-GFP-Beta-lactamase fusion protein was analyzed on an SDS-PAGE gel and was observed to run at an apparent molecular weight of 90 kilodaltons. The expected size of the fusion protein is 85 kDa. The 90 kDa band was confirmed by mass spectrometry to be the correct collagen fusion protein.

Truncated Collagen with DsbA Secretion Tag-His Tag-Linker-Thrombin Cleavage Site and GFP Beta-Lactamase Fusion (Version 2):

A jellyfish collagen with DsbA secretion tag-His tag-Linker-Thrombin cleavage site and GFP Beta-lactamase fusion is disclosed below. The codon-optimized nucleotide sequence encoding this collagen is provided in SEQ ID NO: 11. The amino acid sequence is disclosed in SEQ ID NO: 12. The DsbA secretion tag is encoded by nucleotides 1-72 of SEQ ID NO: 11 and encodes amino acids 1-24 of SEQ ID NO: 12. The His tag is encoded by nucleotides 73-99 of SEQ ID NO: 11 and encodes a 9 histidine tag of amino acids 25-33 of SEQ ID NO: 12. The linker is encoded by nucleotides 100-111 of SEQ ID NO: 11 and encodes amino acids 34-37 of SEQ ID NO: 12. The thrombin cleavage site is encoded by nucleotides 112-135 of SEQ ID NO: 11 and encodes amino acids 38-45 of SEQ ID NO: 12. The green fluorescent protein (GFP) with linker is encoded by nucleotides 136-873 of SEQ ID NO: 11 and encodes amino acids 46-291 of SEQ ID NO: 12. The truncated collagen sequence is encoded by nucleotides 874-1440 of SEQ ID NO: 11 and encodes amino acids 292-480 of SEQ ID NO: 12. The Beta-lactamase with linker is encoded by nucleotides 1441-2232 of SEQ ID NO: 11 and encodes amino acids 481-744 of SEQ ID NO: 12.

(SEQ ID NO: 11)
ATGAAAAAGATTTGGCTGGCGCTGGCTGGTTTAGTTTTAGCGTTTAGCGC
ATCGGCGGCGCAGTATGAAGATCACCATCACCACCACCACCATCACCACT
CTGGCTCGAGCCTGGTGCCGCGCGGCAGCCATATGTCTGGCTCGAGCAGT
AAAGGTGAAGAACTGTTCACCGGTGTTGTTCCGATCCTGGTTGAACTGGA
TGGTGATGTTAACGGCCACAAATTCTCTGTTCGTGGTGAAGGTGAAGGTG
ATGCAACCAACGGTAAACTGACCCTGAAATTCATCTGCACTACCGGTAAA
CTGCCGGTTCCATGGCCGACTCTGGTGACTACCCTGACCTATGGTGTTCA
GTGTTTTTCTCGTTACCCGGATCACATGAAGCAGCATGATTTCTTCAAAT
CTGCAATGCCGGAAGGTTATGTACAGGAGCGCACCATTTCTTTCAAAGAC
GATGGCACCTACAAAACCCGTGCAGAGGTTAAATTTGAAGGTGATACTCT
GGTGAACCGTATTGAACTGAAAGGCATTGATTTCAAAGAGGACGGCAACA
TCCTGGGCCACAAACTGGAATATAACTTCAACTCCCATAACGTTTACATC
ACCGCAGACAAACAGAAGAACGGTATCAAAGCTAACTTCAAAATTCGCCA
TAACGTTGAAGACGGTAGCGTACAGCTGGCGGACCACTACCAGCAGAACA
CTCCGATCGGTGATGGTCCGGTTCTGCTGCCGGATAACCACTACCTGTCC
ACCCAGTCTaaaCTGTCCAAAGACCCGAACGAAAAGCGCGACCACATGGT
GCTGCTGGAGTTCGTTACTGCAGCAGGTATCACGCACGGCATGGATGAAC
TCTACAAATCTGGCGCGCCGGGCGGTCCGCAGGGTGTTGTTGGTGCAGAT
GGTAAAGACGGTACCCCGGGTAATGCAGGTCAGAAAGGTCCGTCAGGTGA
ACCTGGCAGCCCTGGTAAAGCAGGTAGTGCCGGTGAGCAGGGTCCGCCGG
GCAAAGATGGTAGTAATGGTGAGCCGGGTAGCCCTGGCAAAGAAGGTGAA
CGTGGTCTGGCAGGACCGCCGGGTCCTGATGGTCGCCGCGGTGAAACGGG
TTCACCGGGTATTGCCGGTGCCCTGGGTAAACCAGGTCTGGAAGGTCCGA
AAGGTTATCCTGGTCTGCGCGGTCGTGATGGTACCAATGGCAAACGTGGC
GAACAGGGCGAAACCGGTCCAGATGGTGTTCGTGGTATTCCGGGTAACGA
TGGTCAGAGCGGTAAACCGGGCATTGATGGTATTGATGGCACCAATGGTC
AGCCTGGCGAAGCAGGTTATCAGGGTGGTCGCGGTACCCGTGGTCAGCTG
GGTGAAACAGGTGATGTTGGTCAGAATGGTGATCGCGGCGCACCGGGTCC
GGATGGTAGCAAAGGTAGCGCCGGTCGTCCGGGTTTACGTCACCCAGAAA
CGCTGGTGAAAGTAAAAGATGCTGAAGATCAGTTGGGTGCACGAGTGGGT
TACATCGAACTGGATCTCAACAGCGGTAAGATCCTTGAGAGTTTTCGCCC
CGAAGAACGTTTTCCAATGATGAGCACTTTTAAAGTTCTGCTATGTGGCG
CGGTATTATCCCGTATTGACGCCGGGCAAGAGCAACTCGGTCGCCGCATA
CACTATTCTCAGAATGACTTGGTTGAGTACTCACCAGTCACAGAAAAGCA
TCTTACGGATGGCATGACAGTAAGAGAATTATGCAGTGCTGCCATAACCA
TGAGTGATAACACTGCGGCCAACTTACTTCTGACAACGATCGGAGGACCG
AAGGAGCTAACCGCTTTTTTGCACAACATGGGGGATCATGTAACTCGCCT
TGATCGTTGGGAACCGGAGCTGAATGAAGCCATACCAAACGACGAGCGTG
ACACCACGATGCCTGTAGCAATGGCAACAACGTTGCGCAAACTATTAACT
GGCGAACTACTTACTCTAGCTTCCCGGCAACAATTAATAGACTGGATGGA
GGCGGATAAAGTTGCAGGACCACTTCTGCGCTCGGCCCTTCCGGCTGGCT
GGTTTATTGCTGATAAATCTGGAGCCGGTGAGCGTGGGTCTCGCGGTATC
ATTGCAGCACTGGGGCCAGATGGTAAGCCCTCCCGTATCGTAGTTATCTA
CACGACGGGGAGTCAGGCAACTATGGATGAACGAAATAGACAGATCGCTG
AGATAGGTGCCTCACTGATTAAGCATTGGTAA
(SEQ ID NO: 12)
MKKIWLALAGLVLAFSASAAQYEDHHHHHHHHHSGSSLVPRGSHMSGSSS
KGEELFTGVVPILVELDGDVNGHKFSVRGEGEGDATNGKLTLKFICTTGK
LPVPWPTLVTTLTYGVQCFSRYPDHMKQHDFFKSAMPEGYVQERTISFKD
DGTYKTRAEVKFEGDTLVNRIELKGIDFKEDGNILGHKLEYNFNSHNVYI
TADKQKNGIKANFKIRHNVEDGSVQLADHYQQNTPIGDGPVLLPDNHYLS
TQSKLSKDPNEKRDHMVLLEFVTAAGITHGMDELYKSGAPGGPQGVVGAD
GKDGTPGNAGQKGPSGEPGSPGKAGSAGEQGPPGKDGSNGEPGSPGKEGE
RGLAGPPGPDGRRGETGSPGIAGALGKPGLEGPKGYPGLRGRDGTNGKRG
EQGETGPDGVRGIPGNDGQSGKPGIDGIDGTNGQPGEAGYQGGRGTRGQL
GETGDVGQNGDRGAPGPDGSKGSAGRPGLRHPETLVKVKDAEDQLGARVG
YIELDLNSGKILESFRPEERFPMMSTFKVLLCGAVLSRIDAGQEQLGRRI
HYSQNDLVEYSPVTEKHLTDGMTVRELCSAAITMSDNTAANLLLTTIGGP
KELTAFLHNMGDHVTRLDRWEPELNEAIPNDERDTTMPVAMATTLRKLLT
GELLTLASRQQLIDWMEADKVAGPLLRSALPAGWFIADKSGAGERGSRGI
IAALGPDGKPSRIVVIYTTGSQATMDERNRQIAEIGASLIKHW

### Example 2. Human Collagens

Human Collagen Type 21 Alpha 1

A truncated human collagen type 21 alpha 1 (truncated relative to full-length human type 21 alpha 1 collagen (SEQ ID NO: 31)) without a His tag, linker, and thrombin cleavage site is disclosed below. The codon-optimized nucleotide sequence encoding this collagen and the amino acid sequence are disclosed below. The DsbA secretion tag is encoded by nucleotides 1-72 of SEQ ID NO: 13 and encodes amino acids 1-24 of SEQ ID NO: 14. The truncated collagen sequence is encoded by nucleotides 73-633 of SEQ ID NO: 13 and encodes amino acids 25-211 of SEQ ID NO: 14.

The codon-optimized nucleotide sequence encoding this collagen is provided in SEQ ID NO: 13.

(SEQ ID NO: 13)
ATGAAAAAGATTTGGCTGGCGCTGGCTGGTTTAGTTTTAGCGTTTAGCGC
ATCGGCGGCGCAGTATGAAGATGCAGGTTTTCCGGGTCTGCCTGGTCCGG
CAGGCGAACCGGGTCGTCATGGTAAAGATGGTCTGATGGGTAGTCCGGGT
TTTAAAGGTGAAGCAGGTTCACCGGGTGCACCTGGTCAGGATGGCACCCG
TGGTGAACCGGGTATTCCGGGATTTCCGGGTAATCGTGGCCTGATGGGTC
AGAAAGGTGAAATTGGTCCGCCTGGTCAGCAGGGTAAAAAAGGCGCACCG
GGTATGCCAGGACTGATGGGTTCAAATGGCAGTCCGGGTCAGCCAGGCAC
ACCGGGTTCAAAAGGTAGCAAAGGCGAACCTGGTATTCAGGGTATGCCTG
GTGCAAGCGGTCTGAAAGGCGAGCCAGGTGCCACCGGTTCTCCGGGTGAA
CCAGGTTATATGGGTCTGCCAGGTATCCAAGGCAAAAAAGGTGATAAAGG
TAATCAGGGCGAAAAAGGCATTCAGGGCCAGAAAGGCGAAAATGGCCGTC
AGGGTATTCCAGGCCAGCAGGGCATCCAGGGTCATCATGGTGCAAAAGGT
GAACGTGGTGAAAAGGGCGAACCAGGTGTTCGTTTA

The amino acid sequence is disclosed in SEQ ID NO: 14.

(SEQ ID NO: 14)
MKKIWLALAGLVLAFSASAAQYEDAGFPGLPGPAGEPGRHGKDGLMGSPG
FKGEAGSPGAPGQDGTRGEPGIPGFPGNRGLMGQKGEIGPPGQQGKKGAP
GMPGLMGSNGSPGQPGTPGSKGSKGEPGIQGMPGASGLKGEPGATGSPGE
PGYMGLPGIQGKKGDKGNQGEKGIQGQKGENGRQGIPGQQGIQGHHGAKG
ERGEKGEPGVR

The codon-optimized nucleotide sequence encoding the truncated human collagen type 21 alpha 1 without the DsbA secretion tag collagen is provided in SEQ ID NO: 15.

(SEQ ID NO: 15)
TGCAGGTTTTCCGGGTCTGCCTGGTCCGGCAGGCGAACCGGGTCGTCATG
GTAAAGATGGTCTGATGGGTAGTCCGGGTTTTAAAGGTGAAGCAGGTTCA
CCGGGTGCACCTGGTCAGGATGGCACCCGTGGTGAACCGGGTATTCCGGG
ATTTCCGGGTAATCGTGGCCTGATGGGTCAGAAAGGTGAAATTGGTCCGC
CTGGTCAGCAGGGTAAAAAAGGCGCACCGGGTATGCCAGGACTGATGGGT
TCAAATGGCAGTCCGGGTCAGCCAGGCACACCGGGTTCAAAAGGTAGCAA
AGGCGAACCTGGTATTCAGGGTATGCCTGGTGCAAGCGGTCTGAAAGGCG
AGCCAGGTGCCACCGGTTCTCCGGGTGAACCAGGTTATATGGGTCTGCCA
GGTATCCAAGGCAAAAAAGGTGATAAAGGTAATCAGGGCGAAAAAGGCAT
TCAGGGCCAGAAAGGCGAAAATGGCCGTCAGGGTATTCCAGGCCAGCAGG
GCATCCAGGGTCATCATGGTGCAAAAGGTGAACGTGGTGAAAAGGGCGAA
CCAGGTGTTCGTtaa

The amino acid sequence of truncated human collagen type 21 alpha 1 without the DsbA secretion tag is disclosed in SEQ ID NO: 16.

(SEQ ID NO: 16)
AGFPGLPGPAGEPGRHGKDGLMGSPGFKGEAGSPGAPGQDGTRGEPGIPG
FPGNRGLMGQKGEIGPPGQQGKKGAPGMPGLMGSNGSPGQPGTPGSKGSK
GEPGIQGMPGASGLKGEPGATGSPGEPGYMGLPGIQGKKGDKGNQGEKGI
QGQKGENGRQGIPGQQGIQGHHGAKGERGEKGEPGVR

The polynucleotides of SEQ ID NO: 13 were synthesized by Twist Bioscience. Overlaps between the pET28 vector and SEQ ID NO: 15 and SEQ ID NO: 16 were designed to be between 20 and 30 bp long and added using PCR with the enzyme PrimeSTAR® GXL polymerase (www.takarabio.com/products/per/gc-rich-per/primestar-gxl-dna-polymerase). The opened pET28a vector and insert DNA (SEQ ID NO: 13) was then assembled together into the final plasmid using In-Fusion Cloning (www.takarabio.com/products/cloning/in-fusion-cloning). The plasmid sequence was then verified through Sanger sequencing through Genewiz (www.genewiz.com/en).

The transformed cells were cultivated in minimal media and frozen in 1.5 ml aliquots with vegetable glycerin at a ratio of 50:50 of cells to glycerin. One vial of this frozen culture was revived in 50 ml of minimal media overnight at 37° C., 200 rpm. Cells were transferred into 300 ml of minimal media and grown for 6-9 hours to reach an OD600 of 5-10.

Minimal media used in this example and throughout this application is prepared as follows:
1) Autoclave 5 L of 550 g/kg Glucose syrup at concentration in DI water. (VWR, product #97061-170).
2) Autoclave in 3946 mL of DI water:
20 g (NH₄)₂HPO₄. (VWR, product #97061-932);
66.5 g KH₂PO₄. (VWR, product #97062-348);
22.5 g H₃C₆H₅O₇. (VWR, product #BDH9228-2.5KG);
8.85 g MgSO₄·7H₂O. (VWR, product #97062-134);
10 mL of 1000× Trace metals formulation (Table 5).
After autoclaving, add
118 g of (1) to (2);
5 mL of 25 mg/mL Kanamycin Sulfate (VWR-V0408);
Use 28% NH₄OH (VWR, product #BDH3022) to adjust pH to 6.1.

TABLE 5
Trace metals formulation

Ferrous Sulfate Heptahydrate, 27.8 g/L (Spectrum, 7782-63-0)
Zinc Sulfate heptahydrate, 2.88 g/L (Spectrum, 7446-20-0)
Calcium chloride dihydrate, 2.94 g/L (Spectrum, 2971347)
Sodium molybdate dihydrate, 0.48 g/L (Spectrum, 10102-40-6)
Manganese chloride tetrahydrate, 1.26 g/L (Spectrum, 13446-34-9)
Sodium selenite, 0.35 g/L (Spectrum, 10102-18-8)
Boric acid, 0.12 g/L (Spectrum, 10043-35-3

The fermentations were performed at various temperature ranging from 25° to 28° C. For some fermentations, the temperature of the fermentation was maintained at a constant temperature and immediately upon completion of fermentation the collagen was purified. For other fermentations, the temperature of the fermentations was maintained for a desired period of time and when cell densities of OD600 of 10-20 were reached, the temperature was reduced to induce protein production. Typically, the temperature was reduced from 28° C. to 25° C. After the fermentation at 25° C. was continued for 40-60 hours.

The collagen was purified as follows: The pH of the fermentation broth was decreased to between 3-3.5 using 5-50% Sulfuric Acid. The cells were then separated using centrifugation. Supernatant of the acidified broth was tested on a polyacrylamide gel and found to contain collagen in relatively high abundance compared to starting pellet. The collagen slurry thus obtained was high in salts. To obtain volume and salt reduction, concentration and diafiltration steps were performed using an EMD Millipore Tangential Flow Filtration system with ultrafiltration cassettes of 0.1 m2 each. Total area of filtration was 0.2 m2 using 2 cassettes in parallel. A volume reduction of 5× and a salt reduction of 19×was achieved in the TFF stage. Final collagen slurry was run on an SDS-PAGE gel to confirm presence of the collagen.

The purified collagen was analyzed on an SDS-PAGE gel and a thick and clear band was observed at the expected size of 25 kilodaltons. Quantification of collagen titers and purity were conducted using reverse phase and size exclusion HPLC chromatography. Titers are usually between 3 to 8 grams per liter. The purified collagen was also further analyzed by mass spectrometry and it was confirmed to match the published sequence of human type 21 collagen.

### Truncated Human Collagen Type 1 Alpha 2 (1)

A truncated human collagen type 1 alpha 2 (truncated relative to full-length human collagen type 1 alpha 2 (SEQ ID NO: 32)) without a His tag, linker, and thrombin cleavage site is disclosed below. The codon-optimized nucleotide sequence and the amino acid sequences are disclosed below. The DsbA secretion tag is encoded by nucleotides 1-72 of SEQ ID NO: 17 and encodes amino acids 1-24 of SEQ ID NO: 18. The truncated collagen sequence is encoded by nucleotides 73-636 of SEQ ID NO: 17 and encodes amino acids 25-212 of SEQ ID NO: 18.

The codon-optimized nucleotide sequence encoding this collagen is provided in SEQ ID NO: 17.

(SEQ ID NO: 17)
ATGAAAAAGATTTGGCTGGCGCTGGCTGGTTTAGTTTTAGCGTTTAGCGC
ATCGGCGGCGCAGTATGAAGATATGGGTCCGCCTGGTAGCCGTGGTGCAA
GTGGTCCGGCAGGCGTTCGTGGTCCGAATGGTGATGCAGGTCGTCCGGGT
GAACCGGGTCTGATGGGTCCTCGTGGTCTGCCTGGTTCACCGGGTAATAT
TGGTCCTGCAGGTAAAGAAGGTCCGGTTGGTCTGCCAGGTATTGATGGCC
GTCCGGGTCCGATTGGTCCAGCCGGTGCACGTGGTGAACCTGGCAATATT
GGTTTTCCGGGTCCTAAAGGTCCGACCGGTGATCCGGGTAAAAATGGTGA
TAAAGGTCATGCAGGTCTGGCAGGCGCACGCGGTGCACCTGGTCCGGATG
GTAATAATGGTGCACAGGGTCCACCGGGTCCGCAGGGTGTTCAAGGTGGT
AAAGGCGAACAGGGTCCTGCCGGTCCTCCGGGTTTTCAGGGACTGCCTGG
TCCGAGCGGTCCTGCGGGTGAAGTTGGTAAACCTGGTGAACGCGGTCTGC
ATGGTGAATTTGGCCTGCCTGGGCCTGCAGGTCCGCGTGGCGAACGTGGT
CCGCCAGGTGAAAGCGGTGCAGCAGGTCCGACAGGTTAA

The amino acid sequence is disclosed in SEQ ID NO: 18.

(SEQ ID NO: 18)
MKKIWLALAGLVLAFSASAAQYEDMGPPGSRGASGPAGVRGPNGDAGRPG
EPGLMGPRGLPGSPGNIGPAGKEGPVGLPGIDGRPGPIGPAGARGEPGNI
GFPGPKGPTGDPGKNGDKGHAGLAGARGAPGPDGNNGAQGPPGPQGVQGG
KGEQGPAGPPGFQGLPGPSGPAGEVGKPGERGLHGEFGLPGPAGPRGERG
PPGESGAAGPTG

The nucleic acid sequence of truncated human collagen type 1 alpha 2(1) without the DsbA secretion tag is disclosed in SEQ ID NO: 19.

(SEQ ID NO: 19)
ATGGGTCCGCCTGGTAGCCGTGGTGCAAGTGGTCCGGCAGGCGTTCGTGG
TCCGAATGGTGATGCAGGTCGTCCGGGTGAACCGGGTCTGATGGGTCCTC
GTGGTCTGCCTGGTTCACCGGGTAATATTGGTCCTGCAGGTAAAGAAGGT
CCGGTTGGTCTGCCAGGTATTGATGGCCGTCCGGGTCCGATTGGTCCAGC
CGGTGCACGTGGTGAACCTGGCAATATTGGTTTTCCGGGTCCTAAAGGTC
CGACCGGTGATCCGGGTAAAAATGGTGATAAAGGTCATGCAGGTCTGGCA
GGCGCACGCGGTGCACCTGGTCCGGATGGTAATAATGGTGCACAGGGTCC
ACCGGGTCCGCAGGGTGTTCAAGGTGGTAAAGGCGAACAGGGTCCTGCCG
GTCCTCCGGGTTTTCAGGGACTGCCTGGTCCGAGCGGTCCTGCGGGTGAA
GTTGGTAAACCTGGTGAACGCGGTCTGCATGGTGAATTTGGCCTGCCTGG
GCCTGCAGGTCCGCGTGGCGAACGTGGTCCGCCAGGTGAAAGCGGTGCAG
CAGGTCCGACAGGTTAA

The amino acid sequence of truncated human collagen type 1 alpha 2(1) without the DsbA secretion tag is disclosed in SEQ ID NO: 20.

(SEQ ID NO: 20)
MGPPGSRGASGPAGVRGPNGDAGRPGEPGLMGPRGLPGSPGNIGPAGKEG
PVGLPGIDGRPGPIGPAGARGEPGNIGFPGPKGPTGDPGKNGDKGHAGLA
GARGAPGPDGNNGAQGPPGPQGVQGGKGEQGPAGPPGFQGLPGPSGPAGE
VGKPGERGLHGEFGLPGPAGPRGERGPPGESGAAGPTG

Truncated Human Collagen type 1 alpha 2 (2)

A truncated human collagen type 1 alpha 2 (truncated relative to full-length human collagen type 1 alpha 2 (SEQ ID NO: 32)) without a His tag, linker, and thrombin cleavage site is disclosed below. The codon-optimized nucleotide sequence and the amino acid sequences are disclosed below. The DsbA secretion tag is encoded by nucleotides 1-72 of SEQ ID NO: 21 and encodes amino acids 1-24 of SEQ ID NO: 22. The truncated collagen sequence is encoded by nucleotides 73-609 of SEQ ID NO: 21 and encodes amino acids 25-203 of SEQ ID NO: 22.

The codon-optimized nucleotide sequence encoding this collagen is provided in SEQ ID NO: 21.

(SEQ ID NO: 21)
ATGAAAAAGATTTGGCTGGCGCTGGCTGGTTTAGTTTTAGCGTTTAGCGC
ATCGGCGGCGCAGTATGAAGATGGTTTTCAGGGTCCTGCCGGTGAACCGG
GTGAACCTGGTCAGACAGGTCCGGCAGGCGCACGTGGTCCTGCAGGTCCT
CCTGGTAAAGCCGGTGAAGATGGTCATCCGGGTAAACCGGGTCGTCCTGG
TGAACGTGGTGTTGTTGGTCCGCAGGGTGCCCGTGGTTTTCCGGGTACTC
CGGGTCTGCCAGGTTTTAAAGGTATTCGTGGTCATAATGGTCTGGATGGT
CTGAAAGGTCAGCCTGGTGCACCGGGTGTTAAAGGTGAACCAGGTGCTCC
GGGTGAAAATGGCACACCGGGTCAGACCGGTGCGCGTGGTCTGCCTGGCG
AACGCGGTCGTGTTGGTGCACCTGGTCCAGCCGGTGCACGCGGTAGTGAT
GGTAGCGTTGGTCCGGTTGGTCCAGCGGGTCCGATTGGTAGCGCAGGTCC
ACCGGGTTTTCCAGGCGCACCGGGTCCGAAAGGTGAAATTGGTGCAGTTG
GTAATGCAGGCCCTGCCGGTCCAGCAGGACCGCGTGGTGAAGTTGGCCTG
CCTGGTCTGTAA

The amino acid sequence is disclosed in SEQ ID NO: 22.

(SEQ ID NO: 22)
MKKIWLALAGLVLAFSASAAQYEDGFQGPAGEPGEPGQTGPAGARGPAGP
PGKAGEDGHPGKPGRPGERGVVGPQGARGFPGTPGLPGFKGIRGHNGLDG
LKGQPGAPGVKGEPGAPGENGTPGQTGARGLPGERGRVGAPGPAGARGSD
GSVGPVGPAGPIGSAGPPGFPGAPGPKGEIGAVGNAGPAGPAGPRGEVGL
PGL

The nucleic acid sequence of truncated human collagen type 1 alpha 2(2) without the DsbA secretion tag is disclosed in SEQ ID NO: 23.

(SEQ ID NO: 23)
GGTTTTCAGGGTCCTGCCGGTGAACCGGGTGAACCTGGTCAGACAGGTCC
GGCAGGCGCACGTGGTCCTGCAGGTCCTCCTGGTAAAGCCGGTGAAGATG
GTCATCCGGGTAAACCGGGTCGTCCTGGTGAACGTGGTGTTGTTGGTCCG
CAGGGTGCCCGTGGTTTTCCGGGTACTCCGGGTCTGCCAGGTTTTAAAGG
TATTCGTGGTCATAATGGTCTGGATGGTCTGAAAGGTCAGCCTGGTGCAC
CGGGTGTTAAAGGTGAACCAGGTGCTCCGGGTGAAAATGGCACACCGGGT
CAGACCGGTGCGCGTGGTCTGCCTGGCGAACGCGGTCGTGTTGGTGCACC
TGGTCCAGCCGGTGCACGCGGTAGTGATGGTAGCGTTGGTCCGGTTGGTC
CAGCGGGTCCGATTGGTAGCGCAGGTCCACCGGGTTTTCCAGGCGCACCG
GGTCCGAAAGGTGAAATTGGTGCAGTTGGTAATGCAGGCCCTGCCGGTCC
AGCAGGACCGCGTGGTGAAGTTGGCCTGCCTGGTCTGTAA

The amino acid sequence of truncated human collagen type 1 alpha 2(2) without the DsbA secretion tag is disclosed in SEQ ID NO: 24.

(SEQ ID NO: 24)
GFQGPAGEPGEPGQTGPAGARGPAGPPGKAGEDGHPGKPGRPGERGVVGP
QGARGFPGTPGLPGFKGIRGHNGLDGLKGQPGAPGVKGEPGAPGENGTPG
QTGARGLPGERGRVGAPGPAGARGSDGSVGPVGPAGPIGSAGPPGFPGAP
GPKGEIGAVGNAGPAGPAGPRGEVGLPGL

The polynucleotides of SEQ ID NO: 13, 17, or 21 were subcloned in vector pET28a as described herein to prepare a transformation vector. Host cells were transformed with the vector the polynucleotides were expressed as described in Example 1.

After the fermentation was completed, the truncated human collagen was purified from the fermentation broth using the procedures disclosed in Example 2. The purified truncated human collagens were analyzed using SDS-PAGE and HPLC as disclosed in Example 2.

All three truncated human collagens ran at the expected molecular weights in the SDS-PAGE analysis. In analyzing the truncated human collagens using HPLC, a standard curve using the jellyfish collagen of Example 1 was utilized. The retention times of the human collagens were slightly different than the jellyfish collagen. The retention time of SEQ ID NO: 16 was 5.645 minutes, the retention time of SEQ ID NO: 20 was 5.631 minutes, and SEQ ID NO: 24 ran at two peaks and the retention times were 5.531 and 5.7 minutes.

Truncated Human Collagen Type 1 Alpha 2 Truncation 5 with DsbA Secretion and FLAG Tag

The amino acid sequence of truncated human collagen type 1 alpha 2 truncation 5 with DsbA secretion and FLAG tag is disclosed in SEQ ID NO: 25. The DsbA secretion tag is encoded by nucleotides 1-57 of SEQ ID NO: 26 and the amino acid sequences are amino acids 1-19 of SEQ ID NO: 25. The collagen nucleotide sequences are nucleotides 58-657 of SEQ ID NO: 26 and the amino acid sequences are amino acids 20-219 of SEQ ID NO: 25. The FLAG nucleotide sequences are nucleotides 658-684 of SEQ ID NO: 26 and the amino acid sequences are amino acids 220-228 of SEQ ID NO: 25.

(SEQ ID NO: 25)
MKKIWLALAGLVLAFSASAGDQGPVGRTGEVGAVGPPGFAGEKGPSGEAG
TAGPPGTPGPQGLLGAPGILGLPGSRGERGLPGVAGAVGEPGPLGIAGPP
GARGPPGAVGSPGVNGAPGEAGRDGNPGNDGPPGRDGQPGHKGERGYPGN
IGPVGAAGAPGPHGPVGPAGKHGNRGETGPSGPVGPAGAVGPRGPSGPQG
IRGDKGEPGEKGPRGLPGLGDYKDDDDK

The nucleic acid sequence of truncated human collagen type 1 alpha 2 truncation 5 with DsbA secretion and FLAG tag is disclosed in SEQ ID NO: 26.

(SEQ ID NO: 26)
ATGAAAAAGATTTGGCTGGCGCTGGCTGGTTTAGTTTTAGCGTTTAGCGC
ATCGGCGGGTGATCAGGGTCCGGTTGGTCGTACCGGTGAAGTTGGTGCAG
TCGGGCCGCCGGGTTTTGCGGGTGAAAAAGGCCCGTCAGGTGAAGCAGGC
ACCGCTGGCCCTCCTGGCACGCCTGGCCCACAGGGTTTACTGGGCGCACC
TGGAATTCTGGGACTGCCGGGCAGCCGTGGAGAACGCGGTTTACCAGGTG
TTGCCGGTGCCGTTGGTGAACCTGGTCCACTGGGCATTGCAGGGCCGCCT
GGCGCACGGGGACCGCCTGGTGCTGTTGGTAGTCCGGGTGTGAATGGTGC
TCCGGGTGAAGCCGGTCGTGACGGTAATCCGGGAAATGACGGCCCGCCAG
GCCGCGATGGTCAGCCGGGTCATAAAGGTGAGCGTGGTTACCCAGGTAAT
ATTGGTCCAGTCGGTGCCGCCGGTGCGCCGGGTCCTCATGGCCCTGTCGG
TCCAGCCGGTAAACATGGTAATCGCGGTGAGACAGGTCCGTCAGGACCAG
TGGGCCCTGCTGGCGCAGTCGGTCCGCGCGGGCCGAGTGGCCCTCAGGGT
ATTCGTGGCGATAAAGGGGAACCGGGCGAAAAAGGGCCGCGGGGTCTGCC
AGGCCTGGGTGACTACAAAGACGACGACGACAAATAA

The polynucleotide of SEQ ID NO: 26 was subcloned into vector pET28a, expressed in host E. coli cells and the truncated collagen was purified as described herein. The purified collagen produced a clear band on SDS-PAGE and an anti-FLAG western was observed at around 100 kilodaltons. There were no existing bands that appear at that location on the gel in the absence of expression of this protein.

Truncated Human Collagen Type 1 Alpha 2 Truncation 6 with DsbA Secretion and FLAG Tag

The amino acid sequence of truncated human collagen type 1 alpha 2 truncation 6 with DsbA secretion and FLAG tag is disclosed in SEQ ID NO: 27. The DsbA secretion tag is encoded by nucleotides 1-57 of SEQ ID NO: 28 and the amino acid sequences are amino acids 1-19 of SEQ ID NO: 27. The collagen nucleotide sequences are nucleotides 58-657 of SEQ ID NO: 28 and the amino acid sequences are amino acids 20-219 of SEQ ID NO: 27. The FLAG nucleotide sequences are nucleotides 658-684 of SEQ ID NO: 28 and the amino acid sequences are amino acids 220-228 of SEQ ID NO: 27.

(SEQ ID NO: 27)
MKKIWLALAGLVLAFSASAKGHNGLQGLPGIAGHHGDQGAPGSVGPAGPR
GPAGPSGPAGKDGRTGHPGTVGPAGIRGPQGHQGPAGPPGPPGPPGPPGV
SGGGYDFGYDGDFYRADQPRSAPSLRPKDYEVDATLKSLNNQIETLLTPE
GSRKNPARTCRDLRLSHPEWSSGYYWIDPNQGCTMDAIKVYCDFSTGETC
IRAQPENIPAKNWYRSSKDGDYKDDDDK

The nucleic acid sequence of truncated human collagen type 1 alpha 2 truncation 6 with DsbA secretion and FLAG tag is disclosed in SEQ ID NO: 28.

(SEQ ID NO: 28)
ATGAAAAAGATTTGGCTGGCGCTGGCTGGTTTAGTTTTAGCGTTTAGCGC
ATCGGCGAAAGGTCACAATGGACTGCAAGGCCTGCCAGGTATTGCAGGTC
ATCATGGTGATCAAGGTGCCCCGGGAAGCGTTGGTCCGGCGGGGCCGAGA
GGCCCTGCGGGACCTTCAGGTCCGGCAGGCAAAGATGGTCGGACAGGCCA
TCCGGGCACCGTTGGCCCTGCAGGAATTCGTGGACCGCAGGGTCATCAGG
GACCTGCTGGTCCGCCAGGTCCCCCGGGCCCTCCGGGACCACCGGGTGTT
AGTGGTGGTGGTTATGATTTTGGCTATGATGGTGATTTTTATCGTGCAGA
TCAGCCGCGTAGCGCACCGAGCCTGCGTCCTAAAGATTATGAAGTTGATG
CAACCCTGAAAAGCCTGAATAATCAGATTGAAACACTGCTGACACCGGAA
GGTAGCCGTAAAAATCCGGCCCGTACCTGTCGTGATCTGCGTCTGAGCCA
CCCGGAATGGAGCAGCGGTTATTATTGGATTGATCCGAATCAAGGTTGTA
CCATGGATGCAATTAAAGTTTATTGTGATTTTAGCACAGGTGAAACATGT
ATCCGTGCACAGCCGGAAAATATTCCGGCCAAAAATTGGTATCGTAGTAG
CAAAGATGGTGACTACAAAGACGACGACGACAAATAA

The polynucleotide of SEQ ID NO: 28 was subcloned into vector pET28a, expressed in host E. coli cells and the truncated collagen was purified as described herein. The purified collagen produced a clear band on SDS-PAGE and an anti-FLAG western was observed at around 25 kilodaltons. There were no existing bands that appear at that location on the gel in the absence of expression of this protein.

Truncated Human Collagen Type 1 Alpha 2 Truncation 7 with DsbA Secretion and FLAG Tag

The amino acid sequence of truncated human collagen type 1 alpha 2 truncation 7 with DsbA secretion and FLAG tag is disclosed in SEQ ID NO: 29. The DsbA secretion tag is encoded by nucleotides 1-57 of SEQ ID NO: 30 and the amino acid sequences are amino acids 1-19 of SEQ ID NO: 29. The collagen nucleotide sequences are nucleotides 58-759 of SEQ ID NO: 30 and the amino acid sequences are amino acids 20-253 of SEQ ID NO: 29. The FLAG nucleotide sequences are nucleotides 760-786 of SEQ ID NO: 30 and the amino acid sequences are amino acids 254-262 of SEQ ID NO: 29.

(SEQ ID NO: 29)
MKKIWLALAGLVLAFSASAYEVDATLKSLNNQIETLLTPEGSRKNPARTC
RDLRLSHPEWSSGYYWIDPNQGCTMDAIKVYCDFSTGETCIRAQPENIPA
KNWYRSSKDKKHVWLGETINAGSQFEYNVEGVTSKEMATQLAFMRLLANY
ASQNITYHCKNSIAYMDEETGNLKKAVILQGSNDVELVAEGNSRFTYTVL
VDGCSKKTNEWGKTHIEYKTNKPSRLPFLDIAPLDIGGADQEFFVDIGPV
CFKGDYKDDDDK

The nucleic acid sequence of truncated human collagen type 1 alpha 2 truncation 7 with DsbA secretion and FLAG tag is disclosed in SEQ ID NO: 30.

(SEQ ID NO: 30)
TGAAAAAGATTTGGCTGGCGCTGGCTGGTTTAGTTTTAGCGTTTAGCGCA
TCGGCGTATGAAGTTGATGCAACCCTGAAAAGCCTGAATAATCAGATTGA
AACACTGCTGACACCGGAAGGTAGCCGTAAAAATCCGGCCCGTACCTGTC
GTGATCTGCGTCTGAGCCACCCGGAATGGAGCAGCGGTTATTATTGGATT
GATCCGAATCAAGGTTGTACCATGGATGCAATTAAAGTTTATTGTGATTT
TAGCACAGGTGAAACATGTATCCGTGCACAGCCGGAAAATATTCCGGCCA
AAAATTGGTATCGTAGTAGCAAAGATAAAAAACATGTGTGGCTGGGTGAA
ACCATTAATGCAGGTAGCCAGTTTGAATACAATGTTGAAGGTGTTACCAG
CAAAGAAATGGCAACACAGCTGGCATTTATGCGTCTGCTGGCAAATTATG
CAAGCCAGAATATTACATATCATTGTAAAAATAGCATTGCATATATGGAT
GAAGAAACCGGTAATCTGAAAAAAGCAGTTATTCTGCAGGGTAGCAATGA
TGTTGAACTGGTTGCCGAAGGTAATAGCCGTTTTACATATACCGTTCTGG
TTGATGGTTGTAGCAAAAAAACCAATGAATGGGGTAAAACCATCATTGAA
TATAAAACCAACAAACCGAGCCGTCTGCCGTTTCTGGATATCGCTCCGCT
GGATATTGGTGGTGCCGATCAGGAATTTTTTGTCGATATCGGTCCTGTGT
GTTTTAAAGGTGACTACAAAGACGACGACGACAAATAA

The polynucleotide of SEQ ID NO: 30 was subcloned into vector pET28a, expressed in host E. coli cells and the truncated collagen was purified as described herein. The purified collagen produced a clear band on SDS-PAGE and an anti-FLAG western was observed at around 30 kilodaltons. There were no existing bands that appear at that location on the gel in the absence of expression of this protein.

### Example 3. Human Clinical Study of Truncated Human Type 21 Collagen

A clinical study using human subjects to determine the effects of a topical skincare product containing truncated human type 21 collagen (SEQ ID NO: 16) is performed. The research is performed according to U.S. and International standards of Good Clinical Practice (FDA and ICH guidelines) and applicable government regulations.

A base formulation (control formulation) made of water, olive oil glycereth-8 esters, glycerin, coconut alkanes, hydroxyethyl acrylate/sodium acryloyldimethyl taurate copolymer, pentylene glycol, disodium EDTA, caprylyl glycol, chlorphenesin, phenoxyethanol is prepared. A formulation containing the truncated human type 21 collagen is prepared by adding sufficient collagen to prepare a topical formulation containing 0.1% w/w collagen.

### Expert Grading Assessments for Qualification and Efficacy

Visual Analog Scales (VAS) are commonly used in clinical research to measure intensity or frequency of various symptoms, subjective characteristics or attitudes that cannot be directly measured. VAS are a reliable scale and more sensitive to small changes than simple ordinal scales. (A. Paul-Dauphin, F. Guillemin, J. Virion and S. Briancon, “Bias and precision in visual analog scales: A randomized controlled trial,” American Journal of Epidemiology, vol. 150, no. 10, pp. 1117-27, 1999). When responding to a VAS item, the expert grader specifies their level of agreement to a statement by indicating a position along a line (10 cm) between two end-points or anchor responses. Simple VAS is used to evaluate efficacy parameters in which the ends of a 10 cm horizontal line is defined as extreme limits orientated from the left (best) to the right (worst). Signs of photo-aging can be classified as follows: Mild=1-3.9 cm, Moderate=4-6.9 cm, Severe=7-10 cm.

The following VAS was used:

Ordinal scales allow a number to be directly and objectively attached to the quality of a given attribute. When responding to an ordinal scale item, the expert grader specifies their level of agreement to a statement by choosing a set grade, or level.

The appearance of each subject's facial skin redness (erythema) is assessed by an expert grader using the following five-point ordinal scale for qualification at Baseline (Table 6). If qualified, each subject will undergo further erythema assessments at week 2, week 4, week 6 and week 8.

TABLE 6
Five-point ordinal scale for erythema assessment
Erythema

| Grade | Description |
|---|---|
| 0 | No erythema |
| 1 | Very slight erythema (barely perceptible) |
| 2 | Well-defined erythema |
| 3 | Moderate to severe erythema |
| 4 | Severe erythema (beet redness) to slight eschar |
| | formation (injuries in depth) |

The corneometer CM 820 (Courage+Khazaka, Germany) measures the relative degree of hydration of the skin surface by applying an alternating current to the skin with a closely spaced pair of electrodes and measuring the capacitance. Changes in water content of the skin change the conductance of the capacitive circuit.

The corneometer is able to detect slight changes in the hydration level reproducibly with a measurement time of only about one second. The measurement depth is small (approximately 10-20 μm of stratum corneum) which ensures assessment is not influenced by deeper skin layers.

All subjects undergo corneometer measurements of their face at baseline, immediately post-initial-application, and at weeks 2, 4, and 8. Measurements will be taken in triplicate and averaged for each time point. Measurement location is recorded on a face map for assessment consistency at each time point.

The Cutometer MPA 580 (Courage+Khazaka, Germany) measures the viscoelastic properties of the skin by applying suction to the skin surface, drawing the skin into the aperture of the probe and determining the penetration depth using an optical measuring system.

The resistance of the skin to be sucked up by the negative pressure (firmness) and its ability to return to its original position (elasticity) are calculated and displayed as curves. The Cutometer outputs include many parameters of different portions of the measurement curve including of R0 (Uf, firmness), R2 (Ua/Uf, gross elasticity), R5 (Ur/Ue, net elasticity), R7 (Ur/Uf, elastic portion) and R9 (R3 [last max amp]-R0 [Uf], fatigue).

All subjects have Cutometer measurements taken on the left or right cheek (following a prepared randomization code) at baseline, immediately post-initial-application, and at weeks 2, 4, and 8. Skin elasticity is reported using the R5 (Ur/Ue) and R2 (Ua/Uf) parameter. As the skin becomes more elastic, this value will increase. Skin Firmness is reported using the R0 (Uf) parameter. As the skin becomes firmer, this value will decrease. Assessment location is recorded on a face map for each subject for consistency of measurements between visits.

The COSMETRICS™ SIAScope (Astron Clinica, Toft, UK) is a non-invasive optical skin imaging instrument using Spectrophotometric Intracutaneous Analysis (SIA), or chromophore mapping. The technique is based on a unique combination of dermatoscopy and contact remittance spectrophotometry. The hardware consists of a hand-held imaging probe attached to a laptop computer. The unit is placed in contact with the skin surface and high-intensity LED's illuminate the skin as discreet wavelengths of 400 to 1000 nm, spanning the visible spectrum and a small range of the near infrared spectrum. A digital image is captured for each wavelength. Three parametric chromophore maps are retrieved up to 2 mm in depth and 11 mm in circumference, one for each of the following parameters: epidermal melanin, dermal hemoglobin and dermal collagen.

For the purposes of this study, dermal collagen will be measured on the left or right cheek, per a prepared randomization code, at baseline and at weeks 2, 4, and 8. Assessment location will be recorded on a face map for each subject for consistency of measurements between visits.

The DermaScan C USB (Cortex Technology ApS, Hadsund, Denmark) is a compact high resolution ultrasound scanner. The 20 MHz, high definition 60×150 13 mm penetration probe is used which provides linear scanning, high precision operation and true position detection for image clarity and definition

The instrument is provided by cyberDERM, Inc. (Broomall, PA, USA). All subjects have ultrasound assessments taken on the face at baseline and at weeks 2, 4, and 8. The location of assessments is the same at each visit and will be recorded on a face map. Upon acquisition of the ultrasound scans, they are sent to cyberDERM, Inc., for analysis of dermal thickness (density).

All clinical photography is performed in accordance with IRSI's SOP to ensure reproducibility of high quality images throughout the duration of the study. Imaging is conducted in a designated photography suite with a matte black wall and all natural light is blocked out. To prepare subjects for clinical photography, subjects are asked to remove all jewelry, including earrings, necklaces, and any facial jewelry. A trained technician inspects the subjects under a lighted magnification loop to ensure no residual color cosmetics or skincare products are visible on the face, eyes, or lips. Subjects are provided with a black cape and black headband and are instructed on placement to ensure all hair is pulled back neatly and covered.

The Clarity™ 2D Research System Ti (Clarity) (BrighTex Bio-Photonics (BTBP), San Jose CA, USA) captures high quality full face frontal, left, and right lateral images. Three cameras within the system allow for 18 megapixel SLR image capture in 16-bit simultaneously using a live feed display and automated facial alignment checks against baseline images for reproducibility.

Multi-spectral lighting (diffuse white light, cross-polarized, blue and parallel polarized) reveals skin conditions on and beneath the skin's surface layer. The system uses skin feature recognition to apply automated skin segmentation and zone mapping to allow for subsequent skin analysis. Images are analyzed for attributes associated with pigmentation, subsurface pigmentation, radiance, skin color, redness, wrinkles, skin texture, pores, acne, and/or lips.

All subjects have front, left, and right view facial images captured in standard light and parallel polarized light at baseline and at weeks 2, 4, and 8.

Subjective questionnaires allow the Sponsor to gauge the subjects' opinions of their skin, the test product, and its effects. Questions will ask for subjects' agreement to a statement with a five-point scale as well as open-ended response.

Fourteen female subjects are enrolled. The inclusion criteria are Caucasian female subjects with Fitzpatrick skin type III in good general health, and between ages of 35 and 65 years old, inclusive at enrollment. Inclusion criteria also include signs of aging on face as determined by an expert grader at a baseline of: a) Score of >2 cm<6 on 10 cm scale for lines/wrinkles; and b) Score of >1<3 on 5-point ordinal scale for facial redness (erythema). Table 7 discloses the demographics of the study participants.

TABLE 7
Demographics

| Variable | n | Mean ± SD | Min | Max |
|---|---|---|---|---|
| Age (years) | 14 | 57.64 ± 6.03 | 45 | 65 |
| Height (inches) | 14 | 63.35 ± 2.37 | 60 | 68 |
| Weight (pounds) | 14 | 160.14 ± 38.26 | 110 | 250 |
| | | | n | Percent |
| Ethnicity | 14 | Not Hispanic or Latino | 14 | 100% |
| Race | 14 | White | 14 | 100% |
| Fitzpatrick Skin Type | 14 | Skin Type III | 14 | 100% |
| Facial Skin Type | 14 | Combination | 8 | 57.1% |
| | | Normal | 6 | 42.9% |

The results of the expert clinical grader evaluation on lines/wrinkles, firmness (visual), elasticity (tactile), brightness, texture/softness (tactile), texture/smoothness (visual) and erythema after two weeks treatment are shown in Table 8. All of the tested characteristics were improved. The scores for brightness, texture/softness (tactile), texture/smoothness (visual) and erythema improved with statistical significance.

TABLE 8
Expert Clinical Grader Evaluation-Monadic, Comparison to Baseline

| | | | | | Percent of | |
|---|---|---|---|---|---|---|
| | | | | Mean Percent | Subjects Showing | |
| | | | | Improvement | Improvement | P-Value |
| Assessment | Time Point | n | Mean ± SD | From BL mean | From BL | TX vs. BL |
| Lines/ | Baseline | 14 | 5.22 ± 0.75 | | | |
| Wrinkles | Week 2 | 14 | 5.17 ± 0.75 | 0.92% | 57.1% | 0.336 |
| (Global) | | | | | | |
| Firmness | Baseline | 14 | 5.07 ± 0.77 | | | |
| (Visual) | Week 2 | 14 | 5.01 ± 0.75 | 1.08% | 64.3% | 0.120 |
| Elasticity | Baseline | 14 | 4.91 ± 0.53 | | | |
| (Tactile) | Week 2 | 14 | 4.90 ± 0.52 | 0.25% | 35.7% | 0.547 |
| Brightness | Baseline | 14 | 5.51 ± 0.54 | | | |
| | Week 2 | 14 | 5.37 ± 0.62 | 2.57% | 78.6% | 0.010* |
| Texture/ | Baseline | 14 | 4.82 ± 0.82 | | | |
| Softness | Week 2 | 14 | 4.51 ± 0.89 | 6.43% | 71.4% | 0.008* |
| (Tactile) | | | | | | |
| Texture/ | Baseline | 14 | 5.46 ± 0.70 | | | |
| Smoothness | Week 2 | 14 | 5.25 ± 0.68 | 3.63% | 64.3% | 0.009* |
| (Visual) | | | | | | |
| Erythema | Baseline | 14 | 1.60 ± 0.73 | | | |
| | Week 2 | 14 | 1.21 ± 0.80 | 23.81% | 35.7% | 0.021* |

*Indicates a statistically significant improvement compared to baseline, p ≤ 0.05

Instrumental evaluation hydration, firmness elasticity using a corneometer and cutometer are shown in Table 9. Improvements in skin hydration, firmness, and elasticity were statistically significant. In addition, Table 9 shows the stimulation of collagen production by skin cells as demonstrated by Spectrophotometric Intracutaneous Analysis (SIA).

TABLE 9
Instrumental Evaluation-Monadic, Comparison to Baseline

| | | | | | | Percent of | |
|---|---|---|---|---|---|---|---|
| | | | | | Mean Percent | Subjects Showing | |
| | | | | | Improvement | Improvement | P-Value |
| Assessment | | Time Point | n | Mean ± SD | From BL mean | From BL | TX vs. BL |
| Corneometer | | Baseline | 14 | 42.45 ± 10.06 | | | |
| | | Immediate | 14 | 57.22 ± 12.47 | 36.69% | 100% | <0.001* |
| | | Week 2 | 14 | 35.71 ± 10.77 | NI | 14.3% | 0.001** |
| Cutometer | Firmness | Baseline | 14 | 0.35 ± 0.06 | | | |
| | (R0 Uf) | Immediate | 14 | 0.24 ± 0.05 | 30.38% | 100% | <0.001* |
| | | Week 2 | 14 | 0.31 ± 0.07 | 11.27% | 78.6% | 0.028* |
| | Elasticity | Baseline | 14 | 0.64 ± 0.09 | | | |
| | (R2 Ua/Uf) | Immediate | 14 | 0.93 ± 0.13 | 45.85% | 100% | <0.001* |
| | | Week 2 | 14 | 0.80 ± 0.09 | 26.93% | 100% | <0.001* |
| | Elasticity | Baseline | 14 | 0.28 ± 0.06 | | | |
| | (R5 Ur/Ue) | Immediate | 14 | 0.40 ± 0.09 | 44.57% | 92.9% | <0.001* |
| | | Week 2 | 14 | 0.37 ± 0.08 | 33.36% | 92.9% | <0.001* |
| SlAscope | Collagen | Baseline | 14 | 238.95 ± 11.60 | | | |
| | | Week 2 | 14 | 254.89 ± 19.35 | 6.72% | 78.6% | 0.004* |

NI = No Improvement
*Indicates a statistically significant improvement compared to baseline, p ≤ 0.05
**Indicates a statistically significant worsening compared to baseline, p ≤ 0.05

The results demonstrate that truncated human type 21 collagen shows statistically significant improvements in elasticity, brightness, hydration, tactile texture, or visual texture of skin. In addition, the results show that truncated human type 21 collagen shows statistically significant decreases in visible lines or wrinkles as well as significant decreases in erythema.

### Example 4. In Vitro Study of Truncated Human Type 21 Collagen on Skin Cells

### Truncated Human Type 21 Alpha 1 Collagen Stimulates Fibroblast Production of Collagen Type I

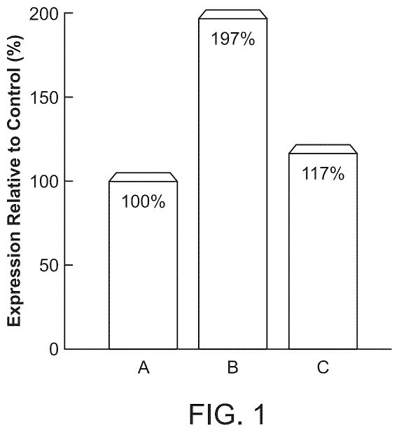

A series of in vitro experiments were conducted to assess the effects of a truncated human type 21 collagen on human skin fibroblasts and keratinocytes. In a first experiment, human primary fibroblasts were evaluated for collagen type I protein secretion. Fibroblasts were cultured with 0.03% of a polypeptide according to SEQ ID NO: 16 for 48 hours. Culture supernatants were analyzed by Enzyme Linked Immunosorbent Assay (ELISA) for pro-collagen type I C-peptide, which is a readout for total secreted collagen type I protein. As shown in FIG. 1, cells treated with a polypeptide of SEQ ID NO: 16 secreted higher levels of collagen type I (FIG. 1; “B”) than untreated cells (FIG. 1; “A”) or cells treated with retinol (FIG. 1; “C”).

### Truncated Human Type 21 Alpha 1 Collagen Stimulates Fibroblast Production of Genes for Extracellular Matrix Proteins

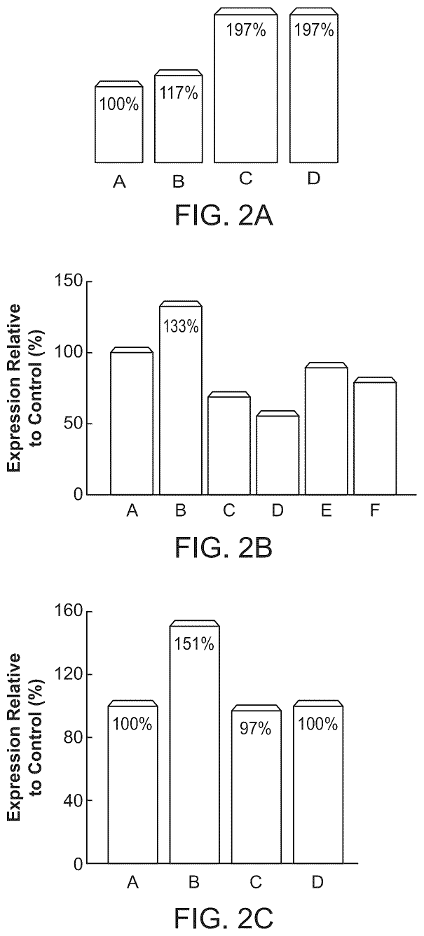

RNA sequencing was performed to analyze global gene expression. After 48 hours of exposure, fibroblasts were incubated with 0.03% of a polypeptide according to SEQ ID NO: 16. These fibroblasts expressed higher levels of several extracellular matrix genes than cells incubated in media alone. As shown in FIG. 2A, fibroblasts treated with a polypeptide of SEQ ID NO: 16 (FIG. 2A; “C”) upregulated the collagen type I gene (COL1A) relative to untreated cells (FIG. 2A; “A”) or fibroblasts treated with retinol (FIG. 2A; “B”). This response was similar to fibroblasts treated with Vitamin C (FIG. 2A; “D”). As shown in FIG. 2B, fibroblasts treated with a polypeptide of SEQ ID NO: 16 (FIG. 2B; “B”) upregulated the elastin gene (ELN) relative to untreated cells (FIG. 2B; “A”), and various marine collagens (FIG. 2B; “C”, “D”, “E”, and “F”). As shown in FIG. 2C, fibroblasts treated with a polypeptide of SEQ ID NO: 16 (FIG. 2C; “B”) upregulated the fibronectin gene (FN1) relative to untreated cells (FIG. 2C; “A”), retinol (FIG. 2C; “C”), and Vitamin C (FIG. 2C; “D”).

Truncated Human Type 21 Alpha 1 Collagen Reduces Inflammation of Keratinocytes Irradiated with UVB Light.

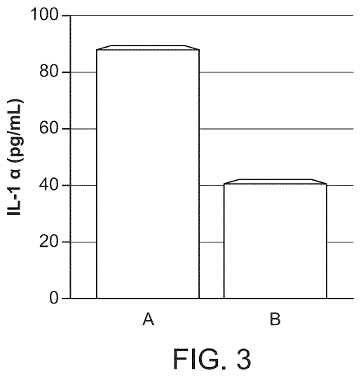

Human primary keratinocytes were irradiated with 40 mJ/cm² UVB light, and then treated with 0.1% of a polypeptide of SEQ ID NO: 16 for 24 hours. Levels of the pro-inflammatory cytokines IL-la were determined by ELISA. As shown in FIG. 3, UVB-irradiated keratinocytes treated with a polypeptide of SEQ ID NO: 16 (FIG. 3; “B”) expressed lower levels of IL-la compared to untreated UVB-irradiated keratinocytes (FIG. 3; “A”).

### Truncated Human Type 21 Alpha 1 Collagen has Anti-Oxidative Capacity.

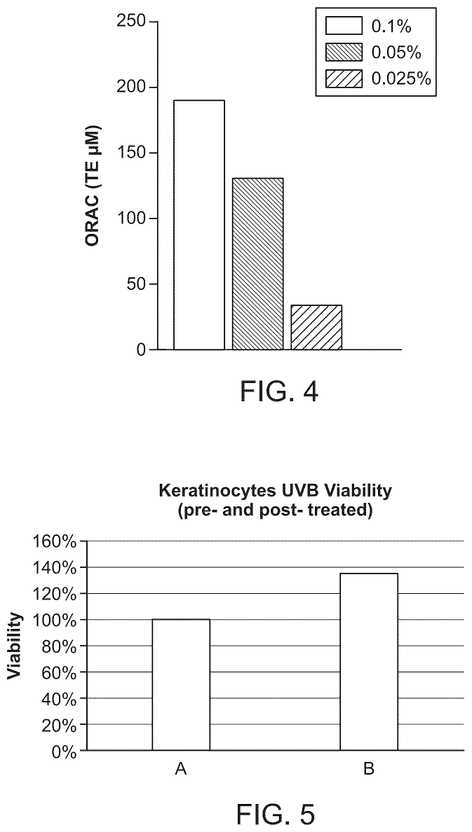

The antioxidant potential of a polypeptide of SEQ ID NO: 16 was assessed using the oxygen free radical absorbance capacity (ORAC) assay. The ORAC assay is a cell-free assay that uses a fluorescent readout to measure a product's antioxidant capacity. Data is reported in Trolox (Vitamin E) equivalents. As shown in FIG. 4, a 0.1% solution of a polypeptide of SEQ ID NO: 16 had antioxidant properties equivalent to 190 μM Trolox.

Truncated Human Type 21 Alpha 1 Collagen Increases Cell Viability of Keratinocytes Irradiated with UVB Light.

To further assess the effects of treatment with a polypeptide of SEQ ID NO: 16 on UVB-irradiated keratinocytes, an experiment was performed with pre- and post-irradiation treatment. Human primary keratinocytes were pre-treated with 0.1% of a polypeptide of SEQ ID NO: 16 for 24 hours, irradiated with 40 mJ/cm² UVB light, and then treated again with 0.1% of a polypeptide of SEQ ID NO: 16 for an additional 24 hours. Cell viability was evaluated using the MTT metabolic colorimetric assay. As shown in FIG. 5, UVB-irradiated keratinocytes treated with a polypeptide of SEQ ID NO: 16 (FIG. 5; “B”) showed higher cell viability than UVB-irradiated keratinocytes without such treatment (FIG. 5; “A”).

### Example 5. Human Clinical Study of Truncated Human Type 21 Collagen

Topical Application of Truncated Human Type 21 Alpha 1 Collagen is Associated with Facial Skin Elasticity Increase

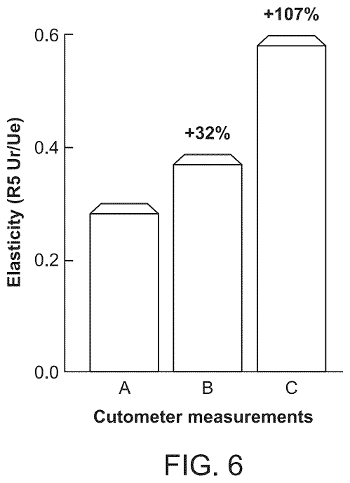

In a clinical study (n=15 subjects), subjects used a topical facial serum containing 0.1% of a polypeptide of SEQ ID NO: 16 for 8 weeks, after using a protein-free base facial serum for a 1-week washout period. Topical application of a polypeptide of SEQ ID NO: 16 was associated with increased skin elasticity, measured using a cutometer. As shown in FIG. 6, 100% of subjects showed improvement with an increase in skin elasticity at 2 weeks (FIG. 6; “B”) and 4 weeks (FIG. 6; “C”) as compared to baseline (FIG. 6; “A”).

Topical Application of Human Type 21 Alpha 1 Collagen is Associated with Facial Skin Collagen Content Increase

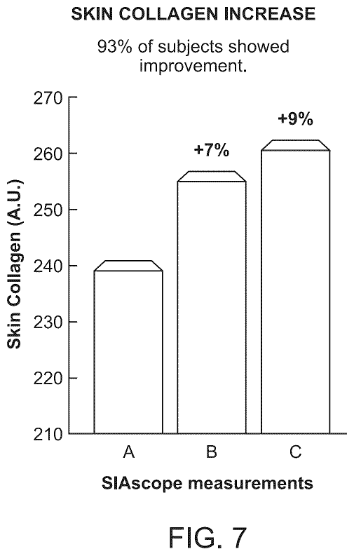

In a clinical study (n=15 subjects), subjects used a topical facial serum containing 0.1% of a polypeptide of SEQ ID NO: 16 for 8 weeks, after using a protein-free base facial serum for a 1-week washout period. Topical application of a polypeptide of SEQ ID NO: 16 was associated with increased skin collagen content, as measured by a SIAscope. As shown in FIG. 7, skin collagen content increased at 2 weeks (FIG. 7; “B”) and at 8 weeks (FIG. 7; “C”) as compared to baseline (FIG. 7; “A”).

Topical Application of Human Type 21 Alpha Collagen is Associated with a Reduction in Facial Skin Redness

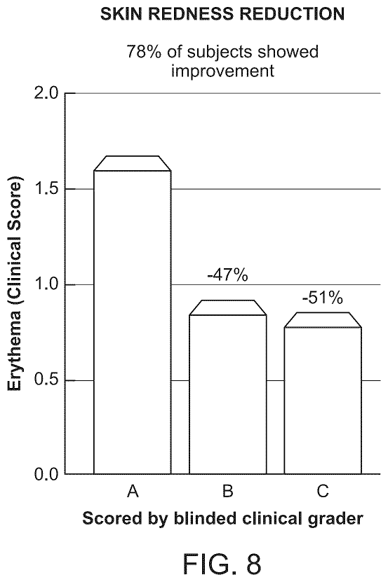

In a clinical study (n=15 subjects), subjects used a topical facial serum containing 0.1% of a polypeptide of SEQ ID NO: 16 for 8 weeks, after using a protein-free base facial serum for a 1-week washout period. As shown in FIG. 8, topical application of a polypeptide of SEQ ID NO: 16 was associated with a decrease in skin redness at 4 weeks (FIG. 8; “B”) and at 8 weeks (FIG. 8; “C”) as compared to baseline (FIG. 8; “A”).

Topical Application of Human Type 21 Alpha 1 Collagen is Associated with a Reduction in Facial Wrinkles

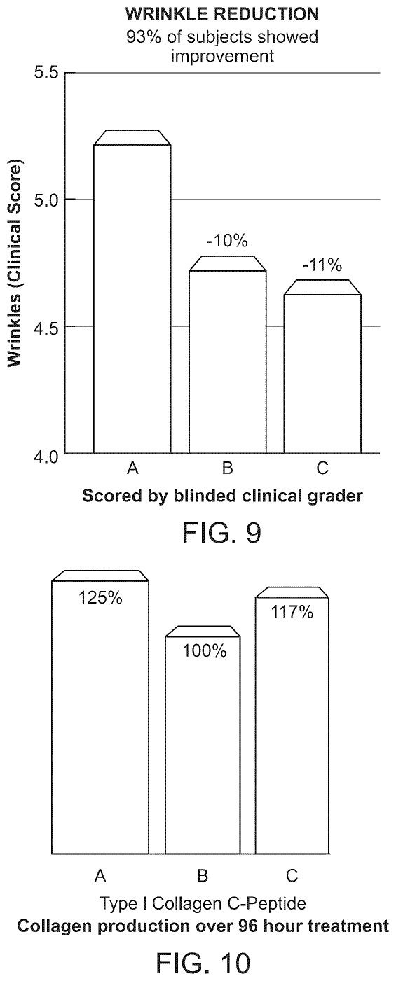

In a clinical study (n=15 subjects), subjects used a topical facial serum containing 0.1% of a polypeptide of SEQ ID NO: 16 for 8 weeks, after using a protein-free base facial serum for a 1-week washout period. As shown in FIG. 9, topical application of a polypeptide of SEQ ID NO: 16 was associated with a reduction in facial wrinkles at 4 weeks (FIG. 9; “B”) and at 8 weeks (FIG. 9; “C”) as compared to baseline (FIG. 9; “A”).

### Example 6. In Vitro Study of Truncated Jellyfish Collagen on Skin Cells

### Truncated Jellyfish Collagen Stimulates Skin Cell Production of Collagen Type I Protein

A series of in vitro experiments were conducted to assess the effects of a truncated jellyfish collagen on human skin fibroblasts and keratinocytes. An in vitro full thickness human skin tissue model (MatTek) which contains fibroblasts and keratinocytes was evaluated for collagen type I secretion after treatment with a polypeptide of SEQ ID NO: 5 for 48 hours. The tissue models were then rinsed and incubated with fresh media for another 48 hours (96 hour timepoint). The culture supernatants were analyzed by ELISA for pro-collagen type I C-peptide (a readout for total secreted collagen type I protein). As shown in FIG. 10, the tissue models treated with a polypeptide of SEQ ID NO: 5 (FIG. 10; “A”) secreted higher levels of collagen type I than untreated tissue models (FIG. 10; “B”) or tissue models treated with the positive control, Vitamin B3 (FIG. 10; “C”).

Truncated Jellyfish Collagen Reduces DNA Damage in Keratinocytes after Exposure to UVB Light

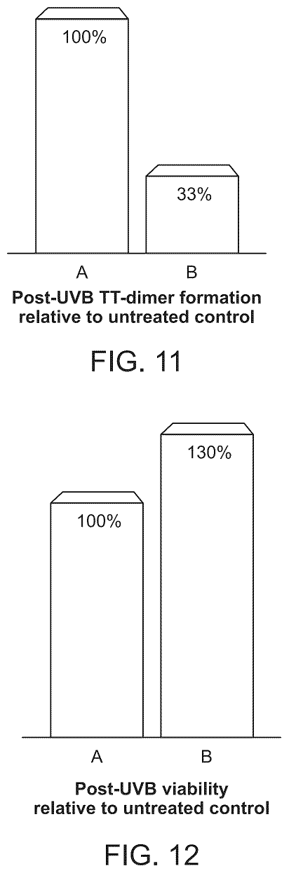

In a further study, human primary keratinocytes were irradiated with 25 mJ/cm² UVB light, then incubated overnight in media with 0.03% of a polypeptide of SEQ ID NO: 5. DNA was extracted from the cells and analyzed for levels of thymine dimers (an indicator of DNA damage) using an OxiSelect UV-Induced DNA Damage ELISA kit. As shown in FIG. 11, cells treated with a polypeptide of SEQ ID NO: 5 (FIG. 11; “B”) showed a lower level of thymine dimers, and thus less DNA damage, than untreated cells (FIG. 11; “A”).

Truncated Jellyfish Collagen Increases Cell Viability of Keratinocytes Irradiated with UVB Light

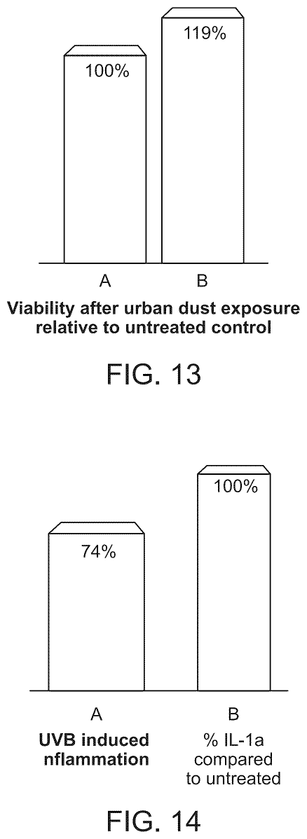

Human primary keratinocytes were irradiated with 40 mJ/cm² UVB light, then incubated for 48 hours in media with 0.03% of a polypeptide of SEQ ID NO: 5. Cell viability was evaluated using the MTT metabolic colorimetric assay. As shown in FIG. 12, UVB-irradiated keratinocytes treated with a polypeptide of SEQ ID NO: 5 (FIG. 12; “B”) showed higher cell viability than untreated UVB-irradiated keratinocytes (FIG. 12; “A”).

### Truncated Jellyfish Collagen Increases Cell Viability of Keratinocytes Exposed to Urban Dust Pollution

To test for protection from urban dust, human primary keratinocytes were pre-treated with 0.03% of a polypeptide of SEQ ID NO: 5 for 24 hours, and then exposed to 2 mg/ml urban dust (NIST 1649B) for 24 hours. Cell viability was evaluated using the MTT metabolic colorimetric assay. As shown in FIG. 13, keratinocytes pre-treated with a polypeptide of SEQ ID NO: 5 (FIG. 13; “B”) showed higher cell viability after urban dust exposure than untreated keratinocytes exposed to urban dust (FIG. 13; “A”).

Truncated Jellyfish Collagen Reduces Inflammation of Keratinocytes Irradiated with UVB Light

In a further study with the in vitro full thickness human skin tissue model (MatTek), the MatTek tissue models were irradiated with 300 mJ/cm² UVB light, then treated with 0.01% of a polypeptide of SEQ ID NO: 5 for 24 hours. Levels of pro-inflammatory cytokine IL-la was determined by ELISA. As seen in FIG. 14, the tissue model treated with a polypeptide of SEQ ID NO: 5 (FIG. 14; “A”) showed lower levels of IL-la compared to the untreated UVB-irradiated (FIG. 14; “B”) control tissue model.

### Truncated Jellyfish Collagen has Anti-Oxidative Capacity

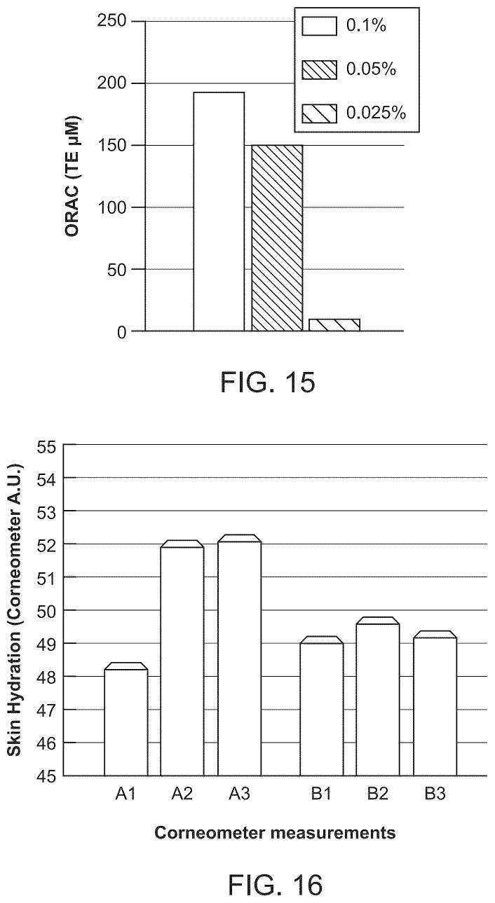

A polypeptide of SEQ ID NO: 5 was also evaluated in the ORAC assay. As shown in FIG. 15, a 0.1% solution of a polypeptide of SEQ ID NO: 5 had anti-oxidative properties equivalent to 193 μM Trolox.

### Example 7. Human Clinical Study of Truncated Jellyfish Collagen

Topical Application of a Truncated Jellyfish Collagen is Associated with an Increase in Facial Skin Moisture

In a clinical study (n=18 subjects), subjects used a topical facial cream containing 0.05% of a polypeptide of SEQ ID NO: 5 for 2 weeks. As shown in FIG. 16, topical application of a polypeptide of SEQ ID NO: 5 was associated with increased skin hydration at 1 week (FIG. 16; “A2”) and at 2 weeks (FIG. 16; “A3”) as compared to baseline (FIG. 16; “A1”). Topical application of a polypeptide of SEQ ID NO: 5 also demonstrated increased skin hydration relative to topical application of marine collagen at baseline (FIG. 16; “B1”), at 1 week (FIG. 16; “B2”), and at 2 weeks (FIG. 16; “B3”).

Topical Application of Truncated Jellyfish Collagen is Associated with an Increase in Facial Skin Elasticity

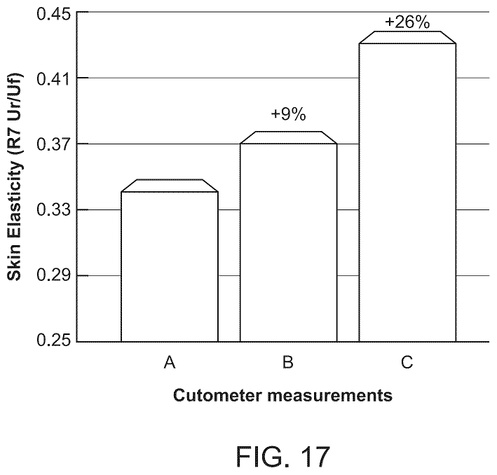

In a clinical study (n=18 subjects), subjects used a topical facial cream containing 0.05% of a polypeptide of SEQ ID NO: 5 for 2 weeks. As shown in FIG. 17, topical application of a polypeptide of SEQ ID NO: 5 was associated with increased skin elasticity, measured using a cutometer, at 1 week (FIG. 17; “B”) and at 2 weeks (FIG. 17; “C”), as compared to baseline (FIG. 17; “A”).

### Example 8. In vitro study of combinations of human type 21 collagen with retinol, ascorbic acid, and hyaluronic acid on skin cells

Human Type 21 Alpha 1 Collagen in Combination with Hyaluronic Acid Synergistically Increases Type I Collagen Production

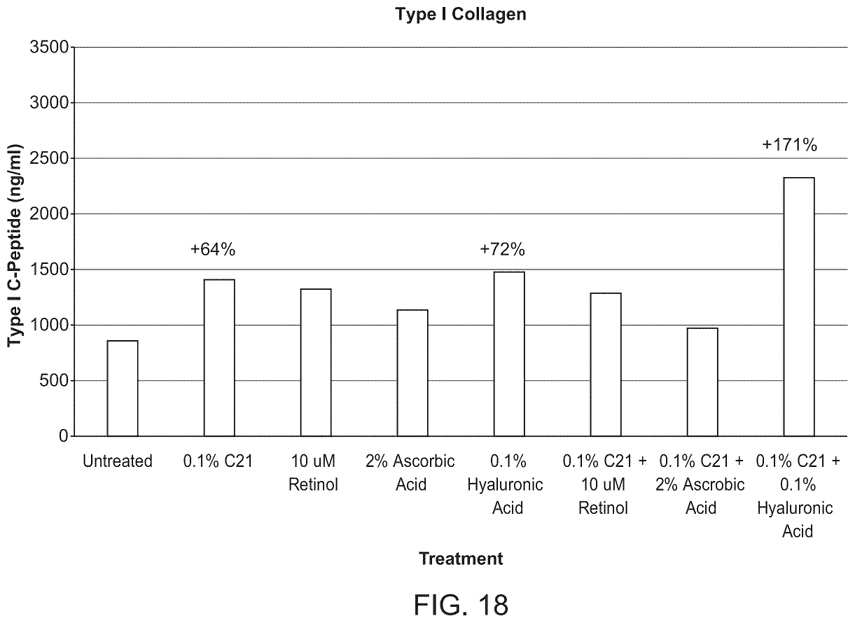

Human full-thickness skin equivalents (MatTek Epiderm FT co-culture tissue models) which contain keratinocytes and fibroblasts were treated with a 0.1% w/w truncated human type 21 collagen polypeptide (having an amino acid sequence of SEQ ID NO: 16) (“C21”; FIG. 18) combined with either retinol (10 ascorbic acid (2% w/w), or hyaluronic acid (0.1% w/w) (low molecular weight, 15-40 kDa). Culture medium was changed every 2 days. On day 4, supernatant was collected and analyzed by enzyme-linked immunosorbent assay (ELISA) for pro-collagen type I C-peptide, which is a readout for total secreted collagen type I protein. As shown in FIG. 18, cells treated with truncated human type 21 collagen polypeptide, retinol, ascorbic acid, or hyaluronic acid alone increased production of type I collagen as compared to untreated cells. The combination of the truncated human type 21 collagen polypeptide with hyaluronic acid increased type I collagen production by 171% relative to the untreated control. This appeared to be a synergistic effect as treatment with truncated human type 21 collagen polypeptide alone increased type I collagen production by 64% and treatment with hyaluronic acid alone increased type I collagen production by 72%.

Human Type 21 Alpha 1 Collagen in Combination with Hyaluronic Acid Increases Fibronectin Production

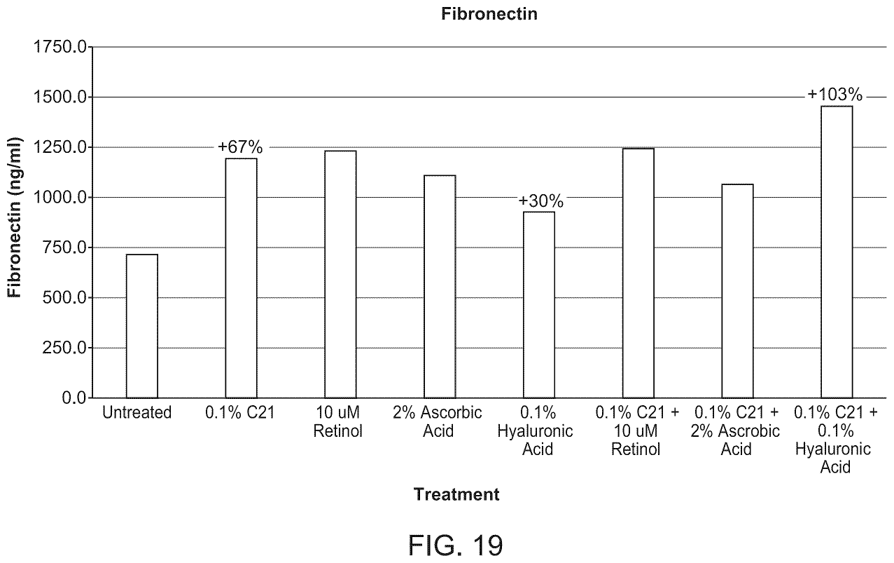

Human full-thickness skin equivalents (MatTek Epiderm FT co-culture tissue models) which contain keratinocytes and fibroblasts were treated with 0.1% w/w truncated human type 21 collagen polypeptide (having an amino acid sequence of SEQ ID NO: 16) (“C21”; FIG. 19) combined with either retinol (10 ascorbic acid (2% w/w), or hyaluronic acid (0.1% w/w) (low molecular weight, 15-40 kDa). Culture medium was changed every 2 days. On day 4, supernatant was collected and analyzed by enzyme-linked immunosorbent assay (ELISA) for fibronectin. As shown in FIG. 19, cells treated with the truncated human type 21 collagen polypeptide, retinol, ascorbic acid, or hyaluronic acid alone increased production of fibronectin as compared to untreated cells. The combination of the truncated human type 21 collagen polypeptide with hyaluronic acid increased fibronectin production by 103% relative to untreated control. This appeared to be an additive effect as treatment with truncated human type 21 collagen polypeptide increased fibronectin production by 67% and treatment with hyaluronic acid alone increased fibronectin production by 30%.

Human Type 21 Alpha 1 Collagen in Combination with Ascorbic Acid or Retinol Increases Elastin Production

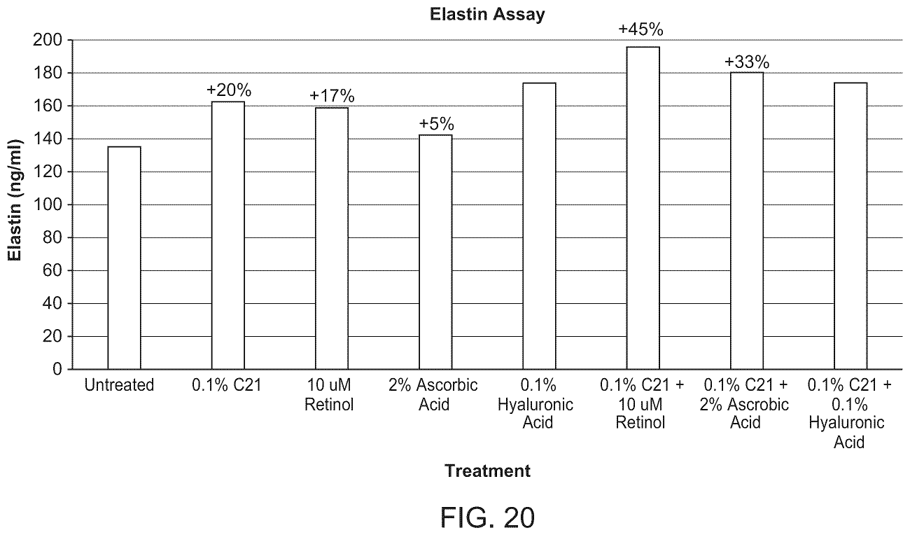

Human full-thickness skin equivalents (MatTek Epiderm FT co-culture tissue models) which contain keratinocytes and fibroblasts were treated with 0.1% w/w truncated human type 21 collagen polypeptide (having an amino acid sequence of SEQ ID NO: 16) (“C21”; FIG. 20) combined with either retinol (10 ascorbic acid (2% w/w), or hyaluronic acid (0.1% w/w) (low molecular weight, 15-40 kDa). On day 2, supernatant was collected and analyzed by enzyme-linked immunosorbent assay (ELISA) for elastin. As shown in FIG. 20, cells treated with the truncated human type 21 collagen polypeptide, retinol, ascorbic acid, or hyaluronic acid alone increased production of elastin as compared to untreated cells. The combination of the truncated human type 21 collagen polypeptide with retinol increased elastin production by 45% relative to untreated control. This appeared to be a synergistic effect as treatment with truncated human type 21 collagen polypeptide alone increased elastin production by 20% and treatment with retinol alone increased elastin production by 17%. The combination of the truncated human type 21 collagen polypeptide with ascorbic acid increased elastin production by 33% relative to untreated control. This appeared to be a synergistic effect as treatment with truncated human type 21 collagen polypeptide alone increased elastin production by 20% and treatment with ascorbic acid alone increased elastin production by 5%.

Human Type 21 Alpha 1 Collagen in Combination with Ascorbic Acid Increases Type III Collagen Production

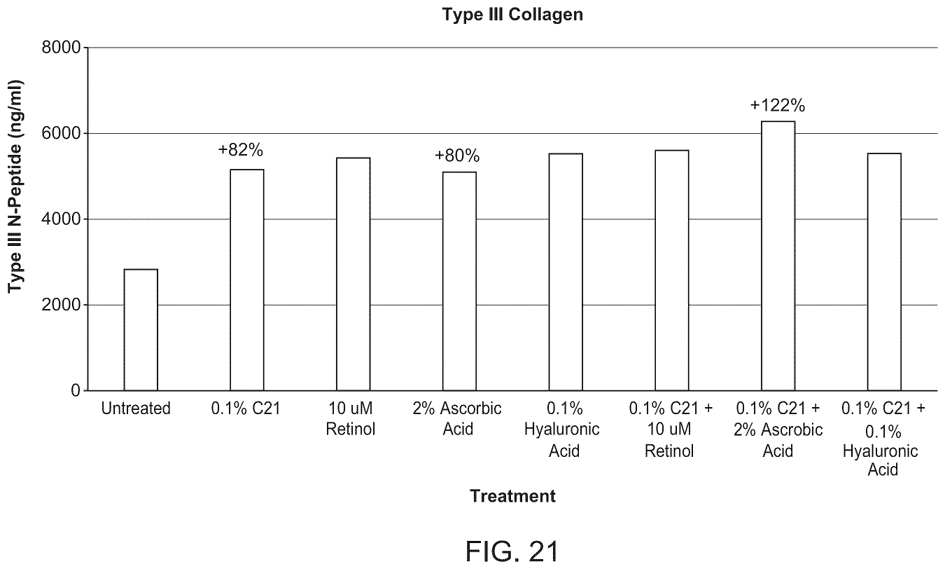

Human full-thickness skin equivalents (MatTek Epiderm FT co-culture tissue models) which contain keratinocytes and fibroblasts were treated with 0.1% w/w truncated human type 21 collagen polypeptide (having an amino acid sequence of SEQ ID NO: 16) (“C21”; FIG. 21) combined with either retinol (10 ascorbic acid (2% w/w), or hyaluronic acid (0.1% w/w) (low molecular weight, 15-40 kDa). On day 2, supernatant was collected and analyzed by enzyme-linked immunosorbent assay (ELISA) for pro-collagen type III N-peptide, which is a readout for total secreted collagen type III protein. As shown in FIG. 21, cells treated with the truncated human type 21 collagen polypeptide, retinol, ascorbic acid, or hyaluronic acid alone increased production of type III collagen as compared to untreated cells. The combination of the truncated human type 21 collagen polypeptide with ascorbic acid increased type III collagen production by 122% relative to untreated control which was greater than treatment with either truncated human type 21 (82%) collagen polypeptide or ascorbic acid (80%) alone.

### Example 9. In Vitro Testing Outcomes of Combinations of Truncated Human Type 21 Collagen Polypeptide and Hyaluronic Acid, Retinol, Ascorbic Acid, Salicylic Acid, Benzoyl Peroxide, Niacinamide, or Alpha Hydroxy Acids (e.g., Glycolic Acid, Lactic Acid)

Combinations of Truncated Human Type 21 Collagen Polypeptide with Ascorbic Acid, Retinol, Hyaluronic Acid, Salicylic Acid, Benzoyl Peroxide, Niacinamide, or Alpha Hydroxy Acids (e.g., Glycolic Acid, Lactic Acid) Increase Keratinocyte and Fibroblast Cell Viability and Proliferation

In an in vitro study, (e.g., human) skin cell cultures, (e.g., human) skin equivalent cultures, or ex vivo (e.g., human) skin is treated with truncated human type 21 collagen polypeptide (e.g., having an amino acid sequence of SEQ ID NO: 16) alone, or in combination with ascorbic acid, retinol, hyaluronic acid, salicylic acid, benzoyl peroxide, niacinamide, or alpha hydroxy acids (e.g., glycolic acid, lactic acid). Treatment with truncated human type 21 collagen polypeptide (e.g., having an amino acid sequence of SEQ ID NO: 16) in combination with ascorbic acid, retinol, hyaluronic acid, salicylic acid, benzoyl peroxide, niacinamide, or alpha hydroxy acids (e.g., glycolic acid, lactic acid) increases (e.g., synergistically, additively) viability and proliferation of keratinocytes and fibroblasts (e.g., as measured by, e.g., MTT viability assay), relative to treatment with the truncated human type 21 collagen, ascorbic acid, retinol, hyaluronic acid, salicylic acid, benzoyl peroxide, niacinamide, or alpha hydroxy acids (e.g., glycolic acid, lactic acid) alone.

Combinations of Truncated Human Type 21 Collagen Polypeptide with Ascorbic Acid, Retinol, Hyaluronic Acid, Salicylic Acid, Benzoyl Peroxide, Niacinamide, or Alpha Hydroxy Acids (e.g., Glycolic Acid, Lactic Acid) Increase Production of Extracellular Matrix Components and Decrease Production of Matrix-Degrading Proteins

In an in vitro study, (e.g., human) skin cell cultures, (e.g., human) skin equivalent cultures, or ex vivo (e.g., human) skin is treated with truncated human type 21 collagen polypeptide (e.g., having an amino acid sequence of SEQ ID NO: 16) alone, or in combination with ascorbic acid, retinol, hyaluronic acid, salicylic acid, benzoyl peroxide, niacinamide, or alpha hydroxy acids (e.g., glycolic acid, lactic acid). Treatment with truncated human type 21 collagen polypeptide (e.g., having an amino acid sequence of SEQ ID NO: 16) in combination with ascorbic acid, retinol, hyaluronic acid, salicylic acid, benzoyl peroxide, niacinamide, or alpha hydroxy acids (e.g., glycolic acid, lactic acid) increases (e.g., synergistically, additively) the production of extracellular matrix components (e.g., collagen, elastin, fibronectin, fibrillin, hyaluronic acid) (e.g., as measured by, e.g., transcriptomic analysis, ELISA) relative to treatment with the truncated human type 21 collagen, ascorbic acid, retinol, hyaluronic acid, salicylic acid, benzoyl peroxide, niacinamide, or alpha hydroxy acids (e.g., glycolic acid, lactic acid) alone. Treatment with truncated human type 21 collagen polypeptide (e.g., having an amino acid sequence of SEQ ID NO: 16) in combination with ascorbic acid, retinol, hyaluronic acid, salicylic acid, benzoyl peroxide, niacinamide, or alpha hydroxy acids (e.g., glycolic acid, lactic acid) decreases (e.g., synergistically, additively) the production of matrix-degrading proteins (e.g., matrix metalloproteases, proteases) (e.g., as measured by, e.g., transcriptomic analysis, ELISA) relative to treatment with the truncated human type 21 collagen, ascorbic acid, retinol, hyaluronic acid, salicylic acid, benzoyl peroxide, niacinamide, or alpha hydroxy acids (e.g., glycolic acid, lactic acid) alone.

Combinations of Truncated Human Type 21 Collagen Polypeptide with Ascorbic Acid, Retinol, Hyaluronic Acid, Salicylic Acid, Benzoyl Peroxide, Niacinamide, or Alpha Hydroxy Acids (e.g., Glycolic Acid, Lactic Acid) Increase Wound Healing Capacity

In an in vitro study, (e.g., human) skin cell cultures, (e.g., human) skin equivalent cultures, or ex vivo (e.g., human) skin is treated with truncated human type 21 collagen polypeptide (e.g., having an amino acid sequence of SEQ ID NO: 16) alone, or in combination with ascorbic acid, retinol, hyaluronic acid, salicylic acid, benzoyl peroxide, niacinamide, or alpha hydroxy acids (e.g., glycolic acid, lactic acid). Treatment with truncated human type 21 collagen polypeptide (e.g., having an amino acid sequence of SEQ ID NO: 16) in combination with ascorbic acid, retinol, hyaluronic acid, salicylic acid, benzoyl peroxide, niacinamide, or alpha hydroxy acids (e.g., glycolic acid, lactic acid) increases (e.g., synergistically, additively) wound healing capacity of skin cells (e.g., keratinocytes, fibroblasts) (e.g., as measured by, e.g., wound healing scratch assay) relative to treatment with the truncated human type 21 collagen, ascorbic acid, retinol, hyaluronic acid, salicylic acid, benzoyl peroxide, niacinamide, or alpha hydroxy acids (e.g., glycolic acid, lactic acid) alone.

Combinations of Truncated Human Type 21 Collagen Polypeptide with Ascorbic Acid, Retinol, Hyaluronic Acid, Salicylic Acid, Benzoyl Peroxide, Niacinamide, or Alpha Hydroxy Acids (e.g., Glycolic Acid, Lactic Acid) have Improved Outcomes after Challenge with Photodamage

In an in vitro study, (e.g., human) skin cell cultures, (e.g., human) skin equivalent cultures, or ex vivo (e.g., human) skin is challenged with photodamage (e.g., UV irradiation, blue light exposure), and then treated with truncated human type 21 collagen polypeptide (e.g., having an amino acid sequence of SEQ ID NO: 16) alone, or in combination with ascorbic acid, retinol, hyaluronic acid, salicylic acid, benzoyl peroxide, niacinamide, or alpha hydroxy acids (e.g., glycolic acid, lactic acid). Treatment with truncated human type 21 collagen polypeptide (e.g., having an amino acid sequence of SEQ ID NO: 16) in combination with ascorbic acid, retinol, hyaluronic acid, salicylic acid, benzoyl peroxide, niacinamide, or alpha hydroxy acids (e.g., glycolic acid, lactic acid) increases (e.g., synergistically, additively) viability and proliferation of skin cells (e.g., keratinocytes, fibroblasts) (e.g., as measured by, e.g., MTT viability assay) relative to treatment with the truncated human type 21 collagen, ascorbic acid, retinol, hyaluronic acid, salicylic acid, benzoyl peroxide, niacinamide, or alpha hydroxy acids (e.g., glycolic acid, lactic acid) alone. Treatment with truncated human type 21 collagen polypeptide (e.g., having an amino acid sequence of SEQ ID NO: 16) in combination with ascorbic acid, retinol, hyaluronic acid, salicylic acid, benzoyl peroxide, niacinamide, or alpha hydroxy acids (e.g., glycolic acid, lactic acid) reduces (e.g., synergistically, additively) cytotoxicity and apoptosis of skin cells (e.g., keratinocytes, fibroblasts) (e.g., as measured by, e.g., fluorescence microscopy) relative to treatment with the truncated human type 21 collagen, ascorbic acid, retinol, hyaluronic acid, salicylic acid, benzoyl peroxide, niacinamide, or alpha hydroxy acids (e.g., glycolic acid, lactic acid) alone. Treatment with truncated human type 21 collagen polypeptide (e.g., having an amino acid sequence of SEQ ID NO: 16) in combination with ascorbic acid, retinol, hyaluronic acid, salicylic acid, benzoyl peroxide, niacinamide, or alpha hydroxy acids (e.g., glycolic acid, lactic acid) increases (e.g., synergistically, additively) DNA repair of skin cells (e.g., keratinocytes, fibroblasts) (e.g., as measured by, e.g., thymidine dimer ELISA detection) relative to treatment with the truncated human type 21 collagen, ascorbic acid, retinol, hyaluronic acid, salicylic acid, benzoyl peroxide, niacinamide, or alpha hydroxy acids (e.g., glycolic acid, lactic acid) alone. Treatment with truncated human type 21 collagen polypeptide (e.g., having an amino acid sequence of SEQ ID NO: 16) in combination with ascorbic acid, retinol, hyaluronic acid, salicylic acid, benzoyl peroxide, niacinamide, or alpha hydroxy acids (e.g., glycolic acid, lactic acid) reduces (e.g., synergistically, additively) inflammation (e.g., as measured by, e.g., ELISA analysis or Luminex detection of cytokines) relative to treatment with the truncated human type 21 collagen, ascorbic acid, retinol, hyaluronic acid, salicylic acid, benzoyl peroxide, niacinamide, or alpha hydroxy acids (e.g., glycolic acid, lactic acid) alone. Treatment with truncated human type 21 collagen polypeptide (e.g., having an amino acid sequence of SEQ ID NO: 16) in combination with ascorbic acid, retinol, hyaluronic acid, salicylic acid, benzoyl peroxide, niacinamide, or alpha hydroxy acids (e.g., glycolic acid, lactic acid) reduces (e.g., synergistically, additively) reactive oxidative stress (e.g., as measured by, e.g., CM-H2DCFDA detection) relative to treatment with the truncated human type 21 collagen, ascorbic acid, retinol, hyaluronic acid, salicylic acid, benzoyl peroxide, niacinamide, or alpha hydroxy acids (e.g., glycolic acid, lactic acid) alone.

Combinations of Truncated Human Type 21 Collagen Polypeptide with Ascorbic Acid, Retinol, Hyaluronic Acid, Salicylic Acid, Benzoyl Peroxide, Niacinamide, or Alpha Hydroxy Acids (e.g., Glycolic Acid, Lactic Acid) have Improved Outcomes after Challenge with Pollution

In an in vitro study, (e.g., human) skin cell cultures, (e.g., human) skin equivalent cultures, or ex vivo (e.g., human) skin is challenged with pollution (e.g., urban dust exposure), and then treated with truncated human type 21 collagen polypeptide (e.g., having an amino acid sequence of SEQ ID NO: 16) alone, or in combination with ascorbic acid, retinol, hyaluronic acid, salicylic acid, benzoyl peroxide, niacinamide, or alpha hydroxy acids (e.g., glycolic acid, lactic acid). Treatment with truncated human type 21 collagen polypeptide (e.g., having an amino acid sequence of SEQ ID NO: 16) in combination with ascorbic acid, retinol, hyaluronic acid, salicylic acid, benzoyl peroxide, niacinamide, or alpha hydroxy acids (e.g., glycolic acid, lactic acid) increases (e.g., synergistically, additively) viability and proliferation of skin cells (e.g., keratinocytes, fibroblasts) (e.g., as measured by, e.g., MTT viability assay) relative to treatment with the truncated human type 21 collagen, ascorbic acid, retinol, hyaluronic acid, salicylic acid, benzoyl peroxide, niacinamide, or alpha hydroxy acids (e.g., glycolic acid, lactic acid) alone. Treatment with truncated human type 21 collagen polypeptide (e.g., having an amino acid sequence of SEQ ID NO: 16) in combination with ascorbic acid, retinol, hyaluronic acid, salicylic acid, benzoyl peroxide, niacinamide, or alpha hydroxy acids (e.g., glycolic acid, lactic acid) reduces (e.g., synergistically, additively) cytotoxicity and apoptosis of skin cells (e.g., keratinocytes, fibroblasts) (e.g., as measured by, e.g., fluorescence microscopy) relative to treatment with the truncated human type 21 collagen, ascorbic acid, retinol, hyaluronic acid, salicylic acid alone, benzoyl peroxide, niacinamide, or alpha hydroxy acids (e.g., glycolic acid, lactic acid). Treatment with truncated human type 21 collagen polypeptide (e.g., having an amino acid sequence of SEQ ID NO: 16) in combination with ascorbic acid, retinol, hyaluronic acid, salicylic acid, benzoyl peroxide, niacinamide, or alpha hydroxy acids (e.g., glycolic acid, lactic acid) increases (e.g., synergistically, additively) DNA repair of skin cells (e.g., keratinocytes, fibroblasts) (e.g., as measured by, e.g., thymidine dimer ELISA detection) relative to treatment with the truncated human type 21 collagen, ascorbic acid, retinol, hyaluronic acid, salicylic acid, benzoyl peroxide, niacinamide, or alpha hydroxy acids (e.g., glycolic acid, lactic acid) alone. Treatment with truncated human type 21 collagen polypeptide (e.g., having an amino acid sequence of SEQ ID NO: 16) in combination with ascorbic acid, retinol, hyaluronic acid, salicylic acid, benzoyl peroxide, niacinamide, or alpha hydroxy acids (e.g., glycolic acid, lactic acid) reduces (e.g., synergistically, additively) inflammation (e.g., as measured by, e.g., ELISA analysis or Luminex detection of cytokines) relative to treatment with the truncated human type 21 collagen, ascorbic acid, retinol, hyaluronic acid, salicylic acid, benzoyl peroxide, niacinamide, or alpha hydroxy acids (e.g., glycolic acid, lactic acid) alone. Treatment with truncated human type 21 collagen polypeptide (e.g., having an amino acid sequence of SEQ ID NO: 16) in combination with ascorbic acid, retinol, hyaluronic acid, salicylic acid, benzoyl peroxide, niacinamide, or alpha hydroxy acids (e.g., glycolic acid, lactic acid) reduces (e.g., synergistically, additively) reactive oxidative stress (e.g., as measured by, e.g., CM-H2DCFDA detection) relative to treatment with the truncated human type 21 collagen, ascorbic acid, retinol, hyaluronic acid, salicylic acid, benzoyl peroxide, niacinamide, or alpha hydroxy acids (e.g., glycolic acid, lactic acid) alone.

Combinations of Truncated Human Type 21 Collagen Polypeptide with Ascorbic Acid, Retinol, Hyaluronic Acid, Salicylic Acid, Benzoyl Peroxide, Niacinamide, or Alpha Hydroxy Acids (e.g., Glycolic Acid, Lactic Acid) have Improved Outcomes after Challenge with Harsh Skin Actives

In an in vitro study, (e.g., human) skin cell cultures, (e.g., human) skin equivalent cultures, or ex vivo (e.g., human) skin is challenged with harsh skin actives (e.g., retinoic acid or retinol, benzoyl peroxide, salicylic acid), and then treated with truncated human type 21 collagen polypeptide (e.g., having an amino acid sequence of SEQ ID NO: 16) alone, or in combination with ascorbic acid, retinol, hyaluronic acid, salicylic acid, benzoyl peroxide, niacinamide, or alpha hydroxy acids (e.g., glycolic acid, lactic acid). Treatment with truncated human type 21 collagen polypeptide (e.g., having an amino acid sequence of SEQ ID NO: 16) in combination with ascorbic acid, retinol, hyaluronic acid, salicylic acid, benzoyl peroxide, niacinamide, or alpha hydroxy acids (e.g., glycolic acid, lactic acid) increases (e.g., synergistically, additively) viability and proliferation of skin cells (e.g., keratinocytes, fibroblasts) (e.g., as measured by, e.g., MTT viability assay) relative to treatment with the truncated human type 21 collagen, ascorbic acid, retinol, hyaluronic acid alone, salicylic acid, benzoyl peroxide, niacinamide, or alpha hydroxy acids (e.g., glycolic acid, lactic acid). Treatment with truncated human type 21 collagen polypeptide (e.g., having an amino acid sequence of SEQ ID NO: 16) in combination with ascorbic acid, retinol, hyaluronic acid, salicylic acid, benzoyl peroxide, niacinamide, or alpha hydroxy acids (e.g., glycolic acid, lactic acid) reduces (e.g., synergistically, additively) cytotoxicity and apoptosis of skin cells (e.g., keratinocytes, fibroblasts) (e.g., as measured by, e.g., fluorescence microscopy) relative to treatment with the truncated human type 21 collagen, ascorbic acid, retinol, hyaluronic acid, salicylic acid, benzoyl peroxide, niacinamide, or alpha hydroxy acids (e.g., glycolic acid, lactic acid) alone. Treatment with truncated human type 21 collagen polypeptide (e.g., having an amino acid sequence of SEQ ID NO: 16) in combination with ascorbic acid, retinol, hyaluronic acid, salicylic acid, benzoyl peroxide, niacinamide, or alpha hydroxy acids (e.g., glycolic acid, lactic acid) increases (e.g., synergistically, additively) DNA repair of skin cells (e.g., keratinocytes, fibroblasts) (e.g., as measured by, e.g., thymidine dimer ELISA detection) relative to treatment with the truncated human type 21 collagen, ascorbic acid, retinol, hyaluronic acid, salicylic acid, benzoyl peroxide, niacinamide, or alpha hydroxy acids (e.g., glycolic acid, lactic acid) alone. Treatment with truncated human type 21 collagen polypeptide (e.g., having an amino acid sequence of SEQ ID NO: 16) in combination with ascorbic acid, retinol, hyaluronic acid, salicylic acid, benzoyl peroxide, niacinamide, or alpha hydroxy acids (e.g., glycolic acid, lactic acid) reduces (e.g., synergistically, additively) inflammation (e.g., as measured by, e.g., ELISA analysis or Luminex detection of cytokines) relative to treatment with the truncated human type 21 collagen, ascorbic acid, retinol, hyaluronic acid, salicylic acid, benzoyl peroxide, niacinamide, or alpha hydroxy acids (e.g., glycolic acid, lactic acid) alone. Treatment with truncated human type 21 collagen polypeptide (e.g., having an amino acid sequence of SEQ ID NO: 16) in combination with ascorbic acid, retinol, hyaluronic acid, salicylic acid, benzoyl peroxide, niacinamide, or alpha hydroxy acids (e.g., glycolic acid, lactic acid) reduces (e.g., synergistically, additively) reactive oxidative stress (e.g., as measured by, e.g., CM-H2DCFDA detection) relative to treatment with the truncated human type 21 collagen, ascorbic acid, retinol, hyaluronic acid, salicylic acid, benzoyl peroxide, niacinamide, or alpha hydroxy acids (e.g., glycolic acid, lactic acid) alone.

### Example 10. Clinical Testing Outcomes of Combinations of Truncated Human Type 21 Collagen Polypeptide and Hyaluronic Acid, Retinol, Ascorbic Acid, Salicylic Acid, Benzoyl Peroxide, Niacinamide, or Alpha Hydroxy Acids (e.g., Glycolic Acid, Lactic Acid)

Topical Application of Combinations of Truncated Human Type 21 Collagen Polypeptide with Ascorbic Acid, Retinol, Hyaluronic Acid, Salicylic Acid, Benzoyl Peroxide, Niacinamide, or Alpha Hydroxy Acids (e.g., Glycolic Acid, Lactic Acid) Improve Skin Appearance and Quality

In a clinical study, subjects use a topical formulation of a truncated human type 21 collagen polypeptide (e.g., having an amino acid sequence of SEQ ID NO: 16) alone, or in combination with ascorbic acid, retinol, hyaluronic acid, salicylic acid, benzoyl peroxide, niacinamide, or alpha hydroxy acids (e.g., glycolic acid, lactic acid). Subjects treated with truncated human type 21 collagen polypeptide (e.g., having an amino acid sequence of SEQ ID NO: 16) in combination with ascorbic acid, retinol, hyaluronic acid, salicylic acid, benzoyl peroxide, niacinamide, or alpha hydroxy acids (e.g., glycolic acid, lactic acid) exhibit a reduction in wrinkles and fine lines, a reduction in skin redness and pigmentation/hyperpigmentation, an increase in skin brightness, a decrease in pore size, a decrease in skin roughness, and/or a reduction in acne (e.g., as measured using, e.g., CLARITY analysis) relative to treatment with the truncated human type 21 collagen, ascorbic acid, retinol, hyaluronic acid, salicylic acid, benzoyl peroxide, niacinamide, or alpha hydroxy acids (e.g., glycolic acid, lactic acid) alone.

Subjects treated with truncated human type 21 collagen polypeptide (e.g., having an amino acid sequence of SEQ ID NO: 16) in combination with ascorbic acid, retinol, hyaluronic acid, salicylic acid, benzoyl peroxide, niacinamide, or alpha hydroxy acids (e.g., glycolic acid, lactic acid) exhibit an increase in skin elasticity, an increase in skin firmness, an increase in skin hydration, an increase in skin barrier function, an increase in skin collagen and elastin content, and/or an increase in dermal density relative to treatment with the truncated human type 21 collagen, ascorbic acid, retinol, hyaluronic acid, salicylic acid, benzoyl peroxide, niacinamide, or alpha hydroxy acids (e.g., glycolic acid, lactic acid) alone.

Subjects treated with truncated human type 21 collagen polypeptide (e.g., having an amino acid sequence of SEQ ID NO: 16) in combination with ascorbic acid, retinol, hyaluronic acid, salicylic acid, benzoyl peroxide, niacinamide, or alpha hydroxy acids (e.g., glycolic acid, lactic acid) exhibit an improvement of skin microflora (e.g., reduced abundance of harmful microbes, promotion of beneficial microbes, and/or increased microbial diversity) relative to treatment with the truncated human type 21 collagen, ascorbic acid, retinol, hyaluronic acid, salicylic acid, benzoyl peroxide, niacinamide, or alpha hydroxy acids (e.g., glycolic acid, lactic acid) alone.

### Example 11. Combinations of Truncated Human Type 21 Collagen Polypeptide and Hyaluronic Acid, Retinol, Ascorbic Acid, Salicylic Acid, Benzoyl Peroxide, Niacinamide, or Alpha Hydroxy Acids (e.g., Glycolic Acid, Lactic Acid) Improve Hair Quality and Appearance

In a clinical study, subjects use a composition of a truncated human type 21 collagen polypeptide (e.g., having an amino acid sequence of SEQ ID NO: 16) alone, or in combination with ascorbic acid, retinol, hyaluronic acid, salicylic acid, benzoyl peroxide, niacinamide, or alpha hydroxy acids (e.g., glycolic acid, lactic acid), formulated for application to the hair. Subjects treated with truncated human type 21 collagen polypeptide (e.g., having an amino acid sequence of SEQ ID NO: 16) in combination with ascorbic acid, retinol, hyaluronic acid, salicylic acid, benzoyl peroxide, niacinamide, or alpha hydroxy acids (e.g., glycolic acid, lactic acid) exhibit improved hair growth, thicker hair fiber diameter, increased combability, reduced hair loss, faster growth rate, and/or increased hair tensile strength relative to treatment with the truncated human type 21 collagen, ascorbic acid, retinol, hyaluronic acid, salicylic acid, benzoyl peroxide, niacinamide, or alpha hydroxy acids (e.g., glycolic acid, lactic acid) alone.

Subjects treated with truncated human type 21 collagen polypeptide (e.g., having an amino acid sequence of SEQ ID NO: 16) in combination with ascorbic acid, retinol, hyaluronic acid, salicylic acid, benzoyl peroxide, niacinamide, or alpha hydroxy acids (e.g., glycolic acid, lactic acid) exhibit improved hair fiber thickness and density, increased moisture, increased hydrophobicity, reduced split ends, reduced frizz/increased static control, improved fiber alignment/shine, increased wet/dry combability, and/or stronger resistance to hair breakage relative to treatment with the truncated human type 21 collagen, ascorbic acid, retinol, hyaluronic acid, salicylic acid, benzoyl peroxide, niacinamide, or alpha hydroxy acids (e.g., glycolic acid, lactic acid) alone.

### Example 12. 25 kDa Truncated Human Elastin

A non-naturally occurring truncated human elastin without a His tag, linker, and/or thrombin cleavage site is disclosed below. The non-naturally occurring truncated human elastin was truncated relative to full-length human elastin (SEQ ID NO: 53). The codon-optimized nucleotide sequence encoding this elastin and the amino acid sequence are disclosed below. In SEQ ID NO: 37, the DsbA secretion tag is encoded by nucleotides 1-57 and encodes amino acids 1-19 of SEQ ID NO: 38. In SEQ ID NO: 37, the non-naturally occurring truncated human elastin sequence is encoded by nucleotides 58-927 and encodes amino acids 20-309 of SEQ ID NO: 38.

The codon-optimized nucleotide sequence encoding this non-naturally occurring truncated elastin is provided in SEQ ID NO: 37.

(SEQ ID NO: 37)
ATGAAAAAGATTTGGCTGGCGCTGGCTGGTTTAGTTTTAGCGTTTAGCGC
ATCGGCGGGTCCGCAACCTGGGGTTCCGTTAGGTTATCCGATTAAAGCAC
CGAAACTGCCCGGCGGTTATGGTCTGCCGTACACAACCGGTAAACTGCCG
TATGGTTATGGCCCGGGTGGAGTTGCGGGTGCAGCAGGTAAAGCGGGTTA
TCCTACCGGAACCGGTGTAGGTCCGCAGGCCGCTGCTGCCGCCGCCGCAA
AAGCAGCGGCTAAATTTGGCGCCGGAGCAGCGGGTGTTCTGCCTGGAGTT
GGTGGTGCGGGCGTGCCAGGGGTACCTGGTGCAATTCCGGGTATTGGTGG
TATTGCCGGTGTCGGCACCCCGGCCGCGGCAGCTGCGGCAGCGGCGGCTG
CCAAAGCTGCTAAATACGGTGCCGCGGCGGGTCTGGTGCCAGGAGGTCCG
GGTTTTGGTCCGGGAGTGGTTGGCGTGCCTGGCGCAGGCGTTCCTGGTGT
GGGCGTTCCAGGTGCAGGGATTCCTGTTGTGCCTGGTGCCGGTATTCCCG
GCGCGGCCGTTCCGGGGGTGGTTAGCCCGGAAGCCGCAGCGAAGGCTGCG
GCAAAGGCAGCAAAGTATGGCGCACGCCCAGGAGTCGGCGTGGGTGGTAT
CCCGACCTATGGGGTGGGCGCAGGGGGTTTTCCTGGTTTCGGCGTAGGTG
TAGGAGGTATACCGGGCGTGGCCGGTGTACCAGGGGTTGGTGGCGTCCCT
GGTGTTGGCGGTGTGCCAGGTGTTGGTATTTCACCGGAAGCACAGGCAGC
AGCCGCAGCTAAGGCAGCGAAATATGGTGCCGCCGGCGCAGGAGTTTTAG
GTGGGCTGGTTCCGGGCCCGCAGGCAGCTGTGCCGGGGGTTCCAGGCACC
GGTGGTGTCCCTGGAGTCGGTACGCCGTAA

The amino acid sequence of the 25 kDa non-naturally occurring truncated human elastin sequence including the DsbA secretion signal is disclosed in SEQ ID NO: 38.

(SEQ ID NO: 38)
MKKIWLALAGLVLAFSASAGPQPGVPLGYPIKAPKLPGGYGLPYTTGKL
PYGYGPGGVAGAAGKAGYPTGTGVGPQAAAAAAAKAAAKFGAGAAGVLP
GVGGAGVPGVPGAIPGIGGIAGVGTPAAAAAAAAAAKAAKYGAAAGLVP
GGPGFGPGVVGVPGAGVPGVGVPGAGIPVVPGAGIPGAAVPGVVSPEAA
AKAAAKAAKYGARPGVGVGGIPTYGVGAGGFPGFGVGVGGIPGVAGVPG
VGGVPGVGGVPGVGISPEAQAAAAAKAAKYGAAGAGVLGGLVPGPQAAV
PGVPGTGGVPGVGTP

The codon-optimized nucleotide sequence encoding the non-naturally occurring truncated human elastin without the DsbA secretion tag is provided in SEQ ID NO: 39.

(SEQ ID NO: 39)
GGTCCGCAACCTGGGGTTCCGTTAGGTTATCCGATTAAAGCACCGAAAC
TGCCCGGCGGTTATGGTCTGCCGTACACAACCGGTAAACTGCCGTATGG
TTATGGCCCGGGTGGAGTTGCGGGTGCAGCAGGTAAAGCGGGTTATCCT
ACCGGAACCGGTGTAGGTCCGCAGGCCGCTGCTGCCGCCGCCGCAAAAG
CAGCGGCTAAATTTGGCGCCGGAGCAGCGGGTGTTCTGCCTGGAGTTGG
TGGTGCGGGCGTGCCAGGGGTACCTGGTGCAATTCCGGGTATTGGTGGT
ATTGCCGGTGTCGGCACCCCGGCCGCGGCAGCTGCGGCAGCGGCGGCTG
CCAAAGCTGCTAAATACGGTGCCGCGGCGGGTCTGGTGCCAGGAGGTCC
GGGTTTTGGTCCGGGAGTGGTTGGCGTGCCTGGCGCAGGCGTTCCTGGT
GTGGGCGTTCCAGGTGCAGGGATTCCTGTTGTGCCTGGTGCCGGTATTC
CCGGCGCGGCCGTTCCGGGGGTGGTTAGCCCGGAAGCCGCAGCGAAGGC
TGCGGCAAAGGCAGCAAAGTATGGCGCACGCCCAGGAGTCGGCGTGGGT
GGTATCCCGACCTATGGGGTGGGCGCAGGGGGTTTTCCTGGTTTCGGCG
TAGGTGTAGGAGGTATACCGGGCGTGGCCGGTGTACCAGGGGTTGGTGG
CGTCCCTGGTGTTGGCGGTGTGCCAGGTGTTGGTATTTCACCGGAAGCA
CAGGCAGCAGCCGCAGCTAAGGCAGCGAAATATGGTGCCGCCGGCGCAG
GAGTTTTAGGTGGGCTGGTTCCGGGCCCGCAGGCAGCTGTGCCGGGGGT
TCCAGGCACCGGTGGTGTCCCTGGAGTCGGTACGCCGTAA

The amino acid sequence of the non-naturally occurring truncated human elastin without the DsbA secretion tag is disclosed in SEQ ID NO: 40.

(SEQ ID NO: 40)
GPQPGVPLGYPIKAPKLPGGYGLPYTTGKLPYGYGPGGVAGAAGKAGYP
TGTGVGPQAAAAAAAKAAAKFGAGAAGVLPGVGGAGVPGVPGAIPGIGG
IAGVGTPAAAAAAAAAAKAAKYGAAAGLVPGGPGFGPGVVGVPGAGVPG
VGVPGAGIPVVPGAGIPGAAVPGVVSPEAAAKAAAKAAKYGARPGVGVG
GIPTYGVGAGGFPGFGVGVGGIPGVAGVPGVGGVPGVGGVPGVGISPEA
QAAAAAKAAKYGAAGAGVLGGLVPGPQAAVPGVPGTGGVPGVGTP

The polynucleotide of SEQ ID NO: 37 was synthesized by Gen9 DNA (now Ginkgo Bioworks) internal DNA synthesis. Overlaps between the pET28 vector and SEQ ID NO: 37 and SEQ ID NO: 39 were designed to be between 20 and 30 bp long and added using PCR with the enzyme PrimeSTAR® GXL polymerase (www.takarabio.com/products/per/gc-rich-per/primestar-gxl-dna-polymerase). The opened pET28a vector and insert DNA (SEQ ID NO: 37) were then assembled together into the final plasmid using In-Fusion Cloning (www.takarabio.com/products/cloning/in-fusion-cloning). Sequence of plasmid was then verified through Sanger sequencing through Genewiz (www.genewiz.com/en).

The transformed cells were cultivated in minimal media and frozen in 1.5 mL aliquots with vegetable glycerin at a ratio of 50:50 of cells to glycerin. One vial of this frozen culture was revived in 50 mL of minimal media overnight at 37° C., 200 rpm. Cells were transferred into 300 mL of minimal media and grown for 6-9 hours to reach an OD600 of 5-10. The minimal media was prepared following the minimal media formulation as described in Example 2, above.

The fermentations were performed at various temperatures ranging from 25° to 28° C. For some fermentations, the temperature of the fermentation was maintained at a constant temperature. For other fermentations, the temperature of the fermentations was maintained for a desired period of time and when cell densities of OD600 of 10-20 were reached, the temperature was reduced to induce protein production. Typically, the temperature was reduced from 28° C. to 25° C.; the fermentation at 25° C. was continued for 40-60 hours.

The purified non-naturally occurring truncated elastin was analyzed on an SDS-PAGE gel and a clear band was observed at the expected size of 25 kilodaltons in a location void of a band in the control strain.

### Example 13. 21 kDa Truncated Human Elastin

A non-naturally occurring truncated human elastin is synthesized according to the method of Example 12.

The amino acid sequence of a 21 kDa non-naturally occurring truncated human elastin truncated at the N-terminal relative to a full-length human elastin (SEQ ID NO: 53) is disclosed in SEQ ID NO: 41. The 21 kDa non-naturally occurring truncated human elastin has amino acids 1-522 deleted from the full length human elastin (SEQ ID NO: 53).

(SEQ ID NO: 41)
GPGGVAAAAKSAAKVAAKAQLRAAAGLGAGIPGLGVGVGVPGLGVGAGV
PGLGVGAGVPGFGAGADEGVRRSLSPELREGDPSSSQHLPSTPSSPRVP
GALAAAKAAKYGAAVPGVLGGLGALGGVGIPGGVVGAGPAAAAAAAKAA
AKAAQFGLVGAAGLGGLGVGGLGVPGVGGLGGIPPAAAAKAAKYGAAGL
GGVLGGAGQFPLGGVAARPGFGLSPIFPGGACLGKACGRKRK

The codon optimized polynucleotide sequence encoding the 21 kDa non-naturally occurring truncated human elastin is disclosed in SEQ ID NO: 42.

(SEQ ID NO: 42)
GGTCCGGGCGGTGTCGCAGCAGCAGCTAAAAGCGCGGCGAAAGTTGCGG
CCAAAGCCCAACTGCGCGCCGCCGCGGGCCTCGGTGCAGGTATTCCGGG
GCTGGGTGTCGGAGTTGGAGTCCCGGGTTTGGGCGTGGGCGCGGGAGTT
CCGGGACTGGGAGTGGGTGCCGGAGTTCCTGGCTTTGGTGCAGGCGCAG
ATGAAGGTGTTCGTCGTAGCCTGAGTCCGGAACTGCGTGAAGGTGATCC
GAGTAGCAGCCAGCATCTGCCGAGCACCCCGAGCAGCCCGCGTGTTCCG
GGTGCATTAGCTGCAGCAAAAGCCGCCAAGTATGGTGCAGCCGTGCCGG
GCGTCTTAGGTGGTCTGGGCGCCCTGGGTGGTGTAGGCATTCCGGGAGG
TGTTGTGGGTGCAGGACCGGCCGCCGCAGCTGCGGCCGCCAAAGCAGCT
GCAAAAGCGGCCCAGTTTGGTTTAGTGGGCGCCGCAGGTTTAGGCGGTT
TAGGTGTGGGTGGACTGGGTGTACCTGGCGTAGGCGGTCTGGGTGGAAT
TCCGCCCGCAGCGGCCGCGAAAGCGGCAAAATATGGCGCGGCAGGCCTG
GGCGGCGTGCTGGGTGGGGCAGGTCAGTTTCCGCTGGGCGGGGTTGCCG
CACGTCCGGGATTTGGTCTGAGCCCGATTTTCCCTGGCGGCGCATGTCT
GGGTAAAGCATGTGGTCGTAAACGTAAATAA

The cells are cultivated and the polynucleotide of SEQ ID NO: 42 is expressed as disclosed in Example 12.

### Example 14. 60 kDa Truncated Human Elastin

The amino acid sequence of a 60.3 kDa non-naturally occurring truncated human elastin truncated internally relative to a full-length human elastin (SEQ ID NO: 53) is disclosed in SEQ ID NO: 43. The 60.3 kDa non-naturally occurring truncated human elastin has amino acids 461-527 deleted from the full length human elastin (SEQ ID NO: 53).

(SEQ ID NO: 43)
GGVPGAIPGGVPGGVFYPGAGLGALGGGALGPGGKPLKPVPGGLAGAGL
GAGLGAFPAVTFPGALVPGGVADAAAAYKAAKAGAGLGGVPGVGGLGVS
AGAVVPQPGAGVKPGKVPGVGLPGVYPGGVLPGARFPGVGVLPGVPTGA
GVKPKAPGVGGAFAGIPGVGPFGGPQPGVPLGYPIKAPKLPGGYGLPYT
TGKLPYGYGPGGVAGAAGKAGYPTGTGVGPQAAAAAAAKAAAKFGAGAA
GVLPGVGGAGVPGVPGAIPGIGGIAGVGTPAAAAAAAAAAKAAKYGAAA
GLVPGGPGFGPGVVGVPGAGVPGVGVPGAGIPVVPGAGIPGAAVPGVVS
PEAAAKAAAKAAKYGARPGVGVGGIPTYGVGAGGFPGFGVGVGGIPGVA
GVPGVGGVPGVGGVPGVGISPEAQAAAAAKAAKYGAAGAGVLGGLVPGP
QAAVPGVPGTGGVPGVGTPAAAAKSAAKVAAKAQLRAAAGLGAGIPGLG
VGVGVPGLGVGAGVPGLGVGAGVPGFGAGADEGVRRSLSPELREGDPSS
SQHLPSTPSSPRVPGALAAAKAAKYGAAVPGVLGGLGALGGVGIPGGVV
GAGPAAAAAAAKAAAKAAQFGLVGAAGLGGLGVGGLGVPGVGGLGGIPP
AAAAKAAKYGAAGLGGVLGGAGQFPLGGVAARPGFGLSPIFPGGACLGK
ACGRKRK

The codon optimized polynucleotide sequence encoding the 60.3 kDa non-naturally occurring truncated human elastin is disclosed in SEQ ID NO: 44.

(SEQ ID NO: 44)
GGTGGCGTACCAGGCGCAATTCCTGGGGGTGTCCCAGGCGGTGTTTTTT
ATCCGGGCGCCGGTCTTGGCGCACTGGGTGGCGGTGCACTGGGCCCGGG
CGGCAAACCGCTGAAACCGGTACCAGGTGGTTTAGCAGGCGCCGGCTTA
GGCGCAGGTCTGGGAGCATTTCCGGCAGTTACCTTTCCAGGGGCACTGG
TTCCTGGAGGTGTGGCCGATGCAGCCGCGGCATATAAAGCCGCTAAAGC
CGGTGCGGGTTTAGGAGGCGTCCCAGGTGTCGGTGGCCTGGGTGTTAGC
GCCGGTGCAGTTGTTCCGCAGCCGGGAGCAGGGGTTAAACCTGGTAAAG
TGCCGGGAGTAGGTCTGCCAGGCGTTTATCCTGGTGGTGTTTTGCCGGG
TGCCCGTTTTCCGGGCGTTGGTGTTCTTCCAGGCGTGCCGACCGGAGCC
GGTGTTAAACCGAAAGCCCCCGGTGTTGGAGGTGCATTTGCAGGCATCC
CGGGAGTTGGCCCGTTTGGTGGTCCGCAACCTGGGGTTCCGTTAGGTTA
TCCGATTAAAGCACCGAAACTGCCCGGCGGTTATGGTCTGCCGTACACA
ACCGGTAAACTGCCGTATGGTTATGGCCCGGGTGGAGTTGCGGGTGCAG
CAGGTAAAGCGGGTTATCCTACCGGAACCGGTGTAGGTCCGCAGGCCGC
TGCTGCCGCCGCCGCAAAAGCAGCGGCTAAATTTGGCGCCGGAGCAGCG
GGTGTTCTGCCTGGAGTTGGTGGTGCGGGCGTGCCAGGGGTACCTGGTG
CAATTCCGGGTATTGGTGGTATTGCCGGTGTCGGCACCCCGGCCGCGGC
AGCTGCGGCAGCGGCGGCTGCCAAAGCTGCTAAATACGGTGCCGCGGCG
GGTCTGGTGCCAGGAGGTCCGGGTTTTGGTCCGGGAGTGGTTGGCGTGC
CTGGCGCAGGCGTTCCTGGTGTGGGCGTTCCAGGTGCAGGGATTCCTGT
TGTGCCTGGTGCCGGTATTCCCGGCGCGGCCGTTCCGGGGGTGGTTAGC
CCGGAAGCCGCAGCGAAGGCTGCGGCAAAGGCAGCAAAGTATGGCGCAC
GCCCAGGAGTCGGCGTGGGTGGTATCCCGACCTATGGGGTGGGCGCAGG
GGGTTTTCCTGGTTTCGGCGTAGGTGTAGGAGGTATACCGGGCGTGGCC
GGTGTACCAGGGGTTGGTGGCGTCCCTGGTGTTGGCGGTGTGCCAGGTG
TTGGTATTTCACCGGAAGCACAGGCAGCAGCCGCAGCTAAGGCAGCGAA
ATATGGTGCCGCCGGCGCAGGAGTTTTAGGTGGGCTGGTTCCGGGCCCG
CAGGCAGCTGTGCCGGGGGTTCCAGGCACCGGTGGTGTCCCTGGAGTCG
GTACGCCGGCAGCAGCAGCTAAAAGCGCGGCGAAAGTTGCGGCCAAAGC
CCAACTGCGCGCCGCCGCGGGCCTCGGTGCAGGTATTCCGGGGCTGGGT
GTCGGAGTTGGAGTCCCGGGTTTGGGCGTGGGCGCGGGAGTTCCGGGAC
TGGGAGTGGGTGCCGGAGTTCCTGGCTTTGGTGCAGGCGCAGATGAAGG
TGTTCGTCGTAGCCTGAGTCCGGAACTGCGTGAAGGTGATCCGAGTAGC
AGCCAGCATCTGCCGAGCACCCCGAGCAGCCCGCGTGTTCCGGGTGCAT
TAGCTGCAGCAAAAGCCGCCAAGTATGGTGCAGCCGTGCCGGGCGTCTT
AGGTGGTCTGGGCGCCCTGGGTGGTGTAGGCATTCCGGGAGGTGTTGTG
GGTGCAGGACCGGCCGCCGCAGCTGCGGCCGCCAAAGCAGCTGCAAAAG
CGGCCCAGTTTGGTTTAGTGGGCGCCGCAGGTTTAGGCGGTTTAGGTGT
GGGTGGACTGGGTGTACCTGGCGTAGGCGGTCTGGGTGGAATTCCGCCC
GCAGCGGCCGCGAAAGCGGCAAAATATGGCGCGGCAGGCCTGGGCGGCG
TGCTGGGTGGGGCAGGTCAGTTTCCGCTGGGCGGGGTTGCCGCACGTCC
GGGATTTGGTCTGAGCCCGATTTTCCCTGGCGGCGCATGTCTGGGTAAA
GCATGTGGTCGTAAACGTAAATAA

The cells are cultivated and the polynucleotide of SEQ ID NO: 44 is expressed as disclosed in Example 12.

### Example 15. Effect of Truncated Elastin on Fibroblast Cell Viability, Procollagen Synthesis, and Tropoelastin Synthesis

A human fibroblast cell culture is used to assess the ability of the non-naturally occurring truncated human elastin molecule of Example 12 to affect procollagen and tropoelastin synthesis. The human fibroblast cell culture is also used to determine the viability of the human fibroblast cells after exposure to the truncated elastin.

A stock solution of 2% w/w truncated elastin is prepared from the truncated elastin of Example 12. Aliquots from the 2% stock truncated elastin solution are then used in the experiments described below.

### Preparation of Fibroblasts

Fibroblasts are seeded into the individual wells of a 24-well plate in 0.5 mL of Fibroblast Growth Media (FGM) and incubated overnight at 37±2° C. and 5±1% CO₂. On the following day, the media is removed via aspiration to eliminate any non-adherent cells and replaced with 0.5 mL of fresh FGM. The cells are grown until confluent, with a media change every 48 to 72 hours. Upon reaching confluency the cells are treated for 24 hours with Dulbecco's Modified Eagle Medium (DMEM) supplemented with 1.5% Fetal Bovine Serum (FBS) to wash out any effects from the growth factors included in the normal culture media. After the 24-hour wash out period the cells are treated with the truncated elastin at specified concentrations dissolved in FGM with 1.5% FBS. Transforming Growth Factor Beta (TGF-β) (20 ng/mL) is used as a positive control for collagen and elastin synthesis. Untreated cells (negative controls) receive only DMEM with 1.5% FBS. The cells are incubated for 48 hours and at the end of the incubation period, cell culture media is collected and either stored frozen (−75° C.) or assayed immediately. Materials are tested in triplicate.

### MTT Assay

The MTT (3-(4,5-Dimethylthiazol-2-yl)-2,5-diphenyltetrazolium bromide, a tetrazole) assay is a colorimetric assay used to determine the metabolic activity of cells. Changes in cell number are assessed via an MTT assay. When cells are exposed to MTT, reduction of MTT by mitochondria in viable cells results in the formation of insoluble purple formazin crystals that are extracted from the cells with isopropanol and quantified spectrophotometrically. Non-living cells cannot reduce MTT and therefore cannot produce the purple formazin crystals. The intensity of the purple color is directly proportional to the number of living cells (metabolically active cells). The intensity of the purple color is directly proportional to the metabolic activity of the cells and is inversely proportional to the toxicity of the test material.

After the 2-day incubation discussed above, the cell culture medium is removed (see above) and the fibroblasts are washed twice with Phosphate Buffered Saline (PBS) to remove any remaining elastin molecules. After the final wash, 500 μL of DMEM supplemented with 0.5 mg/ml MTT is added to each well and the cells are incubated for 1 hour at 37±2° C. and 5±1% CO₂. After the incubation, the DMEM/MTT solution is removed and the cells are washed again once with PBS and then 0.5 mL of isopropyl alcohol is added to the well to extract the purple formazin crystals. Two hundred microliters of the isopropyl extracts is transferred to a 96-well plate and the plate read at 540 nm using isopropyl alcohol as a blank.

The mean MTT absorbance value for the negative control cells is calculated and used to represent 100% cell viability. The individual MTT absorbance values from the cells undergoing the various treatments are then divided by the mean value for the negative control cells and expressed as a percent to determine the change in cell viability caused by each treatment.

### Procollagen Synthesis

Fibroblasts are the main source of the extracellular matrix peptides, including the structural proteins collagen and elastin. Procollagen is a large peptide synthesized by fibroblasts in the dermal layer of the skin and is the precursor for collagen. As the peptide is processed to form a mature collagen protein, the propeptide portion is cleaved off (type I C-peptide). Both the mature collagen protein and the type I C-peptide fragment are then released into the extracellular environment. As collagen is synthesized, the type I C-peptide fragment accumulates into the tissue culture medium. Since there is a 1:1 stoichiometric ratio between the two parts of the procollagen peptide, assaying for type I C-peptide reflects the amount of collagen synthesized. Type I C-peptide can be assayed via an Enzyme Linked Immunosorbent Assay (ELISA)-based method.

A series of type I C-peptide standards are prepared ranging from 0 ng/mL to 640 ng/mL. Next, an ELISA microplate is prepared by removing any unneeded strips from the plate frame followed by the addition of 100 μL of peroxidase-labeled anti-procollagen type I-C peptide antibody to each well used in the assay. Twenty (20) μL of either sample (collected tissue culture media) or standard is then added to appropriate wells and the microplate is covered and allowed to incubate for 3±0.25 hours at 37° C. After the incubation, the wells are aspirated and washed three times with 400 μL of wash buffer. After the last wash is removed, 100 μL of peroxidase substrate solution (hydrogen peroxide+tetramethylbenzidine as a chromagen) is added to each well and the plate is incubated for 15±5 minutes at room temperature. After the incubation, 100 μL of stop solution (1 N sulfuric acid) is added to each well and the plate is read using a microplate reader at 450 nm.

To quantify the amount of each substance present, a standard curve is generated using known concentrations of each substance. A regression analysis is performed to establish the line that best fits these data points. Absorbance values for the test materials and untreated samples are used to estimate the amount of each substance present in each sample.

### Elastin Synthesis

Elastin is the main component of a network of elastic fibers that give tissues their ability to recoil after a transient stretch. This protein is released by fibroblasts (soluble elastin) into the extracellular space where it is then cross-linked to other elastin proteins to form an extensive network of fibers and sheets (insoluble elastin). Soluble elastin can be readily measured from cell culture medium via an ELISA-based method.

Soluble α-elastin is dissolved in 0.1 M sodium carbonate (pH 9.0) at a concentration of 1.25 μm/mL. 150 μL of this solution is then applied to the wells of a 96-well Maxisorp Nunc plate, and the plate is incubated overnight at 4° C. On the following day, the wells are saturated with PBS containing 0.25% Bovine Serum Albumin (BSA) and 0.05% Tween 20. The plate is then incubated with this blocking solution for 1 hour at 37° C. and then washed two times with PBS containing 0.05% Tween 20.

A set of α-elastin standards is generated ranging from 0 to 100 ng/mL. 180 μL of either standard or truncated elastin is then transferred to a 650 μL microcentrifuge tube. An anti-elastin antibody solution is prepared (the antibody is diluted 1:100 in PBS containing 0.25% BSA and 0.05% Tween 20) and 20 μL of the solution is added to the tube. The tubes are then incubated overnight at 4±2° C. On the following day, 150 μL is transferred from each tube to the 96-well elastin ELISA plate, and the plate is incubated for 1 hour at room temperature. The plate is then washed 3 times with PBS containing 0.05% Tween 20. After washing, 200 μL of a solution containing a peroxidase linked secondary antibody diluted in PBS containing 0.25% BSA and 0.05% Tween 20 is added, and the plate is incubated for 1 hour at room temperature. After washing the plate three times, 200 μL of a substrate solution is added and the plate is incubated for 10 to 30 minutes in the dark at room temperature. After this final incubation, the plate is read at 460 nm using a plate reader.

### Example 16. Effect of Truncated Elastin on Keratinocyte Proliferation and UVB Protection

A human keratinocyte cell culture model is used to assess the ability of the test materials to exert an effect on cell proliferation. In addition, the impact of the test materials on the cell viability after an exposure to UVB is also assessed.

A stock solution of 2% w/w truncated elastin is prepared from the truncated elastin of Example 12. Aliquots from the 2% stock truncated elastin solution are then used in the experiments described below.

This study is conducted in two parts. In the first part, cultured keratinocytes are incubated with the test materials for 48 hours, after which, changes in the number of viable cells are assessed using an MTT assay. In the second part of the study, cultured keratinocytes are irradiated with UVB and then treated with the test materials for 48 hours. At the end of the 48 hour period, the number of viable cells is again assessed via an MTT assay.

Changes in cell number of viable cells can be determined using an MTT (3-(4,5-Dimethylthiazol-2-yl)-2,5-diphenyltetrazolium bromide, a tetrazole) assay. The MTT assay is a colorimetric analysis of the metabolic activity of the cell, which is a reflection of the number of viable cells. Reduction of MTT by mitochondria in viable cells results in the formation of insoluble purple formazin crystals that are extracted from the cells with isopropanol and quantified spectrophotometrically. The intensity of the purple color is directly proportional to the number of metabolically active cells.

### Proliferation Assay

For the proliferation assay, the keratinocytes are seeded into 96-well plates without growth factors and incubated for 24 hours at 37±2° C. and 5±1% CO₂. After this initial incubation, the media is replaced with media supplemented with the test materials. Normal media (with growth factors) is used as a positive control. After the addition of the test materials, the cells are cultured for 48 hours, as described above. At the end of the incubation period, changes in the number of viable cells are determined using an MTT assay.

### UVB Protection Assay

For the UVB protection assay, the keratinocytes are seeded into 96-well plates using normal media and incubated for 24 hours at 37±2° C. and 5±1% CO₂. After this initial incubation, the media is replaced with 100 μL of phosphate buffered saline (PBS) and the cells are exposed to UVB (40 mJ/cm²). After the UVB exposure, the PBS is replaced with fresh media supplemented with the test materials (ascorbic acid at 100 μg/mL serves as the positive control) and the cells are cultured for 48 hours at 37±2° C. and 5±1% CO₂. At the end of the 48 hour incubation, cell viability is determined using an MTT assay.

### Example 17. Effect of Truncated Elastin on Thymine Dimer Formation

Upon exposure to ultraviolet radiation, the thymine dimer (TT dimer) content in DNA present in cells increases. Increases in TT dimer formation are correlated with skin damage and certain types of cell proliferative diseases including skin cancer.

The truncated elastin of Example 12 is tested to determine if it can reduce TT dimer formation in human epidermal keratinocytes. Human keratinocytes are seeded into 12-well plates using normal media and incubated for 24 hours at 37±2° C. and 5±1% CO₂. After this initial incubation, the media is replaced with 100 μL of phosphate buffered saline (PBS) and the cells are exposed to UVB (25 mJ/cm²). After the UVB exposure, the PBS is replaced with fresh media supplemented with the test materials or Trolox (100 μg/ml, a positive control) and the cells are cultured overnight at 37±2° C. and 5±1% CO₂. At the end of the incubation, cellular DNA is extracted and assayed for thymine dimer content using an ELISA-based method.

After the overnight incubation, the cell culture media is removed from the wells and replaced with 200 μL of PBS and 20 μL of Proteinase K. After swirling the plate to mix the PBS and Proteinase K, 200 μL of buffer AL is added to each well. After again swirling the plate to mix the reagents, the plates are incubated for 10 minutes at 55±2° C. After cooling the plate to room temperature, the DNA is precipitated by the addition of 200 μL of 100% ethanol. The precipitated DNA mixtures are then transferred to DNEasy Spin Columns in 2 mL collection tubes and centrifuged at 8,000 RPM for 1 minute. The flow through and collection tubes are discarded, and 500 μL of Wash Buffer One is added to the spin column and the column is placed into a new collection tube and centrifuged at 8,000 RPM for 1 minute. The flow through and collection tube are again discarded, and 500 μL of Wash Buffer Two is added to the spin column, and the column is placed into a new collection tube and centrifuged at 14,000 RPM for 3 minutes. The spin column is then placed into a new 1.5 mL centrifuge tube and 110 μL of ultrapure water is added to the column. The column is incubated for 1 minute at room temperature and then centrifuged at 8,000 RPM for 1 minute.

Extracted DNA is quantified via a fluorometric assay. A 2 μL aliquot of the DNA sample is mixed with 100 μL Tris-EDTA (TE) buffer in a 96-well plate. A series of DNA standards is also transferred to wells in a 96-well plate (in duplicate). Finally, 100 μL of dilute CyQUANT Green dye is added to each well, and the fluorescence intensity of each well is determined using an excitation wavelength of 480 nm and an emission wavelength of 520 nm.

Thymine Dimer Detection can be determined using an OxiSelect™ UV-Induced DNA Damage ELISA Kit.

Aliquots of genomic DNA samples or standards are converted to single stranded DNA by incubating the samples at 95° C. for 10 minutes and then chilling on ice. 100 μL of each sample or standard is transferred to a DNA binding ELISA plate and incubated overnight at 4° C. On the following day, the wells are rinsed once with 100 μL of PBS and then blocked with 150 μL of Assay Diluent for one hour at room temperature. After removing the Assay Diluent, 100 μL of anti-CPD antibody is added to each well and the plate is incubated for one hour at room temperature. After this incubation, the plate is washed three times with 250 μL of wash buffer per well, and then 150 μL of Blocking Reagent is added to the plate. The plate is blocked again for one hour at room temperature and then washed three times as described before. 100 μL of Secondary Antibody is then added to each well, and the plate is incubated for 1 hour at room temperature. After washing the plate again, 100 μL of substrate is added to each well and the plate is incubated for 5-20 minutes to allow for color generation in the plate. The color generation reaction is stopped by the addition of 100 μL of stop solution, and the plate is read at 460 nm using a plate reader.

To quantify the amount of DNA present, a standard curve is generated using known concentrations of DNA and their respective fluorescence intensity (measured in RFUs or relative fluorescence units). A regression analysis is performed to establish the line that best fits the data points. The Relative Fluorescence Units (RFU) for each unknown sample is then used to estimate the amount of DNA.

A series of DNA standards with known amounts of thymine dimer content is used to generate a standard curve. This standard curve is used to determine the amount of DNA damage in the sample DNA. Means for each treatment group are calculated and compared using an ANOVA.

### Example 18. Protective Effect of Truncated Human Elastin on Fibroblasts

The effect of non-naturally occurring truncated human elastin on fibroblast cell viability, procollagen synthesis, and elastin synthesis is determined according to the methods of Example 15.

The effect of non-naturally occurring truncated human elastin on Keratinocyte proliferation and UVB protection is determined according to the methods of Example 16.

The effect of non-naturally occurring truncated human elastin on thymine dimer formation after exposure to UV radiation is determined according to the methods of Example 17.

### Example 19. Effect of Truncated Elastin on Inflammatory Cytokines

Keratinocytes and dermal fibroblasts play an important role in the immune response of the skin. In response to irritating chemicals or UV radiation (pro-inflammatory/pro-irritation stimuli), keratinocytes can release a vast array of cytokines. These cytokines are thought to help engage immune cells to the site of inflammation. Cytokines released by the keratinocytes include TNFα, IL-1α, IL-1β, IL-3, IL-6, IL-7, IL-8, IL-10, IL-18, and IL-1RA.

The testing model used for this study is the MatTek EpiDerm. This skin model consists of normal human-derived epidermal keratinocytes that have been cultured to form a multilayered, highly differentiated model of the human epidermis. Ultrastructural analysis has revealed the presence of keratohyalin granules, tonofilament bundles, desmosomes, and a multi-layered stratum corneum containing intercellular lamellar lipid layers arranged in patterns characteristic of in vivo epidermis. Markers of mature epidermis specific differentiation, such as pro-filaggrin, the K1/K10 cytokeratin pair, involucrin, and type I epidermal transglutaminase, have been localized in this model. The MatTek EpiDerm is also mitotically and metabolically active.

The MatTek EpiDerm tissues are used to assess the ability of various test materials to inhibit the release of the inflammatory mediator IL-la. Test materials are compared to an over the counter topical hydrocortisone preparation (positive control) as well as to untreated tissues (negative control 1) and untreated, non-inflamed tissues (negative control 2). This test is also used to assess the viability of the tissues after exposure to the test materials.

IL-1α, IL-6, and IL-8 are synthesized and stored in keratinocytes and have been identified as mediators of skin irritation and inflammation. Release of these cytokines can be directly measured in tissue culture media via a colorimetric based enzyme linked immunosorbent assay (ELISA). Briefly, antibodies covalently linked to a solid support can bind IL-la, IL-6, or IL-8 present in spent culture media samples. A second antibody that is covalently attached to an acetylcholinesterase enzyme can in turn detect the specific bound cytokines. Upon addition of an appropriate color substrate, the acetylcholinesterase enzyme can generate a colored end product that can be measured spectrophotometrically.

MatTek EpiDerm Tissues are purchased from MatTek corporation and stored at 4° C. until used. Prior to use, the tissues to be used are removed from the agarose-shipping tray and placed into a 6-well plate containing 0.9 mL of hydrocortisone free assay medium (37±2° C.). The tissues are allowed to incubate overnight at 37±2° C. and 5±1% CO₂. After this initial incubation, the assay medium is replaced with 0.9 mL of fresh hydrocortisone free medium (37±2° C.). Three tissues are prepared for each test material.

An inflammatory response in the tissues is initiated via UV irradiation (UVB). A UV lamp is used to give a 300 mJ/cm² dose of UVB radiation to the tissues. Immediately after the application of the inflammatory stimuli, 50 μL or mg of test material is applied directly onto the surface of the tissue. An over the counter hydrocortisone cream is used as a positive control. For a negative control, tissues are exposed to the inflammatory stimuli but are not treated with any type of anti-inflammatory material. One additional set of tissues is left without exposure to the inflammatory stimuli to provide a baseline measurement for the cytokines. The tissues are incubated at 37±2° C. and 5±1% CO₂ for 24 hours after exposure to the inflammatory stimuli. After the 24 hour incubation, the cell culture medium is collected and stored at −75° C. until analyzed for cytokines.

The ELISA plates are prepared by diluting the appropriate capture antibody in PBS. Next, 100 μL of the diluted capture antibody is added to the wells of a 96-well ELISA plate and the plate is incubated overnight at room temperature. On the following day, the plate is washed three times with 300 μL wash buffer (0.05% Tween 20 in PBS) and then blocked by adding 300 μL of blocking buffer (1% BSA in PBS) to each well. The plate is incubated with the blocking buffer for at least one hour. After the incubation, the blocking buffer is removed, and the plate is washed three times as described above.

A series of standards is prepared and 100 μL of each of these standards is dispensed into two wells (duplicates) in the appropriate 96-well plate. Subsequently, 100 μL of each sample is added to additional wells and the plate was incubated for two hours at room temperature. After the incubation, the plate is washed three times as described above. Once the last wash is removed, 100 of a biotin conjugated detection antibody is added. After incubating the plate for two hours at room temperature, the plate is washed again as described above. 100 μL of HRP-streptavidin is then added to each well, and the plate is incubated for 20 minutes at room temperature. Next, 100 of substrate solution (hydrogen peroxide+tetramethylbenzidine as a chromagen) is added to each well. Once a sufficient level of color development occurs, 50 μL of stop solution (2 N sulfuric acid) is added to each well and the plate is read at 460 nm.

After the 24 hour incubation, the tissues are rinsed twice with at least 100 μL of phosphate buffered saline to remove the test material and then transferred to a 6-well plate containing 1.0 mL of assay medium supplemented with MTT (1 mg/mL) and allowed to incubate for 3±0.25 hours at 37±2° C. and 5±1% CO₂. After the incubation, the tissues are rinsed at least twice with 100 μL of phosphate buffered saline, blotted dry, and then placed into a 24-well plate containing 2 mL of isopropanol per well. The 24-well plate is covered and allowed to incubate at room temperature for at least 2 hours on a rocking platform to extract the reduced MTT from the tissues. After the extraction, a 200 μL sample of the isopropanol/MTT mixture is transferred to a 96-well plate, and the absorbance of the sample is read at 540 nm with a plate reader using 200 μL of isopropanol as the blank.

### Example 20. Urban Dust Protection by Truncated Elastin

A keratinocyte cell culture model is used to assess the ability of truncated elastin to exert a protective effect by promoting cell survival after exposure to urban dust.

Human epidermal keratinocytes are pretreated with the test materials and then exposed to urban dust. At the end of the treatment period, changes in cell viability are determined via an MTT assay.

Keratinocytes are seeded into the individual wells of a 96-well plate in 100 μL of medium and incubated overnight at 37±2° C. and 5±1% CO₂. On the following day, the media is removed via aspiration to eliminate any non-adherent cells and replaced with 100 μL of fresh medium. The cells are grown until confluent, with a media change every 48 to 72 hours.

Pre-Treatment with Test Material Followed by Urban Dust Treatment

Test materials are prepared at 2×their final desired concentrations in cell culture media. Urban dust (NIST 1649B from Sigma Chemicals) is also prepared at 2×solutions. For the pretreatment, 50 μL of 2×test material is combined with 50 μL of culture media and the cells are incubated for 24 hours. At the end of the pretreatment period, the test material containing culture media is removed and replaced with 50 μL of 2×urban dust and 50 μL of media. Another set of cells is treated with media alone (non-dust exposed) and used as a reference control to represent 100% cell viability. The cells are then incubated for 24 hours and then subjected to an MTT assay to determine changes in cell viability.

At the end of the treatment period, the cell culture medium is removed, and the cells are washed with PBS. After the wash, 100 μL of cell culture media supplemented with 0.5 mg/mL MTT is added to each well and the cells are incubated for 30 minutes at 37±2° C. and 5±1% CO₂. After the incubation, the media/MTT solution is removed and the cells are washed again once with PBS and then 100 μL of isopropyl alcohol is added to the wells to extract the purple formazin crystals. The 96-well plate is then read at 540 nm using isopropyl alcohol as a blank.

The mean MTT absorbance value for the non-dust exposed cells is calculated and used to represent 100% value for cell number. The individual MTT values from the cells undergoing the various treatments is then divided by the mean value for the non-dust exposed cells and expressed as a percent to determine the change in cell number caused by each treatment.

### Example 21. 23 kDa Truncated Human Elastin

A non-naturally occurring truncated human elastin without a His tag, linker, and thrombin cleavage site is disclosed below. The codon-optimized nucleotide sequence encoding this truncated elastin and the amino acid sequence are disclosed below. In SEQ ID NO: 45, the DsbA secretion tag is encoded by nucleotides 1-57 and encodes amino acids 1-19 of SEQ ID NO: 46. In SEQ ID NO: 45, the truncated elastin sequence is encoded by nucleotides 58-843 and encodes amino acids 20-281 of SEQ ID NO: 46.

The codon-optimized nucleotide sequence encoding this non-naturally occurring truncated elastin is provided in SEQ ID NO: 45.

(SEQ ID NO: 45)
ATGAAAAAGATTTGGCTGGCGCTGGCTGGTTTAGTTTTAGCGTTTAGCG
CATCGGCGGCCGGTCTGGGTGGCGTGCCTGGCGTTGGTGGCCTGGGCGT
TAGCGCCGGTGCAGTTGTTCCGCAGCCTGGTGCGGGTGTTAAACCGGGT
AAAGTTCCTGGTGTTGGTCTGCCTGGTGTTTATCCAGGCGGTGTTCTGC
CAGGTGCGCGTTTTCCTGGCGTGGGTGTTCTGCCGGGTGTTCCGACCGG
TGCAGGCGTGAAACCGAAAGCACCAGGTGTTGGTGGTGCATTTGCAGGT
ATTCCAGGTGTGGGTCCGTTTGGTGGTCCGCAGCCAGGCGTTCCGCTGG
GTTATCCGATTAAAGCACCGAAACTGCCTGGCGGTTATGGTCTGCCGTA
TACCACCGGTAAACTGCCGTATGGTTATGGCCCTGGCGGTGTTGCCGGT
GCGGCAGGTAAAGCAGGCTATCCGACCGGTACCGGTGTAGGTCCGCAGG
CAGCAGCCGCAGCAGCGGCAAAAGCAGCAGCGAAATTTGGTGCGGGTGC
AGCCGGTGTGCTGCCAGGCGTAGGTGGCGCTGGTGTACCTGGTGTCCCT
GGCGCAATTCCAGGTATTGGCGGTATTGCAGGCGTTGGTACTCCGGCAG
CTGCAGCCGCTGCCGCAGCCGCAGCTAAAGCAGCCAAATATGGTGCAGC
GGCAGGCCTGGTTCCTGGCGGTCCAGGTTTTGGTCCGGGTGTTGTTGGC
GTCCCAGGTGCCGGTGTGCCTGGTGTGGGTGTTCCAGGTGCGGGTATTC
CGGTTGTTCCTGGCGCAGGCATTCCGGGTGCCGCAGTTCCGGGTGTAGT
TAGCCCGGAATAA

The amino acid sequence of the 23 kDa non-naturally occurring truncated human elastin sequence including the DsbA secretion signal is disclosed in SEQ ID NO: 46.

(SEQ ID NO: 46)
MKKIWLALAGLVLAFSASAAGLGGVPGVGGLGVSAGAVVPQPGAGVKPG
KVPGVGLPGVYPGGVLPGARFPGVGVLPGVPTGAGVKPKAPGVGGAFAG
IPGVGPFGGPQPGVPLGYPIKAPKLPGGYGLPYTTGKLPYGYGPGGVAG
AAGKAGYPTGTGVGPQAAAAAAAKAAAKFGAGAAGVLPGVGGAGVPGVP
GAIPGIGGIAGVGTPAAAAAAAAAAKAAKYGAAAGLVPGGPGFGPGVVG
VPGAGVPGVGVPGAGIPVVPGAGIPGAAVPGVVSPE

The codon-optimized nucleotide sequence encoding the non-naturally occurring truncated human elastin without the DsbA secretion tag elastin is provided in SEQ ID NO: 47.

(SEQ ID NO: 47)
GCCGGTCTGGGTGGCGTGCCTGGCGTTGGTGGCCTGGGCGTTAGCGCCG
GTGCAGTTGTTCCGCAGCCTGGTGCGGGTGTTAAACCGGGTAAAGTTCC
TGGTGTTGGTCTGCCTGGTGTTTATCCAGGCGGTGTTCTGCCAGGTGCG
CGTTTTCCTGGCGTGGGTGTTCTGCCGGGTGTTCCGACCGGTGCAGGCG
TGAAACCGAAAGCACCAGGTGTTGGTGGTGCATTTGCAGGTATTCCAGG
TGTGGGTCCGTTTGGTGGTCCGCAGCCAGGCGTTCCGCTGGGTTATCCG
ATTAAAGCACCGAAACTGCCTGGCGGTTATGGTCTGCCGTATACCACCG
GTAAACTGCCGTATGGTTATGGCCCTGGCGGTGTTGCCGGTGCGGCAGG
TAAAGCAGGCTATCCGACCGGTACCGGTGTAGGTCCGCAGGCAGCAGCC
GCAGCAGCGGCAAAAGCAGCAGCGAAATTTGGTGCGGGTGCAGCCGGTG
TGCTGCCAGGCGTAGGTGGCGCTGGTGTACCTGGTGTCCCTGGCGCAAT
TCCAGGTATTGGCGGTATTGCAGGCGTTGGTACTCCGGCAGCTGCAGCC
GCTGCCGCAGCCGCAGCTAAAGCAGCCAAATATGGTGCAGCGGCAGGCC
TGGTTCCTGGCGGTCCAGGTTTTGGTCCGGGTGTTGTTGGCGTCCCAGG
TGCCGGTGTGCCTGGTGTGGGTGTTCCAGGTGCGGGTATTCCGGTTGTT
CCTGGCGCAGGCATTCCGGGTGCCGCAGTTCCGGGTGTAGTTAGCCCGG
AATAA

The amino acid sequence of the non-naturally occurring truncated human elastin without the DsbA secretion tag is disclosed in SEQ ID NO: 48.

(SEQ ID NO: 48)
AGLGGVPGVGGLGVSAGAVVPQPGAGVKPGKVPGVGLPGVYPGGVLPGA
RFPGVGVLPGVPTGAGVKPKAPGVGGAFAGIPGVGPFGGPQPGVPLGYP
IKAPKLPGGYGLPYTTGKLPYGYGPGGVAGAAGKAGYPTGTGVGPQAAA
AAAAKAAAKFGAGAAGVLPGVGGAGVPGVPGAIPGIGGIAGVGTPAAAA
AAAAAAKAAKYGAAAGLVPGGPGFGPGVVGVPGAGVPGVGVPGAGIPVV
PGAGIPGAAVPGVVSPE

The polynucleotide of SEQ ID NO: 45 was synthesized by Twist DNA. Overlaps between the pET28 vector and SEQ ID NO: 45 were designed to be between 20 and 30 bp long and added using PCR with the enzyme PrimeSTAR® GXL polymerase (www.takarabio.com/products/pcr/gc-rich-pcr/primestar-gxl-dna-polymerase). The opened pET28a vector and insert DNA (SEQ ID NO: 45) were then assembled together into the final plasmid using In-Fusion Cloning (www.takarabio.com/products/cloning/in-fusion-cloning). The sequence of the plasmid was then verified using Sanger sequencing through Genewiz (www.genewiz.com/en).

The transformed cells were cultivated in minimal media of Example 12 and frozen in 1.5 mL aliquots with vegetable glycerin at a ratio of 50:50 of cells to glycerin. One vial of this frozen culture was revived in 50 mL of minimal media overnight at 37° C., 200 rpm. Cells were transferred into 300 mL of minimal media and grown for 6-9 hours to reach an OD600 of 5-10.

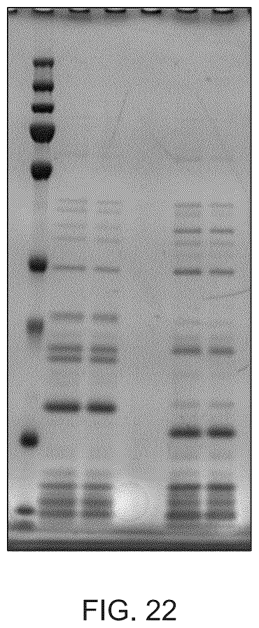

The 23 kDa non-naturally occurring truncated human elastin was detected on 12% BisTris protein gel. A photograph of the gel is depicted in FIG. 22. The 23 kDa non-naturally occurring truncated human elastin was confirmed to be the correct mass by intact mass spectrometry using liquid chromatography—mass spectrometry.

### Example 22. 13 kDa Truncated Human Elastin

A non-naturally occurring truncated human without a His tag, linker, and thrombin cleavage site is disclosed below. The codon-optimized nucleotide sequence encoding this elastin and the amino acid sequence are disclosed below. In SEQ ID NO: 49, the DsbA secretion tag is encoded by nucleotides 1-57 and encodes amino acids 1-19 of SEQ ID NO: 50. In SEQ ID NO: 49, the truncated elastin sequence is encoded by nucleotides 58-489 and encodes amino acids 20-163 of SEQ ID NO: 50.

The codon-optimized nucleotide sequence encoding this elastin is provided in SEQ ID NO: 49.

(SEQ ID NO: 49)
ATGAAAAAGATTTGGCTGGCGCTGGCTGGTTTAGTTTTAGCGTTTAGCG
CATCGGCGGCCGGTCTGGGTGGCGTGCCTGGCGTTGGTGGCCTGGGCGT
TAGCGCCGGTGCAGTTGTTCCGCAGCCTGGTGCGGGTGTTAAACCGGGT
AAAGTTCCTGGTGTTGGTCTGCCTGGTGTTTATCCAGGCGGTGTTCTGC
CAGGTGCGCGTTTTCCTGGCGTGGGTGTTCTGCCGGGTGTTCCGACCGG
TGCAGGCGTGAAACCGAAAGCACCAGGTGTTGGTGGTGCATTTGCAGGT
ATTCCAGGTGTGGGTCCGTTTGGTGGTCCGCAGCCAGGCGTTCCGCTGG
GTTATCCGATTAAAGCACCGAAACTGCCTGGCGGTTATGGTCTGCCGTA
TACCACCGGTAAACTGCCGTATGGTTATGGCCCTGGCGGTGTTGCCGGT
GCGGCAGGTAAAGCAGGCTATCCGACCGGTACCGGTGTAGGTCCGCAGT
AA

The amino acid sequence of the 13 kDa non-naturally occurring truncated human elastin sequence including the DsbA secretion signal is disclosed in SEQ ID NO: 50.

(SEQ ID NO: 50)
MKKIWLALAGLVLAFSASAAGLGGVPGVGGLGVSAGAVVPQPGAGVKPG
KVPGVGLPGVYPGGVLPGARFPGVGVLPGVPTGAGVKPKAPGVGGAFAG
IPGVGPFGGPQPGVPLGYPIKAPKLPGGYGLPYTTGKLPYGYGPGGVAG
AAGKAGYPTGTGVGPQ

The codon-optimized nucleotide sequence encoding the non-naturally occurring truncated human elastin without the DsbA secretion tag is provided in SEQ ID NO: 51.

(SEQ ID NO: 51)
GCCGGTCTGGGTGGCGTGCCTGGCGTTGGTGGCCTGGGCGTTAGCGCCG
GTGCAGTTGTTCCGCAGCCTGGTGCGGGTGTTAAACCGGGTAAAGTTCC
TGGTGTTGGTCTGCCTGGTGTTTATCCAGGCGGTGTTCTGCCAGGTGCG
CGTTTTCCTGGCGTGGGTGTTCTGCCGGGTGTTCCGACCGGTGCAGGCG
TGAAACCGAAAGCACCAGGTGTTGGTGGTGCATTTGCAGGTATTCCAGG
TGTGGGTCCGTTTGGTGGTCCGCAGCCAGGCGTTCCGCTGGGTTATCCG
ATTAAAGCACCGAAACTGCCTGGCGGTTATGGTCTGCCGTATACCACCG
GTAAACTGCCGTATGGTTATGGCCCTGGCGGTGTTGCCGGTGCGGCAGG
TAAAGCAGGCTATCCGACCGGTACCGGTGTAGGTCCGCAGTAA

The amino acid sequence of the non-naturally occurring truncated human elastin without the DsbA secretion tag is disclosed in SEQ ID NO: 52.

(SEQ ID NO: 52)
AGLGGVPGVGGLGVSAGAVVPQPGAGVKPGKVPGVGLPGVYPGGVLPGA
RFPGVGVLPGVPTGAGVKPKAPGVGGAFAGIPGVGPFGGPQPGVPLGYP
IKAPKLPGGYGLPYTTGKLPYGYGPGGVAGAAGKAGYPTGTGVGPQ

The polynucleotide of SEQ ID NO: 49 was synthesized by Twist DNA. Overlaps between the pET28 vector and SEQ ID NO: 49 were designed to be between 20 and 30 bp long and added using PCR with the enzyme PrimeSTAR® GXL polymerase (www.takarabio.com/products/pcr/gc-rich-pcr/primestar-gxl-dna-polymerase). The opened pET28a vector and insert DNA (SEQ ID NO: 49) were then assembled together into the final plasmid using In-Fusion Cloning (www.takarabio.com/products/cloning/in-fusion-cloning). The sequence of the plasmid was then verified through Sanger sequencing through Genewiz (www.genewiz.com/en).

The transformed cells were cultivated in minimal media and frozen in 1.5 mL aliquots with vegetable glycerin at a ratio of 50:50 of cells to glycerin. One vial of this frozen culture was revived in 50 mL of minimal media overnight at 37° C., 200 rpm. Cells were transferred into 300 mL of minimal media and grown for 6-9 hours to reach an OD600 of 5-10.

The 13 kDa non-naturally occurring truncated human elastin was detected on 12% BisTris protein gel. A photograph of the gel is depicted in FIG. 22. The 13 kDa non-naturally occurring truncated human elastin was confirmed to be the correct mass by intact mass spectrometry using liquid chromatography—mass spectrometry

### Example 23. Purity and Uniformity of a 13 kDa Non-Naturally Occurring Truncated Elastin

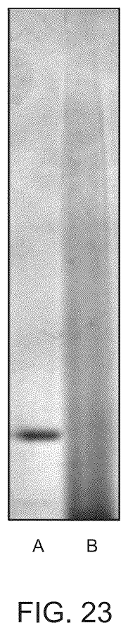

A polypeptide with an amino acid sequence according to SEQ ID NO: 52 was analyzed by SDS-PAGE to assess purity and uniformity. FIG. 23 demonstrates that the polypeptide with an amino acid sequence according to SEQ ID NO: 52 appeared as a single band on an SDS-PAGE protein gel, indicating high purity and homogeneity. In contrast, a commercially-available elastin appeared as a smear on the protein gel indicating high heterogeneity.

### Example 24. In Vitro Study of a Non-Naturally Occurring Truncated Human Elastin

Non-Naturally Occurring Truncated Human Elastin Stimulates Fibroblast Production of Collagen Type I protein

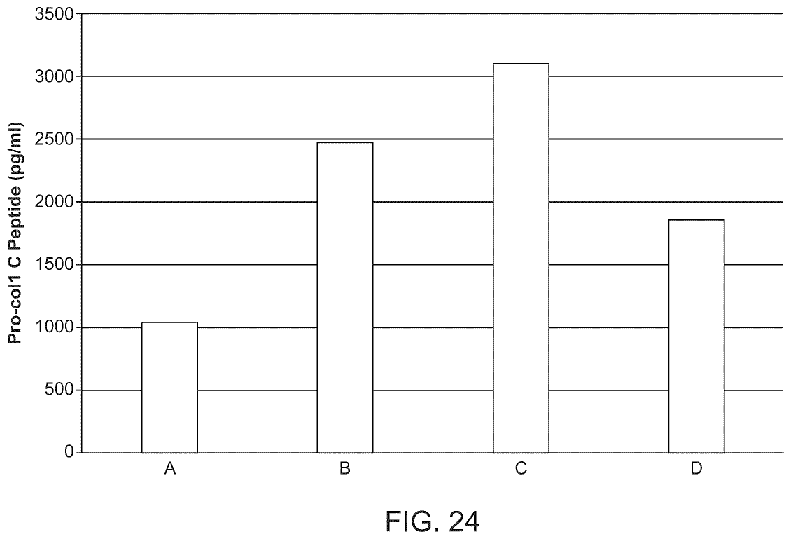

Fibroblasts are the main source of the extracellular matrix peptides, including the structural proteins collagen and elastin. Human primary fibroblasts were evaluated for collagen type I protein secretion. Fibroblasts were cultured with a polypeptide according to SEQ ID NO: 52 for 48 hours. The culture supernatants were analyzed by ELISA for pro-collagen type I C-peptide, a readout for total secreted collagen type I protein. FIG. 24 demonstrates that fibroblasts treated with a 0.1% w/w solution (FIG. 24, “B”), a 0.05% w/w solution (FIG. 24, “C”), and a 0.025% w/w solution (FIG. 24, “D”) of a polypeptide according to SEQ ID NO: 52 secreted higher levels of collagen type I than untreated control fibroblasts (FIG. 24, “A”).

### Non-Naturally Occurring Truncated Human Elastin Demonstrates Anti-Oxidative Capacity

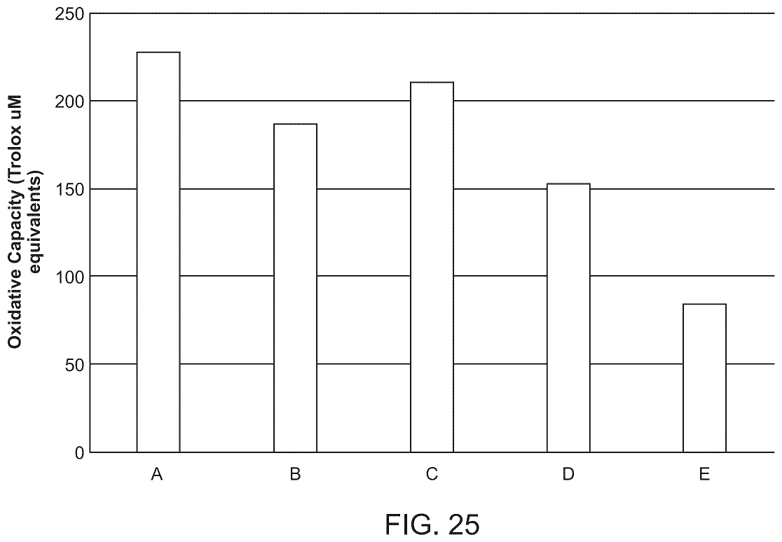

An oxygen radical absorbance capacity (ORAC) assay was performed to analyze the anti-oxidative capacity of a polypeptide according to SEQ ID NO: 52. The ORAC assay is a cell-free assay that uses a fluorescent readout to measure anti-oxidative capacity. Data is reported in Trolox (Vitamin E) equivalents (TEs). As shown in FIG. 25, a 0.0125% w/w solution (FIG. 25, “E”), a 0.025% w/w solution (FIG. 25, “D”), a 0.05% w/w solution (FIG. 25, “C”), a 0.1% w/w solution (FIG. 25, “B”), and a 0.2% w/w solution (FIG. 25, “A”) of a polypeptide according to SEQ ID NO: 52 each demonstrated anti-oxidative properties. For example, a 0.2% w/w solution of a polypeptide according to SEQ ID NO: 52 demonstrated anti-oxidative properties equivalent to 228 μM Trolox.

### Non-Naturally Occurring Truncated Human Elastin Stimulates Antioxidant Genes in Keratinocytes

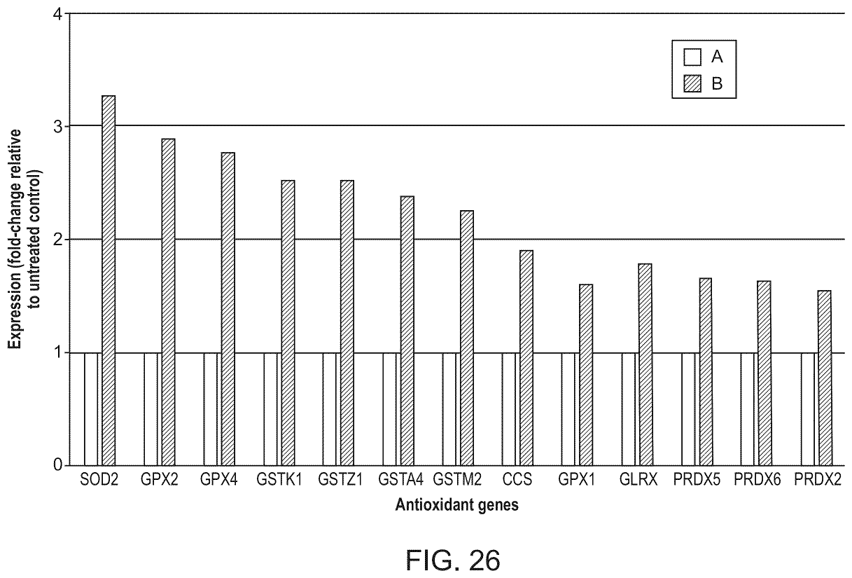

Keratinocytes were incubated for 24 hours with media alone or with 0.03% w/w solution of a polypeptide according to SEQ ID NO: 52. RNA was extracted from the cells and analyzed by Clariom microarray (ThermoFisher) for global gene expression. FIG. 26 demonstrates that many antioxidant genes, including SOD2, GPX2, GPX4, GSTK1, GSTZ1, GSTA4, GSTM2, CCS, GPX1, GLRX, PRDXS, PRDX6, and PRDX2, were upregulated in keratinocytes treated with a polypeptide according to SEQ ID NO: 52 (FIG. 26, “B”), relative to untreated cells (FIG. 26, “A”).

### Non-Naturally Occurring Truncated Human Elastin Reduces Expression of Apoptosis Genes in Keratinocytes

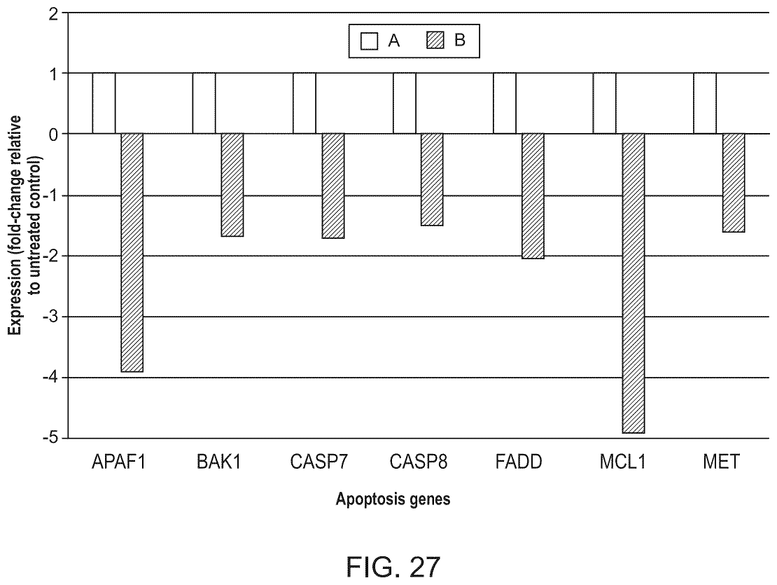

Keratinocytes were incubated for 24 hours with media alone (untreated) or with 0.03% w/w solution of a polypeptide according to SEQ ID NO: 52. RNA was extracted from the cells and analyzed by Clariom microarray (ThermoFisher) for global gene expression. FIG. 27 demonstrates that many pro-apoptotic genes, including APAF1, BAK1, CASP7, CASP8, FADD, MCL1, and MET, were downregulated in keratinocytes treated with a polypeptide according to SEQ ID NO: 52 (FIG. 27, “B”), relative to untreated cells (FIG. 27, “A”).

### Non-Naturally Occurring Truncated Human Elastin Stimulates Fibroblast Production of Elastin

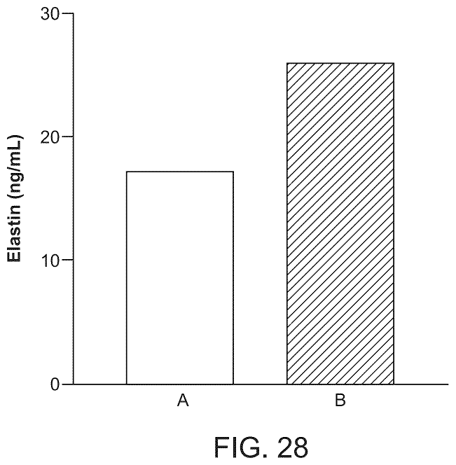

Human primary fibroblast cultures were treated for 24 hours with media alone (untreated) or with 0.03% w/w solution of a polypeptide having an amino acid sequence according to SEQ ID NO: 52. Supernatants were collected and evaluated for elastin production using an ELISA kit. FIG. 28 demonstrates that fibroblasts treated with a polypeptide having an amino acid sequence according to SEQ ID NO: 52 (FIG. 28, “B”) exhibited increased production of elastin, as compared to fibroblasts treated with media alone (FIG. 28, “A”).

### Example 25. In Vitro Testing Outcomes of Combinations of Elastin Polypeptides and Hyaluronic Acid, Retinol, a Collagen Polypeptide or a Truncate Thereof, Ascorbic Acid, Salicylic Acid, Benzoyl Peroxide, Niacinamide, or Alpha Hydroxy Acids (e.g., Glycolic Acid, Lactic Acid)

Combinations of Elastin Polypeptides with Ascorbic Acid, Retinol, a Collagen Polypeptide or a Truncate Thereof, Hyaluronic Acid, Salicylic Acid, Benzoyl Peroxide, Niacinamide, or Alpha Hydroxy Acids (e.g., Glycolic Acid, Lactic Acid) Increase Keratinocyte and Fibroblast Cell Viability and Proliferation

In an in vitro study, (e.g., human) skin cell cultures, (e.g., human) skin equivalent cultures, or ex vivo (e.g., human) skin is treated with an elastin polypeptide as provided herein (e.g., an (e.g., human) elastin polypeptide or a truncate thereof (e.g., having an amino acid sequence of SEQ ID NO: 52)) alone, or in combination with ascorbic acid, retinol, hyaluronic acid, a collagen polypeptide or a truncate thereof, salicylic acid, benzoyl peroxide, niacinamide, or alpha hydroxy acids (e.g., glycolic acid, lactic acid). Treatment with the (e.g., human) elastin polypeptide or truncate thereof (e.g., having an amino acid sequence of SEQ ID NO: 52) in combination with ascorbic acid, retinol, hyaluronic acid, a collagen polypeptide or a truncate thereof, salicylic acid, benzoyl peroxide, niacinamide, or alpha hydroxy acids (e.g., glycolic acid, lactic acid) increases (e.g., synergistically, additively) viability and proliferation of keratinocytes and fibroblasts (e.g., as measured by, e.g., MTT viability assay), relative to treatment with the elastin polypeptide or truncate thereof, ascorbic acid, retinol, hyaluronic acid, a collagen polypeptide or a truncate thereof, salicylic acid, benzoyl peroxide, niacinamide, or alpha hydroxy acids (e.g., glycolic acid, lactic acid) alone.

Combinations of Elastin Polypeptides with Ascorbic Acid, Retinol, Hyaluronic Acid, a Collagen Polypeptide or a Truncate Thereof, Salicylic Acid, Benzoyl Peroxide, Niacinamide, or Alpha Hydroxy Acids (e.g., Glycolic Acid, Lactic Acid) Increase Production of Extracellular Matrix Components and Decrease Production of Matrix-Degrading Proteins

In an in vitro study, (e.g., human) skin cell cultures, (e.g., human) skin equivalent cultures, or ex vivo (e.g., human) skin is treated with an elastin polypeptide as provided herein (e.g., an (e.g., human) elastin polypeptide or a truncate thereof (e.g., having an amino acid sequence of SEQ ID NO: 52)) alone, or in combination with ascorbic acid, retinol, hyaluronic acid, a collagen polypeptide or a truncate thereof, salicylic acid, benzoyl peroxide, niacinamide, or alpha hydroxy acids (e.g., glycolic acid, lactic acid). Treatment with the (e.g., human) elastin polypeptide or truncate thereof (e.g., having an amino acid sequence of SEQ ID NO: 52) in combination with ascorbic acid, retinol, hyaluronic acid, a collagen polypeptide or a truncate thereof, salicylic acid, benzoyl peroxide, niacinamide, or alpha hydroxy acids (e.g., glycolic acid, lactic acid) increases (e.g., synergistically, additively) the production of extracellular matrix components (e.g., collagen, elastin, fibronectin, fibrillin, hyaluronic acid) (e.g., as measured by, e.g., transcriptomic analysis, ELISA) relative to treatment with the (e.g., human) elastin polypeptide or truncate thereof, ascorbic acid, retinol, hyaluronic acid, a collagen polypeptide or a truncate thereof, salicylic acid, benzoyl peroxide, niacinamide, or alpha hydroxy acids (e.g., glycolic acid, lactic acid) alone. Treatment with the (e.g., human) elastin polypeptide or truncate thereof (e.g., having an amino acid sequence of SEQ ID NO: 52) in combination with ascorbic acid, retinol, hyaluronic acid, a collagen polypeptide or a truncate thereof, salicylic acid, benzoyl peroxide, niacinamide, or alpha hydroxy acids (e.g., glycolic acid, lactic acid) decreases (e.g., synergistically, additively) the production of matrix-degrading proteins (e.g., matrix metalloproteases, proteases) (e.g., as measured by, e.g., transcriptomic analysis, ELISA) relative to treatment with the (e.g., human) elastin polypeptide or truncate thereof, ascorbic acid, retinol, hyaluronic acid, a collagen polypeptide or a truncate thereof, salicylic acid, benzoyl peroxide, niacinamide, or alpha hydroxy acids (e.g., glycolic acid, lactic acid) alone.

Combinations of Elastin Polypeptides with Ascorbic Acid, Retinol, Hyaluronic Acid, a Collagen Polypeptide or a Truncate Thereof, Salicylic Acid, Benzoyl Peroxide, Niacinamide, or Alpha Hydroxy Acids (e.g., Glycolic Acid, Lactic Acid) Increase Wound Healing Capacity

In an in vitro study, (e.g., human) skin cell cultures, (e.g., human) skin equivalent cultures, or ex vivo (e.g., human) skin is treated with an elastin polypeptide as provided herein (e.g., an (e.g., human) elastin polypeptide or a truncate thereof (e.g., having an amino acid sequence of SEQ ID NO: 52)) alone, or in combination with ascorbic acid, retinol, hyaluronic acid, a collagen polypeptide or a truncate thereof, salicylic acid, benzoyl peroxide, niacinamide, or alpha hydroxy acids (e.g., glycolic acid, lactic acid). Treatment with the (e.g., human) elastin polypeptide or truncate thereof (e.g., having an amino acid sequence of SEQ ID NO: 52) in combination with ascorbic acid, retinol, hyaluronic acid, a collagen polypeptide or a truncate thereof, salicylic acid, benzoyl peroxide, niacinamide, or alpha hydroxy acids (e.g., glycolic acid, lactic acid) increases (e.g., synergistically, additively) wound healing capacity of skin cells (e.g., keratinocytes, fibroblasts) (e.g., as measured by, e.g., wound healing scratch assay) relative to treatment with the (e.g., human) elastin polypeptide or truncate thereof, ascorbic acid, retinol, hyaluronic acid, a collagen polypeptide or a truncate thereof, salicylic acid, benzoyl peroxide, niacinamide, or alpha hydroxy acids (e.g., glycolic acid, lactic acid) alone.

Combinations of Elastin Polypeptides with Ascorbic Acid, Retinol, Hyaluronic Acid, a Collagen Polypeptide or a Truncate Thereof, Salicylic Acid, Benzoyl Peroxide, Niacinamide, or Alpha Hydroxy Acids (e.g., Glycolic Acid, Lactic Acid) have Improved Outcomes after Challenge with Photodamage

In an in vitro study, (e.g., human) skin cell cultures, (e.g., human) skin equivalent cultures, or ex vivo (e.g., human) skin is challenged with photodamage (e.g., UV irradiation, blue light exposure), and then treated with an elastin polypeptide as provided herein (e.g., an (e.g., human) elastin polypeptide or a truncate thereof (e.g., having an amino acid sequence of SEQ ID NO: 52)) alone, or in combination with ascorbic acid, retinol, hyaluronic acid, a collagen polypeptide or a truncate thereof, salicylic acid, benzoyl peroxide, niacinamide, or alpha hydroxy acids (e.g., glycolic acid, lactic acid). Treatment with the (e.g., human) elastin polypeptide or truncate thereof (e.g., having an amino acid sequence of SEQ ID NO: 52) in combination with ascorbic acid, retinol, hyaluronic acid, a collagen polypeptide or a truncate thereof, salicylic acid, benzoyl peroxide, niacinamide, or alpha hydroxy acids (e.g., glycolic acid, lactic acid) increases (e.g., synergistically, additively) viability and proliferation of skin cells (e.g., keratinocytes, fibroblasts) (e.g., as measured by, e.g., MTT viability assay) relative to treatment with the (e.g., human) elastin polypeptide or truncate thereof, ascorbic acid, retinol, hyaluronic acid, a collagen polypeptide or a truncate thereof, salicylic acid, benzoyl peroxide, niacinamide, or alpha hydroxy acids (e.g., glycolic acid, lactic acid) alone. Treatment with the (e.g., human) elastin polypeptide or truncate thereof (e.g., having an amino acid sequence of SEQ ID NO: 52) in combination with ascorbic acid, retinol, hyaluronic acid, a collagen polypeptide or a truncate thereof, salicylic acid, benzoyl peroxide, niacinamide, or alpha hydroxy acids (e.g., glycolic acid, lactic acid) reduces (e.g., synergistically, additively) cytotoxicity and apoptosis of skin cells (e.g., keratinocytes, fibroblasts) (e.g., as measured by, e.g., fluorescence microscopy) relative to treatment with the (e.g., human) elastin polypeptide or truncate thereof, ascorbic acid, retinol, hyaluronic acid, a collagen polypeptide or a truncate thereof, salicylic acid, benzoyl peroxide, niacinamide, or alpha hydroxy acids (e.g., glycolic acid, lactic acid) alone. Treatment with the (e.g., human) elastin polypeptide or truncate thereof (e.g., having an amino acid sequence of SEQ ID NO: 52) in combination with ascorbic acid, retinol, hyaluronic acid, a collagen polypeptide or a truncate thereof, salicylic acid, benzoyl peroxide, niacinamide, or alpha hydroxy acids (e.g., glycolic acid, lactic acid) increases (e.g., synergistically, additively) DNA repair of skin cells (e.g., keratinocytes, fibroblasts) (e.g., as measured by, e.g., thymidine dimer ELISA detection) relative to treatment with the (e.g., human) elastin polypeptide or truncate thereof, ascorbic acid, retinol, hyaluronic acid, a collagen polypeptide or a truncate thereof, salicylic acid, benzoyl peroxide, niacinamide, or alpha hydroxy acids (e.g., glycolic acid, lactic acid) alone. Treatment with the (e.g., human) elastin polypeptide or truncate thereof (e.g., having an amino acid sequence of SEQ ID NO: 52) in combination with ascorbic acid, retinol, hyaluronic acid, a collagen polypeptide or a truncate thereof, salicylic acid, benzoyl peroxide, niacinamide, or alpha hydroxy acids (e.g., glycolic acid, lactic acid) reduces (e.g., synergistically, additively) inflammation (e.g., as measured by, e.g., ELISA analysis or Luminex detection of cytokines) relative to treatment with the (e.g., human) elastin polypeptide or truncate thereof, ascorbic acid, retinol, hyaluronic acid, a collagen polypeptide or a truncate thereof, salicylic acid, benzoyl peroxide, niacinamide, or alpha hydroxy acids (e.g., glycolic acid, lactic acid) alone. Treatment with the (e.g., human) elastin polypeptide or truncate thereof (e.g., having an amino acid sequence of SEQ ID NO: 52) in combination with ascorbic acid, retinol, hyaluronic acid, a collagen polypeptide or a truncate thereof, salicylic acid, benzoyl peroxide, niacinamide, or alpha hydroxy acids (e.g., glycolic acid, lactic acid) reduces (e.g., synergistically, additively) reactive oxidative stress (e.g., as measured by, e.g., CM-H2DCFDA detection) relative to treatment with the (e.g., human) elastin polypeptide or truncate thereof, ascorbic acid, retinol, hyaluronic acid, a collagen polypeptide or a truncate thereof, salicylic acid, benzoyl peroxide, niacinamide, or alpha hydroxy acids (e.g., glycolic acid, lactic acid) alone.

Combinations of Elastin Polypeptides with Ascorbic Acid, Retinol, Hyaluronic Acid, a Collagen Polypeptide or a Truncate Thereof, Salicylic Acid, Benzoyl Peroxide, Niacinamide, or Alpha Hydroxy Acids (e.g., Glycolic Acid, Lactic Acid) have Improved Outcomes after Challenge with Pollution

In an in vitro study, (e.g., human) skin cell cultures, (e.g., human) skin equivalent cultures, or ex vivo (e.g., human) skin is challenged with pollution (e.g., urban dust exposure), and then treated with an elastin polypeptide as provided herein (e.g., an (e.g., human) elastin polypeptide or a truncate thereof (e.g., having an amino acid sequence of SEQ ID NO: 52)) alone, or in combination with ascorbic acid, retinol, hyaluronic acid, a collagen polypeptide or a truncate thereof, salicylic acid, benzoyl peroxide, niacinamide, or alpha hydroxy acids (e.g., glycolic acid, lactic acid). Treatment with the (e.g., human) elastin polypeptide or truncate thereof (e.g., having an amino acid sequence of SEQ ID NO: 52) in combination with ascorbic acid, retinol, hyaluronic acid, a collagen polypeptide or a truncate thereof, salicylic acid, benzoyl peroxide, niacinamide, or alpha hydroxy acids (e.g., glycolic acid, lactic acid) increases (e.g., synergistically, additively) viability and proliferation of skin cells (e.g., keratinocytes, fibroblasts) (e.g., as measured by, e.g., MTT viability assay) relative to treatment with the (e.g., human) elastin polypeptide or truncate thereof, ascorbic acid, retinol, hyaluronic acid, a collagen polypeptide or a truncate thereof, salicylic acid, benzoyl peroxide, niacinamide, or alpha hydroxy acids (e.g., glycolic acid, lactic acid) alone. Treatment with the (e.g., human) elastin polypeptide or truncate thereof (e.g., having an amino acid sequence of SEQ ID NO: 52) in combination with ascorbic acid, retinol, hyaluronic acid, a collagen polypeptide or a truncate thereof, salicylic acid, benzoyl peroxide, niacinamide, or alpha hydroxy acids (e.g., glycolic acid, lactic acid) reduces (e.g., synergistically, additively) cytotoxicity and apoptosis of skin cells (e.g., keratinocytes, fibroblasts) (e.g., as measured by, e.g., fluorescence microscopy) relative to treatment with the (e.g., human) elastin polypeptide or truncate thereof, ascorbic acid, retinol, hyaluronic acid, a collagen polypeptide or a truncate thereof, salicylic acid, benzoyl peroxide, niacinamide, or alpha hydroxy acids (e.g., glycolic acid, lactic acid) alone. Treatment with the (e.g., human) elastin polypeptide or truncate thereof (e.g., having an amino acid sequence of SEQ ID NO: 52) in combination with ascorbic acid, retinol, hyaluronic acid, a collagen polypeptide or a truncate thereof, salicylic acid, benzoyl peroxide, niacinamide, or alpha hydroxy acids (e.g., glycolic acid, lactic acid) increases (e.g., synergistically, additively) DNA repair of skin cells (e.g., keratinocytes, fibroblasts) (e.g., as measured by, e.g., thymidine dimer ELISA detection) relative to treatment with the (e.g., human) elastin polypeptide or truncate thereof, ascorbic acid, retinol, hyaluronic acid, a collagen polypeptide or a truncate thereof, salicylic acid, benzoyl peroxide, niacinamide, or alpha hydroxy acids (e.g., glycolic acid, lactic acid) alone. Treatment with the (e.g., human) elastin polypeptide or truncate thereof (e.g., having an amino acid sequence of SEQ ID NO: 52) in combination with ascorbic acid, retinol, hyaluronic acid, a collagen polypeptide or a truncate thereof, salicylic acid, benzoyl peroxide, niacinamide, or alpha hydroxy acids (e.g., glycolic acid, lactic acid) reduces (e.g., synergistically, additively) inflammation (e.g., as measured by, e.g., ELISA analysis or Luminex detection of cytokines) relative to treatment with the (e.g., human) elastin polypeptide or truncate thereof, ascorbic acid, retinol, hyaluronic acid, a collagen polypeptide or a truncate thereof, salicylic acid, benzoyl peroxide, niacinamide, or alpha hydroxy acids (e.g., glycolic acid, lactic acid) alone. Treatment with the (e.g., human) elastin polypeptide or truncate thereof (e.g., having an amino acid sequence of SEQ ID NO: 52) in combination with ascorbic acid, retinol, hyaluronic acid, a collagen polypeptide or a truncate thereof, salicylic acid, benzoyl peroxide, niacinamide, or alpha hydroxy acids (e.g., glycolic acid, lactic acid) reduces (e.g., synergistically, additively) reactive oxidative stress (e.g., as measured by, e.g., CM-H2DCFDA detection) relative to treatment with the (e.g., human) elastin polypeptide or truncate thereof, ascorbic acid, retinol, hyaluronic acid, a collagen polypeptide or a truncate thereof, salicylic acid, benzoyl peroxide, niacinamide, or alpha hydroxy acids (e.g., glycolic acid, lactic acid) alone.

Combinations of Elastin Polypeptides with Ascorbic Acid, Retinol, Hyaluronic Acid, a Collagen Polypeptide or a Truncate Thereof, Salicylic Acid, Benzoyl Peroxide, Niacinamide, or Alpha Hydroxy Acids (e.g., Glycolic Acid, Lactic Acid) have Improved Outcomes after Challenge with Harsh Skin Actives

In an in vitro study, (e.g., human) skin cell cultures, (e.g., human) skin equivalent cultures, or ex vivo (e.g., human) skin is challenged with harsh skin actives (e.g., retinoic acid or retinol, benzoyl peroxide, salicylic acid), and then treated with an elastin polypeptide as provided herein (e.g., an (e.g., human) elastin polypeptide or a truncate thereof (e.g., having an amino acid sequence of SEQ ID NO: 52)) alone, or in combination with ascorbic acid, retinol, hyaluronic acid, a collagen polypeptide or a truncate thereof, salicylic acid, benzoyl peroxide, niacinamide, or alpha hydroxy acids (e.g., glycolic acid, lactic acid). Treatment with the (e.g., human) elastin polypeptide or truncate thereof (e.g., having an amino acid sequence of SEQ ID NO: 52) in combination with ascorbic acid, retinol, hyaluronic acid, a collagen polypeptide or a truncate thereof, salicylic acid, benzoyl peroxide, niacinamide, or alpha hydroxy acids (e.g., glycolic acid, lactic acid) increases (e.g., synergistically, additively) viability and proliferation of skin cells (e.g., keratinocytes, fibroblasts) (e.g., as measured by, e.g., MTT viability assay) relative to treatment with the (e.g., human) elastin polypeptide or truncate thereof, ascorbic acid, retinol, hyaluronic acid, a collagen polypeptide or a truncate thereof, salicylic acid, benzoyl peroxide, niacinamide, or alpha hydroxy acids (e.g., glycolic acid, lactic acid) alone. Treatment with the (e.g., human) elastin polypeptide or truncate thereof (e.g., having an amino acid sequence of SEQ ID NO: 52) in combination with ascorbic acid, retinol, hyaluronic acid, a collagen polypeptide or a truncate thereof, salicylic acid, benzoyl peroxide, niacinamide, or alpha hydroxy acids (e.g., glycolic acid, lactic acid) reduces (e.g., synergistically, additively) cytotoxicity and apoptosis of skin cells (e.g., keratinocytes, fibroblasts) (e.g., as measured by, e.g., fluorescence microscopy) relative to treatment with the (e.g., human) elastin polypeptide or truncate thereof, ascorbic acid, retinol, hyaluronic acid, a collagen polypeptide or a truncate thereof, salicylic acid, benzoyl peroxide, niacinamide, or alpha hydroxy acids (e.g., glycolic acid, lactic acid) alone. Treatment with the (e.g., human) elastin polypeptide or truncate thereof (e.g., having an amino acid sequence of SEQ ID NO: 52) in combination with ascorbic acid, retinol, hyaluronic acid, a collagen polypeptide or a truncate thereof, salicylic acid, benzoyl peroxide, niacinamide, or alpha hydroxy acids (e.g., glycolic acid, lactic acid) increases (e.g., synergistically, additively) DNA repair of skin cells (e.g., keratinocytes, fibroblasts) (e.g., as measured by, e.g., thymidine dimer ELISA detection) relative to treatment with the (e.g., human) elastin polypeptide or truncate thereof, ascorbic acid, retinol, hyaluronic acid, a collagen polypeptide or a truncate thereof, salicylic acid, benzoyl peroxide, niacinamide, or alpha hydroxy acids (e.g., glycolic acid, lactic acid) alone. Treatment with the (e.g., human) elastin polypeptide or truncate thereof (e.g., having an amino acid sequence of SEQ ID NO: 52) in combination with ascorbic acid, retinol, hyaluronic acid, a collagen polypeptide or a truncate thereof, salicylic acid, benzoyl peroxide, niacinamide, or alpha hydroxy acids (e.g., glycolic acid, lactic acid) reduces (e.g., synergistically, additively) inflammation (e.g., as measured by, e.g., ELISA analysis or Luminex detection of cytokines) relative to treatment with the (e.g., human) elastin polypeptide or truncate thereof, ascorbic acid, retinol, hyaluronic acid, a collagen polypeptide or a truncate thereof, salicylic acid, benzoyl peroxide, niacinamide, or alpha hydroxy acids (e.g., glycolic acid, lactic acid) alone. Treatment with the (e.g., human) elastin polypeptide or truncate thereof (e.g., having an amino acid sequence of SEQ ID NO: 52) in combination with ascorbic acid, retinol, hyaluronic acid, a collagen polypeptide or a truncate thereof, salicylic acid, benzoyl peroxide, niacinamide, or alpha hydroxy acids (e.g., glycolic acid, lactic acid) reduces (e.g., synergistically, additively) reactive oxidative stress (e.g., as measured by, e.g., CM-H2DCFDA detection) relative to treatment with the (e.g., human) elastin polypeptide or truncate thereof, ascorbic acid, retinol, hyaluronic acid, a collagen polypeptide or a truncate thereof, salicylic acid, benzoyl peroxide, niacinamide, or alpha hydroxy acids (e.g., glycolic acid, lactic acid) alone.

### Example 26. Clinical Testing Outcomes of Combinations of Elastin Polypeptides and Hyaluronic Acid, Retinol, Ascorbic Acid, a Collagen Polypeptide or a Truncate Thereof, Salicylic Acid, Benzoyl Peroxide, Niacinamide, or Alpha Hydroxy Acids (e.g., Glycolic Acid, Lactic Acid)

Topical Application of Combinations of Elastin Polypeptides with Ascorbic Acid, Retinol, Hyaluronic Acid, a Collagen Polypeptide or a Truncate Thereof, Salicylic Acid, Benzoyl Peroxide, Niacinamide, or Alpha Hydroxy Acids (e.g., Glycolic Acid, Lactic Acid) Improve Skin Appearance and Quality

In a clinical study, subjects use a topical formulation of an elastin polypeptide as provided herein (e.g., an (e.g., human) elastin polypeptide or a truncate thereof (e.g., having an amino acid sequence of SEQ ID NO: 52)) alone, or in combination with ascorbic acid, retinol, hyaluronic acid, a collagen polypeptide or a truncate thereof, salicylic acid, benzoyl peroxide, niacinamide, or alpha hydroxy acids (e.g., glycolic acid, lactic acid). Subjects treated with the (e.g., human) elastin polypeptide or truncate thereof (e.g., having an amino acid sequence of SEQ ID NO: 52) in combination with ascorbic acid, retinol, hyaluronic acid, a collagen polypeptide or a truncate thereof, salicylic acid, benzoyl peroxide, niacinamide, or alpha hydroxy acids (e.g., glycolic acid, lactic acid) exhibit a reduction in wrinkles and fine lines, a reduction in skin redness and pigmentation/hyperpigmentation, an increase in skin brightness, a decrease in pore size, a decrease in skin roughness, and/or a reduction in acne (e.g., as measured using, e.g., CLARITY analysis) relative to treatment with the (e.g., human) elastin polypeptide or truncate thereof, ascorbic acid, retinol, hyaluronic acid, a collagen polypeptide or a truncate thereof, salicylic acid, benzoyl peroxide, niacinamide, or alpha hydroxy acids (e.g., glycolic acid, lactic acid) alone.

Subjects treated with the (e.g., human) elastin polypeptide or truncate thereof (e.g., having an amino acid sequence of SEQ ID NO: 52) in combination with ascorbic acid, retinol, hyaluronic acid, a collagen polypeptide or a truncate thereof, salicylic acid, benzoyl peroxide, niacinamide, or alpha hydroxy acids (e.g., glycolic acid, lactic acid) exhibit an increase in skin elasticity, an increase in skin firmness, an increase in skin hydration, an increase in skin barrier function, an increase in skin collagen and elastin content, and/or an increase in dermal density relative to treatment with the (e.g., human) elastin polypeptide or truncate thereof, ascorbic acid, retinol, hyaluronic acid, a collagen polypeptide or a truncate thereof, salicylic acid, benzoyl peroxide, niacinamide, or alpha hydroxy acids (e.g., glycolic acid, lactic acid) alone.

Subjects treated with the (e.g., human) elastin polypeptide or truncate thereof (e.g., having an amino acid sequence of SEQ ID NO: 52) in combination with ascorbic acid, retinol, hyaluronic acid, a collagen polypeptide or a truncate thereof, salicylic acid, benzoyl peroxide, niacinamide, or alpha hydroxy acids (e.g., glycolic acid, lactic acid) exhibit an improvement of skin microflora (e.g., reduced abundance of harmful microbes, promotion of beneficial microbes, and/or increased microbial diversity) relative to treatment with the (e.g., human) elastin polypeptide or truncate thereof, ascorbic acid, retinol, hyaluronic acid, a collagen polypeptide or a truncate thereof, salicylic acid, benzoyl peroxide, niacinamide, or alpha hydroxy acids (e.g., glycolic acid, lactic acid) alone.

### Example 27. Combinations of Elastin Polypeptides and Hyaluronic Acid, Retinol, Ascorbic Acid, a Collagen Polypeptide or a Truncate Thereof, Salicylic Acid, Benzoyl Peroxide, Niacinamide, or Alpha Hydroxy Acids (e.g., Glycolic Acid, Lactic Acid) Improve Hair Quality and Appearance

In a clinical study, subjects use a composition of an elastin polypeptide as provided herein (e.g., an (e.g., human) elastin polypeptide or a truncate thereof (e.g., having an amino acid sequence of SEQ ID NO: 52)) alone, or in combination with ascorbic acid, retinol, hyaluronic acid, a collagen polypeptide or a truncate thereof, salicylic acid, benzoyl peroxide, niacinamide, or alpha hydroxy acids (e.g., glycolic acid, lactic acid), formulated for application to the hair. Subjects treated with the (e.g., human) elastin polypeptide or truncate thereof (e.g., having an amino acid sequence of SEQ ID NO: 52) in combination with ascorbic acid, retinol, hyaluronic acid, a collagen polypeptide or a truncate thereof, salicylic acid, benzoyl peroxide, niacinamide, or alpha hydroxy acids (e.g., glycolic acid, lactic acid) exhibit improved hair growth, thicker hair fiber diameter, increased combability, reduced hair loss, faster growth rate, and/or increased hair tensile strength relative to treatment with the (e.g., human) elastin polypeptide or truncate thereof, ascorbic acid, retinol, hyaluronic acid, a collagen polypeptide or a truncate thereof, salicylic acid, benzoyl peroxide, niacinamide, or alpha hydroxy acids (e.g., glycolic acid, lactic acid) alone.

Subjects treated with the (e.g., human) elastin polypeptide or truncate thereof (e.g., having an amino acid sequence of SEQ ID NO: 52) in combination with ascorbic acid, retinol, hyaluronic acid, a collagen polypeptide or a truncate thereof, salicylic acid, benzoyl peroxide, niacinamide, or alpha hydroxy acids (e.g., glycolic acid, lactic acid) exhibit improved hair fiber thickness and density, increased moisture, increased hydrophobicity, reduced split ends, reduced frizz/increased static control, improved fiber alignment/shine, increased wet/dry combability, and/or stronger resistance to hair breakage relative to treatment with the (e.g., human) elastin polypeptide or truncate thereof, ascorbic acid, retinol, hyaluronic acid, a collagen polypeptide or a truncate thereof, salicylic acid, benzoyl peroxide, niacinamide, or alpha hydroxy acids (e.g., glycolic acid, lactic acid) alone.

### Example 28. Combination of Collagen and Hyaluronic Acid as an Ingestible Nutraceutical

A powder form of the non-naturally occurring collagen of the disclosure is combined with hyaluronic acid at a ratio of from 1:10, 2:5, 1:1, 5:2, 10:1, 50:1, and 100:1. The ratio and amount of product is adjusted so that the ingestion of the hyaluronic acid component is from about 100 mg to about 1 g per day. Liquid is added, and the composition is formed into a tea, tonic, broth, or other liquid. The composition can also be added to a protein drink, milk, nut milk, fruit shake, or smoothie. The user consumes 1-2 drinks per day. Alternatively, the formulation can be prepared in a pill form, taken daily. Health effects on the user are logged daily for 1 month.

By use of this method, the user observes healthier skin, firmer skin, more hydrated skin, and fewer wrinkles.

### Example 29. Combination of Collagen and Hyaluronic Acid as an Injectable Skin Treatment

An injectable, sterile combination of the non-naturally occurring collagen of the disclosure in combination with hyaluronic acid at a ratio of from 1:10, 2:5, 1:1, 5:2, 10:1, 50:1, and 100:1, in a sterile saline solution. The composition is injected under the skin as a dermatology treatment for certain types of wound scars. The composition is also used for injection into wrinkles or used as a lip plumping agent.

By use of this method, the individual having been treated observes faster healing wound scars, increased plumping of the lips, or fewer wrinkles upon treatment.

It is understood that the examples and embodiments described herein are for illustrative purposes only and that various modifications or changes in light thereof will be suggested to persons skilled in the art and are to be included within the spirit and purview of this application and scope of the appended claims. The disclosed embodiment herein may be embodied in other specific forms without departing from the structures, methods, or other characteristics as broadly described herein and claimed hereinafter. All changes that come within the meaning and range of equivalency of the claims are to be embraced within their scope. All publications, patents, and patent applications cited herein are hereby incorporated by reference in their entirety for all purposes.

1. A composition comprising:
(a) a non-naturally occurring recombinant polypeptide selected from the group consisting of a human collagen or a truncate thereof, a jellyfish collagen or a truncate thereof, and an elastin or truncate thereof; and
(b) one or more active ingredient,
wherein the non-naturally occurring recombinant polypeptide and the one or more active ingredient are present in the composition in amounts effective to restore or improve the appearance of a subject.

2. The composition of claim 1, wherein the one or more active ingredient is selected from the group consisting of a retinoid or vitamin A compound, a vitamin C compound, an alpha hydroxy acid, hyaluronic acid, coenzyme Q10, a tea extract, a grape seed extract, niacinamide, an antibacterial or acne preventative, and any combination of two or more thereof.

3. (canceled)

4. The composition of claim 1, wherein the non-naturally occurring recombinant polypeptide comprises an amino acid sequence truncated at the C-terminal end, the N-terminal end, internally truncated, or truncated at both the C-terminal end and the N-terminal end, relative to a naturally occurring collagen or elastin.

5. (canceled)

6. (canceled)

7. The composition of claim 1, wherein the non-naturally occurring recombinant polypeptide is a human collagen or a jellyfish collagen.

8. The composition of claim 7, wherein the non-naturally occurring recombinant polypeptide is 50 to 900 amino acids in length, 50 to 800 amino acids in length, 50 to 700 amino acids in length, 50 to 600 amino acids in length, 50 to 500 amino acids in length, 50 to 400 amino acids in length, 50 to 300 amino acids in length, 50 to 200 amino acids in length, or 50 to 100 amino acids in length.

9. The composition of claim 7, wherein the non-naturally occurring recombinant polypeptide is or comprises a polypeptide having an N-terminal truncation of 50 to 650 amino acids and a C-terminal truncation of 50 to 250 amino acids relative to a naturally occurring human collagen or a naturally occurring jellyfish collagen.

10. The composition of claim 7, wherein the human collagen is a human type 21 collagen or a human type 1 collagen.

11. The composition of claim 7, wherein the non-naturally occurring recombinant polypeptide comprises or consists of an amino acid sequence having at least 80% sequence identity relative to one or more amino acid sequences selected from the group consisting of: SEQ ID NO: 31, SEQ ID NO: 32, SEQ ID NO: 33, SEQ ID NO: 14, SEQ ID NO: 16, SEQ ID NO: 18, SEQ ID NO: 20, SEQ ID NO: 22, SEQ ID NO: 24, SEQ ID NO: 25, SEQ ID NO: 27, SEQ ID NO: 29, SEQ ID NO: 2, SEQ ID NO: 4, SEQ ID NO: 5, SEQ ID NO: 7, SEQ ID NO: 10, and SEQ ID NO: 12.

12. (canceled)

13. The composition of claim 7, wherein the non-naturally occurring recombinant polypeptide comprises or consists of one or more amino acid sequences selected from the group consisting of: SEQ ID NO: 16, SEQ ID NO: 31, SEQ ID NO: 32, SEQ ID NO: 33, SEQ ID NO: 14, SEQ ID NO: 18, SEQ ID NO: 20, SEQ ID NO: 22, SEQ ID NO: 24, SEQ ID NO: 25, SEQ ID NO: 27, SEQ ID NO: 29, SEQ ID NO: 2, SEQ ID NO: 4, SEQ ID NO: 5, SEQ ID NO: 7, SEQ ID NO: 10, and SEQ ID NO: 12.

14.-23. (canceled)

24. The composition of claim 4, wherein the non-naturally occurring recombinant polypeptide is a human elastin.

25. The composition of claim 24, wherein the non-naturally occurring recombinant polypeptide is 50 to 700 amino acids in length, 50 to 600 amino acids in length, 50 to 500 amino acids in length, 50 to 400 amino acids in length, 50 to 300 amino acids in length, 50 to 200 amino acids in length, or 50 to 100 amino acids in length.

26. (canceled)

27. The composition of claim 24, wherein the non-naturally occurring recombinant polypeptide is a polypeptide having an N-terminal truncation of 50 to 200 amino acids and a C-terminal truncation of 50 to 600 amino acids relative to a naturally occurring elastin polypeptide.

28. The composition of claim 24, wherein the non-naturally occurring recombinant polypeptide comprises or consists of a polypeptide having at least 80% sequence identity relative to one or more amino acid sequences selected from the group consisting of: SEQ ID NO: 38, SEQ ID NO: 40, SEQ ID NO: 41, SEQ ID NO: 43, SEQ ID NO: 46, SEQ ID NO: 48, SEQ ID NO: 50, SEQ ID NO: 52 and SEQ ID NO: 53.

29. (canceled)

30. (canceled)

31. The composition of claim 24, wherein the non-naturally occurring recombinant polypeptide comprises or consists of a polypeptide comprising one or more amino acid sequences selected from the group consisting of: SEQ ID NO: 53, SEQ ID NO: 38, SEQ ID NO: 40, SEQ ID NO: 41, SEQ ID NO: 43, SEQ ID NO: 46, SEQ ID NO: 48, SEQ ID NO: 50, and SEQ ID NO: 52.

32. A formulation comprising the composition of claim 1, and one or more additional ingredients or carriers.

33. (canceled)

34. The formulation of claim 32, wherein the formulation is formulated for application to the skin, to the hair, or to both, of a subject.

35.-37. (canceled)

38. The formulation of claim 32, wherein the non-naturally occurring recombinant polypeptide is present in the formulation in an amount from about 0.001% (w/w) to about 30% (w/w).

39. (canceled)

40. The formulation of claim 32, wherein the one or more active ingredient comprises:
(i) hyaluronic acid in an amount from about 0.001% (w/w) to about 10% (w/w);
(ii) retinol in an amount from about 0.001% (w/w) to about 5% (w/w);
(iii) ascorbic acid in an amount from about 0.01% (w/w) to about 50% (w/w);
(iv) salicylic acid in an amount from about 0.1% (w/w) to about 50% (w/w);
(v) benzoyl peroxide in an amount from about 0.1% (w/w) to about 20% (w/w);
(vi) niacinamide in an amount from about 0.1% (w/w) to about 50% (w/w);
(vii) alpha hydroxy acid in an amount from about 0.1% (w/w) to about 50% (w/w); or
(viii) any combination thereof.

41.-47. (canceled)

48. The formulation of claim 32, wherein the formulation is a personal care product selected from the group consisting of: a mask, a skin cleaner a cleansing cream, a cleansing lotion, a facial cleanser, a cleansing milk, a cleansing pad, a facial wash, a facial cream, a body cream, a facial moisturizer, a body moisturizer, a facial serum, a facial mask, a body mask, a facial toner, a facial mist, an eye cream, an eye treatment, an exfoliator formula, a lip balm, a lipstick, a hair shampoo, a hair conditioner, a body shampoo, a hair serum, a scalp serum, a hair mist, a hair spray, an eye shadow, a concealer, a mascara, and a color cosmetic.

49.-56. (canceled)

57. The formulation of claim 32, wherein the formulation is an injectable formulation.

58. The formulation of claim 57, wherein the injectable formulation is a soft tissue filler, a dermal filler, a subdermal filler, or a lip plumping agent.

59. The formulation of claim 57, wherein the injectable formulation is formulated for injection into wrinkles or for treating wound scars.
